(19) United States
(12) Patent Application Publication
(10) Pub. No.: US 2010/0086921 A1
(43) Pub. Date: Apr. 8, 2010

(54) GENETIC SUSCEPTIBILITY VARIANTS OF TYPE 2 DIABETES MELLITUS

(76) Inventors: Valgerdur Steinthorsdottir, Reykjavik (IS); Gudmar Thorleifsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/442,233

(22) PCT Filed: Nov. 30, 2007
(86) PCT No.: PCT/IS07/00020

§ 371 Date: Aug. 12, 2009

(30) Foreign Application Priority Data
Nov. 30, 2006 (IS) 8572
Apr. 4, 2007 (IS) 8630

(51) Int. Cl. C12Q 1/68; G06F 19/00

(52) U.S. Cl. 435/6; 702/19

(57) ABSTRACT

Association analysis has shown that certain genetic variants are susceptibility variants for Type 2 diabetes. The invention relates to diagnostic applications of such susceptibility variants, including methods of determining increased susceptibility to Type 2 diabetes, as well as methods of determining decreased susceptibility to Type 2 diabetes in an individual. The invention further relates to kits for determining a susceptibility to Type 2 diabetes based on the variants described herein.

### BACKGROUND OF THE INVENTION

Diabetes mellitus, a metabolic disease wherein carbohydrate utilization is reduced and lipid and protein utilization is enhanced, is caused by an absolute or relative deficiency of insulin. In the more severe cases, diabetes is characterized by chronic hyperglycemia, glycosuria, water and electrolyte loss, ketoacidosis and coma. Long term complications include development of neuropathy, retinopathy, nephropathy, generalized degenerative changes in large and small blood vessels and increased susceptibility to infection. The most common form of diabetes is Type II, non-insulin-dependent diabetes that is characterized by hyperglycemia due to impaired insulin secretion and insulin resistance in target tissues. Both genetic and environmental factors contribute to the disease. For example, obesity plays a major role in the development of the disease. Type 2 diabetes is often a mild form of diabetes mellitus of gradual onset.

The health implications of Type 2 diabetes are enormous. In 1995, there were 135 million adults with diabetes worldwide. It is estimated that close to 300 million will have diabetes in the year 2025. (King, H., et al., Diabetes Care, 21(9): 1414-1431 (1998)). The prevalence of Type 2 diabetes in the adult population in Iceland is 2.5% (Vilbergsson, S., et al., Diabet. Med., 14(6): 491-498 (1997)), which comprises approximately 5,000 people over the age of 34 who have the disease.

Type 2 diabetes is characterized by hyperglycemia, which can occur through mechanisms such as impaired insulin secretion, insulin resistance in peripheral tissues and increased glucose output by the liver. Most Type 2 diabetes patients suffer serious complications of chronic hyperglycemia including nephropathy, neuropathy, retinopathy and accelerated development of cardiovascular disease. The prevalence of Type 2 diabetes worldwide is currently 6% but is projected to rise over the next decade (Amos, A. F., McCarty, D. J., Zimmet, P., Diabet Med 14 Suppl 5, S1 (1997)). This increase in prevalence of Type 2 diabetes is attributed to increasing age of the population and rise in obesity.

There is evidence for a genetic component to the risk of Type 2 diabetes, including prevalence differences between various racial groups (Zimmet, P. et al., Am J Epidemiol 118, 673 (1983), Knowler, W. C., Pettitt, D. J., Saad, M. F., Bennett, P. H., Diabetes Metab Rev 6, 1 (1990)), higher concordance rates among monozygotic than dizygotic twins (Newman, B. et al., Diabetologia 30, 763 (1987), Barnett, A. H., Eff, C., Leslie, R. D., Pyke, D. A., Diabetologia 20, 87 (1981)) and a sibling relative risk (λₛ) for Type 2 diabetes in European populations of approximately 3.5 (Gloyn, A. L., Ageing Res Rev 2, 111 (2003)).

Two approaches have thus far been used to search for genes associated with Type 2 diabetes. Single nucleotide polymorphisms (SNPs) within candidate genes have been tested for association and have, in general, not been replicated or confer only a modest risk of Type 2 diabetes—the most widely reported being a protective Pro12Ala polymorphism in the peroxisome proliferator activated receptor gamma gene (PPARG2) (Altshuler, D. et al., Nat Genet 26, 76 (2000)) and an at risk polymorphism in the potassium inwardly-rectifying channel, subfamily 3, member 11 gene (KIR6.2) (Gloyn A. L. et al., Diabetes 52, 568 (2003)).

Genome-wide linkage scans in families with the common form of Type 2 diabetes have yielded several loci, and the primary focus of international research consortia has been on loci on chromosomes 1, 12 and 20 observed in many populations (Gloyn, A. L., Ageing Res Rev 2, 111 (2003)). The genes in these loci have yet to be uncovered. However, in Mexican Americans, the calpain 10 (CAPN10) gene was isolated out of a locus on chromosome 2q (Horikawa, Y. et al., Nat Genet 26, 163 (2000)). The rare Mendelian forms of Type 2 diabetes, namely maturity-onset diabetes of the young (MODY), have yielded six genes by positional cloning (Gloyn, A. L., Ageing Res Rev 2, 111 (2003)).

Genome-wide significant linkage to chromosome 5q for Type 2 diabetes mellitus in the Icelandic population has been reported (Reynisdottir, I. et al., Am J Hum Genet 73, 323 (2003)); in the same study, suggestive evidence of linkage to 10q and 12q was also reported. Linkage to the 10q region has also been observed in Mexican Americans (Duggirala, R. et al., Am J Hum Genet 64, 1127 (1999)).

The transcription factor 7-like 2 gene (TCF7L2; formerly TCF4) has been associated with Type 2 diabetes (P=2.1×10(−9)) (Grant, S. F. et al., Nat Genet 38, 320 (2006)). The original finding in an Icelandic cohort of association of the microsatellite marker DG10S478 within intron 3 of the gene (P=2.1×10(−9)) was replicated in a Danish cohort (P=4.8×10(−3)) and in a US cohort (P=3.3×10(−9)). Compared with non-carriers, heterozygous and homozygous carriers of the at-risk alleles (38% and 7% of the population, respectively) have relative risks of 1.45 and 2.41. This corresponds to a population attributable risk of 21%. %. Association of the TCF7L2 variant has now been replicated in 10 independent studies with similar relative risk found in the different populations studied. The TCF7L2 gene product is a high mobility group box-containing transcription factor previously implicated in blood glucose homeostasis. It is thought to act through regulation of proglucagon gene expression in enteroendocrine cells via the Wnt signaling pathway.

Despite the advances in unraveling the genetics of Type 2 diabetes, the high prevalence of the disease and increasing population affected shows an unmet medical need to define additional genetic factors involved in Type 2 diabetes to more precisely define the associated risk factors. People with impaired fasting glucose or impaired glucose tolerance are asymptomatic but are at a high risk of developing Type 2 diabetes. Currently there is very little information to distinguish those within this high risk group, where lifestyle intervention would be the best choice for disease prevention, from those individuals for whom preventive medication would be more appropriate. Identification of susceptibility genes will allow a better understanding of the pathophysiology of the disease and as a direct benefit for the patient it will facilitate better approaches for diagnosis and treatment. Also needed are therapeutic agents for prevention of Type 2 diabetes.

### SUMMARY OF THE INVENTION

The present invention relates to methods of diagnosing an increased susceptibility to Type 2 diabetes, as well as methods of diagnosing a decreased susceptibility to Type 2 diabetes or diagnosing a protection against Type 2 diabetes, by evaluating certain markers or haplotypes that have been found to be associated with increased or decreased susceptibility of Type 2 diabetes.

In a first aspect, the present invention relates to a method of determining a susceptibility to Type 2 diabetes in a human individual, comprising determining the presence or absence of at least one allele of at least one polymorphic marker in a nucleic acid sample obtained from the individual, wherein the at least one polymorphic marker is selected from the markers set forth in Tables 10-12, and markers in linkage disequilibrium therewith, and wherein determination of the presence or absence of the at least one allele is indicative of a susceptibility to Type 2 diabetes. In one embodiment, the at least one polymorphic marker is selected from the markers set forth in Tables 10-12 and 14. In an alternative aspect the method of determining a susceptibility to Type 2 diabetes is a method of diagnosing a susceptibility to Type 2 diabetes.

In one embodiment, the at least one polymorphic marker is present within SEQ ID NO:1, SEQ ID NO:2 or SEQ ID NO:3. In another embodiment, the at least one polymorphic marker comprises at least one marker selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith. In another embodiment, the at least one polymorphic marker comprises at least one marker in strong linkage disequilibrium, as defined by numeric values for |D′| of greater than 0.8 and/or r² of greater than 0.2, with one or more markers selected from the group consisting of the markers set forth in Table 22, Table 23 and Table 24. In one preferred embodiment, the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), and markers in linkage disequilibrium therewith. In another preferred embodiment, the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), and rs6931514 (SEQ ID NO:37). In one embodiment, the at least one marker is selected from marker rs7756992 (SEQ ID NO: 21), and markers in linkage disequilibrium therewith. In another embodiment, the at least one markers is selected from the markers set forth in Table 22. In another embodiment, the at least one marker is selected from marker rs10882091 (SEQ ID NO: 4), and markers in linkage disequilibrium therewith. In another embodiment, the at least one markers is selected from the markers set forth in Table 23. In yet another embodiment, the at least one marker is selected from marker rs2191113 (SEQ ID NO: 13), and markers in linkage disequilibrium therewith. In another embodiment, the at least one markers is selected from the markers set forth in Table 24.

In one embodiment, the method of determining a susceptibility, or diagnosing a susceptibility, of Type 2 diabetes, further comprises assessing the frequency of at least one haplotype in the individual. In one such embodiment, the at least one haplotype is selected from the haplotypes that comprise at least one polymorphic marker as set forth in Tables 1-6, and polymorphic markers in linkage disequilibrium therewith. In another embodiment, the at least one haplotype is selected from the haplotypes that comprise at least one polymorphic marker selected from at least one marker selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith. In another embodiment, the at least one haplotype is selected from the haplotypes set forth in Tables 1-6 and 14.

In a second aspect, the invention relates to a method of determining a susceptibility to Type 2 diabetes in a human individual, comprising determining whether at least one at-risk allele in at least one polymorphic marker is present in a genotype dataset derived from the individual, wherein the at least one polymorphic marker is selected from the markers set forth in Tables 10-12, and markers in linkage disequilibrium therewith, and wherein determination of the presence of the at least one at-risk allele is indicative of increased susceptibility to Type 2 diabetes in the individual. The genotype dataset comprises in one embodiment information about marker identity, and the allelic status of the individual, i.e. information about the identity of the two alleles carried by the individual for the marker. The genotype dataset may comprise allelic information about one or more marker, including two or more markers, three or more markers, five or more markers, one hundred or more markers, etc. In some embodiments, the genotype dataset comprises genotype information from a whole-genome assessment of the individual including hundreds of thousands of markers, or even one million or more markers.

In one embodiment, the at least one polymorphic marker is present within SEQ ID NO:1, SEQ ID NO:2 or SEQ ID NO:3. In another embodiment, the at least one polymorphic marker comprises at least one marker selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith. In another embodiment, the at least one polymorphic marker comprises at least one marker in strong linkage disequilibrium, as defined by numeric values for |D′| of greater than 0.8 and/or r² of greater than 0.2, with one or more markers selected from the group consisting of the markers set forth in Table 22, Table 23 and Table 24. In one preferred embodiment, the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), and markers in linkage disequilibrium therewith. In another preferred embodiment, the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), and rs6931514 (SEQ ID NO:37). In one embodiment, the at least one marker is selected from marker rs7756992 (SEQ ID NO: 21), and markers in linkage disequilibrium therewith. In another embodiment, the at least one markers is selected from the markers set forth in Table 22. In another embodiment, the at least one marker is selected from marker rs10882091 (SEQ ID NO: 4), and markers in linkage disequilibrium therewith. In another embodiment, the at least one markers is selected from the markers set forth in Table 23. In yet another embodiment, the at least one marker is selected from marker rs2191113 (SEQ ID NO: 13), and markers in linkage disequilibrium therewith. In another embodiment, the at least one markers is selected from the markers set forth in Table 24. In yet another embodiment, the at least one marker is selected from markers in linkage disequilibrium with the SLC30A gene on chromosome 8, between position 118,032,398 and 118,258,134 (NCBI Build 36 of the Human genome assembly). In one such embodiment, the at least one marker is located within the SLC30A gene.

In one embodiment, the method of determining a susceptibility, or diagnosing a susceptibility, of Type 2 diabetes, further comprises assessing the frequency of at least one haplotype in the individual. In one such embodiment, the at least one haplotype is selected from the haplotypes that comprise at least one polymorphic marker as set forth in Tables 1-6, and polymorphic markers in linkage disequilibrium therewith. In another embodiment, the at least one haplotype is selected from the haplotypes that comprise at least one polymorphic marker selected from at least one marker selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith. In another embodiment, the at least one haplotype is selected from the haplotypes set forth in Tables 1-6 and 14.

In certain embodiments of the invention, determination of the presence of at least one at-risk allele of at least one polymorphic marker in a nucleic acid sample from the individual is indicative of an increased susceptibility to Type 2 diabetes. In one embodiment, the increased susceptibility is characterized by a relative risk (RR) or odds ratio (OR) of at least 1.15. In another embodiment, the increased susceptibility is characterized by a relative risk (RR) or odds ratio (OR) of at least 1.20.

In some embodiments, the presence of rs2497304 allele A, rs947591 allele A, rs10882091 allele C rs7914814 allele T, rs6583830 allele A, rs2421943 allele G, rs6583826 allele G, rs7752906 allele A, rs1569699 allele C, rs7756992 allele G, rs9350271 allele A, rs9356744 allele C, rs9368222 allele A, rs10440833 allele A, rs6931514 allele G, rs1860316 allele A, rs1981647 allele C, rs1843622 allele T, rs2191113 allele A, and/or rs9890889 allele A is indicative of increased susceptibility of Type 2 diabetes.

In particular embodiments, the presence of at least one protective allele in a nucleic acid sample from the individual is indicative of a decreased susceptibility of Type 2 diabetes. In another embodiment, the absence of at least one at-risk allele in a nucleic acid sample from the individual is indicative of a decreased susceptibility of Type 2 diabetes.

Particular embodiments of the methods of the invention relate to the at least one marker or haplotype being further associated with insulin response and/or impaired glucose tolerance in an individual.

In other embodiments, the presence of, or the determination of, at least one allele or haplotype in an at-risk marker is indicative of an increased susceptibility to Type 2 diabetes, and wherein the at least one allele or haplotype is further indicative of decreased insulin response and/or impaired glucose tolerance.

In certain embodiments of the invention, linkage disequilibrium is characterized by numeric values for |D′| of greater than 0.8 and/or r² of greater than 0.2. However, other values for the r² and |D′| measures are also possible in other embodiments, and such embodiments are also within the scope of the claimed invention, as described in further detail herein.

Another aspect of the invention relates to a method of assessing a susceptibility to Type 2 diabetes in a human individual, comprising screening a nucleic acid from the individual for at least one polymorphic marker or haplotype in SEQ ID NO:1, SEQ ID NO:2 or SEQ ID NO:3, that correlates with increased occurrence of Type 2 diabetes in a human population, wherein the presence of an at-risk marker allele in the at least one polymorphism or an at-risk haplotype in the nucleic acid identifies the individual as having elevated susceptibility to diabetes, and wherein the absence of the at least one at-risk marker allele or at-risk haplotype in the nucleic acid identifies the individual as not having the elevated susceptibility.

In one embodiment, the polymorphism or haplotype is selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, as characterized by numeric values for |D′| of greater than 0.8 and/or r² of greater than 0.2.

Certain embodiments of the invention further comprise a step of screening the nucleic acid for the presence of at least one at-risk genetic variant for Type 2 diabetes not associated with LD Block C06 (SEQ ID NO:1), LD Block C10 (SEQ ID NO:2) and LD Block C17 (SEQ ID NO:3). Such additional genetic variants can in specific embodiments include any variant that has been identified as a susceptibility or risk variant for Type 2 diabetes, including other variants described herein. In one embodiment, the step comprises screening the nucleic acid for the presence or absence of at least one at-risk allele of at least one at-risk variant for Type 2 diabetes in the TCF7L2 gene, wherein determination of the presence of the at least one at-risk allele is indicative of increased susceptibility of Type 2 diabetes. In another embodiment, the at least one at-risk variant in the TCF7L2 gene is selected from marker DG10S478, rs12255372, rs7895340, rs11196205, rs7901695, rs7903146, rs12243326 and rs4506565, and markers in linkage disequilibrium therewith.

In another aspect of the present invention, the presence of the marker or haplotype found to be associated with Type 2 diabetes, and as such useful for determining a susceptibility to Type 2 diabetes, is indicative of a different response rate of the subject to a particular treatment modality for Type 2 diabetes.

In another aspect, the invention relates to a method of identification of a marker for use in assessing susceptibility to Type 2 diabetes in human individuals, the method comprising:
identifying at least one polymorphic marker within SEQ ID NO:1, SEQ ID NO:2 or SEQ ID NO:3, or at least one polymorphic marker in linkage disequilibrium therewith;
determining the genotype status of a sample of individuals diagnosed with, or having a susceptibility to, Type 2 diabetes; and
determining the genotype status of a sample of control individuals;
   wherein a significant difference in frequency of at least one allele in at least one polymorphism in individuals diagnosed with, or having a susceptibility to, Type 2 diabetes, as compared with the frequency of the at least one allele in the control sample is indicative of the at least one polymorphism being useful for assessing susceptibility to Type 2 diabetes.

In one embodiment, “significant” is determined by statistical means, e.g. the difference is statistically significant. In one such embodiment, statistical significance is characterized by a P-value of less than 0.05. In other embodiments, the statistical significance is characterized a P-value of less than 0.01, less than 0.001, less than 0.0001, less than 0.00001, less than 0.000001, less than 0.0000001, less than 0.0000000001, or less than 0.00000001.

In one embodiment, the at least one polymorphic marker is in linkage disequilibrium, as characterized by numerical values of r² of greater than 0.2 and/or |D′| of greater than 0.8 with at least one marker selected from marker rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31).

In one embodiment, an increase in frequency of the at least one allele in the at least one polymorphism in individuals diagnosed with, or having a susceptibility to, Type 2 diabetes, as compared with the frequency of the at least one allele in the control sample, is indicative of the at least one polymorphism being useful for assessing increased susceptibility to Type 2 diabetes. In another embodiment, a decrease in frequency of the at least one allele in the at least one polymorphism in individuals diagnosed with, or having a susceptibility to, Type 2 diabetes, as compared with the frequency of the at least one allele in the control sample is indicative of the at least one polymorphism being useful for assessing decreased susceptibility to, or protection against, Type 2 diabetes.

Another aspect of the invention relates to a method of genotyping a nucleic acid sample obtained from a human individual, comprising determining the presence or absence of at least one allele of at least one polymorphic marker in the sample, wherein the at least one marker is selected rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, and wherein determination of the presence or absence of the at least one allele of the at least one polymorphic marker is predictive of a susceptibility of Type 2 diabetes.

In one embodiment, genotyping comprises amplifying a segment of a nucleic acid that comprises the at least one polymorphic marker by Polymerase Chain Reaction (PCR), using a nucleotide primer pair flanking the at least one polymorphic marker. In another embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis. In one particular embodiment, the process comprises allele-specific probe hybridization. In another embodiment, the process comprises DNA sequencing. In a preferred embodiment, the method comprises:
1) contacting copies of the nucleic acid with a detection oligonucleotide probe and an enhancer oligonucleotide probe under conditions for specific hybridization of the oligonucleotide probe with the nucleic acid;
   wherein
   a) the detection oligonucleotide probe is from 5-100 nucleotides in length and specifically hybridizes to a first segment of the nucleic acid whose nucleotide sequence is given by SEQ ID NO:1, SEQ ID NO:2 or SEQ ID NO:3 that comprises at least one polymorphic site;
   b) the detection oligonucleotide probe comprises a detectable label at its 3′ terminus and a quenching moiety at its 5′ terminus;
   c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
   d) a single base gap exists between the first segment and the second segment, such that when the oligonucleotide probe and the enhancer oligonucleotide probe are both hybridized to the nucleic acid, a single base gap exists between the oligonucleotides;
2) treating the nucleic acid with an endonuclease that will cleave the detectable label from the 3′ terminus of the detection probe to release free detectable label when the detection probe is hybridized to the nucleic acid; and
3) measuring free detectable label, wherein the presence of the free detectable label indicates that the detection probe specifically hybridizes to the first segment of the nucleic acid, and indicates the sequence of the polymorphic site as the complement of the detection probe.

In a particular embodiment, the copies of the nucleic acid are provided by amplification by Polymerase Chain Reaction (PCR). In another embodiment, the susceptibility determined is increased susceptibility. In another embodiment, the susceptibility determined is decreased susceptibility.

Another aspect of the invention relates to a method of assessing an individual for probability of response to a therapeutic agent for preventing and/or ameliorating symptoms associated with Type 2 diabetes, comprising: determining the presence or absence of at least one allele of at least one polymorphic marker in a nucleic acid sample obtained from the individual, wherein the at least one polymorphic marker is selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele of the at least one marker is indicative of a probability of a positive response to the Type 2 diabetes therapeutic agent. In one embodiment, the Type 2 diabetes therapeutic agent is selected from the agents set forth in Agent Table 1 and Agent Table 2.

Yet another aspect of the invention relates to a method of predicting prognosis of an individual diagnosed with, Type 2 diabetes, the method comprising determining the presence or absence of at least one allele of at least one polymorphic marker in a nucleic acid sample obtained from the individual, wherein the at least one polymorphic marker is selected from the group consisting of rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele is indicative of a worse prognosis of the Type 2 diabetes in the individual.

A further aspect of the invention relates to a method of monitoring progress of a treatment of an individual undergoing treatment for Type 2 diabetes, the method comprising determining the presence or absence of at least one allele of at least one polymorphic marker in a nucleic acid sample obtained from the individual, wherein the at least one polymorphic marker is selected from the group consisting of rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele is indicative of the treatment outcome of the individual.

In one embodiment, the method further comprises assessing at least one biomarker in a sample from the individual. In another embodiment, the method further comprises analyzing non-genetic information to make risk assessment, diagnosis, or prognosis of the individual. The non-genetic information is in one embodiment selected from age, gender, ethnicity, socioeconomic status, previous disease diagnosis, medical history of subject, family history of Type 2 diabetes, biochemical measurements, and clinical measurements. In a particular preferred embodiment, a further step comprising calculating overall risk is employed.

The invention also relates to a kit for assessing susceptibility to Type 2 diabetes in a human individual, the kit comprising reagents for selectively detecting the presence or absence of at least one allele of at least one polymorphic marker in the genome of the individual, wherein the polymorphic marker is selected from the group consisting of polymorphic markers within the nucleic acid segments whose sequences are set forth in SEQ ID NO:1, SEQ ID NO:2 and SEQ ID NO:3, and markers in linkage disequilibrium therewith, and wherein the presence of the at least one allele is indicative of a susceptibility to Type 2 diabetes.

In one embodiment, the at least one polymorphic marker is selected from the group of markers set forth in Tables 10-12, and markers in linkage disequilibrium therewith. In another embodiment, the at least one polymorphic marker is selected from the group of markers set forth in Tables 10-12 and Table 14, and markers in linkage disequilibrium therewith. In another embodiment, the at least one polymorphic markers is selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith. In another embodiment, the at least one polymorphic markers is selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), and rs9890889 (SEQ ID NO:31).

In one embodiment, the reagents comprise at least one contiguous oligonucleotide that hybridizes to a fragment of the genome of the individual comprising the at least one polymorphic marker, a buffer and a detectable label. In one embodiment, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. In a particular embodiment the at least one oligonucleotide is completely complementary to the genome of the individual. In another embodiment, the at least one oligonucleotide can comprise at least one mismatch to the genome of the individual. In one embodiment, the oligonucleotide is about 18 to about 50 nucleotides in length. In another embodiment, the oligonucleotide is 20-30 nucleotides in length.

In one preferred embodiment, the kit comprises:
a detection oligonucleotide probe that is from 5-100 nucleotides in length; an enhancer oligonucleotide probe that is from 5-100 nucleotides in length; and an endonuclease enzyme;
wherein the detection oligonucleotide probe specifically hybridizes to a first segment of the nucleic acid whose nucleotide sequence is given by SEQ ID NO:1, SEQ ID NO:2 or SEQ ID NO:3 that comprises at least one polymorphic site; and wherein the detection oligonucleotide probe comprises a detectable label at its 3′ terminus and a quenching moiety at its 5′ terminus; wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides; and wherein treating the nucleic acid with the endonuclease will cleave the detectable label from the 3′ terminus of the detection probe to release free detectable label when the detection probe is hybridized to the nucleic acid.

A further aspect of the invention relates to the use of an oligonucleotide probe in the manufacture of a diagnostic reagent for diagnosing and/or assessing susceptibility to Type 2 diabetes in a human individual, wherein the probe hybridizes to a segment of a nucleic acid whose nucleotide sequence is given by SEQ ID NO: 1, SEQ ID NO:2 or SEQ ID NO:3 that comprises at least one polymorphic site, wherein the fragment is 15-500 nucleotides in length. In one embodiment, the polymorphic site is selected from the polymorphic markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and polymorphisms in linkage disequilibrium therewith.

Yet another aspect of the invention relates to a computer-readable medium on which is stored: an identifier for at least one polymorphic marker; an indicator of the frequency of at least one allele of said at least one polymorphic marker in a plurality of individuals diagnosed with Type 2 diabetes; and an indicator of the frequency of the least one allele of said at least one polymorphic markers in a plurality of reference individuals; wherein the at least one polymorphic marker is selected from the polymorphic markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and polymorphisms in linkage disequilibrium therewith. In one embodiment, linkage disequilibrium is defined as defined by numerical values of r² of at least 0.2 and/or values of |D′| of at least 0.8.

In one embodiment, information about the ancestry of the plurality of individuals is included. In another embodiment, the plurality of individuals diagnosed with Type 2 diabetes and the plurality of reference individuals is of a specific ancestry.

Another aspect relates to an apparatus for determining a genetic indicator for Type 2 diabetes in a human individual, comprising: a computer readable memory; and a routine stored on the computer readable memory; wherein the routine is adapted to be executed on a processor to analyze marker and/or haplotype information for at least one human individual with respect to at least one polymorphic marker selected from the markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, as defined by numerical values of r² of at least 0.2 and/or values of |D′| of at least 0.8, and generate an output based on the marker or haplotype information, wherein the output comprises a risk measure of the at least one marker or haplotype as a genetic indicator of Type 2 diabetes for the human individual.

In one embodiment, the routine further comprises an indicator of the frequency of at least one allele of at least one polymorphic marker or at least one haplotype in a plurality of individuals diagnosed with Type 2 diabetes, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with Type 2 diabetes.

In certain embodiments of the methods, uses, apparatus or kits of the invention, linkage disequilibrium is characterized by numeric values for |D′| of greater than 0.8 and/or r² of greater than 0.2. However, other values for the r² and |D′| measures are also possible in other embodiments and such embodiments are also within the scope of the claimed invention, as described in further detail herein.

In certain other embodiments of the methods, uses, apparatus or kits of the invention, the individual is of a specific human ancestry. In one embodiment, the ancestry is selected from black African ancestry, Caucasian ancestry and Chinese ancestry. In another embodiment, the ancestry is black African ancestry. In another embodiment, the ancestry is European ancestry. In another embodiment, the ancestry is Caucasian ancestry. The ancestry is in certain embodiment self-reported by the individual who undergoes genetic analysis or genotyping. In other embodiments, the ancestry is determined by genetic determination comprising detecting at least one allele of at least one polymorphic marker in a nucleic acid sample from the individual, wherein the presence or absence of the allele is indicative of the ancestry of the individual.

In particular other embodiments of the methods, uses, apparatus or kits of the invention, the individual is obese. In other embodiments, the individual is non-obese. Obesity is in one embodiment determined by values of BMI (Body Mass Index) of greater than 25. In another embodiment, obesity is defined by values of BMI greater than 30. Other cutoff integer or fractional values of BMI are also possible and within scope of the invention, including, but not limited to BMI of greater than 23, 24, 25.5, 26, 26.5, 27, 27.5 and so on. Non-obese individuals are in one embodiment defined as all those individuals who do not fulfill the criteria of obesity by BMI. In other embodiments, non-obese individuals are those with a particular cutoff of BMI, such as BMI less than 25, less than 24, less than 23, less than 22, less than 21 or less than 20. Non-integer cutoff values of BMI values are also useful for defining non-obese individuals. In general, the obese and non-obese groups do not overlap in terms of their BMI values. In certain embodiments, the cutoff employed to define the groups is the same, e.g., greater than or smaller than BMI of 25. In other embodiments, a different cutoff is used, e.g., greater than 27 for obese individuals and smaller than 23 for non-obese individuals. All relevant ranges of BMI that are suitable for defining obese and non-obese individuals are also possible and within scope of the invention.

### BRIEF DESCRIPTION OF THE DRAWINGS

The foregoing and other objects, features and advantages of the invention will be apparent from the following more particular description of preferred embodiments of the invention.

FIG. 1 shows a plot linkage disequilibrium pattern in the region of chromosome 6p22.3 containing markers associated with Type 2 diabetes. (a) The X-axis shows positions with respect to NCBI Build 35 genome assembly (identical to Build 36), and the Y-axis shows a measure of linkage disequilibrium in the region. The span of the CDKAL1 gene is indicated by the arrows, and the locations of exons by black bars perpendicular to the diagonal line. The SNP markers are plotted equidistantly rather than according to their physical positions. The figure shows the r² measure of linkage disequilibrium, wherein the shading is proportional to pair-wise values of r² between markers. (b) A close-up of the 5′ end of the CDKAL gene, showing the LD Block C06 region (SEQ ID NO:1) within which several markers have been found to be associated with Type 2 diabetes. The location of several of the associated SNP markers is indicated on the figure.

FIG. 2 shows linkage disequilibrium in the region of chromosome 10q23.33 containing markers associated with Type 2 diabetes. The X-axis shows positions with respect to NCBI Build 35 genome assembly, and the Y-axis shows a measure of linkage disequilibrium in the region. The location of four associated SNP markers rs2497304, rs947591, rs10882091 and rs7914814 is indicated as well as the span and exons of the three genes within the LD block, IDE, KIF11 and HHEX. The figure shows the r² measure of linkage disequilibrium, wherein the shading is proportional to pair-wise values of r² between markers.

FIG. 3 shows linkage disequilibrium in the region of chromosome 17q24.3 containing markers associated with diabetes in non-obese and all patients. The location of five SNP markers, rs1860316, rs1981647, rs1843622, rs2191113 and rs9890889, is indicated. The figure shows the r² measure of linkage disequilibrium, wherein the shading is proportional to pair-wise values of r² between markers.

FIG. 4 shows a Q-Q plot of the 653,025 adjusted Chi²-statistics (circles) from the analysis of single SNPs and two marker haplotypes. The equiangular line (black line) is included in the plot for reference purpose. The dashed horizontal line indicates the threshold for genome-wide significance assuming a Bonferroni correction for the 653,025 SNPs/haplotypes and three phenotypes tested.

FIG. 5 presents a schematic view of the association of T2D to 6p22.3. a) The pair-wise correlation structure in a 1 Mb interval (20.5-21.5 Mb, NCBI Build34) on chromosome 6. The upper plot includes pair-wise D′ for 1047 common SNPs (with MAF>5%) from the HapMap release 19 for the CEU population, while the lower plot includes pair-wise r² values for the same set of SNPs. b) Location of recombination hot-spots in this interval based on the HapMap dataset (Nature 437, 1299-1320 (27 Oct. 2005))). c) Location of exons (vertical bars) of the two genes, E2F3 and CDKAL1, that map to the interval. d) Schematic view of the genome-wide association results in the interval for all T2D cases (black dots), non-obese T2D cases (open circles) and obese T2D cases (open triangles), respectively. Plotted is −log P, where P is the adjusted P value, against the chromosomal location of the markers. All four panels use the same horizontal Mb scale indicated at the bottom of panel d).

FIG. 6 shows CDKAL1 cDNA from INS-1 cells. Lanes 1 and 2 contain CDKAL1 cDNA amplified from exons 2 to 8 and exons 7 to 13, giving a band size of 596 bp and 738 bp, respectively. β-actin (837 bp) serves as a positive control in lane 3 and lane 4 is a negative control reaction without primers. Size standard is given on the left.

FIG. 7 shows the association of rs7756992 and rs13266634 to insulin secretion. Mean log-transformed insulin secretion levels, estimated by corrected insulin response (see Methods), for the three different genotypes of the two SNPs, rs7756992 and rs13266634. Results are shown for 3982 individuals (231 T2D cases and 3751 controls) from the Danish Inter99 study that had an oral glucose tolerance test. The number of individuals is included under each column, and the standard error (s.e.m.) is indicated as horizontal bars. The included P values are from regression of the log-transformed insulin secretion levels on genotype status, adjusting for age, sex and affection status, assuming either an additive model (P_{add}) or a recessive model (P_{rec}).

FIG. 8 presents further analysis of association of rs7756992 and rs13266634 with insulin secretion. a) Mean log-transformed insulin secretion levels, estimated by corrected insulin response (CIR) for the three different genotypes for the SNP rs7756992. The insulin secretion levels are estimated for a group of 3938 individuals from the Danish Inter99 cohort (223 T2D cases and 3715 controls) that had an OGTT. Results are shown for all individuals (leftmost bars) and males (middle bars) and females (rightmost bars) separately. The number of individuals behind each estimate is indicated in parenthesis below the columns together with the corresponding genotype. The standard error of the mean is indicated with a bar on top of each column. b) Corresponding estimates for the different genotypes of the SNP rs13266634 for 3926 individuals (228 T2D cases and 3698 controls).

### DETAILED DESCRIPTION OF THE INVENTION

A description of preferred embodiments of the invention follows.

The present invention discloses polymorphic markers and haplotypes that have been found to be associated with Type 2 diabetes. Particular alleles at certain polymorphic SNP markers and haplotypes comprising such alleles have been found to be associated with Type 2 diabetes. Such markers and haplotypes are useful for assessing susceptibility to Type 2 diabetes, as described in further detail herein. Further applications of the present invention include methods for assessing response to Type 2 diabetes therapeutic agents utilizing the polymorphic markers of the invention, as well as kits for assessing susceptibility of an individual to Type 2 diabetes.

### DEFINITIONS

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, microsatellites, insertions, deletions, duplications and translocations.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. Sequence codes for nucleotides used herein are: A=1, C=2, G=3, T=4.

Sequence conucleotide ambiguity as described herein is as proposed by IUPAC-IUB. These codes are compatible with the codes used by the EMBL, GenBank, and PIR databases.

| IUB | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs7758851” refers to the 3 allele of marker rs7758851 being in the haplotype, and is equivalent to “rs7758851 allele 3”. Furthermore, allelic codes in haplotypes are as for individual markers, i.e. 1=A, 2=C, 3=G and 4=T.

The term “susceptibility”, as described herein, encompasses both increased susceptibility and decreased susceptibility. Thus, particular alleles at polymorphic markers and/or haplotypes of the invention may be characteristic of increased susceptibility (i.e., increased risk) of Type 2 diabetes, as characterized by a relative risk (RR) or odds ratio (OR) of greater than one for the particular allele or haplotype. Alternatively, the markers and/or haplotypes of the invention are characteristic of decreased susceptibility (i.e., decreased risk) of Type 2 diabetes, as characterized by a relative risk of less than one.

A “nucleic acid sample” is a sample obtained from an individuals that contains nucleic acid. In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa, placenta, gastrointestinal tract or other organs.

The term “Type 2 diabetes therapeutic agent” refers to an agent that can be used to ameliorate or prevent symptoms associated with Type 2 diabetes.

The term “Type 2 diabetes-associated nucleic acid”, as described herein, refers to a nucleic acid that has been found to be associated to Type 2 diabetes. This includes, but is not limited to, the markers and haplotypes described herein and markers and haplotypes in strong linkage disequilibrium (LD) therewith. In one embodiment, a Type 2 diabetes-associated nucleic acid refers to an LD-block found to be associated with Type 2 diabetes through at least one polymorphic marker located within the LD block.

The term “non-obese” refers, as described herein, to an individual with calculated Body Mass Index (BMI) below a pre-determined threshold, such as a threshold of 30 or lower. Other thresholds useful for defining the term are also possible, as described in more detail herein. The formula for calculating BMI is given by [body weight (in kg)]/[height (in m)]². The term “obese” refers to an individual with BMI above a certain pre-determined threshold, such as a threshold of 30.

The term “LD Block C06”, as described herein, refers to the Linkage Disequilibrium (LD) block on Chromosome 6 between markers rs4429936 and rs6908425, corresponding to position 20,634,996-20,836,710 of NCBI (National Center for Biotechnology Information) Build 35 (SEQ ID NO:1).

The term “LD Block C10”, as described herein, refers to the Linkage Disequilibrium (LD) block on Chromosome 10 between markers rs2798253 and rs11187152, corresponding to position 94,192,885-94,490,091 of NCBI (National Center for Biotechnology Information) Build 35 (SEQ ID NO:2).

The term “LD Block C17”, as described herein, refers to the Linkage Disequilibrium (LD) block on Chromosome 17 between markers rs11077501 and rs4793497, corresponding to position 66,037,656-66,163,076 of NCBI (National Center for Biotechnology Information) Build 35 (SEQ ID NO:3).

The term “CDKAL1”, as described herein, refers to the CDK5 regulatory subunit associated protein 1-like 1 gene, which spans locations 20,642,736-21,340,611 in NCBI Build 35 of the human genome.

The term “SLC30A8”, as described herein, refers to the Solute Carrier Family 30, member 8, gene. This gene is located on chromosome 8, its longest isoform spanning as much as 225 kb between positions 118,032,398 and 118,258,134 in NCBI Build 36 of the human genome assembly, corresponding to position 117,919,805 and 118,145,541, respectively in NCBI Build 34. In both these builds, the gene spans 225,736 by of genomic sequence.

Through genotyping of Icelandic Type 2 diabetes patients and population control individuals using the Illumina 330K chip that can be used to measure over 300,000 SNPs in the genome simultaneously, a number of variants associated with Type 2 diabetes have been identified by the present invention. Association analysis using single SNPs, two marker haplotypes and extended haplotypes within areas of extensive linkage disequilibrium (LD blocks) was performed across the genome. After correcting the p-value for relatedness, 49 single markers and two marker haplotypes were initially identified at 21 loci (i.e. genetic susceptibility locations in the genome) that had a p-value less than 5×10⁻⁵ (Table 1). In addition, 10 extended haplotypes at 8 additional loci were selected by the same criteria (Table 2). Within the patient group, 700 individuals were non-obese (BMI<30) and those were tested separately for association. After correcting the p-value for relatedness, 36 single markers and two marker haplotypes at 20 loci had a p-value less than 5×10⁻⁵ (Table 3). Three of those loci were also identified when the total group was analyzed. In addition, 6 extended haplotypes at 4 additional loci were selected by the same criteria (Table 4). The obese group of 531 patients (BMI>30) was also analyzed separately for association. After correcting the p-value for relatedness, 38 single markers and two marker haplotypes at 16 loci had a p-value less than 5×10⁻⁵ (Table 5). One of those loci was also identified when the total group was analyzed but no overlap was found between the non-obese and obese groups using this criteria. In addition 10 extended haplotypes at 7 additional loci had a p-value less than 5×10⁻⁵ in association analysis of obese diabetics (Table 6).

These single-marker association and two-marker and extended haplotype association results represent evidence for multiple susceptibility variants for Type 2 diabetes. It should be noted that for single-marker SNP analysis as presented herein, susceptibility variants can be represented by increased risk, wherein one allele is overrepresented in the patient group compared with controls. Alternatively, the susceptibility variants can be represented by the other allele of the SNP in question—for that allele, under-representation in patients compared with controls is expected. This is a natural consequence of association analysis to genetic elements comprising two alleles. For multi-marker haplotypes or for polymorphic markers comprising more than one marker, at-risk association may be observed to one (or more) at-risk allele or haplotype. Protective variants in form of association (with RR-values less than unity) to one (or more) protective variants or haplotypes may also be observed, depending on the genetic composition and haplotype structure in the genetic region in question.

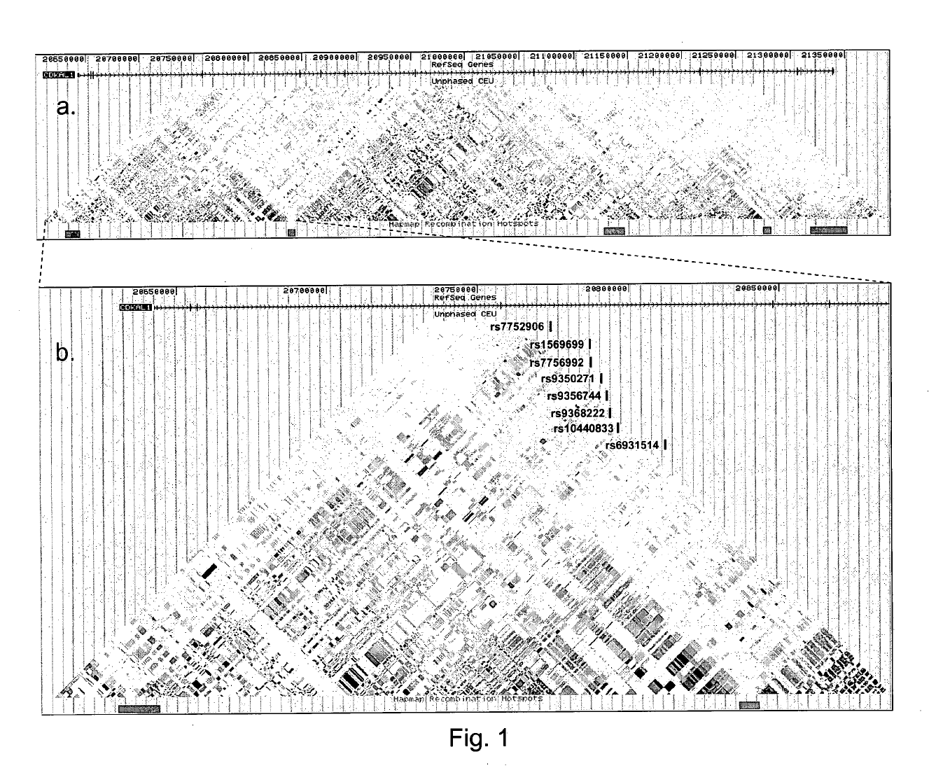

One of the most significant association signals was identified by two single markers (rs1569699 and rs7756992) and three 2 marker haplotypes mapping to chromosome 6p22.3 (3 rs7758851 2 rs1569699, 1 rs4712527 3 rs7756992, 1 rs7756992 3 rs9295478; see Table 3). These markers are located within an area of extensive LD (LD block) between position 20634996 and 20836710 on chromosome 6 (NCBI Build 35; SEQ ID NO:1) between markers rs4429936 and rs6908425 (FIG. 1). This region contains the 5′ end including exons 1-5 of the gene CDK5 regulatory subunit associated protein 1-like 1 (CDKAL1) (NM_{—}017774). The association of these markers was verified in two additional Type 2 diabetes cohorts (see Table 7).

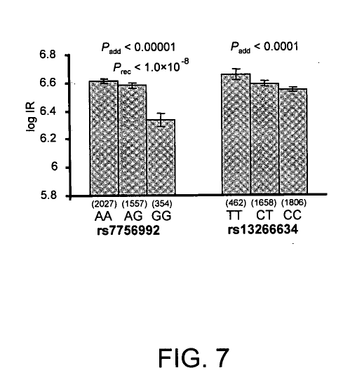

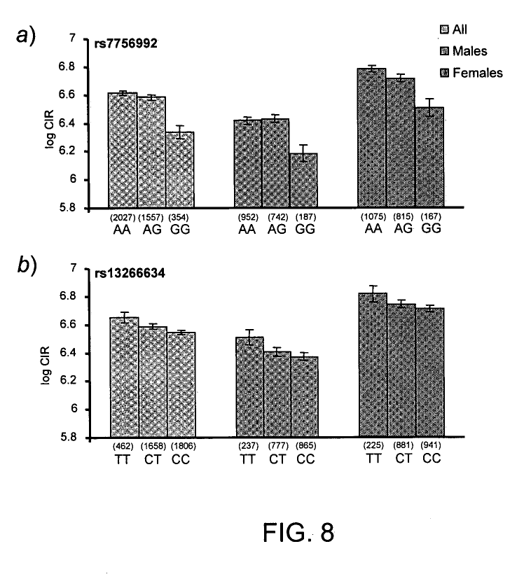

Follow up studies of the association of rs7756992 allele G with increased risk of Type 2 diabetes have established association of the marker to Type 2 diabetes in individuals of European ancestry (allele specific odds ratio (OR)=1.16; P=3.9×10⁻¹⁰), in individuals from Hong Kong of Han Chinese ancestry (OR=1.25; P=0.00018) (see Tables 14, 15 and 17). Additional variants within LD block C06 (SEQ ID NO:1) in LD with rs7756992 that have also been shown to be associated with Type 2 diabetes in European and Chinese populations include rs1569699, rs7752906, rs9350271, rs9356744, rs9368222, rs10440833 and rs6931514 (Table 18). The genotype odds ratio of the rs77566992 allele G variant supports a nearly recessive mode of inheritance (Table 20). In particular, the OR for the homozygote is 1.45 and 1.55 in the European and Hong Kong groups, respectively. The rs77566992 allele G at-risk variant has been found to be correlated with decreased insulin response in carriers (Table 21, FIGS. 7 and 8). Homozygous carriers of the variant have been found to have an estimated 24% less insulin response than heterozygotes or non-carriers suggesting that this variant confers risk of T2D through reduced insulin secretion. The rs7756992 marker, and markers in linkage disequilibrium therewith (including, but not limited to, rs1569699, rs7752906, rs9350271, rs9356744, rs9368222, rs10440833 and rs6931514) can therefore be used to assess increased susceptibility to Type 2 diabetes in an individual.

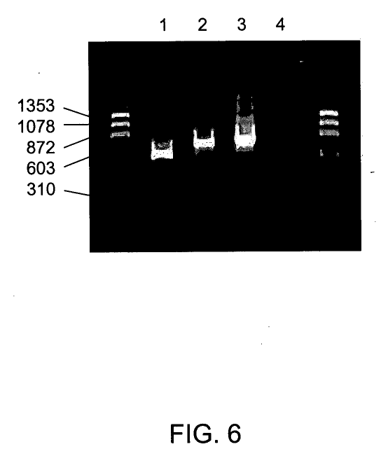

The function of the gene product of CDKAL1 is not known. However, as implied in the gene name the protein product is similar to another protein, CDK5 regulatory subunit associated protein 1 (CDK5RAP1). CDK5RAP1 is expressed in neuronal tissues where it inhibits cyclin dependent kinase 5 (CDK5) activity by binding to the CDK5 regulatory subunit p35 (Ching, Y. P., Pang, A. S., Lam, W. H., Qi, R. Z. & Wang, J. H. J Biol Chem 277, 15237-40 (2002)). In pancreatic beta cells, CDK5 has been shown to play a role in the loss of beta cell function under glucotoxic conditions (Wei, F. Y. et al. Nat Med 11, 1104-8 (2005). Furthermore, inhibition of the CDK5/p35 complex prevents decrease of insulin gene expression that results from glucotoxicity (Ubeda, M., Rukstalis, J. M. & Habener, J. F. J Biol Chem 281, 28858-64 (2006)). CDKAL1 might play a role in the inhibition of CDK5/p35 in pancreatic beta cells similar to that of CDK5RAP1 in neuronal tissue. Reduced expression of CDKAL1 or reduced inhibitory function thus could lead to an impaired response to glucotoxicity. The present data shows that CDKAL1 is expressed in the rat pancreatic beta cell line INS-1 (FIG. 6).

Based on the predicted function of CDKAL1 and known function of SLC30A8 we would expect both rs7756992 and rs13266634 to affect insulin secretion. To evaluate the effects of the two SNPs on insulin secretion we analyzed the effect of genotype status on corrected insulin response (CIR) in a set of individuals from the Inter99 study (part of Denmark B) that had undergone an oral glucose tolerance test (OGTT). For rs7756992, we demonstrated that the homozygote carriers of the risk allele had an estimated 24% less CIR than the heterozygote carriers or non-carriers (P<0.00001, FIG. 7). This observation is consistent with the variant's nearly recessive mode of inheritance with respect to disease risk. Furthermore, the effect observed on CIR is present in both males and females (FIG. 8) and in T2D patients as well as controls, and adjusting for BMI status did not affect the results (Table 21). The effect of rs13266634 on insulin response was smaller but significant and for this risk variant the reduction in CIR was consistent with an additive effect. No effect on insulin sensitivity was observed for either variant (Table 21).

The identification of CDKAL1 as a susceptibility gene for T2D adds a new piece to the puzzle of how genetic factors may predispose to T2D. Although the function of this gene remains to be elucidated we have shown that it is expressed in pancreatic beta cells and that a variant within the gene is correlated with insulin secretion. The similarity to CDK5RAP1 further indicates that CDKAL1 may facilitate insulin production under glucotoxic conditions through interaction with CDK5. In conclusion, we have identified a variant in the CDKAL1 gene that in a nearly recessive manner blunts the insulin response and predisposes to T2D.

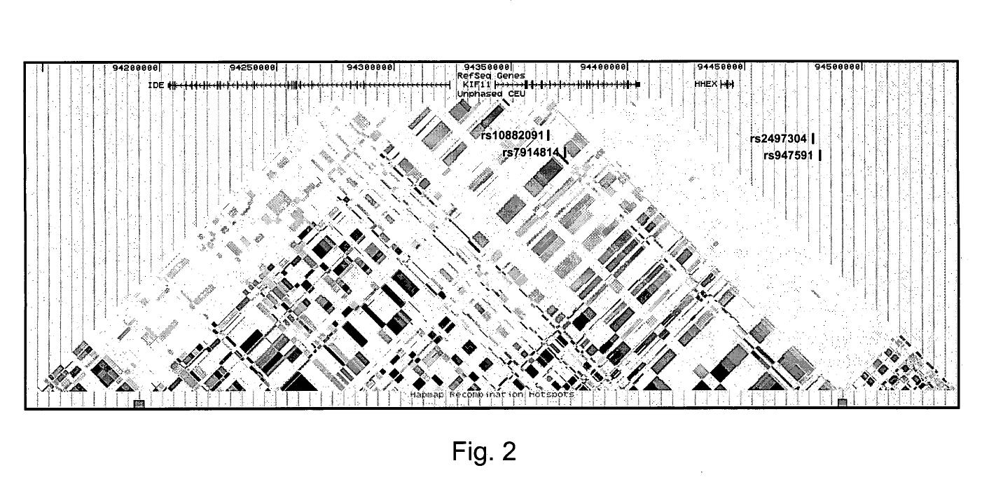

The present invention has identified seven single markers and seven two marker haplotypes in a region on chromosome 10q23.33 to be associated with Type 2 diabetes (Table 1). Most of those markers are also associated to diabetes with elevated RR values when obese patients are analyzed separately (Table 5). These markers are located within one LD block between positions 94192885 and 94490091 (NCBI Build 35), corresponding to the genomic segment bridged by markers rs2798253 and rs11187152 (FIG. 2). This LD block contains three genes, Insulin-degrading enzyme (IDE) (NM_{—}004969), Kinesin family member 11 (KIF11) (NM_{—}004523) and Homeobox, hematopoietically expressed (HHEX) (NM_{—}002729).

IDE may belong to a protease family responsible for intercellular peptide signaling. Though its role in the cellular processing of insulin has not yet been defined, insulin-degrading enzyme is thought to be involved in the termination of the insulin response (Fakhrai-Rad et al, Human Molecular Genetics 9:2149-2158, 2000). Genetic analysis of the diabetic GK rat has revealed 2 amino acid substitutions in the IDE gene (H18R and A890V) in the GK allele which reduced insulin-degrading activity by 31% in transfected cells. However, when the H18R and A890V variants were studied separately, no effects were observed, suggesting a synergistic effect of the 2 variants on insulin degradation. No effect on insulin degradation was observed in cell lysates, suggesting that the effect may be coupled to receptor-mediated internalization of insulin. Congenic rats with the IDE GK allele displayed postprandial hyperglycemia, reduced lipogenesis in fat cells, blunted insulin-stimulated glucose transmembrane uptake, and reduced insulin degradation in isolated muscle. Analysis of additional rat strains demonstrated that the dysfunctional IDE allele was unique to GK rats. The authors concluded that IDE plays an important role in the diabetic phenotype in GK rats. IDE has been studied as a candidate gene for Type 2 diabetes in humans with inconsistent results. Two large studies have recently analyzed the association of IDE to Type 2 diabetes by mutation screening and haplotype analysis using tagging SNPs over the gene (Groves et al, Diabetes 52:1300-1305, 2003; Florez et al, Diabetes 55:128-135, 2006). Both studies conclude that common variants in IDE are unlikely to confer significant risk of Type 2 diabetes. These studies did however, not include the whole LD block as defined in FIG. 2 and at least some of the markers identified in our study as associated with Type 2 diabetes are outside the regions analyzed in those previous studies. Based on the results reported here, markers in LD with IDE are associated with Type 2 diabetes, providing genetic evidence for the role of IDE in the etiology of Type 2 diabetes.

KIF11 encodes a motor protein that belongs to the kinesin-like protein family. Members of this protein family are known to be involved in various kinds of spindle dynamics. The function of this gene product includes chromosome positioning, centrosome separation and establishing a bipolar spindle during cell mitosis. This gene is not a good functional candidate for diabetes but has to be considered as a positional candidate due to its location within the associated LD block.

HHEX encodes a member of the homeobox family of transcription factors, many of which are involved in developmental processes. Expression in specific hematopoietic lineages suggests that this protein may play a role in hematopoietic differentiation. HHEX is essential for pancreatic development; in HHEX negative mouse embryos there is a complete failure in ventral pancreatic specification (Bort et al, Development 131, 797-806, 2004). Other transcription factors involved in pancreatic development include the MODY genes as well as other factors that have been implicated in late onset diabetes. HHEX is also an essential effector of Wnt antagonist for heart induction (Foley and Mercola, GENES & DEVELOPMENT 19:387-396, 2005). This puts HHEX in the same pathway as the recently established Type 2 diabetes gene TCF7L2 and together these data make HHEX a functional as well as positional candidate for Type 2 diabetes.

The association of rs2497304, rs947591, rs10882091 and rs7914814 to Type 2 diabetes was verified in a Danish Type 2 diabetes case—control cohort and also in a US Caucasian cohort Type 2 diabetes cohort from the PENN CATH study (Table 8). When the two cohorts are combined the association of rs947591 reaches significance at the 0.05 level, with a risk of 1.1 in the combined cohort. When all the cohorts are combined the risk is 1.15 for the rs947591 marker. These results indicate that variants within the LD block on Chromosome 10 that includes IDE and HHEX are susceptibility variants for Type 2 diabetes.

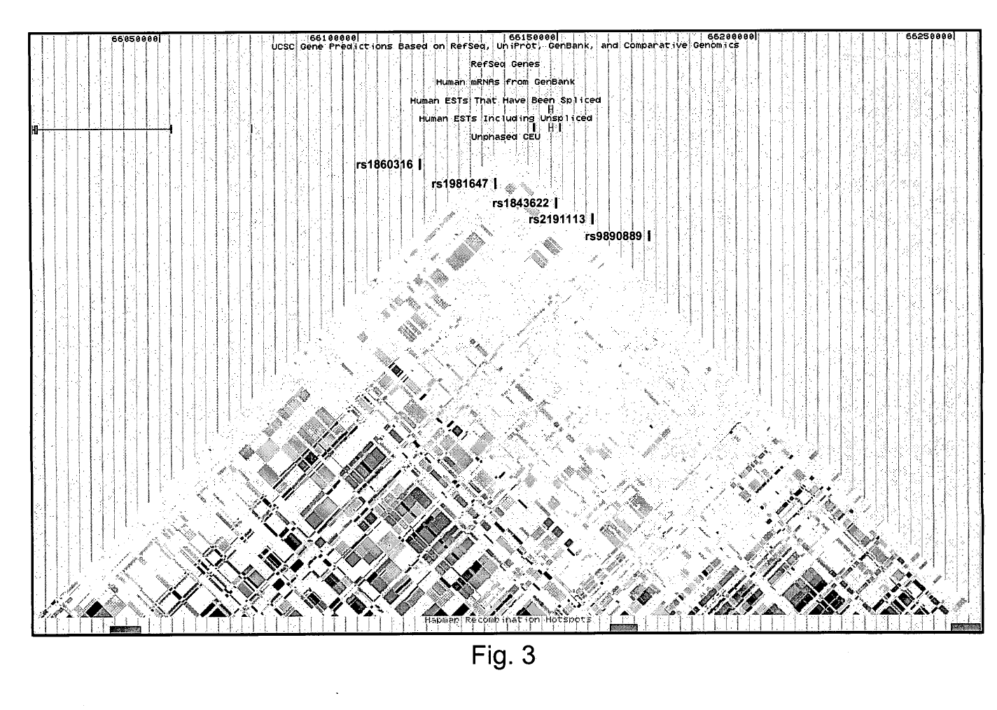

Five single markers and two marker haplotypes in a region of chromosome 17q24.3 were furthermore found to be associated with Type 2 diabetes in non-obese patients (Table 3). Some of these markers show the strongest association reported in Table 3 and association to this region was also observed when all diabetics were analyzed (Table 1). These markers are located within two adjacent LD blocks located between positions 66037656 and 66163076 (NCBI Build 35) on chromosome 17, between markers rs11077501 and rs4793497 (FIG. 3). The association is significant at the genome-wide level. No known genes are located within these LD blocks. However, it is possible that variants in this region affect genes in neighboring regions including KCNJ2 and KCNJ16. Alternatively these variants may affect unknown genes within these LD blocks.

Further evidence for the association of rs7756992, and correlated markers within the LD block C06 that contains the 5′ end including exons 1-5 of the CDKAL1gene (NM_{—}017774) on chromosome 6p22.3, with Type 2 diabetes has come from additional association studies. Two equivalent markers, rs7754840 and rs10946398, highly correlated with rs7756992 (r2 0,68; D′ 0,95) were shown to be significantly associated with Type II diabetes in three large studies (Saxena, R et al. Science 2007; 316:1331-6; Zeggini, E et al. Science 2007; 316:1336-41; Scott, U et al. Science 2007; 316:1341-5). These studies thus further support the involvement of the CDKAL gene in Type 2 diabetes.

Association of rs10882091 and correlated markers on chromosome 10q23.33 with Type II diabetes is also supported by recent publications. A highly correlated marker, rs1111875 (r2 0,51; D′=1) was found to be significantly associated with Type II diabetes in four large studies (Sladek, R et al. Nature. 2007; 445:828-30; Saxena, R et al. Science 2007; 316:1331-6; Zeggini, E et al. Science 2007; 316:1336-41; Scott, U et al. Science 2007; 316:1341-5). Thus, recent studies provide additional support to the discoveries by the present inventors that markers in the LD Block C10 region as described herein are risk factors for Type 2 diabetes.

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNP site; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+ strand or − strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Additional variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMan genotyping assays and SNPlex platforms (Applied Biosystems), mass spectrometry (e.g., MassARRAY system from Sequenom), mini-sequencing methods, real-time PCR, Bio-Plex system (BioRad), CEQ and SNPstream systems (Beckman), Molecular Inversion Probe array technology (e.g., Affymetrix GeneChip), BeadArray Technologies (e.g., Illumina GoldenGate and Infinium assays) and Centaurus assay (Nanogen). By these or other methods available to the person skilled in the art, one or more alleles at polymorphic markers, including microsatellites, SNPs or other types of polymorphic markers, can be identified.

In certain methods described herein, an individual who is at an increased susceptibility (i.e., increased risk) for Type 2 diabetes, is an individual in whom at least one specific allele at one or more polymorphic marker or haplotype conferring increased susceptibility for Type 2 diabetes is identified (i.e., at-risk marker alleles or haplotypes). In one aspect, the at-risk marker or haplotype is one that confers a significant increased risk (or susceptibility) of Type 2 diabetes. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least about 1.5 is significant. In another further embodiment, a significant increase in risk is at least about 1.7 is significant. However, other cutoffs are also contemplated, e.g. at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention.

An at-risk polymorphic marker or haplotype of the present invention is one where at least one allele of at least one marker or haplotype is more frequently present in an individual at risk for the disease or trait (affected), compared to the frequency of its presence in a comparison group (control), and wherein the presence of the marker or haplotype is indicative of susceptibility to the disease or trait. The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes.

In other embodiments of the invention, an individual who is at a decreased susceptibility (i.e., at a decreased risk) for Type 2 diabetes is an individual in whom at least one specific allele at one or more polymorphic marker or haplotype conferring decreased susceptibility for Type 2 diabetes is identified. The marker alleles and/or haplotypes conferring decreased risk are also said to be protective. In one aspect, the protective marker or haplotype is one that confers a significant decreased risk (or susceptibility) of the disease or trait. In another embodiment, the absence of an at-risk allele in a nucleic acid sample from the individual is also indicative of a protection against disease, by virtue of the absence of at-risk alleles. In one embodiment, significant decreased risk is measured as a relative risk of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 ('complete' disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods, kits, procedures, media and apparati described herein, a significant r² value can be at least 0.05, such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also possible, and within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, japanese, chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were identical at the population level, then every single one of them would need to be investigated in association studies. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. Such variants may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

It is possible that certain polymorphic markers in linkage disequilibrium with the markers shown herein to be associated with Type 2 diabetes are located outside the physical boundaries of the LD block as defined. This is a consequence of the historical recombination rates in the region in question, which may have led to a region of strong LD (the LD block), with residual markers outside the block in LD with markers within the block. Such markers are also within scope of the present invention, as they are equally useful for practicing the invention by virtue of their genetic relationship with the markers shown herein to be associated with Type 2 diabetes. Examples are shown in Table 22 (rs17234378; SEQ ID NO:44), Table 23 (rs7086285; SEQ ID NO:43) and Table 24 (rs9890889; SEQ ID NO:31; rs2009802; SEQ ID NO:38; rs17718938; SEQ ID NO:39; rs2109050; SEQ ID NO:41; rs1962801; SEQ ID NO:42.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a region of interest, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease or trait (e.g., Type 2 diabetes), the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Risk Assessment and Diagnostics

As described herein, certain polymorphic markers and haplotypes comprising such markers are found to be useful for risk assessment of Type 2 diabetes. Risk assessment can involve the use of the markers for diagnosing a susceptibility to Type 2 diabetes. Particular alleles of polymorphic markers are found more frequently in individuals with Type 2 diabetes, than in individuals without diagnosis of Type 2 diabetes. Therefore, these marker alleles have predictive value for detecting Type 2 diabetes, or a susceptibility to Type 2 diabetes, in an individual. Tagging markers within haplotype blocks or LD blocks comprising at-risk markers, such as the markers of the present invention, can be used as surrogates for other markers and/or haplotypes within the haplotype block or LD block. Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant.

The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases (e.g., dbSNP), as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to Type 2 diabetes, or a susceptibility to Type 2 diabetes, in an individual.

The markers and haplotypes as described herein, e.g., the markers presented in Tables 1-24, may be useful for risk assessment and diagnostic purposes for, either alone or in combination. The markers and haplotypes can also be combined with other markers conferring increased risk for Type 2 diabetes. Even in cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease. The markers described herein to be associated with Type 2 diabetes can therefore be combined with other polymorphic markers or haplotypes reported or found to be associated with Type 2 diabetes, so as to obtain an overall risk of the disease based on a plurality of genetic markers.

In one such embodiment, the polymorphic markers or haplotypes described herein are assessed together with information about markers within the TCF7L2 gene. Association of variants within this gene is well established (Grant S. F., et al., Nat Genet. 38:320-3 (2006)) and has been replicated in a large number of populations (Florez, J. C., Curr Opin Clin Nutr Metabol Care 10:391-396 (2007). The marker rs7903146 within the TCF7L2 gene, or other markers in LD with the marker (e.g., rs12255372) can be used to determine the genetic risk conferred by the at-risk variant in the gene (OR about 1.44).

Markers in other genes have recently been implicated in the etiology of Type 2 diabetes as risk factors, including PPARG (rs1801282), KCNJ11 (rs5215), TCF2 (rs4430796), WFS1 (rs10010131), CDKN2A-2B (rs1081161), IGF2BP2 (rs4402960) and FTO (rs805136) (Frayling, T. M. Nature Reviews Genetics 8:657-662 (2007). These markers, or markers in linkage disequilibrium therewith can likewise also be used in methods combining determination of the presence or absence of at-risk variants for Type 2 diabetes with the variants reported herein, so as to obtain an overall risk assessment of Type 2 diabetes.

Thus, in one embodiment of the invention, a plurality of variants (genetic markers and/or biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to Type 2 diabetes. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g., markers listed in Tables 22, 23 and 24), but may also include other markers that are in linkage disequilibrium (e.g., characterized by r² greater than 0.2 and/or |D′|>0.8) with one or more of the markers listed in Tables 22, 23 and 24.

For the SNP markers described herein, the opposite allele to the allele found to be in excess in patients (at-risk allele) is found in decreased frequency in Type 2 diabetes. These markers and haplotypes in LD and/or comprising such markers, are thus protective for Type 2 diabetes, i.e. they confer a decreased risk or susceptibility of individuals carrying these markers and/or haplotypes developing Type 2 diabetes. Alternatively speaking, the absence of at-risk alleles of at-risk variants implies the presence of the alternate allele for biallelic markers such as SNPs. Thus, the absence of at-risk variants as described herein is indicative of a protection against Type 2 diabetes.

As described herein, haplotypes comprising a combination of genetic markers, e.g., SNPs and microsatellites, can be useful for risk assessment. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker or haplotype found to be associated with Type 2 diabetes, is one in which a marker or haplotype is more frequently present in an individual at risk for Type 2 diabetes (e.g., an affected person), compared to the frequency of its presence in a healthy individual (control) or in a randomly selected individual from the population (population control), wherein the presence of the marker allele or haplotype is indicative of Type 2 diabetes or a susceptibility to Type 2 diabetes. In other embodiments, at-risk markers in linkage disequilibrium with one or more markers found to be associated with Type 2 diabetes are tagging markers that are more frequently present in an individual at risk for Type 2 diabetes (e.g., affected individuals), compared to the frequency of their presence in controls, wherein the presence of the tagging markers is indicative of increased susceptibility to Type 2 diabetes. In a further embodiment, at-risk markers alleles (i.e. conferring increased susceptibility) in linkage disequilibrium with one or more markers found to be associated with Type 2 diabetes are markers comprising one or more allele that is more frequently present in an individual at risk for Type 2 diabetes, compared to the frequency of their presence in controls, wherein the presence of the markers is indicative of increased susceptibility to Type 2 diabetes.

### Study Population

In a general sense, the methods and kits of the invention can be utilized from samples containing genomic DNA from any source, i.e. any individual. In preferred embodiments, the individual is a human individual. The individual can be an adult, child, or fetus. The present invention also provides for assessing markers and/or haplotypes in individuals who are members of a target population. Such a target population is in one embodiment a population or group of individuals at risk of developing the disease, based on other genetic factors, biomarkers, biophysical parameters (e.g., weight, BMD, blood pressure), or general health and/or lifestyle parameters (e.g., history of disease or related diseases, previous diagnosis of disease, family history of disease).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the disease in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Stacey, S. N., et al., Nat Genet. May 27, 2007 (Epub ahead of print; Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia. The variants described herein to be associated to the CDKAL gene, in particular the LD Block C06 (SEQ ID NO:1) have been replicated in several populations of European, American, and Chinese (Hong Kong) origin. This supports the belief that these variants (rs7756992 and markers in linkage disequilibrium therewith) are at-risk variants for Type 2 diabetes in most populations.

Particular embodiments comprising individual human populations are thus also contemplated and within the scope of the present invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Orogen, She, Tujia, Tu, Xibo, Yi, Mongolan, Naxi, Cambodian, Japanese, Yakut, Melanesian, Papuan, Karitianan, Surui, Columbian, Maya and Pima.

In one preferred embodiment, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry.

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The knowledge about a genetic variant that confers a risk of developing Type 2 diabetes offers the opportunity to apply a genetic test to distinguish between individuals with increased risk of developing the disease (i.e. carriers of the at-risk variant) and those with decreased risk of developing the disease (i.e. carriers of the protective variant). The core values of genetic testing, for individuals belonging to both of the above mentioned groups, are the possibilities of being able to diagnose the disease at an early stage and provide information to the clinician about prognosis/aggressiveness of the disease in order to be able to apply the most appropriate treatment.

For example, the application of a genetic test for Type 2 diabetes can identify high risk individuals among people with impaired fasting glucose (IFG) or impaired glucose tolerance (IGT). It is well established that while around a third of people who are found to have IFG/IGT develop Type 2 diabetes, glucose levels return to normal for an equal proportion of individuals. Identification of individuals within this group that are carriers of genetic risk variants will allow targeting of those individuals by preventive measures. For example, these individuals may benefit from a closer monitoring of blood glucose levels to aid in early diagnosis. They may also need more stringent lifestyle intervention advice since individuals with certain genetic risk factors develop Type 2 diabetes at lower BMI levels than those without those factors.

Individuals with a family history of Type 2 diabetes and carriers of at-risk variants may benefit from genetic testing since the knowledge of the presence of a genetic risk factor, or evidence for increased risk of being a carrier of one or more risk factors, may provide increased incentive for implementing a healthier lifestyle. Furthermore, closer monitoring of glucose levels should be advised for such individuals, facilitating early diagnosis and/or preventative treatment.

Genetic testing of Type 2 diabetes patients may furthermore give valuable information about the primary cause of the disease and can aid the clinician in selecting the best treatment options and medication for each individual. For instance, patients with genetic risk factors for reduced insulin secretion may be likely to benefit from medication increasing insulin secretion while increasing insulin sensitivity in those individuals may be less effective.

### METHODS OF THE INVENTION

Methods for risk assessment of Type 2 diabetes are described herein and are encompassed by the invention. The invention also encompasses methods of assessing an individual for probability of response to a therapeutic agent for Type 2 diabetes, as well as methods for predicting the effectiveness of a therapeutic agent for Type 2 diabetes. Kits for assaying a sample from a subject to detect susceptibility to Type 2 diabetes are also encompassed by the invention.

### DIAGNOSTIC AND SCREENING ASSAYS OF THE INVENTION

In certain embodiments, the present invention pertains to methods of assessing risk or diagnosing, or aiding in risk assessment or diagnosis of, Type 2 diabetes or a susceptibility to Type 2 diabetes, by detecting particular alleles at genetic markers that appear more frequently in Type 2 diabetes subjects or subjects who are susceptible to Type 2 diabetes. In a particular embodiment, the invention is a method of assessing susceptibility to Type 2 diabetes by detecting at least one allele, of at least one polymorphic marker (e.g., the markers described herein). The present invention describes methods whereby detection of particular alleles of particular markers or haplotypes is indicative of a susceptibility to Type 2 diabetes. Such prognostic or predictive assays can also be used to determine prophylactic treatment of a subject prior to the onset of symptoms of Type 2 diabetes.

The present invention pertains in some embodiments to methods of clinical applications of diagnosis, e.g., diagnosis performed by a medical professional, which may include an assessment or determination of genetic risk variants. In other embodiments, the invention pertains to methods of risk assessment (or diagnosis) performed by a layman. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs. The resulting genotype information, made available to the individual can be compared to information from the public literature about disease or trait risk associated with various SNPs. The diagnostic application of disease-associated alleles as described herein, can thus be performed either by a health professional based on results of a clinical test or by a layman, including an individual providing service for performing an whole-genome assessment of SNPs. In other words, the diagnosis or assessment of a susceptibility based on genetic risk can be made by health professionals, genetic counselors, genotype services providers or by the layman, based on information about his/her genotype and publications on various risk factors. In the present context, the term “diagnosing”, and “diagnose a susceptibility”, is meant to refer to any available diagnostic method, including those mentioned above.

In addition, in certain other embodiments, the present invention pertains to methods of diagnosing, or aiding in the diagnosis of, a decreased susceptibility to Type 2 diabetes, by detecting particular genetic marker alleles or haplotypes that appear less frequently in Type 2 diabetes patients than in individual not diagnosed with Type 2 diabetes or in the general population.

As described and exemplified herein, particular marker alleles or haplotypes (e.g. the markers and haplotypes as listed in Tables 1-24, e.g., the markers and haplotypes as listed in Tables 1-6 and Tables 9-12, and markers in linkage disequilibrium therewith) are associated with Type 2 diabetes. In one embodiment, the marker allele or haplotype is one that confers a significant risk or susceptibility to Type 2 diabetes. In another embodiment, the invention relates to a method of diagnosing a susceptibility to Type 2 diabetes in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 9, Table 10, Table 11, and Table 12, and markers in linkage disequilibrium (defined as r²>0.2) therewith. In another embodiment, the invention pertains to methods of diagnosing or assessing a susceptibility to Type 2 diabetes in a human individual, by screening for at least one marker allele or haplotype as listed in Tables 1-6 and 9-12, or markers in linkage disequilibrium therewith. In another embodiment, the marker allele or haplotype is more frequently present in a subject having, or who is susceptible to, Type 2 diabetes (affected), as compared to the frequency of its presence in a healthy subject (control, such as population controls). In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to Type 2 diabetes. These diagnostic methods involve detecting the presence or absence of at least one marker allele or haplotype that is associated with Type 2 diabetes. The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a Type 2 diabetes-associated nucleic acid (e.g., by protein sequencing or by immunoassays using antibodies that recognize such a protein). The marker alleles or haplotypes of the present invention correspond to fragments of a genomic DNA sequence associated with Type 2 diabetes. Such fragments encompass the DNA sequence of the polymorphic marker or haplotype in question, but may also include DNA segments in strong LD (linkage disequilibrium) with the marker or haplotype (e.g., as determined by a value of r² greater than 0.2 and/or |D′|>0.8).

In one embodiment, diagnosis or assessment of a susceptibility to Type 2 diabetes can be accomplished using hybridization methods, such as Southern analysis, Northern analysis, and/or in situ hybridizations (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to Type 2 diabetes, a hybridization sample is formed by contacting the test sample containing an Type 2 diabetes-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of LD Block C06 (SEQ ID NO:1), LD Block C10 (SEQ ID NO:2) (e.g., the nucleotide sequence encoding the IDE, KIF11 and/or the HHEX genes), LD Block C17 (SEQ ID NO:3) or the CDKAL1 gene, or the SLC30A8 gene, as described herein, optionally comprising at least one allele of a marker described herein, or at least one haplotype described herein, or the probe can be the complementary sequence of such a sequence. In a particular embodiment, the nucleic acid probe is a portion of the nucleotide sequence of LD Block C06 (SEQ ID NO:1), LD Block C10 (SEQ ID NO:2) (e.g., the nucleotide sequence encoding the IDE, KIF11 and/or the HHEX genes), LD Block C17 (SEQ ID NO:3) or the CDKAL1 gene, or the SLC30A8 gene as described herein, optionally comprising at least one allele of a marker described herein, or at least one allele contained in the haplotypes described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to DISEASE.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art. In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with Type 2 diabetes. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with Type 2 diabetes. Hybridization of the PNA probe is thus diagnostic for Type 2 diabetes or a susceptibility to Type 2 diabetes.

In one embodiment of the methods of the invention, diagnosis of Type 2 diabetes or a susceptibility to Type 2 diabetes is accomplished through enzymatic amplification of a nucleic acid from the subject. For example, a test sample containing genomic DNA can be obtained from the subject and the polymerase chain reaction (PCR) can be used to amplify a fragment comprising one or more markers or haplotypes of the present invention found to be associated with Type 2 diabetes. As described herein, identification of a particular marker allele or haplotype associated with Type 2 diabetes can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.), to allow the identification of polymorphisms and haplotypes. The technique can assess the presence of an alteration in the expression or composition of a polypeptide or splicing variant(s) that is encoded by a Type 2 diabetes-associated nucleic acid. Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. A test sample containing genomic DNA is obtained from the subject. PCR can be used to amplify particular regions that are associated with Type 2 diabetes (e.g. the polymorphic markers and haplotypes of Tables 1-21, e.g., the polymorphic markers and haplotypes of Tables 1-6 and Tables 9-12, and markers in linkage disequilibrium therewith) nucleic acid in the test sample from the test subject. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with Type 2 diabetes (e.g. the polymorphic markers and haplotypes of Tables 1-24, e.g., the polymorphic markers and haplotypes of Tables 1-6 and Tables 9-12, and markers in linkage disequilibrium therewith, e.g., the markers set forth in Tables 22, 23 and 24). Therefore, in one embodiment, determination of the presence or absence of a particular marker alleles or haplotypes comprises sequence analysis. For example, a test sample of DNA or RNA can be obtained from the test subject. PCR or other appropriate methods can be used to amplify a portion of a Type 2 diabetes-associated nucleic acid, and the presence of a specific allele can then be detected directly by sequencing the polymorphic site (or multiple polymorphic sites) of the genomic DNA in the sample.

Allele-specific oligonucleotides can also be used to detect the presence of a particular allele at a Type 2 diabetes-associated nucleic acid (e.g. the polymorphic markers and haplotypes of Tables 1-21, e.g., the polymorphic markers and haplotypes of Tables 1-6 and Tables 9-12, and markers in linkage disequilibrium therewith), through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a Type 2 diabetes-associated nucleic acid, and which contains a specific allele at a polymorphic site (e.g., a polymorphism described herein). An allele-specific oligonucleotide probe that is specific for one or more particular a Type 2 diabetes-associated nucleic acid can be prepared using standard methods (see, e.g., Current Protocols in Molecular Biology, supra). PCR can be used to amplify the desired region a Type 2 diabetes-associated nucleic acid. The DNA containing the amplified region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with Type 2 diabetes (see, e.g., Gibbs, R. et al., Nucleic Acids Res., 17:2437-2448 (1989) and WO 93/22456).

In another embodiment, arrays of oligonucleotide probes that are complementary to target nucleic acid sequence segments from a subject, can be used to identify polymorphisms in a Type 2 diabetes-associated nucleic acid (e.g. the polymorphic markers and haplotypes of Tables 1-24, e.g. the polymorphic markers and haplotypes of Tables 1-6 and Tables 9-12, and markers in linkage disequilibrium therewith). For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods or light directed synthesis methods that incorporate a combination of photolithographic methods and solid phase oligonucleotide synthesis methods (Fodor, S. et al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Additional descriptions of use of oligonucleotide arrays for detection of polymorphisms can be found, for example, in U.S. Pat. Nos. 5,858,659 and 5,837,832, the entire teachings of both of which are incorporated by reference herein. Other methods of nucleic acid analysis can be used to detect a particular allele at a polymorphic site associated with Type 2 diabetes (e.g. the polymorphic markers and haplotypes of Tables 1-24, e.g. the polymorphic markers and haplotypes of Tables 1-6 and Tables 9-12, and markers in linkage disequilibrium therewith). Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of Type 2 diabetes or a susceptibility to Type 2 diabetes can be made by examining expression and/or composition of a polypeptide encoded by Type 2 diabetes-associated nucleic acid in those instances where the genetic marker(s) or haplotype(s) of the present invention result in a change in the composition or expression of the polypeptide. Thus, diagnosis of a susceptibility to Type 2 diabetes can be made by examining expression and/or composition of one of these polypeptides, or another polypeptide encoded by a Type 2 diabetes-associated nucleic acid, in those instances where the genetic marker or haplotype of the present invention results in a change in the composition or expression of the polypeptide. The haplotypes and markers of the present invention that show association to Type 2 diabetes may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used to make such a detection, including enzyme linked immunosorbent assays (ELISA), Western blots, immunoprecipitation and immunofluorescence. A test sample from a subject is assessed for the presence of an alteration in the expression and/or an alteration in composition of the polypeptide encoded by a Type 2 diabetes-associated nucleic acid. An alteration in expression of a polypeptide encoded by a Type 2 diabetes-associated nucleic acid can be, for example, an alteration in the quantitative polypeptide expression (i.e., the amount of polypeptide produced). An alteration in the composition of a polypeptide encoded by a Type 2 diabetes-associated nucleic acid is an alteration in the qualitative polypeptide expression (e.g., expression of a mutant polypeptide or of a different splicing variant). In one embodiment, diagnosis of a susceptibility to Type 2 diabetes is made by detecting a particular splicing variant encoded by a Type 2 diabetes-associated nucleic acid, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of polypeptide encoded by a Type 2 diabetes-associated nucleic acid in a control sample. A control sample is a sample that corresponds to the test sample (e.g., is from the same type of cells), and is from a subject who is not affected by, and/or who does not have a susceptibility to, Type 2 diabetes (e.g., a subject that does not possess a marker allele or haplotype as described herein). Similarly, the presence of one or more different splicing variants in the test sample, or the presence of significantly different amounts of different splicing variants in the test sample, as compared with the control sample, can be indicative of a susceptibility to Type 2 diabetes. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a Type 2 diabetes-associated nucleic acid can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a Type 2 diabetes-associated nucleic acid can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a Type 2 diabetes-associated nucleic acid in a test sample is compared with the level or amount of the polypeptide encoded by a Type 2 diabetes-associated nucleic acid in a control sample. A level or amount of the polypeptide in the test sample that is higher or lower than the level or amount of the polypeptide in the control sample, such that the difference is statistically significant, is indicative of an alteration in the expression of the polypeptide encoded by the Type 2 diabetes-associated nucleic acid, and is diagnostic for a particular allele or haplotype responsible for causing the difference in expression. Alternatively, the composition of the polypeptide encoded by a Type 2 diabetes-associated nucleic acid in a test sample is compared with the composition of the polypeptide encoded by a Type 2 diabetes-associated nucleic acid in a control sample. In another embodiment, both the level or amount and the composition of the polypeptide can be assessed in the test sample and in the control sample.

In another embodiment, the diagnosis of a susceptibility to Type 2 diabetes is made by detecting at least one Type 2 diabetes-associated marker allele or haplotype (e.g., associated alleles or haplotypes of the markers listed in Tables 1-21, such as Tables 1-6 and Tables 9-12), in combination with an additional protein-based, RNA-based or DNA-based assay. The methods of the invention can also be used in combination with an analysis of a subject's family history and risk factors (e.g., environmental risk factors, lifestyle risk factors).

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with Type 2 diabetes, means for analyzing the nucleic acid sequence of a nucleic acid associated with Type 2 diabetes, means for analyzing the amino acid sequence of a polypeptide encoded by a nucleic acid associated with Type 2 diabetes (e.g., the Type 2 diabetes protein encoded by the Type 2 diabetes gene), etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other Type 2 diabetes diagnostic assays.

In one embodiment, the invention is a kit for assaying a sample from a subject to detect the presence of Type 2 diabetes, symptoms associated with Type 2 diabetes, or a susceptibility to Type 2 diabetes in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from the group consisting of the polymorphisms as listed in Tables 1-6 and 9-12, and polymorphic markers in linkage disequilibrium therewith (e.g., the markers set forth in Tables 22, 23 and 24). In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are indicative of Type 2 diabetes. In another embodiment, the kit comprises one or more labeled nucleic acids capable of allele-specific detection of one or more specific polymorphic markers or haplotypes associated with Type 2 diabetes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers set forth in Tables 9-12. In another embodiment, the marker or haplotype to be detected comprises the markers set forth in Tables 22-24. In another embodiment, the marker or haplotype to be detected comprises markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), and rs9890889 (SEQ ID NO:31), and markers in linkage disequilibrium therewith. In one such embodiment, linkage disequilibrium is defined by values of r² greater than 0.2.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one such embodiments, the presence of the marker or haplotype is indicative of a susceptibility (increased susceptibility or decreased susceptibility) to Type 2 diabetes. In another embodiment, the presence of the marker or haplotype is indicative of response to a Type 2 diabetes therapeutic-agent. In another embodiment, the presence of the marker or haplotype is indicative of prognosis of Type 2 diabetes. In yet another embodiment, the presence of the marker or haplotype is indicative of progress of treatment of Type 2 diabetes. Such treatment may include intervention by surgery, medication or by other means (e.g., lifestyle changes).

### Therapeutic Agents for Type 2 Diabetes

Currently available Type 2 diabetes medication (apart from insulin) falls into six main classes of drugs: sulfonylureas, meglitinides, biguanides, thiazolidinediones, alpha-glucosidase inhibitors and a new class of drugs called DPP-4 inhibitors. These classes of drugs work in different ways to lower blood glucose levels.

1. Sulfonylureas. Sulfonylureas stimulate the beta cells of the pancreas to release more insulin.
2. Meglitinides. Meglitinides are drugs that also stimulate the beta cells to release insulin.
3. Biguanides. Biguanides lower blood glucose levels primarily by decreasing the amount of glucose produced by the liver. Metformin also helps to lower blood glucose levels by making muscle tissue more sensitive to insulin so glucose can be absorbed.
4. Thiazolidinediones. These drugs help insulin work better in the muscle and fat and also reduce glucose production in the liver.
5. Alpha-glucosidase inhibitors. These drugs help the body to lower blood glucose levels by blocking the breakdown of starches, such as bread, potatoes, and pasta in the intestine. They also slow the breakdown of some sugars, such as table sugar. Their action slows the rise in blood glucose levels after a meal. They should be taken with the first bite of a meal.
6. DPP-4 Inhibitors. A new class of medications called DPP-4 inhibitors help improve A1C without causing hypoglycemia. They work by preventing the breakdown of a naturally occurring compound in the body, GLP-1. GLP-1 reduces blood glucose levels in the body, but is broken down very quickly so it does not work well when injected as a drug itself. By interfering in the process that breaks down GLP-1, DPP-4 inhibitors allow it to remain active in the body longer, lowering blood glucose levels only when they are elevated.

Examples of available drugs in these classes are listed in Agent Table 1.

AGENT TABLE 1

| Drug Class | Generic name | Brand name |
|---|---|---|
| Biguanides | metformin | Glucophage, |
| | | Glucophage XR, |
| | | Glycon |
| | metformin plus | Glucovance |
| | glyburide | |
| Thiazolidinediones | pioglitazone | Actos |
| | rosiglitazone | Avandia |
| Sulfonylureas | acetohexamide | Dymelor |
| | chlorpropamide | Diabinese |
| | gliclazide Diamicron | Diamicron MR |
| | glimepiride | Amaryl |
| | glipizide | Glucotrol, Glucotrol XL |
| | glyburide | Micronase, DiaBeta, |
| | | Glynase PresTab |
| | glyburide plus metformin | Glucovance |
| | tolazamide | Tolinase |
| | tolbutamide | Orinase, Tol-Tab |
| Meglitinides | nateglinide | Starlix |
| | repaglinide | Prandin, Gluconorm |
| Alpha-glucosidase | acarbose | Precose, Prandase |
| inhibitors | | |
| | miglitol | Glyset |
| DPP-4 Inhibitors | sitagliptin | Januvia |

Additionally, a combination therapy comprising Biguanide and Sulphonylureas has bee used for treatment of Type 2 diabetes.

Additional Type 2 diabetes drugs are listed Agent Table 2.

AGENT TABLE 2

| | Compound name (generated using | | | |
|---|---|---|---|---|
| Compound | Autonom, ISIS Draw version 2.5 | | Compound | |
| name(s) | from MDL Information Systems) | Company | Reference | Indications |
| AR-0133418 | 1-(4-Methoxy-benzyl)-3-(5- | AstraZeneca | | AD |
| (SN-4521) | nitro-thiazol-2-yl)-urea | | | |
| AR-025028 | NSD | AstraZeneca | | |
| CT-98023 | N-[4-(2,4-Dichloro-phenyl)-5- | Chiron Corp | | non-insulin |
| | (1H-imidazol-2-yl)-pyrimidin- | | | dependent diabetes |
| | 2-yl]-N′-(5-nitro-pyridin-2-yl)- | | | |
| | ethane-1,2-diamine | | | |
| CT-20026 | NSD | Chiron Corp | Wagman et al., | non-insulin |
| | | | Curr Pharm. Des | dependent diabetes |
| | | | 2004: 10(10) | |
| | | | 1105-37 | |
| CT-21022 | NSD | Chiron Corp | | non-insulin |
| | | | | dependent diabetes |
| CT-20014 | NSD | Chiron Corp | | non-insulin |
| | | | | dependent diabetes |
| CT-21018 | NSD | Chiron Corp | | non-insulin |
| | | | | dependent diabetes |
| CHIR-98025 | NSD | Chiron Corp | | non-insulin |
| | | | | dependent diabetes |
| CHIR-99021 | NSD | Chiron Corp | Wagman et al., | non-insulin |
| | | | Curr Pharm. Des | dependent diabetes |
| | | | 2004: 10(10) | |
| | | | 1105-37 | |
| CG-100179 | NSD | CrystalGenomics | WO-2004065370 | diabetes mellitus |
| | | and Yuyu | | (Korea) |
| | 4-[2-(4-Dimethylamino-3- | Cyclacel Ltd. | | non-insulin |
| | nitro-phenylamino)-pyrimidin- | | | dependent diabetes, |
| | 4-yl]-3,5-dimethyl-1H- | | | among others. |
| | pyrrole-2-carbonitrile | | | |
| NP-01139, | 4-Benzyl-2-methyl- | Neuropharma SA | | CNS disorders, AD |
| NP-031112, | [1,2,4]thiadiazolidine-3,5- | | | |
| NP-03112, | dione | | | |
| NP-00361 | | | | |
| | 3-[9-Fluoro-2-(piperidine-1- | Eli Lilly & Co | | non-insulin |
| | carbonyl)-1,2,3,4-tetrahydro- | | | dependent diabetes |
| | [1,4]diazepino[6,7,1-hi]indol- | | | |
| | 7-yl]-4-imidazo[1,2-a]pyridin- | | | |
| | 3-yl-pyrrole-2,5-dione | | | |
| GW-784752x, | Cyclopentanecarboxylic acid | GSK | WO-03024447 | non-insulin |
| GW-784775, | (6-pyridin-3-yl-furo[2,3- | | (compound | dependent diabetes, |
| SB-216763, | d]pyrimidin-4-yl)-amide | | referenced: 4- | neurodegenerative |
| SB-415286 | | | [2-(2- | disease |
| | | | bromophenyl)-4- | |
| | | | (4-fluorophenyl)- | |
| | | | 1H-imidazol-5- | |
| | | | yl]pyridine | |
| NNC-57-0511, | 1-(4-Amino-furazan-3-yl)-5- | Novo Nordisk | | non-insulin |
| NNC-57-0545, | piperidin-1-ylmethyl-1H- | | | dependent diabetes, |
| NNC-57-0588 | [1,2,3]triazole-4-carboxylic | | | |
| | acid[1-pyridin-4-yl-meth-(E)- | | | |
| | ylidene]-hydrazide | | | |
| CP-70949 | NSD | Pfizer | | Hypoglycemic agent |
| VX-608 | NSD | | | Cerebrovascular |
| | | | | ischemia, non-insulin |
| | | | | dependent diabetes |
| KP-403 | NSD | Kinetek | | Nuclear factor kappa |
| class | | | | B modulator, Anti- |
| | | | | inflammatory, Cell |
| | | | | cycle inhibitor, |
| | | | | Glycogen synthase |
| | | | | kinase-3 beta |
| | | | | inhibitor |
| BYETTA | Exenatide: C₁₈₄H₂₈₂N₅₀O₆₀S - | Amylin/Eli Lilly | | non-insulin |
| (exenatide) | Amino acid sequence: H-His- | & Co | | dependent diabetes |
| | Gly-Glu-Gly-Thr-Phe-Thr- | | | |
| | Ser-Asp-Leu-Ser-Lys-Gln- | | | |
| | Met-Glu-Glu-Glu-Ala-Val- | | | |
| | Arg-Leu-Phe-Ile-Glu-Trp- | | | |
| | Leu-Lys-Asn-Gly-Gly-Pro- | | | |
| | Ser-Ser-Gly-Ala-Pro-Pro- | | | |
| | Pro-Ser-NH₂ | | | |
| Vildagliptin | NSD | Novartis | | non-insulin |
| (LAF237) | | | | dependent diabetes - |
| | | | | DPP-4 inhibitor |

### Therapeutic Agents of the Invention

Variants of the present invention (e.g., the markers and/or haplotypes as described herein) can be used to identify novel therapeutic targets for Type 2 diabetes. For example, genes containing, or in linkage disequilibrium with, variants (markers and/or haplotypes) associated with Type 2 diabetes, or their products, as well as genes or their products that are directly or indirectly regulated by or interact with these variant genes or their products, can be targeted for the development of therapeutic agents to treat Type 2 diabetes, or prevent or delay onset of symptoms associated with Type 2 diabetes. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in Antisense Drug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ther. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat a disease or disorder, such as Type 2 diabetes. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in, length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., the markers and haplotypes as described herein) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a disease, including Type 2 diabetes, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat Type 2 diabetes. Thus, the variants of the invention are useful as targets for the identification and/or development of therapeutic agents. Such methods may include assaying the ability of an agent or compound to modulate the activity and/or expression of a nucleic acid that includes at least one of the variants (markers and/or haplotypes) of the present invention, or the encoded product of the nucleic acid. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating Type 2 diabetes can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different, e.g. a different response rate, to a particular treatment modality. This means that a patient diagnosed with Type 2 diabetes, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype at (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

In some embodiments, the treatment modality comprises administering at least one of the therapeutic agents set forth in Agent Table 1 and Agent Table 2. In one embodiment, the therapeutic agent is selected from Biguanides, Thiazolidinediones, Sulfonylureas, Meglitinides, Alpha-glucosidase inhibitors and DPP-4 inhibitors. In one embodiment, the Biguanide is metformin or metformin plus glyburide. Other combination therapies comprising metformin, including combinations with thiazolidinediones, are also contemplated and within the scope of the invention. In another embodiment, the Sulfunylurea is selected from acetohexamide, chlorpropamide, gliclazide Diamicron, glimepiride, glipizide, glyburide, tolazamide and tolbutamide. In another embodiment, the Thiazolidinedione is selected from pioglitazone, rosiglitazone and mitoglitazone or other thiazolidinedione derivatives. In another embodiment, the therapeutic agent is selected from the agents set forth in Agent Table 2.

The present invention also relates to methods of monitoring progress or effectiveness of a treatment for Type 2 diabetes. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for Type 2 diabetes presented herein is determined before and during treatment to monitor its effectiveness. Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

The progress of therapy in individuals carrying at least one at-risk allele of at least one marker found to be associated with increased susceptibility or risk of Type 2 diabetes is thus monitored based on the genotype status of the individual. Individuals carrying at-risk variants as described herein may benefit from closer or more frequent monitoring of progress of therapy than non-carriers, alternatively in combination with a particular treatment modality or therapeutic agent being adminstered, as described in the above.

In a further aspect, the markers of the present invention can be used to increase power and effectiveness of clinical trials. Thus, individuals who are carriers of at least one at-risk variant of the present invention, i.e. individuals who are carriers of at least one allele of at least one polymorphic marker conferring increased risk of developing Type 2 diabetes may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population, e.g., individuals that are either carriers or non-carriers of the at-risk variants described herein. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with Type 2 diabetes when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

In addition to the diagnostic and therapeutic uses of the variants of the present invention, the variants (markers and haplotypes) can also be useful markers for human identification, and as such be useful in forensics, paternity testing and in biometrics. The specific use of SNPs for forensic purposes is reviewed by Gill (Int. J. Legal Med. 114:204-10 (2001)). Genetic variations in genomic DNA between individuals can be used as genetic markers to identify individuals and to associate a biological sample with an individual. Genetic markers, including SNPs and microsatellites, can be useful to distinguish individuals. The more markers that are analyzed, the lower the probability that the allelic combination of the markers in any given individual is the same as in an unrelated individual (assuming that the markers are unrelated, i.e. that the markers are in perfect linkage equilibrium). Thus, the variants used for these purposes are preferably unrelated, i.e. they are inherited independently. Thus, preferred markers can be selected from available markers, such as the markers of the present invention, and the selected markers may comprise markers from different regions in the human genome, including markers on different chromosomes.

In certain applications, the SNPs useful for forensic testing are from degenerate codon positions (i.e., the third position in certain codons such that the variation of the SNP does not affect the amino acid encoded by the codon). In other applications, such for applications for predicting phenotypic characteristics including race, ancestry or physical characteristics, it may be more useful and desirable to utilize SNPs that affect the amino acid sequence of the encoded protein. In other such embodiments, the variant (SNP or other polymorphic marker) affects the expression level of a nearby gene, thus leading to altered protein expression.

The present invention also relates to computer-implemented applications of the polymorphic markers and haplotypes described herein to be associated with Type 2 diabetes. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual), or for deriving information from the genotype data, e.g., by comparing the genotype data to information about genetic risk factors contributing to increased susceptibility to Type 2 diabetes, and reporting results based on such comparison.

One such aspect relates to computer-readable media. In general terms, such medium has capabilities of storing (i) identifier information for at least one polymorphic marker or a haplotype; (ii) an indicator of the frequency of at least one allele of said at least one marker, or the frequency of a haplotype, in individuals with Type 2 diabetes; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

Additional information about the individual can be stored on the medium, such as ancestry information, information about sex, physical attributes or characteristics (including height and weight), biochemical measurements (such as blood pressure, blood lipid levels, fasting glucose levels, insulin response measurements), or other useful information that is desirable to store or manipulate in the context of the genotype status of a particular individual.

The invention furthermore relates to an apparatus that is suitable for determination or manipulation of genetic data useful for determining a susceptibility to Type 2 diabetes in a human individual. Such an apparatus can include a computer-readable memory, a routine for manipulating data stored on the computer-readable memory, and a routine for generating an output that includes a measure of the genetic data. Such measure can include values such as allelic or haplotype frequencies, genotype counts, sex, age, phenotype information, values for odds ratio (OR) or relative risk (RR), population attributable risk (PAR), or other useful information that is either a direct statistic of the original genotype data or based on calculations based on the genetic data.

The above-described applications can all be practiced with the markers and haplotypes of the invention that have in more detail been described with respect to methods of assessing susceptibility to Type 2 diabetes. Thus, these applications can in general be reduced to practice using markers listed in Tables 1-6, and markers in linkage disequilibrium therewith, e.g. the markers set forth in Tables 22, 23 and 24. In one embodiment, the markers or haplotypes are present within the genomic segments whose sequences are set forth in SEQ ID NO:1, SEQ ID NO:2 or SEQ ID NO:3. In another embodiment, the markers and haplotypes comprise at least one marker selected from rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), and rs9890889 (SEQ ID NO:31), optionally including markers in linkage disequilibrium therewith, wherein linkage disequilibrium is defined by numerical values for r² of greater than 0.2. In another embodiment, the marker or haplotype comprises at least one marker selected from rs2497304 allele A, rs947591 allele A, rs10882091 allele C rs7914814 allele T, rs6583830 allele A, rs2421943 allele G, rs6583826 allele G, rs7752906 allele A, rs1569699 allele C, rs7756992 allele G, rs9350271 allele A, rs9356744 allele C, rs9368222 allele A, rs10440833 allele A, rs6931514 allele G, rs1860316 allele A, rs1981647 allele C, rs1843622 allele T, rs2191113 allele A, and rs9890889 allele A. In yet another embodiment, the at least one marker or haplotype comprises at least one marker selected from the markers set forth in Tables 22, 23 and 24.

### Nucleic Acids and Polypeptides

The nucleic acids and polypeptides described herein can be used in methods of diagnosis of a susceptibility to Type 2 diabetes, as well as in kits useful for such diagnosis.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a haplotype described herein). In one embodiment, the invention includes variants that hybridize under high stringency hybridization and wash conditions (e.g., for selective hybridization) to a nucleotide sequence that comprises the nucleotide sequence of LD Block C06 (SEQ ID NO:1), LD Block C10 (SEQ ID NO:2; e.g., the nucleotide sequence encoding the IDE, KIF11 and/or the HHEX genes) and LD Block C17 (SEQ ID NO:3), or the CDKAL1 gene or a fragment thereof (or a nucleotide sequence comprising the complement of the nucleotide sequence of LD Block C06 (SEQ ID NO:1), LD Block C10 (SEQ ID NO:2; e.g., the nucleotide sequence encoding the IDE, KIF11 and/or the HHEX genes) and LD Block C17 (SEQ ID NO:3), or the CDKAL1 gene or a fragment thereof), wherein the nucleotide sequence comprises at least one polymorphic allele contained in the haplotypes (e.g., haplotypes) described herein.

Such nucleic acid molecules can be detected and/or isolated by allele- or sequence-specific hybridization (e.g., under high stringency conditions). Stringency conditions and methods for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998)), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of LD Block C06 (SEQ ID NO:1), LD Block C10 (SEQ ID NO:2; e.g., the nucleotide sequence encoding the IDE, KIF11 and/or the HHEX genes) and LD Block C17 (SEQ ID NO:3), or the CDKAL1 gene or a fragment thereof (or a nucleotide sequence comprising, or consisting of, the complement of the nucleotide sequence of LD Block C06 (SEQ ID NO:1), LD Block C10 (SEQ ID NO:2; e.g., the nucleotide sequence encoding the IDE, KIF11 and/or the HHEX genes) and LD Block C17 (SEQ ID NO:3), or the CDKAL1 gene or a fragment thereof), wherein the nucleotide sequence comprises at least one polymorphic allele contained in the haplotypes (e.g., haplotypes) described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify Type 2 diabetes or a susceptibility to Type 2 diabetes, and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunisation techniques, and/or as an antigen to raise anti-DNA antibodies or elicit immune responses.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. N. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids as described herein, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of Type 2 diabetes, or in an individual with a predisposition to Type 2 diabetes that is related to the function of the protein. Antibodies specific for a variant protein of the present invention that is encoded by a nucleic acid that comprises at least one polymorphic marker or haplotype as described herein can be used to screen for the presence of the variant protein, for example to screen for a predisposition to Type 2 diabetes as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention will now be exemplified by the following non-limiting examples.

### EXEMPLIFICATION

### Example 1

The following contains description of the identification of susceptibility factors found to be associated with Type 2 diabetes through single-point and haplotype analysis of SNP markers.

### Methods

### Icelandic Cohort

The Data Protection Authority of Iceland and the National Bioethics Committee of Iceland approved the study. All participants in the study gave informed consent. All personal identifiers associated with blood samples, medical information and genealogy were first encrypted by the Data Protection Authority, using a third-party encryption system.

For this study, 2400 Type 2 diabetes patients were identified who were diagnosed either through a long-term epidemiologic study done at the Icelandic Heart Association over the past 30 years or at one of two major hospitals in Reykjavik over the past 12 years. Two-thirds of these patients were alive, representing about half of the population of known Type 2 diabetes patients in Iceland today. The majority of these patients were contacted for this study, and the cooperation rate exceeded 80%. All participants in the study visited the Icelandic Heart Association where they answered a questionnaire, had blood drawn and a fasting plasma glucose measurements taken. Questions about medication and age at diagnosis were included. The Type 2 diabetes patients in this study were diagnosed as described in our previously published linkage study (Reynisdottir et al., Am J Hum Genet 73, 323 (2003). In brief, the diagnosis of Type 2 diabetes was confirmed by study physicians through previous medical records, medication history, and/or new laboratory measurements. For previously diagnosed Type 2 diabetes patients, reporting of the use of oral glucose-lowering agent confirmed Type 2 diabetes. Individuals who were currently treated with insulin were classified as having Type 2 diabetes if they were also using or had previously used oral glucose-lowering agents. In this cohort the majority of patients on medication take oral glucose-lowering agents and only a small portion (9%) require insulin. For hitherto undiagnosed individuals, the diagnosis of Type 2 diabetes and impaired fasting glucose (IFG) was based on the criteria set by the American Diabetes Association (Expert Committee on the Diagnosis and Classification of Diabetes Mellitus 1997). The average age of the Type 2 diabetes patients in this study was 69.7 years.

### Replication Cohorts

The Danish study group was a set of Type 2 diabetes patients from the Steno Diabetes Center in Copenhagen (N=1,018) and from the Inter99 population-based sample of 30-60 year old individuals living in the greater Copenhagen area and sampled at Research Centre for Prevention and Health28 (N=359). Diabetes and pre-diabetes categories were diagnosed according to the 1999 World Health Organization (WHO) criteria. An effectively random subset (N=2,400) of Danish controls with BMI measurements were obtained from the Inter99 collection. Informed written consent was obtained from all subjects before participation. The study was approved by the Ethical Committee of Copenhagen County and was in accordance with the principles of the Helsinki Declaration.

The PENN CATH study in the US is a cross sectional study of the association of biochemical and genetic factors with coronary atherosclerosis in a consecutive cohort of patients undergoing cardiac catheterization at the University of Pennsylvania Medical Center between July 1998 and March 2003. Type 2 diabetes was defined as history of fasting blood glucose≧26 mg/dl, 2-hour post-prandial glucose≧200 mg/dl, use of oral hypoglycemic agents, or insulin and oral hypoglycemic in a subject greater than age 40. The University of Pennsylvania Institutional Review Board approved the study protocol and all subjects gave written informed consent. Ethnicity was determined through self-report. A total of 468 Caucasian Type 2 diabetes cases were derived from this cohort. Additionally, 1024 unaffected (with respect to Type 2 diabetes) Caucasian controls were randomly drawn from the same study.

The DNA used for genotyping was the product of whole-genome amplification, by use of the GenomiPhi Amplification kit (Amersham), of DNA isolated from the peripheral blood of the Danish and US Type 2 diabetes patients and controls.

### Genotyping

A genome-wide scan of 1399 Icelandic diabetes patients was performed using Infinium HumanHap300 SNP chips from Illumina for assaying approximately 317,000 single nucleotide polymorphisms (SNPs) on a single chip (Illumina, San Diego, Calif., USA). SNP genotyping for replication in other case-control cohorts was carried using the Centaurus platform (Nanogen).

### Statistical Methods for Association Analysis

For single marker association to Type 2 diabetes, we used a likelihood ratio test to calculate a two-sided p-value for each allele. We calculated relative risk (RR) and population attributable risk (PAR) assuming a multiplicative model (C. T. Falk, P. Rubinstein, Ann Hum Genet 51 (Pt 3), 227 (1987); J. D. Terwilliger, J. Ott, Hum Hered 42, 337 (1992)). For the CEPH Caucasian HapMap data, we calculated LD between pairs of SNPs using the standard definition of D' (R. C. Lewontin, Genetics 50, 757 (1964)) and R² W. G. Hill, A. Robertson, Genetics 60, 615 (November, 1968). When plotting all SNP combinations to elucidate the LD structure in a particular region, we plotted D′ in the upper left corner and p-values in the lower right corner. In the LD plots we present, the markers are plotted equidistantly rather than according to their physical positions.

### Results

### Genome-Wide Association Study

We successfully genotyped 1399 Icelandic Type 2 diabetes patients and 5275 population control individuals using the Illumina 330K chip. Association analysis was performed using single SNPs, two marker haplotypes and extended haplotypes within LD blocks. After correcting the p-value for relatedness we identified 49 single markers and two marker haplotypes at 21 loci (i.e. genetic susceptibility locations in the genome) that had a p-value less than 5×10⁻⁵ (Table 1). In addition, 10 extended haplotypes at 8 additional loci were selected by the same criteria (Table 2). Within the patient group, 700 individuals were non-obese (BMI<30) and those were tested separately for association. After correcting the p-value for relatedness, 36 single markers and two marker haplotypes at 20 loci had a p-value less than 5×10⁻⁵ (Table 3). Three of those loci were also identified when the total group was analysed. In addition 6 extended haplotypes at 4 additional loci were selected by the same criteria (Table 4). The obese group of 531 patients (BMI>30) was also analysed separately for association. After correcting the p-value for relatedness 38 single markers and two marker haplotypes at 16 loci had a p-value less than 5×10⁻⁵ (Table 5). One of those loci was also identified when the total group was analysed but no overlap was found between the non-obese and obese groups using this criteria. In addition 10 extended haplotypes at 7 additional loci had a p-value less than 5×10⁻⁵ in association analysis of obese diabetics (Table 6).

The single-marker association and two-marker and extended haplotype association analysis presented in Tables 1-6 thus represents evidence for multiple susceptibility variants for Type 2 diabetes. It should be noted that for single-marker SNP analysis as presented herein, susceptibility variants can either be represented by increased risk, wherein one allele is overrepresented in the patient group compared with controls. Alternatively, the susceptibility variants can be represented by the other allele of the SNP in question for that allele, under-representation in patients compared with controls is expected. This is a natural consequence of association analysis to genetic elements comprising two alleles. For multi-marker haplotypes or for polymorphic markers comprising more than one marker, at-risk association may be observed to one (or more) at-risk allele or haplotype. Protective variants in form of association (with RR-values less than unity) to one (or more) protective variants or haplotypes may also be observed, depending on the genetic composition and haplotype structure in the genetic region in question.

TABLE 1
Single markers and two marker haplotypes associated with Type 2
Diabetes.

| Chr | Pos | Punadj | Padj | Rrisk | Aff. frq | Ctrl. frq | Haplotype |
|---|---|---|---|---|---|---|---|
| chr1 | 151511890 | 4.01E−06 | 4.49E−05 | 1.223 | 0.407 | 0.360 | 3 rs3738028 |
| chr2 | 40560735 | 2.41E−06 | 3.06E−05 | 1.225 | 0.593 | 0.543 | 1 rs13414307 |
| chr2 | 40560735 | 4.59E−07 | 8.27E−06 | 1.243 | 0.571 | 0.517 | 1 rs13414307 3 rs1990609 |
| chr2 | 54969849 | 5.53E−08 | 1.56E−06 | 1.287 | 0.335 | 0.281 | 3 rs930493 4 rs10173697 |
| chr2 | 54977961 | 3.12E−06 | 3.75E−05 | 1.224 | 0.553 | 0.503 | 4 rs10173697 |
| chr3 | 89323970 | 2.60E−06 | 3.25E−05 | 1.380 | 0.904 | 0.872 | 4 rs12486049 |
| chr6 | 6965113 | 1.00E−06 | 1.53E−05 | 1.705 | 0.072 | 0.044 | 1 rs490213 3 rs814174 |
| chr6 | 31556294 | 3.22E−06 | 3.78E−05 | 1.232 | 0.372 | 0.325 | 2 rs2516424 |
| chr6 | 31556294 | 1.93E−06 | 2.57E−05 | 1.240 | 0.368 | 0.320 | 2 rs2516424 2 rs4947324 |
| chr6 | 132422361 | 3.10E−06 | 3.74E−05 | 1.262 | 0.278 | 0.234 | 3 rs9483377 2 rs997607 |
| chr6 | 132422361 | 3.97E−06 | 4.54E−05 | 1.252 | 0.276 | 0.233 | 3 rs9483377 3 rs7745875 |
| chr6 | 132422361 | 7.98E−07 | 1.25E−05 | 1.249 | 0.356 | 0.307 | 3 rs9483377 |
| chr6 | 150460378 | 5.01E−07 | 8.86E−06 | 1.293 | 0.794 | 0.749 | 1 rs11155700 |
| chr6 | 150461077 | 5.15E−07 | 9.05E−06 | 1.292 | 0.794 | 0.749 | 2 rs12213837 |
| chr6 | 164474219 | 3.07E−06 | 3.63E−05 | 0.813 | 0.479 | 0.531 | 4 rs206732 2 rs933251 |
| chr7 | 87951463 | 4.36E−06 | 4.89E−05 | 1.273 | 0.753 | 0.705 | 1 rs2192319 |
| chr8 | 124196776 | 1.21E−06 | 1.78E−05 | 1.253 | 0.721 | 0.673 | 3 rs952656 |
| chr8 | 124202699 | 5.97E−07 | 9.96E−06 | 0.722 | 0.108 | 0.143 | 4 rs13252935 3 rs7824293 |
| chr9 | 90164936 | 2.03E−06 | 2.62E−05 | 1.304 | 0.192 | 0.154 | 1 rs10993008 |
| chr9 | 95493692 | 2.38E−06 | 3.03E−05 | 1.253 | 0.309 | 0.263 | 3 rs10990568 3 rs4743148 |
| chr9 | 95510129 | 5.85E−07 | 9.80E−06 | 1.252 | 0.365 | 0.315 | 3 rs4743148 |
| chr10 | 53058229 | 1.39E−06 | 1.98E−05 | 1.240 | 0.377 | 0.328 | 4 rs7915186 4 rs3829170 |
| chr10 | 53063104 | 1.37E−06 | 1.96E−05 | 1.239 | 0.386 | 0.336 | 4 rs3829170 3 rs7922112 |
| chr10 | 94301795 | 2.54E−08 | 8.44E−07 | 1.276 | 0.614 | 0.555 | 3 rs2421943 |
| chr10 | 94301795 | 2.11E−09 | 1.19E−07 | 1.297 | 0.585 | 0.521 | 3 rs2421943 2 rs7917359 |
| chr10 | 94304784 | 1.49E−07 | 3.32E−06 | 0.797 | 0.443 | 0.499 | 3 rs7908111 3 rs2497304 |
| chr10 | 94309972 | 6.60E−09 | 2.85E−07 | 0.779 | 0.455 | 0.517 | 3 rs1999763 4 rs10882091 |
| chr10 | 94309972 | 6.60E−09 | 2.85E−07 | 0.779 | 0.455 | 0.517 | 3 rs1999763 3 rs6583830 |
| chr10 | 94337810 | 1.36E−06 | 1.91E−05 | 1.228 | 0.518 | 0.467 | 3 rs6583826 |
| chr10 | 94337810 | 7.18E−08 | 1.91E−06 | 1.262 | 0.449 | 0.393 | 3 rs6583826 2 rs10882091 |
| chr10 | 94364357 | 7.76E−08 | 2.04E−06 | 1.259 | 0.466 | 0.410 | 2 rs10882091 3 rs7923837 |
| chr10 | 94364357 | 9.33E−08 | 2.30E−06 | 1.256 | 0.472 | 0.415 | 2 rs10882091 |
| chr10 | 94372930 | 9.81E−08 | 2.40E−06 | 1.256 | 0.472 | 0.415 | 4 rs7914814 |
| chr10 | 94388098 | 9.33E−08 | 2.30E−06 | 1.256 | 0.472 | 0.415 | 1 rs6583830 |
| chr10 | 94442410 | 8.41E−08 | 2.17E−06 | 1.256 | 0.527 | 0.470 | 1 rs2275729 3 rs1111875 |
| chr10 | 94482696 | 7.56E−08 | 1.95E−06 | 1.258 | 0.542 | 0.485 | 1 rs2497304 |
| chr10 | 94485733 | 1.64E−06 | 2.21E−05 | 1.225 | 0.526 | 0.475 | 1 rs947591 |
| chr12 | 33373479 | 3.87E−06 | 4.37E−05 | 1.391 | 0.110 | 0.082 | 4 rs1905421 |
| chr15 | 98156854 | 3.80E−06 | 4.30E−05 | 0.815 | 0.469 | 0.521 | 1 rs9920347 3 rs11635811 |
| chr16 | 22705353 | 2.93E−06 | 3.57E−05 | 1.264 | 0.781 | 0.738 | 4 rs724466 |
| chr16 | 72066252 | 4.23E−06 | 4.68E−05 | 0.625 | 0.038 | 0.059 | 2 rs1862773 4 rs825842 |
| chr16 | 72086481 | 5.86E−07 | 9.82E−06 | 0.612 | 0.043 | 0.069 | 4 rs2432543 3 rs4887826 |
| chr17 | 66072384 | 7.34E−07 | 1.20E−05 | 1.236 | 0.564 | 0.511 | 3 rs17763769 1 rs1860316 |
| chr17 | 66117911 | 1.18E−07 | 2.77E−06 | 0.781 | 0.282 | 0.335 | 3 rs1860316 2 rs17763811 |
| chr17 | 66117911 | 6.79E−08 | 1.83E−06 | 1.281 | 0.707 | 0.653 | 1 rs1860316 |
| chr17 | 66132788 | 1.80E−06 | 2.43E−05 | 1.226 | 0.563 | 0.513 | 2 rs1981647 |
| chr17 | 66149102 | 1.39E−06 | 1.99E−05 | 1.239 | 0.665 | 0.615 | 4 rs1843622 |
| chr17 | 66159416 | 7.32E−07 | 1.19E−05 | 1.266 | 0.744 | 0.696 | 1 rs2191113 |
| chr20 | 36391335 | 2.09E−07 | 4.45E−06 | 1.250 | 0.550 | 0.495 | 3 rs4592915 2 rs2232580 |

Associating alleles are indicated in front of each marker (Allelic code: A = 1, C = 2, G = 3, T = 4)

TABLE 2
Multi-marker haplotypes associated with Type 2 Diabetes.

| Chr | Pos | Punadj | Padj | Rrisk | Aff. frq | Ctrl. frq | Haplotype* |
|---|---|---|---|---|---|---|---|
| chr2 | 19652497 | 2.00E−08 | 6.98E−07 | 2.492 | 0.027 | 0.011 | 3 rs1593746 3 rs4666491 3 rs12710718 4 rs1579204 |
| | | | | | | | 1 rs824506 2 rs1344652 1 rs4109456 3 rs1427547 |
| | | | | | | | 2 rs1522490 1 rs6757410 4 rs1863776 |
| chr2 | 74747736 | 1.95E−06 | 2.59E−05 | 1.903 | 0.036 | 0.019 | 2 rs363674 2 rs759075 1 rs4853033 1 rs205651 |
| | | | | | | | 4 rs363608 1 rs1063588 2 rs363612 1 rs150139 |
| | | | | | | | 2 rs363617 4 rs1137 4 rs828902 1 rs205627 |
| chr9 | 29300367 | 5.32E−07 | 9.29E−06 | 1.813 | 0.042 | 0.024 | 1 rs4879332 2 rs1928663 4 rs2183357 2 rs10813050 |
| | | | | | | | 2 rs1928661 4 rs10491662 2 rs1169758 2 rs1169757 |
| | | | | | | | 3 rs12378755 |
| chr9 | 32290296 | 4.13E−06 | 4.68E−05 | 1.489 | 0.075 | 0.052 | 3 rs1537156 2 rs7024902 4 rs7037573 4 rs3928808 |
| | | | | | | | 4 rs10970902 3 rs1331226 3 rs10758127 1 rs1331231 |
| | | | | | | | 1 rs992710 2 rs1411866 3 rs10511901 3 rs2094703 |
| | | | | | | | 1 rs7854942 4 rs2150637 |
| chr11 | 22912998 | 7.25E−07 | 1.19E−05 | 1.687 | 0.059 | 0.036 | 3 rs11026796 1 rs1019216 2 rs2302423 4 rs4923035 |
| | | | | | | | 1 rs2429777 4 rs12575930 3 rs887567 2 rs733295 |
| | | | | | | | 3 rs7113718 1 rs7934814 4 rs3909703 4 rs3862134 |
| | | | | | | | 3 rs10833917 1 rs6483890 2 rs2433454 |
| chr13 | 60726830 | 1.52E−06 | 2.12E−05 | 1.481 | 0.108 | 0.075 | 4 rs1411145 4 rs9539100 3 rs991666 3 rs1026924 |
| | | | | | | | 3 rs4886330 3 rs1411568 3 rs1028965 1 rs9670441 |
| chr16 | 72082296 | 1.71E−06 | 2.29E−05 | 0.595 | 0.033 | 0.054 | 4 rs1424011 2 rs1862778 1 rs4888373 4 rs8053178 |
| | | | | | | | 4 rs825842 4 rs2432543 2 rs6564272 3 rs4887826 |
| | | | | | | | 3 rs825851 |
| chr17 | 66118095 | 3.46E−08 | 1.05E−06 | 0.762 | 0.229 | 0.281 | 2 rs16913 2 rs10512540 3 rs17763769 1 rs2109051 |
| | | | | | | | 3 rs1860316 3 rs9904090 4 rs1981647 2 rs1843622 |
| | | | | | | | 2 rs4584866 3 rs17791650 3 rs9891997 3 rs2191113 |
| chr18 | 67477090 | 1.12E−06 | 1.64E−05 | 0.547 | 0.033 | 0.059 | 2 rs9956771 4 rs8088887 2 rs10514019 4 rs719328 |
| | | | | | | | 4 rs1942399 2 rs1942396 4 rs948665 3 rs11151691 |
| chrX | 56884473 | 4.32E−06 | 4.85E−05 | 1.184 | 0.709 | 0.673 | 1 rs12858633 1 rs5960235 3 rs5914036 3 rs6612746 |

*Associating alleles are indicated in front of each marker (Allelic code: A = 1, C = 2, G = 3, T = 4)

TABLE 3
Single markers and two marker haplotypes associated with Type 2
Diabetes in non-obese patients

| Chr | Pos | Punadj | Padj | Rrisk | Aff. frq | Ctrl. frq | Haplotype* |
|---|---|---|---|---|---|---|---|
| chr1 | 29759353 | 5.23E−06 | 3.18E−05 | 0.661 | 0.104 | 0.149 | 4 rs4949283 2 rs502545 |
| chr2 | 53360168 | 8.51E−06 | 4.70E−05 | 1.411 | 0.855 | 0.807 | 1 rs1424963 |
| chr5 | 87772535 | 1.95E−06 | 1.36E−05 | 1.394 | 0.244 | 0.188 | 3 rs10505855 2 rs12514611 |
| chr6 | 6965113 | 5.76E−06 | 3.39E−05 | 1.891 | 0.080 | 0.044 | 1 rs490213 3 rs814174 |
| chr6 | 20650200 | 8.46E−06 | 4.68E−05 | 1.327 | 0.307 | 0.250 | 3 rs7758851 2 rs1569699 |
| chr6 | 20771314 | 1.06E−06 | 8.14E−06 | 1.369 | 0.292 | 0.232 | 1 rs4712527 3 rs7756992 |
| chr6 | 20787289 | 4.47E−06 | 2.79E−05 | 1.333 | 0.315 | 0.256 | 2 rs1569699 |
| chr6 | 20787688 | 1.78E−06 | 1.28E−05 | 0.741 | 0.682 | 0.743 | 1 rs7756992 3 rs9295478 |
| chr6 | 20787688 | 1.11E−06 | 8.61E−06 | 1.368 | 0.292 | 0.232 | 3 rs7756992 |
| chr9 | 95447272 | 6.08E−06 | 3.61E−05 | 0.764 | 0.469 | 0.536 | 2 rs10818991 2 rs10990303 |
| chr11 | 23939149 | 3.05E−06 | 2.02E−05 | 1.525 | 0.128 | 0.088 | 4 rs1879230 |
| chr11 | 130184827 | 9.00E−06 | 4.93E−05 | 1.303 | 0.416 | 0.353 | 4 rs11222327 1 rs1939905 |
| chr13 | 26578564 | 2.15E−06 | 1.51E−05 | 0.723 | 0.220 | 0.281 | 1 rs565707 1 rs6491198 |
| chr13 | 26578564 | 8.29E−07 | 6.63E−06 | 1.381 | 0.763 | 0.700 | 2 rs565707 |
| chr13 | 26635031 | 3.14E−06 | 2.03E−05 | 1.309 | 0.606 | 0.540 | 2 rs7984685 |
| chr13 | 26637643 | 3.37E−06 | 2.15E−05 | 1.308 | 0.606 | 0.540 | 2 rs7998347 |
| chr13 | 26801814 | 9.09E−06 | 4.97E−05 | 1.340 | 0.771 | 0.716 | 1 rs1333350 |
| chr13 | 26801814 | 1.29E−06 | 9.76E−06 | 0.709 | 0.195 | 0.254 | 3 rs1333350 4 rs7987436 |
| chr13 | 108034018 | 9.08E−06 | 4.97E−05 | 1.322 | 0.732 | 0.674 | 2 rs4771591 |
| chr16 | 12697094 | 8.10E−06 | 4.59E−05 | 0.616 | 0.068 | 0.105 | 2 rs6498353 3 rs9941146 |
| chr17 | 66072384 | 2.10E−07 | 2.09E−06 | 1.347 | 0.585 | 0.511 | 3 rs17763769 1 rs1860316 |
| chr17 | 66117911 | 1.01E−09 | 2.42E−08 | 0.677 | 0.254 | 0.335 | 3 rs1860316 2 rs17763811 |
| chr17 | 66117911 | 1.20E−09 | 2.73E−08 | 1.462 | 0.734 | 0.653 | 1 rs1860316 |
| chr17 | 66132788 | 7.18E−07 | 5.88E−06 | 1.329 | 0.583 | 0.513 | 2 rs1981647 |
| chr17 | 66149102 | 4.33E−07 | 3.84E−06 | 1.355 | 0.684 | 0.615 | 4 rs1843622 |
| chr17 | 66159416 | 4.49E−09 | 8.28E−08 | 1.467 | 0.771 | 0.696 | 1 rs2191113 |
| chr17 | 66173475 | 4.75E−06 | 2.88E−05 | 1.472 | 0.885 | 0.839 | 1 rs9890889 |
| chr18 | 41053807 | 4.27E−06 | 2.68E−05 | 1.389 | 0.218 | 0.167 | 3 rs10502860 |
| chr18 | 63441694 | 8.25E−06 | 4.66E−05 | 0.687 | 0.121 | 0.167 | 4 rs764133 4 rs7237209 |
| chr18 | 63465082 | 4.35E−06 | 2.67E−05 | 1.443 | 0.867 | 0.819 | 2 rs7237209 |
| chr19 | 3316583 | 7.55E−06 | 4.33E−05 | 1.370 | 0.227 | 0.176 | 1 rs3810420 |
| chr20 | 36391335 | 8.38E−06 | 4.65E−05 | 1.292 | 0.558 | 0.495 | 3 rs4592915 2 rs2232580 |
| chr21 | 13769165 | 3.83E−06 | 2.40E−05 | 1.599 | 0.927 | 0.888 | 1 rs468601 |
| chr21 | 33298252 | 1.17E−06 | 9.03E−06 | 1.358 | 0.311 | 0.249 | 3 rs2834061 |
| chr21 | 39374906 | 4.04E−06 | 2.51E−05 | 1.308 | 0.631 | 0.566 | 4 rs369906 |
| chr22 | 29580921 | 8.60E−06 | 4.75E−05 | 1.347 | 0.265 | 0.212 | 3 rs8142410 3 rs5994353 |

*Associating alleles are indicated in front of each marker (Allelic code: A = 1, C = 2, G = 3, T = 4)

TABLE 4
Multi-marker haplotypes associated with Type 2 Diabetes in non-obese patients

| Chr | Pos | Punadj | Padj | Rrisk | Aff. frq | Ctrl. frq | Haplotype* |
|---|---|---|---|---|---|---|---|
| chr2 | 19652497 | 3.14E−07 | 2.93E−06 | 2.859 | 0.031 | 0.011 | 3 rs1593746 3 rs4666491 3 rs12710718 4 rs1579204 |
| | | | | | | | 1 rs824506 2 rs1344652 1 rs4109456 3 rs1427547 |
| | | | | | | | 2 rs1522490 1 rs6757410 4 rs1863776 |
| chr5 | 2458281 | 6.12E−06 | 3.62E−05 | 0.077 | 0.001 | 0.017 | 3 rs931283 1 rs160730 3 rs468085 4 rs464716 |
| | | | | | | | 3 rs10052956 2 rs160729 3 rs315914 1 rs1039096 |
| chr6 | 137323498 | 6.46E−06 | 3.73E−05 | 2.566 | 0.040 | 0.016 | 2 rs6570118 4 rs7743308 3 rs6928748 2 rs12214917 |
| | | | | | | | 2 rs6936698 2 rs4896224 2 rs10872468 |
| chr11 | 32116221 | 4.15E−06 | 2.57E−05 | 1.362 | 0.266 | 0.211 | 1 rs224633 3 rs581573 4 rs223070 4 rs10488686 |
| | | | | | | | 4 rs4922579 2 rs110688 4 rs1605271 3 rs4922901 |
| | | | | | | | 3 rs7950374 1 rs1033584 1 rs12788147 3 rs11031625 |
| | | | | | | | 2 rs880587 4 rs989570 2 rs10835861 |
| chr17 | 66118095 | 7.82E−10 | 1.95E−08 | 0.660 | 0.205 | 0.281 | 2 rs16913 2 rs10512540 3 rs17763769 1 rs2109051 |
| | | | | | | | 3 rs1860316 3 rs9904090 4 rs1981647 2 rs1843622 |
| | | | | | | | 2 rs4584866 3 rs17791650 3 rs9891997 3 rs2191113 |
| chr17 | 66204022 | 6.39E−06 | 3.76E−05 | 0.683 | 0.115 | 0.160 | 2 rs9890889 4 rs2367005 2 rs2109054 3 rs17792120 |
| | | | | | | | 1 rs7221340 4 rs1486293 2 rs1486296 2 rs17763811 |
| | | | | | | | 4 rs9807096 3 rs10512541 3 rs8065001 2 rs4793501 |
| | | | | | | | 3 rs929474 3 rs9907514 |

*Associating alleles are indicated in front of each marker (Allelic code: A = 1, C = 2, G = 3, T = 4)

TABLE 5
Single markers and two marker haplotypes associated with Type 2
Diabetes in obese patients

| Chr | Pos | Punadj | Padj | Rrisk | Aff. frq | Ctrl. frq | Haplotype* |
|---|---|---|---|---|---|---|---|
| chr1 | 104818519 | 5.60E−06 | 2.85E−05 | 1.343 | 0.466 | 0.394 | 2 rs7553985 |
| chr1 | 104824377 | 4.76E−06 | 2.48E−05 | 1.346 | 0.466 | 0.393 | 4 rs2166890 |
| chr1 | 104825870 | 6.28E−06 | 3.14E−05 | 1.355 | 0.396 | 0.317 | 4 rs7552405 |
| chr3 | 147025256 | 7.11E−06 | 3.49E−05 | 1.696 | 0.097 | 0.059 | 3 rs7630694 |
| chr3 | 197065940 | 2.81E−06 | 1.58E−05 | 1.396 | 0.737 | 0.668 | 1 rs9858622 |
| chr4 | 140287637 | 4.41E−06 | 2.32E−05 | 1.431 | 0.804 | 0.741 | 1 rs13116075 1 rs6824182 |
| chr4 | 140364285 | 1.05E−05 | 4.86E−05 | 0.708 | 0.194 | 0.254 | 4 rs2292837 2 rs11725721 |
| chr4 | 140397800 | 8.21E−06 | 3.95E−05 | 0.704 | 0.194 | 0.254 | 3 rs3762864 2 rs11725721 |
| chr5 | 76586085 | 9.46E−06 | 4.46E−05 | 0.750 | 0.438 | 0.510 | 1 rs832785 1 rs2859576 |
| chr5 | 76586766 | 8.97E−06 | 4.26E−05 | 1.333 | 0.562 | 0.491 | 4 rs4704400 |
| chr6 | 9509965 | 7.50E−06 | 3.66E−05 | 1.335 | 0.495 | 0.424 | 4 rs214447 |
| chr6 | 22837279 | 1.03E−05 | 4.80E−05 | 1.430 | 0.824 | 0.766 | 2 rs10498713 3 rs4426986 |
| chr6 | 41191330 | 3.22E−06 | 1.77E−05 | 1.360 | 0.637 | 0.563 | 1 rs10456499 |
| chr8 | 128358773 | 4.94E−06 | 2.56E−05 | 0.692 | 0.190 | 0.254 | 2 rs283710 2 rs412835 |
| chr8 | 128362648 | 6.35E−07 | 4.42E−06 | 1.495 | 0.822 | 0.755 | 3 rs185852 |
| chr8 | 128376264 | 1.57E−06 | 9.59E−06 | 0.680 | 0.189 | 0.255 | 2 rs283718 1 rs283720 |
| chr9 | 126494483 | 2.67E−06 | 1.51E−05 | 1.591 | 0.139 | 0.092 | 4 rs3814120 |
| chr10 | 94301795 | 5.53E−07 | 3.93E−06 | 1.393 | 0.602 | 0.521 | 3 rs2421943 2 rs7917359 |
| chr10 | 94304784 | 8.39E−06 | 4.02E−05 | 0.747 | 0.427 | 0.499 | 3 rs7908111 3 rs2497304 |
| chr10 | 94309972 | 3.74E−06 | 2.01E−05 | 0.739 | 0.442 | 0.518 | 3 rs1999763 4 rs10882091 |
| chr10 | 94309972 | 3.74E−06 | 2.01E−05 | 0.739 | 0.442 | 0.518 | 3 rs1999763 3 rs6583830 |
| chr10 | 94337810 | 1.89E−06 | 1.12E−05 | 1.364 | 0.469 | 0.393 | 3 rs6583826 2 rs10882091 |
| chr10 | 94364357 | 1.76E−06 | 1.05E−05 | 1.363 | 0.486 | 0.410 | 2 rs10882091 3 rs7923837 |
| chr10 | 94364357 | 2.58E−06 | 1.47E−05 | 1.355 | 0.491 | 0.415 | 2 rs10882091 |
| chr10 | 94372930 | 2.66E−06 | 1.51E−05 | 1.355 | 0.491 | 0.416 | 4 rs7914814 |
| chr10 | 94388098 | 2.58E−06 | 1.47E−05 | 1.355 | 0.491 | 0.415 | 1 rs6583830 |
| chr10 | 94482696 | 1.62E−06 | 9.85E−06 | 1.363 | 0.562 | 0.485 | 1 rs2497304 |
| chr10 | 118562511 | 8.21E−06 | 3.95E−05 | 1.384 | 0.302 | 0.238 | 4 rs1681748 4 rs2170862 |
| chr10 | 118610986 | 9.43E−06 | 4.45E−05 | 1.367 | 0.320 | 0.256 | 4 rs2170862 |
| chr10 | 118880683 | 3.29E−06 | 1.80E−05 | 1.379 | 0.347 | 0.278 | 3 rs10787760 |
| chr11 | 106441899 | 8.79E−06 | 4.18E−05 | 1.533 | 0.142 | 0.097 | 4 rs1455593 |
| chr12 | 30340321 | 4.54E−06 | 2.38E−05 | 0.723 | 0.296 | 0.368 | 1 rs1429622 3 rs1506382 |
| chr14 | 81787150 | 3.94E−06 | 2.10E−05 | 1.363 | 0.439 | 0.365 | 1 rs799099 3 rs4899801 |
| chr14 | 81843593 | 8.25E−06 | 3.97E−05 | 1.339 | 0.437 | 0.367 | 3 rs2066041 |
| chr14 | 81899972 | 9.32E−06 | 4.40E−05 | 1.331 | 0.530 | 0.459 | 1 rs10483957 |
| chr14 | 87823315 | 9.69E−07 | 6.35E−06 | 1.605 | 0.891 | 0.836 | 3 rs419028 |
| chr16 | 24287484 | 6.15E−06 | 3.08E−05 | 1.388 | 0.372 | 0.300 | 1 rs11074618 2 rs985729 |
| chr19 | 3065864 | 1.02E−05 | 4.77E−05 | 1.433 | 0.825 | 0.767 | 3 rs3746069 |

*Associating alleles are indicated in front of each marker (Allelic code: A = 1, C = 2, G = 3, T = 4)

TABLE 6
Multi-marker haplotypes associated with Type 2 Diabetes in obese patients

| Chr | Pos | Punadj | Padj | Rrisk | Aff. frq | Ctrl. frq | Haplotype* |
|---|---|---|---|---|---|---|---|
| chr2 | 2591675 | 4.35E−06 | 2.29E−05 | 0.654 | 0.126 | 0.181 | 4 rs7576292 4 rs6548079 4 rs1451199 1 rs2385306 |
| | | | | | | | 2 rs1020530 1 rs12714359 2 rs7556672 3 rs1451198 |
| chr4 | 112032007 | 7.13E−06 | 3.50E−05 | 1.699 | 0.097 | 0.060 | 2 rs16997168 4 rs2723316 1 rs6419178 3 rs1448817 |
| | | | | | | | 3 rs2634073 2 rs2200733 2 rs2220427 2 rs13105878 |
| | | | | | | | 3 rs10033464 |
| chr8 | 128361033 | 7.34E−07 | 5.01E−06 | 0.671 | 0.178 | 0.244 | 3 rs283709 2 rs283710 2 rs4871780 1 rs185852 |
| | | | | | | | 2 rs412835 |
| chr10 | 68829632 | 4.50E−06 | 2.36E−05 | 2.428 | 0.039 | 0.017 | 4 rs7094426 1 rs1904614 3 rs10823028 3 rs2620924 |
| | | | | | | | 1 rs12359451 2 rs11815372 3 rs7083570 3 rs2394375 |
| | | | | | | | 2 rs1875151 4 rs10823057 4 rs6480272 3 rs1911356 |
| chr11 | 106076550 | 9.88E−06 | 4.63E−05 | 0.655 | 0.114 | 0.164 | 3 rs1791587 3 rs1793083 2 rs1791597 4 rs7104111 |
| | | | | | | | 2 rs1793064 1 rs4523664 2 rs623018 4 rs631214 |
| | | | | | | | 3 rs602159 2 rs10890568 2 rs4553343 4 rs1487906 |
| | | | | | | | 3 rs4121676 1 rs4121677 4 rs6588924 |
| chr13 | 94045239 | 4.93E−06 | 2.55E−05 | 0.058 | 0.001 | 0.012 | 1 rs726298 2 rs7339106 1 rs9556403 2 rs9590039 |
| | | | | | | | 2 rs6492722 1 rs1572935 3 rs6492725 |
| chr14 | 81810554 | 9.82E−07 | 6.42E−06 | 1.408 | 0.341 | 0.269 | 4 rs9323719 2 rs7143860 3 rs709900 2 rs10135954 |
| | | | | | | | 1 rs799103 1 rs799099 3 rs8018202 4 rs709915 |
| | | | | | | | 3 rs709918 3 rs2066041 1 rs1457990 3 rs4899801 |
| | | | | | | | 1 rs10483957 |
| chr15 | 63410029 | 6.68E−06 | 3.31E−05 | 2.395 | 0.047 | 0.020 | 4 rs2019185 2 rs920688 1 rs894494 3 rs665287 |
| | | | | | | | 1 rs626163 2 rs639812 2 rs894491 1 rs581427 |
| | | | | | | | 4 rs603439 1 rs678113 2 rs602192 3 rs7182756 |
| | | | | | | | 1 rs2280345 3 rs11071841 1 rs2277582 |
| chr15 | 95944049 | 4.24E−06 | 2.25E−05 | 0.593 | 0.079 | 0.127 | 2 rs8029926 4 rs649034 4 rs2036348 |
| chr18 | 38114511 | 4.94E−06 | 2.56E−05 | 0.555 | 0.055 | 0.094 | 4 rs9304267 3 rs3763494 1 rs882291 2 rs898785 |
| | | | | | | | 3 rs11082268 4 rs8088748 2 rs10502781 3 rs717127 |
| chr20 | 45233401 | 3.10E−06 | 1.71E−05 | 1.397 | 0.322 | 0.254 | 1 rs6063073 4 rs6066209 3 rs2018876 2 rs3092781 |
| | | | | | | | 4 rs6122563 3 rs8126262 1 rs6063083 3 rs6018337 |
| | | | | | | | 4 rs7262634 |

*Associating alleles are indicated in front of each marker (Allelic code: A = 1, C = 2, G = 3, T = 4)

### Chromosome 6p22.3 Locus

One of the most significant association signals for non-obese diabetic patients was identified by two single markers (rs1569699 and rs7756992) and two 2 marker haplotypes mapping to chromosome 6p22.3 (Table 3). These markers are located within one LD block at position 20634996-20836710 bases (NCBI Build 35) between markers rs4429936 and rs6908425 (SEQ ID NO:1; FIG. 1). This LD block contains the 5′ end including exons 1-5 of the gene CDK5 regulatory subunit associated protein 1-like 1 (CDKAL1) (NM_{—}017774). The CDKAL1 protein has catalytic activity as well as iron ion binding activity but the in vivo function in unknown. It is widely expressed including expression in pancreas.

To verify the association of rs1569699 and rs7756992 to Type 2 diabetes the two markers were genotyped in a Danish Type 2 diabetes case—control cohort and also in a US Caucasian cohort Type 2 diabetes cohort from the PENN CATH study (Table 7). The results show that the two markers are significantly associated with Type 2 diabetes in the Danish cohort and that it confers a similar risk in the US UPenn. cohort although the results do not reach statistical significance. When the two replication cohorts are combined the results are significant with a risk of around 1.2. When all the cohorts are combined the risk for each marker is over 1.2 comparing a group of nearly 3000 Type 2 diabetes patients (not accounting for BMI) and over 8000 controls. These results are genome wide significant.

TABLE 7
Association of rs1569699 and rs7756992 to Type 2 diabetes
Iceland

| rs-Name | Allele | Chr | Pos (B35) | Aff. n | Aff. frq | Ctrl. n | Ctrl. frq | Rrisk | Padj |
|---|---|---|---|---|---|---|---|---|---|
| rs1569699 | 2 | chr6 | 20787289 | 1397 | 0.297 | 5264 | 0.256 | 1.224 | 0.000158 |
| rs7756992 | 3 | chr6 | 20787688 | 1398 | 0.270 | 5271 | 0.232 | 1.228 | 0.000204 |

Denmark (Steno)

| rs-Name | Allele | Chr | Pos (B35) | Aff. n | Aff. frq | Ctrl. n | Ctrl. frq | Rrisk | P |
|---|---|---|---|---|---|---|---|---|---|
| rs1569699 | 2 | chr6 | 20787289 | 1108 | 0.361 | 2346 | 0.321 | 1.200 | 0.00079 |
| rs7756992 | 3 | chr6 | 20787688 | 1131 | 0.320 | 2361 | 0.274 | 1.247 | 0.000078 |

Upenn

| rs-Name | Allele | Chr | Pos (B35) | Aff. n | Aff. frq | Ctrl. n | Ctrl. frq | Rrisk | P |
|---|---|---|---|---|---|---|---|---|---|
| rs1569699 | 2 | chr6 | 20787289 | 360 | 0.346 | 522 | 0.308 | 1.185 | 0.09944 |
| rs7756992 | 3 | chr6 | 20787688 | 392 | 0.293 | 690 | 0.261 | 1.176 | 0.103824 |

Combined replication cohorts

| rs-Name | Allele | Chr | Pos (B35) | Aff. n | Aff. frq | Ctrl. n | Ctrl. frq | Rrisk | Pmh |
|---|---|---|---|---|---|---|---|---|---|
| rs1569699 | 2 | chr6 | 20787289 | 1468 | — | 2868 | — | 1.195 | 0.00002 |
| rs7756992 | 3 | chr6 | 20787688 | 1523 | — | 3051 | — | 1.221 | 2.8E−06 |

Combined all cohorts

| rs-Name | Allele | Chr | Pos (B35) | Aff. n | Aff. frq | Ctrl. n | Ctrl. frq | Rrisk | Pmh |
|---|---|---|---|---|---|---|---|---|---|
| rs1569699 | 2 | chr6 | 20787289 | 2865 | — | 8132 | — | 1.207 | 1.1E−07 |
| rs7756992 | 3 | chr6 | 20787688 | 2921 | — | 8322 | — | 1.224 | 1.9E−09 |

These results show significant association to the 20634996-20836710 by region (Build 34) on chromosome 6, between markers rs4429936 and rs6908425, in Type 2 diabetes. Values for relative risk (RR) are comparable in all three cohorts; the lack of significant association at the 0.05-level in the UPenn cohort is mainly due to lack of power compared with the other cohorts, although the RR value is slightly lower in this cohort as compared with Iceland (RR of 1.185 compared with 1.224 for rs1569699). Furthermore, RR-values for non-obese Type 2 diabetes patients in Iceland are even higher (RR=1.33 for rs1569699).

### Chromosome 10q23.33 Locus

Seven single markers and seven two marker haplotypes in a region on chromosome 10q23.33 were found to be associated with Type 2 diabetes (Table 1). Most of those markers are also associated to diabetes with elevated RR values when obese patients are analysed separately (Table 5). These markers are located within one LD block between positions 94192885 and 94490091 (NCBI Build 35), corresponding to the genomic segment bridged by markers rs2798253 and rs11187152 (FIG. 2). This LD block contains three genes, Insulin-degrading enzyme (IDE) (NM_{—}004969), Kinesin family member 11 (KIF11) (NM_{—}004523) and Homeobox, hematopoietically expressed (HHEX) (NM_{—}002729).

IDE may belong to a protease family responsible for intercellular peptide signalling. Though its role in the cellular processing of insulin has not yet been defined, insulin-degrading enzyme is thought to be involved in the termination of the insulin response (Fakhrai-Rad et al, Human Molecular Genetics 9:2149-2158, 2000). Genetic analysis of the diabetic GK rat has revealed 2 amino acid substitutions in the IDE gene (H18R and A890V) in the GK allele which reduced insulin-degrading activity by 31% in transfected cells. However, when the H18R and A890V variants were studied separately, no effects were observed, suggesting a synergistic effect of the 2 variants on insulin degradation. No effect on insulin degradation was observed in cell lysates, suggesting that the effect may be coupled to receptor-mediated internalization of insulin. Congenic rats with the IDE GK allele displayed postprandial hyperglycemia, reduced lipogenesis in fat cells, blunted insulin-stimulated glucose transmembrane uptake, and reduced insulin degradation in isolated muscle. Analysis of additional rat strains demonstrated that the dysfunctional IDE allele was unique to GK rats. The authors concluded that IDE plays an important role in the diabetic phenotype in GK rats. IDE has been studied as a candidate gene for Type 2 diabetes in humans with inconsistent results. Two large studies have recently analysed the association of IDE to Type 2 diabetes by mutation screening and haplotype analysis using tagging SNPs over the gene (Groves et al, Diabetes 52:1300-1305, 2003; Florez et al, Diabetes 55:128-135, 2006). Both studies conclude that common variants in IDE are unlikely to confer significant risk of Type 2 diabetes. These studies did however, not include the whole LD block as defined in FIG. 2 and at least some of the markers identified in our study as associated with Type 2 diabetes are outside the regions analysed in those previous studies. Based on the results reported here, markers in LD with IDE are associated with Type 2 diabetes, providing genetic evidence for the role of IDE in the etiology of Type 2 diabetes.

KIF11 encodes a motor protein that belongs to the kinesin-like protein family. Members of this protein family are known to be involved in various kinds of spindle dynamics. The function of this gene product includes chromosome positioning, centrosome separation and establishing a bipolar spindle during cell mitosis. This gene is not a good functional candidate for diabetes but has to be considered as a positional candidate due to its location within the associated LD block.

HHEX encodes a member of the homeobox family of transcription factors, many of which are involved in developmental processes. Expression in specific hematopoietic lineages suggests that this protein may play a role in hematopoietic differentiation. HHEX is essential for pancreatic development; in HHEX negative mouse embryos there is a complete failure in ventral pancreatic specification (Bort et al, Development 131, 797-806, 2004). Other transcription factors involved in pancreatic development include the MODY genes as well as other factors that have been implicated in late onset diabetes. HHEX is also an essential effector of Wnt antagonist for heart induction (Foley and Mercola, GENES & DEVELOPMENT 19:387-396, 2005). This puts HHEX in the same pathway as the recently established Type 2 diabetes gene TCF7L2 and together these data make HHEX a functional as well as positional candidate for Type 2 diabetes.

To verify the association of rs2497304, rs947591, rs10882091 and rs7914814 to Type 2 diabetes, the markers were genotyped in a Danish Type 2 diabetes case—control cohort and also in a US Caucasian cohort Type 2 diabetes cohort from the PENN CATH study (Table 8). The results show that the association is not replicated in either cohort independently. However, when the two cohorts are combined the association of rs947591 reaches significance at the 0.05 level, with a risk of 1.1 in the combined cohort. When all the cohorts are combined the risk is 1.15 for the rs947591 marker.

These results indicate that variants within the LD block on Chromosome 10 that includes IDE and HHEX are susceptibility variants for Type 2 diabetes.

TABLE 8
Association analysis of markers on Chromosome 10 to Type 2 diabetes in
Iceland, Denmark, and the US.
Iceland

| rs-Name | Allele | Chr | Pos (B35) | Aff. n | Aff. frq | Ctrl. n | Ctrl. frq | Rrisk | Padj |
|---|---|---|---|---|---|---|---|---|---|
| rs10882091 | 2 | chr10 | 94364357 | 1399 | 0.472 | 5275 | 0.415 | 1.257 | 0.0000023 |
| rs7914814 | 4 | chr10 | 94372930 | 1399 | 0.472 | 5275 | 0.416 | 1.256 | 0.0000024 |
| rs2497304 | 1 | chr10 | 94482696 | 1399 | 0.542 | 5275 | 0.485 | 1.257 | 0.0000019 |
| rs947591 | 1 | chr10 | 94485733 | 1399 | 0.526 | 5273 | 0.475 | 1.226 | 0.0000221 |

Denmark (Steno)

| rs-Name | Allele | Chr | Pos (B35) | Aff. n | Aff. frq | Ctrl. n | Ctrl. frq | Rrisk | P |
|---|---|---|---|---|---|---|---|---|---|
| rs10882091 | 2 | chr10 | 94364357 | 1115 | 0.431 | 2341 | 0.413 | 1.077 | 0.15 |
| rs7914814 | 4 | chr10 | 94372930 | 1141 | 0.430 | 2360 | 0.410 | 1.088 | 0.10 |
| rs2497304 | 1 | chr10 | 94482696 | 1145 | 0.528 | 2348 | 0.509 | 1.079 | 0.14 |
| rs947591 | 1 | chr10 | 94485733 | 1140 | 0.502 | 2361 | 0.478 | 1.103 | 0.055 |

Upenn

| rs-Name | Allele | Chr | Pos (B35) | Aff. n | Aff. frq | Ctrl. n | Ctrl. frq | Rrisk | P |
|---|---|---|---|---|---|---|---|---|---|
| rs10882091 | 2 | chr10 | 94364357 | 386 | 0.377 | 640 | 0.375 | 1.008 | 0.93 |
| rs7914814 | 4 | chr10 | 94372930 | 394 | 0.379 | 683 | 0.381 | 0.995 | 0.95 |
| rs2497304 | 1 | chr10 | 94482696 | 408 | 0.460 | 778 | 0.454 | 1.021 | 0.81 |
| rs947591 | 1 | chr10 | 94485733 | 393 | 0.480 | 687 | 0.459 | 1.089 | 0.34 |

Combined replication cohorts

| rs-Name | Allele | Chr | Pos (B35) | Aff. n | Aff. frq | Ctrl. n | Ctrl. frq | Rrisk | Pmh |
|---|---|---|---|---|---|---|---|---|---|
| rs10882091 | 2 | chr10 | 94364357 | 1501 | — | 2981 | — | 1.052 | 0.19 |
| rs7914814 | 4 | chr10 | 94372930 | 1535 | — | 3043 | — | 1.053 | 0.16 |
| rs2497304 | 1 | chr10 | 94482696 | 1553 | — | 3126 | — | 1.057 | 0.16 |
| rs947591 | 1 | chr10 | 94485733 | 1533 | — | 3048 | — | 1.098 | 0.032 |

Combined all cohorts

| rs-Name | Allele | Chr | Pos (B35) | Aff. n | Aff. frq | Ctrl. n | Ctrl. frq | Rrisk | Pmh |
|---|---|---|---|---|---|---|---|---|---|
| rs10882091 | 2 | chr10 | 94364357 | 2900 | — | 8256 | — | 1.136 | 0.000017 |
| rs7914814 | 4 | chr10 | 94372930 | 2934 | — | 8318 | — | 1.137 | 0.000012 |
| rs2497304 | 1 | chr10 | 94482696 | 2952 | — | 8401 | — | 1.139 | 0.000011 |
| rs947591 | 1 | chr10 | 94485733 | 2932 | — | 8321 | — | 1.152 | 9.7E−07 |

### Chromosome 17q24.3 Locus

Five single markers and two two marker haplotypes in a region of chromosome 17q24.3 were found to be associated with Type 2 diabetes in non-obese patients (Table 3). Some of these markers show the strongest association reported in Table 3 and association to this region was also observed when all diabetics were analysed (Table 1). These markers are located within two adjacent LD blocks located between positions 66037656 and 66163076 (NCBI Build 35) on chromosome 17, between markers rs11077501 and rs4793497 (FIG. 3). The association is significant after correction for the number of tests performed in the single marker association analysis; i.e., the association is significant at the genome-wide level. No known genes are located within these LD blocks. However, it is possible that variants in this region affect genes in neighboring regions including KCNJ2 and KCNJ16. Alternatively these variants may affect unknown genes within these LD block regions.

TABLE 9
SNPs located within the CDKAL1 gene (Located between position
20,642,736 and 21,340,611 bp on Chromosome 6 in NCBI Build
35 and NCBI Build 36)

| Pos | |
|---|---|
| Build | |
| 35/36 | Marker ID |
| 20642787 | rs41271303 |
| 20642953 | rs11963450 |
| 20643397 | rs981043 |
| 20643513 | rs981042 |
| 20643675 | rs16883895 |
| 20643753 | rs17512225 |
| 20643840 | rs35035071 |
| 20643949 | rs6904566 |
| 20644073 | rs6927356 |
| 20644093 | rs35281412 |
| 20644313 | rs35915788 |
| 20644314 | rs34025398 |
| 20644319 | rs34361235 |
| 20644335 | rs6905138 |
| 20644499 | rs13194858 |
| 20644717 | rs2179551 |
| 20644727 | rs2179550 |
| 20644787 | rs9465794 |
| 20644787 | rs9465795 |
| 20644848 | rs7747962 |
| 20644858 | rs6910725 |
| 20644918 | rs965054 |
| 20644971 | rs2143407 |
| 20645032 | rs10619380 |
| 20645431 | rs2328525 |
| 20645661 | rs13199286 |
| 20645841 | rs10611252 |
| 20645940 | rs7753499 |
| 20646023 | rs7753956 |
| 20646024 | rs34811195 |
| 20646024 | rs7753670 |
| 20646107 | rs3060613 |
| 20646109 | rs11277970 |
| 20646110 | rs11280099 |
| 20646114 | rs6149468 |
| 20646139 | rs16883900 |
| 20646175 | rs7774291 |
| 20646441 | rs10612082 |
| 20646476 | rs9368198 |
| 20646502 | rs13203336 |
| 20646504 | rs13203631 |
| 20646619 | rs6456355 |
| 20646644 | rs10484635 |
| 20647190 | rs12204173 |
| 20647320 | rs13207544 |
| 20647851 | rs12198728 |
| 20647984 | rs28396084 |
| 20648327 | rs12199073 |
| 20648500 | rs9465796 |
| 20648561 | rs12212600 |
| 20648596 | rs13212040 |
| 20648663 | rs35291340 |
| 20648722 | rs12199324 |
| 20649085 | rs12200871 |
| 20649159 | rs9348432 |
| 20649183 | rs12200834 |
| 20649236 | rs34860173 |
| 20649324 | rs11754872 |
| 20649498 | rs6456356 |
| 20649517 | rs9368199 |
| 20649682 | rs2143406 |
| 20650176 | rs10484634 |
| 20650200 | rs7758851 |
| 20650398 | rs34677076 |
| 20651447 | rs6928571 |
| 20651461 | rs12192584 |
| 20651608 | rs34856684 |
| 20652015 | rs9350255 |
| 20652091 | rs9368200 |
| 20652136 | rs12214002 |
| 20652245 | rs9465797 |
| 20652300 | rs9465798 |
| 20652574 | rs28699301 |
| 20652650 | rs13215844 |
| 20652678 | rs12214315 |
| 20652722 | rs11759517 |
| 20652786 | rs13218957 |
| 20652806 | rs13218962 |
| 20653186 | rs10543744 |
| 20653201 | rs12216047 |
| 20653447 | rs9366354 |
| 20653890 | rs9358342 |
| 20654091 | rs9368201 |
| 20654382 | rs34206163 |
| 20654506 | rs9465799 |
| 20654794 | rs34187071 |
| 20654867 | rs9465800 |
| 20654890 | rs6908974 |
| 20654992 | rs13197372 |
| 20655361 | rs13214145 |
| 20655793 | rs16883910 |
| 20655968 | rs12194705 |
| 20656271 | rs35080661 |
| 20656465 | rs7753467 |
| 20656466 | rs7773488 |
| 20656986 | rs34182285 |
| 20657084 | rs34242699 |
| 20657780 | rs9348433 |
| 20657942 | rs9460519 |
| 20658083 | rs12198377 |
| 20658096 | rs9465801 |
| 20658195 | rs9465802 |
| 20658822 | rs28458932 |
| 20658823 | rs9465803 |
| 20658981 | rs2103682 |
| 20659321 | rs9465804 |
| 20659580 | rs34611621 |
| 20660058 | rs12055423 |
| 20660653 | rs9465805 |
| 20660829 | rs11365187 |
| 20660836 | rs11320714 |
| 20660918 | rs9350256 |
| 20661764 | rs7756211 |
| 20662069 | rs9460520 |
| 20662498 | rs34245467 |
| 20662930 | rs9350257 |
| 20663855 | rs11964554 |
| 20663990 | rs9465806 |
| 20664109 | rs11964635 |
| 20664190 | rs13199421 |
| 20664314 | rs6932320 |
| 20664570 | rs12200078 |
| 20664659 | rs13437555 |
| 20664884 | rs9350258 |
| 20665256 | rs12176441 |
| 20665260 | rs12183826 |
| 20665264 | rs9356738 |
| 20665272 | rs9348434 |
| 20665343 | rs9465807 |
| 20665804 | rs4458667 |
| 20665995 | rs7739402 |
| 20667590 | rs16883914 |
| 20667591 | rs16883916 |
| 20667900 | rs9654584 |
| 20667999 | rs9465808 |
| 20668414 | rs17584626 |
| 20668565 | rs7751682 |
| 20669667 | rs11361279 |
| 20669681 | rs34634263 |
| 20670059 | rs12214549 |
| 20670364 | rs7753519 |
| 20670575 | rs28567007 |
| 20670597 | rs7772137 |
| 20670719 | rs12208597 |
| 20670998 | rs9368202 |
| 20671877 | rs2328526 |
| 20672452 | rs34823358 |
| 20673287 | rs28639914 |
| 20673363 | rs34233572 |
| 20673415 | rs4712506 |
| 20673935 | rs13203450 |
| 20674280 | rs9350259 |
| 20674435 | rs6918457 |
| 20674595 | rs35210537 |
| 20674749 | rs11329887 |
| 20675016 | rs9348435 |
| 20675068 | rs35366106 |
| 20675342 | rs16901563 |
| 20675352 | rs12333229 |
| 20675520 | rs9460521 |
| 20676092 | rs10589899 |
| 20676351 | rs2876573 |
| 20676957 | rs6935461 |
| 20676963 | rs6935465 |
| 20676968 | rs10603174 |
| 20677060 | rs12333291 |
| 20677967 | rs2064321 |
| 20677985 | rs35546893 |
| 20678018 | rs4291090 |
| 20678121 | rs2064320 |
| 20678268 | rs9465810 |
| 20678275 | rs9465811 |
| 20678423 | rs9358344 |
| 20678756 | rs10946390 |
| 20679114 | rs6905281 |
| 20679339 | rs16883932 |
| 20679612 | rs34904067 |
| 20679660 | rs7744002 |
| 20679763 | rs35142564 |
| 20680095 | rs9465812 |
| 20680678 | rs7759094 |
| 20680784 | rs9460522 |
| 20681538 | rs7764551 |
| 20681585 | rs10541455 |
| 20682409 | rs16883935 |
| 20682542 | rs13215603 |
| 20682568 | rs962576 |
| 20683235 | rs1474720 |
| 20683797 | rs16883944 |
| 20684155 | rs34538343 |
| 20684269 | rs9350260 |
| 20684353 | rs16883951 |
| 20684645 | rs9358345 |
| 20684862 | rs1012627 |
| 20684890 | rs9368203 |
| 20684939 | rs35894322 |
| 20684965 | rs4710932 |
| 20684984 | rs6909117 |
| 20685540 | rs1012626 |
| 20685748 | rs1012625 |
| 20685760 | rs7752194 |
| 20685958 | rs9465813 |
| 20686014 | rs12207923 |
| 20686355 | rs16883963 |
| 20686831 | rs13205786 |
| 20686887 | rs35205364 |
| 20687102 | rs10456232 |
| 20687189 | rs9465814 |
| 20687201 | rs35571892 |
| 20687740 | rs9465815 |
| 20687753 | rs36119371 |
| 20687921 | rs28621813 |
| 20687926 | rs9350261 |
| 20687928 | rs7341226 |
| 20688175 | rs6927481 |
| 20688323 | rs35313444 |
| 20688373 | rs6928198 |
| 20688404 | rs6907897 |
| 20688545 | rs6928586 |
| 20688872 | rs9368204 |
| 20689021 | rs9358346 |
| 20689589 | rs11967546 |
| 20689593 | rs34134803 |
| 20689772 | rs10456233 |
| 20689807 | rs7744833 |
| 20690122 | rs9460523 |
| 20690123 | rs9465816 |
| 20690432 | rs6908077 |
| 20690630 | rs9465817 |
| 20691069 | rs11967445 |
| 20691263 | rs34022950 |
| 20691793 | rs9460524 |
| 20691994 | rs34020592 |
| 20692003 | rs11448102 |
| 20692339 | rs9465818 |
| 20692402 | rs9350262 |
| 20692513 | rs13205241 |
| 20693000 | rs12153939 |
| 20693100 | rs6925593 |
| 20693119 | rs4712507 |
| 20693225 | rs10558806 |
| 20693267 | rs35982532 |
| 20693276 | rs11385529 |
| 20693360 | rs9348436 |
| 20693416 | rs9368206 |
| 20693438 | rs13209542 |
| 20693452 | rs9368207 |
| 20693630 | rs13209907 |
| 20693635 | rs6926658 |
| 20694018 | rs12213132 |
| 20694182 | rs4357125 |
| 20694554 | rs6932944 |
| 20694607 | rs6932962 |
| 20695026 | rs9348437 |
| 20695332 | rs12201857 |
| 20695356 | rs9465819 |
| 20695447 | rs6938955 |
| 20695539 | rs9460525 |
| 20695827 | rs9465820 |
| 20695964 | rs10946391 |
| 20695968 | rs9368208 |
| 20696003 | rs9465821 |
| 20696183 | rs6923790 |
| 20696399 | rs10558139 |
| 20697309 | rs6907459 |
| 20697320 | rs6907767 |
| 20697321 | rs9465822 |
| 20697349 | rs6930283 |
| 20697706 | rs6908042 |
| 20697741 | rs6935317 |
| 20697761 | rs35370102 |
| 20698266 | rs9368209 |
| 20698366 | rs13216746 |
| 20698367 | rs13216747 |
| 20699007 | rs35485532 |
| 20699747 | rs13216324 |
| 20699817 | rs4336434 |
| 20700046 | rs4509107 |
| 20700428 | rs9465823 |
| 20700465 | rs6936705 |
| 20700679 | rs34023799 |
| 20700929 | rs6942313 |
| 20701057 | rs28869917 |
| 20701318 | rs34982231 |
| 20701631 | rs9358349 |
| 20701770 | rs9460526 |
| 20701829 | rs9366356 |
| 20702163 | rs36120092 |
| 20702181 | rs9465824 |
| 20702519 | rs4712512 |
| 20702561 | rs4712513 |
| 20702646 | rs4710934 |
| 20702658 | rs9348438 |
| 20702902 | rs9460529 |
| 20703363 | rs13199587 |
| 20703470 | rs13199384 |
| 20703526 | rs10223680 |
| 20703606 | rs9350263 |
| 20703768 | rs9465825 |
| 20703832 | rs10223876 |
| 20704100 | rs35702271 |
| 20704171 | rs9358350 |
| 20704432 | rs12208985 |
| 20704771 | rs12210459 |
| 20704892 | rs35431707 |
| 20705144 | rs36039523 |
| 20705297 | rs11758281 |
| 20705350 | rs28893199 |
| 20705757 | rs34256347 |
| 20706019 | rs12192740 |
| 20706282 | rs13212326 |
| 20706486 | rs12199184 |
| 20706753 | rs10456234 |
| 20707009 | rs4712514 |
| 20707422 | rs9465826 |
| 20707607 | rs9366357 |
| 20707867 | rs2294809 |
| 20708549 | rs2294808 |
| 20708813 | rs7762750 |
| 20708976 | rs4712515 |
| 20708998 | rs10522824 |
| 20708999 | rs35660518 |
| 20709002 | rs10679950 |
| 20709003 | rs34870864 |
| 20709022 | rs4712516 |
| 20709145 | rs4710935 |
| 20709386 | rs9465827 |
| 20709388 | rs12204865 |
| 20709672 | rs10946393 |
| 20709764 | rs12209806 |
| 20709894 | rs10946394 |
| 20709921 | rs1997778 |
| 20709971 | rs35878587 |
| 20710359 | rs1997777 |
| 20710378 | rs2223622 |
| 20710776 | rs11964057 |
| 20711246 | rs9460530 |
| 20711344 | rs9460531 |
| 20711376 | rs34329159 |
| 20711640 | rs7764558 |
| 20711804 | rs4710936 |
| 20712056 | rs12213940 |
| 20712228 | rs13215038 |
| 20712739 | rs10946395 |
| 20712832 | rs6939917 |
| 20712975 | rs9358351 |
| 20713800 | rs6925097 |
| 20713924 | rs9465828 |
| 20713955 | rs9465829 |
| 20713961 | rs6902661 |
| 20714057 | rs34373680 |
| 20714281 | rs35051096 |
| 20714508 | rs932405 |
| 20714591 | rs6926585 |
| 20714635 | rs3938395 |
| 20715464 | rs11964664 |
| 20715551 | rs35964987 |
| 20715663 | rs12206413 |
| 20715758 | rs35990187 |
| 20715763 | rs4991654 |
| 20715910 | rs9460532 |
| 20715991 | rs13328250 |
| 20716030 | rs13328252 |
| 20716194 | rs4712517 |
| 20716257 | rs4712518 |
| 20717220 | rs7758129 |
| 20717475 | rs13206462 |
| 20717483 | rs13192442 |
| 20717486 | rs13206468 |
| 20717492 | rs13192445 |
| 20717498 | rs13192450 |
| 20717504 | rs13206477 |
| 20717510 | rs13206483 |
| 20717577 | rs12179168 |
| 20717586 | rs12180975 |
| 20717611 | rs12179172 |
| 20717860 | rs12179563 |
| 20718357 | rs11355836 |
| 20718696 | rs2328527 |
| 20718709 | rs11452882 |
| 20718920 | rs2876574 |
| 20719905 | rs9465831 |
| 20720031 | rs34877824 |
| 20720290 | rs13212501 |
| 20720647 | rs9358352 |
| 20720703 | rs9358353 |
| 20720761 | rs28756205 |
| 20720889 | rs9350265 |
| 20720989 | rs7750508 |
| 20721130 | rs7771052 |
| 20721141 | rs9460533 |
| 20721195 | rs13200415 |
| 20721216 | rs10550932 |
| 20721312 | rs9465832 |
| 20721463 | rs9368211 |
| 20721471 | rs9350266 |
| 20721507 | rs11752592 |
| 20721515 | rs9350267 |
| 20721754 | rs978988 |
| 20721898 | rs978987 |
| 20721906 | rs978986 |
| 20722036 | rs5874773 |
| 20722196 | rs5874774 |
| 20722500 | rs7756788 |
| 20722659 | rs9348439 |
| 20722661 | rs9356739 |
| 20722693 | rs9356740 |
| 20722738 | rs7760894 |
| 20723021 | rs2796913 |
| 20723046 | rs2608613 |
| 20723121 | rs10456710 |
| 20723139 | rs9476286 |
| 20723140 | rs28368538 |
| 20723140 | rs9476287 |
| 20723141 | rs28612622 |
| 20723142 | rs9463660 |
| 20723146 | rs9463661 |
| 20723162 | rs9461022 |
| 20723193 | rs9461021 |
| 20723235 | rs34980442 |
| 20723239 | rs34049080 |
| 20723258 | rs35218684 |
| 20723260 | rs12175876 |
| 20723270 | rs10948323 |
| 20723287 | rs34400313 |
| 20723292 | rs36081550 |
| 20723304 | rs12175878 |
| 20723305 | rs34520184 |
| 20723305 | rs34756989 |
| 20723322 | rs4629736 |
| 20723322 | rs9296917 |
| 20723324 | rs28562027 |
| 20723333 | rs9381823 |
| 20723346 | rs34769771 |
| 20723346 | rs34774640 |
| 20723346 | rs36038896 |
| 20723365 | rs13209195 |
| 20723369 | rs12213541 |
| 20723369 | rs34112320 |
| 20723371 | rs34615869 |
| 20723379 | rs35949519 |
| 20723381 | rs9257498 |
| 20723383 | rs34200576 |
| 20723393 | rs4960519 |
| 20723396 | rs9261905 |
| 20723402 | rs9267103 |
| 20723404 | rs17367677 |
| 20723416 | rs9261906 |
| 20723421 | rs9267104 |
| 20723424 | rs9267105 |
| 20723434 | rs28810763 |
| 20723438 | rs12179121 |
| 20723438 | rs4714959 |
| 20723439 | rs10946636 |
| 20723439 | rs28763327 |
| 20723439 | rs28847950 |
| 20723439 | rs3933247 |
| 20723449 | rs28808723 |
| 20723451 | rs9767082 |
| 20723452 | rs35132675 |
| 20723452 | rs35517166 |
| 20723452 | rs4714297 |
| 20723456 | rs12182737 |
| 20723456 | rs36163804 |
| 20723460 | rs35790973 |
| 20723464 | rs35236694 |
| 20723469 | rs35567559 |
| 20723469 | rs9267106 |
| 20723471 | rs9800557 |
| 20723484 | rs12182463 |
| 20723489 | rs10948322 |
| 20723489 | rs34052284 |
| 20723489 | rs9268999 |
| 20723490 | rs9258377 |
| 20723495 | rs9257499 |
| 20723498 | rs9265816 |
| 20723500 | rs28771402 |
| 20723501 | rs28771401 |
| 20723502 | rs12194731 |
| 20723502 | rs4451188 |
| 20723502 | rs9717323 |
| 20723502 | rs9765920 |
| 20723503 | rs9265815 |
| 20723504 | rs28771400 |
| 20723506 | rs12190813 |
| 20723506 | rs12215416 |
| 20723507 | rs12178527 |
| 20723510 | rs35234761 |
| 20723510 | rs35887156 |
| 20723512 | rs28797321 |
| 20723515 | rs28771399 |
| 20723522 | rs13207682 |
| 20723522 | rs36099432 |
| 20723531 | rs28749543 |
| 20723535 | rs13196506 |
| 20723535 | rs35716308 |
| 20723536 | rs28831180 |
| 20723536 | rs34938144 |
| 20723537 | rs36142967 |
| 20723541 | rs35313792 |
| 20723542 | rs9260904 |
| 20723543 | rs34597832 |
| 20723545 | rs9767740 |
| 20723546 | rs28771398 |
| 20723550 | rs34384951 |
| 20723554 | rs12524128 |
| 20723554 | rs12665124 |
| 20723554 | rs34922643 |
| 20723555 | rs9260903 |
| 20723557 | rs28771397 |
| 20723557 | rs6915279 |
| 20723559 | rs9261907 |
| 20723559 | rs9766798 |
| 20723559 | rs9767242 |
| 20723560 | rs6914835 |
| 20723560 | rs9260902 |
| 20723560 | rs9767101 |
| 20723562 | rs9261908 |
| 20723563 | rs12207064 |
| 20723563 | rs12213193 |
| 20723563 | rs35750154 |
| 20723566 | rs9260901 |
| 20723570 | rs9269000 |
| 20723579 | rs12178368 |
| 20723579 | rs13197714 |
| 20723579 | rs28771396 |
| 20723579 | rs34443697 |
| 20723579 | rs9267107 |
| 20723582 | rs35113301 |
| 20723582 | rs3933248 |
| 20723583 | rs28819830 |
| 20723586 | rs10947899 |
| 20723586 | rs9260900 |
| 20723588 | rs13204671 |
| 20723588 | rs34094007 |
| 20723588 | rs34963756 |
| 20723588 | rs4304158 |
| 20723589 | rs4298351 |
| 20723590 | rs12182307 |
| 20723590 | rs12201487 |
| 20723590 | rs34097573 |
| 20723595 | rs13219021 |
| 20723596 | rs34456153 |
| 20723596 | rs9767597 |
| 20723597 | rs9767102 |
| 20723598 | rs9269001 |
| 20723601 | rs12180097 |
| 20723603 | rs12178465 |
| 20723603 | rs35128115 |
| 20723607 | rs4273681 |
| 20723609 | rs12193754 |
| 20723617 | rs813814 |
| 20723617 | rs9689672 |
| 20723619 | rs36173068 |
| 20723623 | rs12202172 |
| 20723623 | rs28862376 |
| 20723625 | rs12178884 |
| 20723625 | rs12203117 |
| 20723625 | rs34250588 |
| 20723626 | rs12182581 |
| 20723632 | rs34147797 |
| 20723635 | rs9688484 |
| 20723636 | rs9269002 |
| 20723636 | rs9767733 |
| 20723637 | rs10948989 |
| 20723637 | rs34732676 |
| 20723637 | rs9260899 |
| 20723638 | rs35686233 |
| 20723639 | rs11753098 |
| 20723639 | rs35156647 |
| 20723646 | rs34719653 |
| 20723646 | rs35575623 |
| 20723648 | rs12192046 |
| 20723649 | rs11759854 |
| 20723649 | rs28771395 |
| 20723649 | rs35563402 |
| 20723649 | rs36016334 |
| 20723649 | rs4555911 |
| 20723653 | rs11965757 |
| 20723654 | rs10948990 |
| 20723654 | rs28808296 |
| 20723654 | rs34995142 |
| 20723654 | rs809919 |
| 20723657 | rs11571978 |
| 20723659 | rs12208488 |
| 20723662 | rs11751374 |
| 20723663 | rs13217613 |
| 20723667 | rs12528735 |
| 20723668 | rs35160656 |
| 20723668 | rs35322569 |
| 20723668 | rs9766221 |
| 20723670 | rs35120225 |
| 20723670 | rs511868 |
| 20723674 | rs9269003 |
| 20723675 | rs34290316 |
| 20723675 | rs35735496 |
| 20723675 | rs9689102 |
| 20723676 | rs10948991 |
| 20723676 | rs9269004 |
| 20723677 | rs13220607 |
| 20723677 | rs34700647 |
| 20723677 | rs9260898 |
| 20723678 | rs9260897 |
| 20723684 | rs28771394 |
| 20723686 | rs12213200 |
| 20723687 | rs9717987 |
| 20723688 | rs28771393 |
| 20723688 | rs9717716 |
| 20723689 | rs28771392 |
| 20723690 | rs11758009 |
| 20723690 | rs34159662 |
| 20723690 | rs34683172 |
| 20723690 | rs9717331 |
| 20723691 | rs9717853 |
| 20723692 | rs9765875 |
| 20723696 | rs12178974 |
| 20723696 | rs36003577 |
| 20723698 | rs9767747 |
| 20723700 | rs28771391 |
| 20723704 | rs13216352 |
| 20723707 | rs12200762 |
| 20723707 | rs12206373 |
| 20723707 | rs34570202 |
| 20723707 | rs36197940 |
| 20723707 | rs9265814 |
| 20723714 | rs34131282 |
| 20723717 | rs12175478 |
| 20723720 | rs28772692 |
| 20723722 | rs35516674 |
| 20723722 | rs35704013 |
| 20723727 | rs34691406 |
| 20723728 | rs28771390 |
| 20723728 | rs4374863 |
| 20723730 | rs34826149 |
| 20723734 | rs12207894 |
| 20723734 | rs12662476 |
| 20723736 | rs9260896 |
| 20723739 | rs28771389 |
| 20723740 | rs28771388 |
| 20723741 | rs9382592 |
| 20723745 | rs12173375 |
| 20723745 | rs9268072 |
| 20723745 | rs9382110 |
| 20723746 | rs4715211 |
| 20723747 | rs35961188 |
| 20723747 | rs34128950 |
| 20723747 | rs4620119 |
| 20723749 | rs9767236 |
| 20723752 | rs34717143 |
| 20723755 | rs13201202 |
| 20723756 | rs13201503 |
| 20723762 | rs13197088 |
| 20723762 | rs35347849 |
| 20723766 | rs35650828 |
| 20723766 | rs34663083 |
| 20723767 | rs13199241 |
| 20723767 | rs34205031 |
| 20723768 | rs34469031 |
| 20723768 | rs28749541 |
| 20723768 | rs9279137 |
| 20723769 | rs9260895 |
| 20723770 | rs35446958 |
| 20723770 | rs12212483 |
| 20723775 | rs28380829 |
| 20723777 | rs12525384 |
| 20723777 | rs34576984 |
| 20723782 | rs283541 |
| 20723785 | rs28380828 |
| 20723788 | rs10948698 |
| 20723790 | rs9265813 |
| 20723791 | rs9689173 |
| 20723792 | rs28380827 |
| 20723795 | rs35399169 |
| 20723799 | rs9260894 |
| 20723800 | rs12173388 |
| 20723800 | rs9269005 |
| 20723802 | rs28359816 |
| 20723803 | rs28380826 |
| 20723807 | rs280297 |
| 20723807 | rs34507582 |
| 20723807 | rs9261623 |
| 20723810 | rs12174621 |
| 20723813 | rs28380825 |
| 20723815 | rs35197377 |
| 20723816 | rs17362870 |
| 20723816 | rs35071522 |
| 20723816 | rs34562190 |
| 20723816 | rs35179751 |
| 20723816 | rs4458721 |
| 20723817 | rs12180385 |
| 20723817 | rs12206581 |
| 20723817 | rs12333308 |
| 20723817 | rs9269006 |
| 20723821 | rs9688475 |
| 20723829 | rs9269007 |
| 20723832 | rs9269008 |
| 20723832 | rs9269009 |
| 20723834 | rs35899754 |
| 20723836 | rs13211190 |
| 20723837 | rs12216274 |
| 20723838 | rs12191544 |
| 20723838 | rs4337934 |
| 20723841 | rs9279295 |
| 20723842 | rs12178577 |
| 20723849 | rs12215604 |
| 20723853 | rs9395360 |
| 20723854 | rs28749540 |
| 20723855 | rs28895226 |
| 20723855 | rs9260893 |
| 20723861 | rs4365925 |
| 20723864 | rs9261912 |
| 20723865 | rs28895227 |
| 20723870 | rs9261913 |
| 20723873 | rs12183502 |
| 20723873 | rs34332895 |
| 20723873 | rs9472692 |
| 20723876 | rs35640475 |
| 20723884 | rs9269010 |
| 20723888 | rs12192337 |
| 20723888 | rs35349786 |
| 20723891 | rs34978372 |
| 20723893 | rs280296 |
| 20723894 | rs11755576 |
| 20723903 | rs35553517 |
| 20723903 | rs28803616 |
| 20723904 | rs4358654 |
| 20723910 | rs9260892 |
| 20723914 | rs13196941 |
| 20723914 | rs9717238 |
| 20723915 | rs12175553 |
| 20723915 | rs12191408 |
| 20723915 | rs4460210 |
| 20723915 | rs9260891 |
| 20723918 | rs9395361 |
| 20723920 | rs9766917 |
| 20723926 | rs35073779 |
| 20723931 | rs9765884 |
| 20723931 | rs9767229 |
| 20723932 | rs35247846 |
| 20723932 | rs9717328 |
| 20723936 | rs28805674 |
| 20723936 | rs9260890 |
| 20723938 | rs34926348 |
| 20723938 | rs9717233 |
| 20723945 | rs35869128 |
| 20723946 | rs12175941 |
| 20723951 | rs9260889 |
| 20723952 | rs9766686 |
| 20723956 | rs10046203 |
| 20723957 | rs9260888 |
| 20723958 | rs12180540 |
| 20723961 | rs9766115 |
| 20723968 | rs6912019 |
| 20723969 | rs9767458 |
| 20723976 | rs13202161 |
| 20723989 | rs13218048 |
| 20724000 | rs13218040 |
| 20724001 | rs13206557 |
| 20724002 | rs13202152 |
| 20724013 | rs12210049 |
| 20724013 | rs34552032 |
| 20724018 | rs28890881 |
| 20724018 | rs34478537 |
| 20724025 | rs35191657 |
| 20724038 | rs34450517 |
| 20724043 | rs9689655 |
| 20724064 | rs12180172 |
| 20724068 | rs28865015 |
| 20724071 | rs9717839 |
| 20724072 | rs9717836 |
| 20724074 | rs9885593 |
| 20724082 | rs28887572 |
| 20724083 | rs13216113 |
| 20724083 | rs35254115 |
| 20724084 | rs10949190 |
| 20724085 | rs6923503 |
| 20724087 | rs12214411 |
| 20724091 | rs28747986 |
| 20724096 | rs1087363 |
| 20724096 | rs1091092 |
| 20724104 | rs12180759 |
| 20724107 | rs11758052 |
| 20724112 | rs9261321 |
| 20724113 | rs28861738 |
| 20724113 | rs36109104 |
| 20724123 | rs13208115 |
| 20724129 | rs9472693 |
| 20724130 | rs12208570 |
| 20724130 | rs28832660 |
| 20724131 | rs13200482 |
| 20724131 | rs9260886 |
| 20724131 | rs9472694 |
| 20724159 | rs9260885 |
| 20724172 | rs280295 |
| 20724172 | rs28749538 |
| 20724174 | rs13201041 |
| 20724184 | rs1087362 |
| 20724187 | rs12183850 |
| 20724190 | rs12202552 |
| 20724208 | rs9260884 |
| 20724210 | rs9474341 |
| 20724211 | rs9260883 |
| 20724222 | rs34669820 |
| 20724225 | rs12189886 |
| 20724234 | rs12182114 |
| 20724266 | rs9688643 |
| 20724272 | rs12195237 |
| 20724281 | rs35293595 |
| 20724281 | rs9381428 |
| 20724306 | rs34665644 |
| 20724309 | rs12189599 |
| 20724311 | rs12208503 |
| 20724312 | rs9800791 |
| 20724330 | rs12173681 |
| 20724337 | rs12182094 |
| 20724343 | rs12182119 |
| 20724352 | rs12182102 |
| 20724392 | rs12174506 |
| 20724393 | rs9767809 |
| 20724394 | rs12173703 |
| 20724401 | rs12202889 |
| 20724435 | rs28840538 |
| 20724466 | rs9473983 |
| 20724467 | rs34220946 |
| 20724487 | rs9261322 |
| 20724491 | rs9261323 |
| 20724517 | rs526092 |
| 20724564 | rs9473982 |
| 20724566 | rs34307764 |
| 20724626 | rs34490559 |
| 20724665 | rs4449621 |
| 20724685 | rs4714818 |
| 20724698 | rs4714817 |
| 20724722 | rs682384 |
| 20724738 | rs682051 |
| 20724753 | rs12530056 |
| 20724756 | rs35086683 |
| 20724888 | rs12528468 |
| 20725092 | rs10650195 |
| 20725092 | rs35956126 |
| 20725109 | rs6937578 |
| 20725159 | rs12530107 |
| 20725173 | rs720448 |
| 20725176 | rs9368212 |
| 20725250 | rs35481531 |
| 20725253 | rs720449 |
| 20725262 | rs10650196 |
| 20725495 | rs2064 |
| 20725499 | rs2065 |
| 20725638 | rs9368213 |
| 20725732 | rs41455744 |
| 20726077 | rs34960654 |
| 20726154 | rs6900954 |
| 20726168 | rs6456359 |
| 20726176 | rs7739405 |
| 20726601 | rs7760508 |
| 20726684 | rs10456007 |
| 20726987 | rs9460534 |
| 20727168 | rs11966749 |
| 20727353 | rs9358354 |
| 20727809 | rs9368214 |
| 20727982 | rs9350268 |
| 20728095 | rs2069013 |
| 20728160 | rs2069014 |
| 20728290 | rs2069015 |
| 20728292 | rs11967475 |
| 20729096 | rs10214549 |
| 20729217 | rs2223621 |
| 20729281 | rs9465835 |
| 20729984 | rs35404829 |
| 20729995 | rs10687080 |
| 20730233 | rs6915936 |
| 20730331 | rs9295472 |
| 20730732 | rs1569660 |
| 20730929 | rs7455009 |
| 20730945 | rs1569659 |
| 20730946 | rs5874775 |
| 20730948 | rs10601252 |
| 20730983 | rs1569658 |
| 20731030 | rs34142400 |
| 20731063 | rs16883996 |
| 20731181 | rs35073110 |
| 20731184 | rs5028948 |
| 20731263 | rs34201758 |
| 20731371 | rs6456360 |
| 20731373 | rs6456361 |
| 20731380 | rs6456362 |
| 20731584 | rs6922571 |
| 20731925 | rs35145358 |
| 20731950 | rs9465836 |
| 20732013 | rs7763304 |
| 20732158 | rs9465837 |
| 20732218 | rs7743314 |
| 20732337 | rs714831 |
| 20732361 | rs714830 |
| 20732484 | rs7743789 |
| 20732584 | rs5874776 |
| 20732589 | rs5874777 |
| 20732965 | rs16884003 |
| 20733079 | rs9356741 |
| 20733080 | rs9366358 |
| 20733246 | rs9356742 |
| 20733430 | rs11756987 |
| 20733470 | rs9465838 |
| 20733613 | rs2206579 |
| 20733920 | rs35111339 |
| 20734361 | rs6917583 |
| 20734388 | rs6917599 |
| 20734630 | rs13437429 |
| 20735418 | rs34307011 |
| 20735420 | rs4515379 |
| 20735756 | rs2223620 |
| 20735870 | rs9465839 |
| 20735993 | rs12664972 |
| 20736790 | rs28402356 |
| 20736798 | rs6934727 |
| 20736873 | rs35493429 |
| 20736881 | rs11369825 |
| 20737261 | rs4523079 |
| 20737470 | rs7771213 |
| 20737568 | rs9465840 |
| 20737687 | rs9465841 |
| 20737767 | rs9465842 |
| 20737837 | rs9460535 |
| 20737992 | rs34835908 |
| 20738060 | rs9465843 |
| 20738139 | rs13220465 |
| 20738159 | rs13220352 |
| 20738321 | rs4710937 |
| 20738375 | rs9460536 |
| 20738376 | rs13190734 |
| 20738451 | rs9465844 |
| 20738712 | rs35976895 |
| 20738713 | rs11327958 |
| 20738932 | rs13194407 |
| 20738990 | rs10484633 |
| 20739524 | rs2328528 |
| 20739789 | rs35958155 |
| 20739809 | rs4421185 |
| 20739932 | rs2328529 |
| 20740089 | rs9460537 |
| 20740745 | rs17224527 |
| 20740886 | rs34166991 |
| 20741024 | rs17823073 |
| 20741276 | rs12183074 |
| 20741316 | rs9465845 |
| 20741844 | rs35148963 |
| 20741886 | rs7768642 |
| 20742320 | rs9465846 |
| 20742407 | rs9465847 |
| 20742551 | rs17823127 |
| 20742594 | rs16884021 |
| 20742630 | rs4533976 |
| 20742865 | rs7755830 |
| 20743005 | rs7756031 |
| 20743139 | rs35650451 |
| 20743188 | rs10561117 |
| 20743241 | rs6940200 |
| 20743799 | rs9465848 |
| 20743808 | rs34351919 |
| 20745083 | rs4712519 |
| 20745285 | rs9368215 |
| 20745566 | rs12206285 |
| 20745853 | rs34049994 |
| 20745988 | rs7751957 |
| 20746702 | rs9465849 |
| 20746857 | rs7341291 |
| 20746957 | rs6921014 |
| 20747388 | rs9465850 |
| 20747583 | rs7758612 |
| 20747681 | rs35602526 |
| 20748295 | rs9465851 |
| 20748399 | rs34257578 |
| 20748403 | rs11339738 |
| 20748516 | rs9460538 |
| 20748850 | rs4712520 |
| 20748883 | rs4710938 |
| 20749084 | rs35889049 |
| 20749315 | rs9348440 |
| 20749879 | rs9465852 |
| 20750054 | rs12196009 |
| 20750306 | rs11968248 |
| 20750353 | rs34797179 |
| 20750764 | rs6925328 |
| 20750913 | rs36073053 |
| 20751140 | rs36224625 |
| 20751145 | rs35462488 |
| 20751150 | rs3060659 |
| 20751153 | rs5874778 |
| 20751155 | rs11267861 |
| 20751201 | rs4235999 |
| 20751230 | rs16884038 |
| 20751706 | rs2328530 |
| 20751731 | rs2328531 |
| 20751750 | rs28360636 |
| 20752162 | rs6932676 |
| 20752183 | rs9460539 |
| 20752303 | rs6932876 |
| 20752346 | rs34929755 |
| 20752359 | rs28733367 |
| 20752360 | rs11348111 |
| 20752473 | rs6933219 |
| 20752702 | rs6933165 |
| 20752872 | rs35255583 |
| 20752901 | rs34332316 |
| 20752923 | rs4710939 |
| 20753486 | rs11970417 |
| 20753659 | rs11963217 |
| 20753670 | rs11965473 |
| 20753833 | rs2876575 |
| 20754049 | rs7739974 |
| 20754976 | rs7745175 |
| 20755173 | rs7765784 |
| 20755178 | rs6907731 |
| 20755250 | rs35057896 |
| 20755313 | rs7746072 |
| 20755639 | rs10484632 |
| 20755770 | rs35444529 |
| 20755849 | rs17823571 |
| 20755941 | rs11965062 |
| 20755962 | rs6936199 |
| 20755965 | rs6913126 |
| 20756178 | rs6913509 |
| 20756613 | rs34638218 |
| 20756673 | rs35746011 |
| 20756741 | rs9460540 |
| 20757090 | rs36045545 |
| 20757129 | rs35392790 |
| 20757233 | rs6456364 |
| 20757260 | rs2179553 |
| 20757513 | rs9350269 |
| 20757531 | rs9465854 |
| 20757787 | rs2179552 |
| 20757936 | rs6925233 |
| 20758033 | rs2328532 |
| 20758248 | rs7743970 |
| 20758317 | rs13209457 |
| 20758318 | rs34641285 |
| 20758344 | rs13209538 |
| 20758387 | rs2876576 |
| 20758479 | rs13209572 |
| 20758653 | rs28783153 |
| 20758712 | rs9969037 |
| 20758800 | rs7766844 |
| 20758969 | rs7767133 |
| 20759291 | rs7749464 |
| 20760100 | rs2050225 |
| 20760696 | rs9295474 |
| 20760744 | rs9295475 |
| 20761529 | rs2328545 |
| 20761548 | rs28846771 |
| 20761779 | rs2876582 |
| 20761899 | rs10223446 |
| 20762094 | rs13219723 |
| 20762095 | rs13203489 |
| 20762154 | rs13203583 |
| 20762172 | rs13203887 |
| 20762279 | rs6456366 |
| 20762876 | rs9358355 |
| 20763089 | rs9368216 |
| 20763375 | rs9465855 |
| 20763384 | rs9465856 |
| 20763463 | rs16884070 |
| 20763482 | rs16884072 |
| 20763647 | rs13208604 |
| 20764169 | rs9465857 |
| 20764307 | rs9368217 |
| 20764559 | rs9460541 |
| 20764746 | rs9460542 |
| 20764765 | rs11969955 |
| 20764779 | rs4712522 |
| 20764924 | rs16884074 |
| 20765172 | rs34489684 |
| 20765324 | rs2328546 |
| 20765543 | rs4712523 |
| 20765844 | rs4712524 |
| 20765898 | rs35397753 |
| 20765991 | rs4710940 |
| 20766197 | rs13190727 |
| 20766215 | rs35136485 |
| 20766311 | rs35260725 |
| 20766335 | rs35939620 |
| 20766566 | rs17823996 |
| 20766713 | rs16884082 |
| 20767438 | rs6906327 |
| 20767566 | rs6456367 |
| 20767785 | rs6456368 |
| 20768202 | rs7749083 |
| 20768344 | rs6456369 |
| 20768669 | rs10946396 |
| 20768672 | rs10946397 |
| 20768710 | rs11759505 |
| 20769000 | rs13203361 |
| 20769013 | rs10946398 |
| 20769122 | rs7774594 |
| 20769229 | rs7754840 |
| 20769249 | rs9460543 |
| 20769508 | rs9460544 |
| 20769529 | rs9460545 |
| 20769711 | rs2206740 |
| 20769806 | rs5874779 |
| 20769807 | rs33970890 |
| 20769815 | rs5874780 |
| 20769816 | rs35014292 |
| 20769816 | rs35363501 |
| 20770092 | rs6456370 |
| 20770102 | rs979614 |
| 20770196 | rs35456723 |
| 20770571 | rs9368218 |
| 20770945 | rs4712525 |
| 20771014 | rs4712526 |
| 20771314 | rs4712527 |
| 20771442 | rs35191644 |
| 20771443 | rs34470647 |
| 20771611 | rs9460546 |
| 20771938 | rs9465859 |
| 20772079 | rs9465860 |
| 20772291 | rs736425 |
| 20772508 | rs3060776 |
| 20772509 | rs34941928 |
| 20772512 | rs5874781 |
| 20772761 | rs35778487 |
| 20773060 | rs742642 |
| 20773305 | rs35248697 |
| 20773436 | rs11967127 |
| 20773528 | rs7748382 |
| 20773547 | rs9688549 |
| 20773548 | rs9689351 |
| 20773570 | rs28665000 |
| 20773886 | rs7752236 |
| 20773925 | rs7772603 |
| 20774001 | rs7752780 |
| 20774034 | rs7752906 |
| 20774160 | rs34184260 |
| 20774223 | rs2206739 |
| 20774225 | rs2206738 |
| 20774250 | rs2206737 |
| 20774436 | rs11970425 |
| 20774484 | rs36034806 |
| 20774899 | rs35042364 |
| 20775218 | rs35540121 |
| 20775361 | rs9358356 |
| 20775667 | rs9356743 |
| 20775778 | rs9350270 |
| 20776366 | rs34929853 |
| 20778035 | rs34971765 |
| 20778443 | rs11970596 |
| 20779261 | rs12527373 |
| 20779367 | rs35916847 |
| 20780262 | rs11968224 |
| 20780271 | rs11968225 |
| 20780276 | rs9465861 |
| 20780296 | rs11968264 |
| 20780406 | rs12189849 |
| 20780413 | rs12209627 |
| 20780432 | rs12189895 |
| 20780855 | rs11968848 |
| 20781135 | rs11963945 |
| 20781601 | rs35677128 |
| 20781859 | rs7451008 |
| 20782670 | rs9368219 |
| 20782790 | rs1012636 |
| 20782945 | rs13217846 |
| 20783274 | rs1012635 |
| 20783700 | rs35665197 |
| 20783771 | rs35261542 |
| 20783828 | rs28823314 |
| 20783899 | rs28890810 |
| 20784051 | rs28871991 |
| 20784393 | rs34499031 |
| 20784650 | rs13208763 |
| 20784747 | rs28719685 |
| 20784789 | rs28856096 |
| 20785042 | rs11961863 |
| 20785211 | rs17824302 |
| 20785289 | rs12660618 |
| 20786302 | rs11371206 |
| 20786303 | rs34152621 |
| 20786409 | rs4712528 |
| 20786463 | rs13217082 |
| 20786470 | rs13217085 |
| 20786481 | rs13217090 |
| 20786483 | rs13217091 |
| 20786523 | rs13200946 |
| 20786772 | rs11968032 |
| 20786954 | rs9465863 |
| 20787289 | rs1569699 |
| 20787386 | rs34168173 |
| 20787688 | rs7756992 |
| 20788045 | rs35312717 |
| 20788657 | rs9348441 |
| 20788843 | rs9368220 |
| 20788941 | rs6931254 |
| 20789327 | rs6911742 |
| 20790601 | rs35612982 |
| 20791039 | rs35816514 |
| 20791123 | rs34612860 |
| 20791143 | rs9350271 |
| 20791162 | rs35657899 |
| 20791179 | rs11364854 |
| 20791249 | rs9460547 |
| 20791646 | rs16884103 |
| 20791961 | rs2206736 |
| 20793465 | rs9356744 |
| 20794295 | rs34987372 |
| 20794427 | rs36005020 |
| 20794552 | rs7766070 |
| 20794975 | rs9368222 |
| 20795290 | rs35566695 |
| 20795781 | rs10440832 |
| 20796100 | rs10440833 |
| 20796237 | rs35747076 |
| 20796578 | rs6900217 |
| 20797102 | rs34433496 |
| 20797924 | rs7748720 |
| 20797928 | rs34175709 |
| 20798290 | rs6911357 |
| 20800493 | rs12200791 |
| 20800955 | rs5874782 |
| 20800957 | rs36119385 |
| 20801341 | rs13219682 |
| 20802207 | rs4710941 |
| 20802270 | rs4620109 |
| 20802272 | rs28459626 |
| 20802273 | rs4712529 |
| 20802292 | rs10577753 |
| 20802504 | rs2223683 |
| 20802573 | rs2206735 |
| 20802863 | rs2206734 |
| 20802910 | rs34530846 |
| 20803458 | rs16884131 |
| 20804127 | rs10806921 |
| 20805104 | rs16884133 |
| 20805571 | rs17824500 |
| 20805652 | rs10946401 |
| 20806114 | rs16884135 |
| 20806582 | rs35711395 |
| 20807220 | rs11969783 |
| 20807364 | rs16884137 |
| 20808600 | rs11970626 |
| 20809092 | rs12190713 |
| 20809106 | rs11398905 |
| 20809415 | rs11961445 |
| 20809470 | rs35982077 |
| 20809486 | rs11305935 |
| 20810952 | rs9356745 |
| 20811700 | rs35043644 |
| 20811842 | rs16884140 |
| 20811931 | rs6931514 |
| 20812147 | rs35443650 |
| 20813281 | rs34671712 |
| 20813569 | rs11753081 |
| 20814081 | rs7739516 |
| 20814209 | rs6901559 |
| 20815176 | rs13196379 |
| 20815177 | rs13212234 |
| 20816204 | rs10536170 |
| 20817155 | rs9465869 |
| 20817688 | rs36070002 |
| 20818288 | rs17226450 |
| 20818905 | rs1073247 |
| 20819131 | rs9465870 |
| 20819386 | rs17226492 |
| 20819433 | rs13213613 |
| 20819567 | rs16884146 |
| 20819958 | rs2206733 |
| 20820440 | rs3749925 |
| 20821121 | rs9460548 |
| 20821619 | rs9460549 |
| 20821685 | rs1040558 |
| 20821893 | rs4712530 |
| 20822083 | rs35629277 |
| 20822362 | rs7451928 |
| 20822445 | rs6456371 |
| 20822589 | rs13220116 |
| 20822823 | rs2206732 |
| 20823169 | rs2179633 |
| 20823483 | rs11963770 |
| 20823805 | rs10946402 |
| 20823840 | rs4712531 |
| 20824098 | rs35738288 |
| 20824232 | rs9295478 |
| 20824549 | rs2328547 |
| 20824763 | rs3060781 |
| 20824764 | rs34686252 |
| 20824856 | rs13215905 |
| 20824884 | rs9368223 |
| 20824937 | rs2328548 |
| 20825025 | rs11427712 |
| 20825074 | rs6935599 |
| 20825100 | rs13216165 |
| 20825234 | rs9465871 |
| 20825383 | rs10946403 |
| 20826219 | rs2328549 |
| 20826449 | rs17226774 |
| 20827124 | rs9358357 |
| 20827211 | rs9368224 |
| 20827321 | rs11756271 |
| 20827372 | rs9358358 |
| 20827540 | rs9460550 |
| 20827858 | rs12110493 |
| 20827866 | rs12193125 |
| 20828258 | rs9356746 |
| 20828797 | rs9350272 |
| 20829322 | rs13219444 |
| 20829342 | rs12111216 |
| 20829562 | rs9350273 |
| 20829700 | rs9368225 |
| 20830399 | rs17825025 |
| 20831036 | rs9368226 |
| 20832213 | rs6903175 |
| 20832229 | rs6903744 |
| 20832537 | rs12111351 |
| 20832754 | rs4712536 |
| 20832986 | rs9356747 |
| 20833076 | rs9356748 |
| 20833219 | rs7767391 |
| 20833402 | rs7747752 |
| 20833511 | rs9350274 |
| 20833853 | rs34170041 |
| 20833919 | rs6915155 |
| 20834014 | rs6914868 |
| 20834472 | rs4538697 |
| 20835549 | rs4712537 |
| 20836048 | rs34097377 |
| 20836492 | rs6928012 |
| 20836710 | rs6908425 |
| 20837083 | rs28713236 |
| 20837085 | rs28570482 |
| 20837098 | rs35373857 |
| 20837704 | rs35980729 |
| 20838039 | rs9465872 |
| 20838780 | rs9942459 |
| 20838787 | rs9942429 |
| 20838794 | rs12197544 |
| 20839053 | rs34407999 |
| 20839503 | rs7741604 |
| 20840493 | rs35573456 |
| 20840651 | rs35633408 |
| 20841368 | rs9465873 |
| 20841433 | rs12211466 |
| 20842055 | rs4712538 |
| 20842055 | rs41413744 |
| 20842592 | rs13213979 |
| 20842973 | rs13217519 |
| 20843073 | rs34169067 |
| 20843074 | rs34773978 |
| 20843075 | rs34104100 |
| 20843076 | rs34790688 |
| 20843373 | rs11753021 |
| 20843473 | rs11753041 |
| 20843666 | rs6938978 |
| 20844017 | rs9295479 |
| 20844137 | rs10484631 |
| 20844216 | rs9358361 |
| 20844796 | rs9688471 |
| 20844922 | rs7744943 |
| 20844996 | rs9295480 |
| 20845006 | rs9368227 |
| 20845333 | rs9465874 |
| 20845619 | rs9358362 |
| 20845736 | rs9368228 |
| 20845737 | rs17839864 |
| 20845816 | rs34427164 |
| 20846388 | rs9368229 |
| 20846555 | rs9295481 |
| 20846750 | rs9368230 |
| 20846907 | rs9356750 |
| 20846992 | rs9366359 |
| 20847710 | rs41272361 |
| 20847886 | rs4710942 |
| 20847986 | rs9465875 |
| 20847998 | rs4710943 |
| 20848275 | rs9350276 |
| 20848458 | rs9465876 |
| 20848854 | rs34604097 |
| 20849362 | rs9688952 |
| 20849423 | rs34933309 |
| 20849720 | rs9350277 |
| 20850306 | rs13204257 |
| 20850665 | rs35106298 |
| 20851446 | rs13194434 |
| 20851931 | rs9358363 |
| 20852254 | rs34929383 |
| 20852706 | rs4710944 |
| 20852720 | rs12529627 |
| 20853053 | rs4710945 |
| 20853694 | rs9350278 |
| 20854215 | rs7738382 |
| 20854499 | rs1474739 |
| 20854568 | rs35432334 |
| 20854686 | rs16884185 |
| 20855689 | rs35345966 |
| 20856120 | rs2745928 |
| 20856124 | rs2745927 |
| 20856166 | rs2473649 |
| 20856236 | rs13215601 |
| 20856258 | rs35386836 |
| 20856392 | rs2819991 |
| 20856706 | rs2819995 |
| 20856744 | rs11752936 |
| 20856762 | rs1543364 |
| 20856765 | rs13203288 |
| 20856879 | rs2473648 |
| 20856966 | rs2473647 |
| 20856967 | rs2473646 |
| 20857002 | rs1543363 |
| 20857270 | rs16884190 |
| 20857656 | rs4644038 |
| 20858009 | rs2819988 |
| 20858162 | rs4475313 |
| 20858693 | rs2745933 |
| 20858865 | rs9460552 |
| 20859007 | rs9460553 |
| 20859031 | rs9350279 |
| 20859173 | rs10568019 |
| 20859187 | rs10598071 |
| 20859199 | rs12199271 |
| 20859220 | rs7765413 |
| 20859294 | rs7747724 |
| 20859674 | rs11756862 |
| 20859995 | rs4712539 |
| 20860175 | rs2819989 |
| 20860310 | rs2819990 |
| 20860358 | rs9465877 |
| 20860448 | rs34996587 |
| 20860459 | rs34744837 |
| 20861169 | rs2819992 |
| 20861420 | rs10946405 |
| 20861729 | rs2745932 |
| 20861866 | rs2819993 |
| 20861911 | rs9465879 |
| 20861915 | rs2819994 |
| 20861965 | rs34164249 |
| 20862098 | rs2493995 |
| 20862168 | rs35908672 |
| 20862169 | rs35525968 |
| 20862500 | rs9358364 |
| 20862605 | rs2745931 |
| 20862716 | rs2745930 |
| 20862740 | rs2745929 |
| 20863911 | rs2819996 |
| 20864423 | rs1012114 |
| 20864756 | rs2473651 |
| 20864955 | rs16901574 |
| 20865045 | rs9358365 |
| 20865049 | rs6913831 |
| 20865061 | rs10652206 |
| 20865064 | rs10652207 |
| 20865081 | rs11281792 |
| 20865121 | rs6934147 |
| 20865127 | rs13200804 |
| 20865134 | rs6934300 |
| 20865135 | rs2473650 |
| 20865136 | rs6913999 |
| 20865625 | rs9465880 |
| 20866301 | rs34767457 |
| 20866310 | rs35499882 |
| 20866326 | rs2819997 |
| 20866505 | rs2819998 |
| 20866612 | rs2819999 |
| 20866683 | rs2820000 |
| 20866739 | rs10946406 |
| 20866788 | rs34291318 |
| 20866813 | rs9465881 |
| 20866829 | rs9465882 |
| 20866922 | rs2820001 |
| 20867101 | rs2820002 |
| 20867254 | rs9350280 |
| 20867255 | rs9356751 |
| 20868156 | rs6909558 |
| 20868333 | rs11415727 |
| 20868591 | rs6910326 |
| 20869059 | rs12664021 |
| 20869213 | rs9295482 |
| 20869314 | rs9465883 |
| 20869658 | rs9465884 |
| 20870485 | rs4437452 |
| 20871061 | rs34235608 |
| 20871150 | rs4712540 |
| 20871244 | rs35450702 |
| 20872229 | rs4351239 |
| 20872296 | rs12190376 |
| 20872578 | rs4710946 |
| 20873095 | rs9465885 |
| 20873251 | rs12192328 |
| 20873365 | rs10536083 |
| 20873375 | rs36210777 |
| 20873677 | rs6149469 |
| 20874082 | rs4585542 |
| 20874261 | rs7751154 |
| 20874410 | rs9356752 |
| 20874676 | rs4510656 |
| 20875450 | rs7452218 |
| 20875680 | rs4320364 |
| 20876442 | rs9465886 |
| 20876454 | rs6926436 |
| 20877469 | rs34127549 |
| 20877508 | rs13214325 |
| 20877580 | rs7750031 |
| 20877596 | rs34801493 |
| 20877974 | rs7772390 |
| 20878762 | rs6900375 |
| 20879028 | rs4712541 |
| 20879194 | rs4710947 |
| 20879323 | rs34893361 |
| 20879617 | rs35938647 |
| 20879690 | rs9465887 |
| 20879842 | rs9368232 |
| 20879939 | rs9350281 |
| 20880120 | rs4132894 |
| 20880272 | rs12195490 |
| 20880344 | rs9465888 |
| 20880553 | rs6941727 |
| 20880797 | rs4712542 |
| 20880982 | rs6923622 |
| 20881085 | rs4712543 |
| 20881539 | rs9358366 |
| 20881700 | rs12211178 |
| 20881731 | rs7453352 |
| 20881858 | rs6929760 |
| 20881957 | rs6929918 |
| 20882292 | rs9465889 |
| 20882389 | rs4712545 |
| 20882575 | rs12193239 |
| 20882738 | rs9465890 |
| 20883200 | rs6936300 |
| 20883430 | rs6936840 |
| 20883639 | rs4493738 |
| 20883701 | rs11965197 |
| 20884250 | rs6899514 |
| 20884329 | rs6456372 |
| 20885570 | rs34358176 |
| 20886538 | rs9465891 |
| 20886687 | rs10946407 |
| 20887060 | rs35581397 |
| 20887457 | rs9358367 |
| 20887829 | rs4076137 |
| 20888220 | rs13207747 |
| 20888221 | rs13208059 |
| 20888329 | rs4074058 |
| 20888884 | rs9350282 |
| 20888894 | rs3193655 |
| 20888919 | rs11545101 |
| 20888919 | rs3193654 |
| 20889269 | rs12199265 |
| 20889428 | rs34113701 |
| 20889600 | rs41272363 |
| 20890872 | rs4478387 |
| 20891222 | rs11752139 |
| 20891373 | rs6910215 |
| 20891415 | rs10690190 |
| 20891417 | rs10647996 |
| 20891638 | rs10946408 |
| 20892162 | rs4710949 |
| 20892292 | rs4712547 |
| 20892371 | rs4712548 |
| 20892641 | rs10550462 |
| 20892642 | rs34372186 |
| 20893118 | rs9368234 |
| 20893144 | rs9348443 |
| 20893525 | rs5874783 |
| 20893543 | rs11344905 |
| 20893671 | rs12210335 |
| 20893673 | rs12210336 |
| 20893711 | rs6902757 |
| 20893943 | rs6456373 |
| 20894027 | rs6927892 |
| 20894054 | rs13210599 |
| 20894573 | rs34563996 |
| 20895285 | rs35066145 |
| 20897153 | rs10498698 |
| 20898456 | rs10806923 |
| 20898488 | rs10806924 |
| 20898554 | rs9368235 |
| 20898785 | rs9368236 |
| 20898787 | rs9368237 |
| 20899054 | rs9356753 |
| 20899265 | rs12111182 |
| 20899490 | rs11752772 |
| 20899516 | rs12661145 |
| 20899518 | rs12665631 |
| 20899691 | rs12208378 |
| 20900193 | rs34964322 |
| 20901075 | rs34430352 |
| 20901172 | rs4299818 |
| 20901416 | rs4495263 |
| 20901588 | rs7773697 |
| 20901981 | rs10684385 |
| 20901982 | rs33967126 |
| 20902035 | rs34171574 |
| 20902604 | rs9460557 |
| 20902661 | rs4492184 |
| 20902662 | rs35025612 |
| 20903233 | rs10946409 |
| 20903353 | rs9295483 |
| 20903427 | rs11751916 |
| 20904975 | rs36025037 |
| 20905571 | rs7738948 |
| 20905701 | rs9465892 |
| 20905830 | rs12211211 |
| 20906051 | rs11968853 |
| 20906198 | rs10456236 |
| 20906864 | rs4712549 |
| 20906867 | rs36194803 |
| 20906929 | rs4712550 |
| 20907200 | rs12213259 |
| 20907404 | rs4496779 |
| 20907564 | rs34432145 |
| 20907866 | rs9358368 |
| 20908149 | rs9368238 |
| 20908386 | rs4524599 |
| 20908448 | rs9465893 |
| 20908614 | rs12215519 |
| 20909486 | rs5874784 |
| 20909709 | rs34686362 |
| 20910367 | rs9460558 |
| 20910522 | rs12664231 |
| 20910525 | rs10946410 |
| 20911078 | rs4710950 |
| 20911442 | rs9465894 |
| 20911666 | rs6925153 |
| 20911791 | rs6456374 |
| 20912542 | rs9465895 |
| 20913109 | rs4712551 |
| 20913695 | rs4607421 |
| 20913962 | rs4293992 |
| 20913984 | rs9460559 |
| 20914140 | rs6942203 |
| 20914465 | rs12189708 |
| 20914479 | rs12660120 |
| 20914641 | rs9465897 |
| 20914675 | rs9465898 |
| 20915109 | rs6905567 |
| 20915183 | rs4580857 |
| 20915571 | rs12191782 |
| 20915711 | rs9358370 |
| 20916218 | rs7748911 |
| 20916690 | rs9350287 |
| 20916721 | rs9356754 |
| 20917615 | rs12525153 |
| 20917713 | rs6456375 |
| 20917744 | rs35117828 |
| 20917773 | rs9348444 |
| 20917833 | rs9465899 |
| 20917869 | rs6902880 |
| 20918009 | rs11756029 |
| 20918026 | rs9348445 |
| 20918173 | rs9358371 |
| 20918193 | rs9348446 |
| 20918577 | rs5874785 |
| 20918578 | rs35532605 |
| 20918604 | rs5874786 |
| 20918655 | rs5874787 |
| 20918710 | rs4373348 |
| 20918839 | rs11966932 |
| 20918888 | rs35887473 |
| 20918888 | rs34206478 |
| 20919070 | rs4330530 |
| 20919476 | rs13198015 |
| 20919599 | rs9465900 |
| 20919670 | rs9465901 |
| 20919827 | rs4710951 |
| 20919932 | rs4710952 |
| 20920567 | rs9358372 |
| 20920927 | rs9358373 |
| 20920992 | rs9368239 |
| 20921191 | rs12192912 |
| 20921354 | rs7747989 |
| 20921744 | rs7752140 |
| 20921896 | rs7752310 |
| 20922035 | rs4710953 |
| 20922189 | rs12662683 |
| 20922395 | rs9465902 |
| 20922954 | rs35040258 |
| 20922958 | rs4710954 |
| 20923239 | rs12661606 |
| 20923432 | rs10946411 |
| 20923828 | rs36037446 |
| 20924516 | rs12212105 |
| 20925219 | rs12213857 |
| 20925336 | rs12213956 |
| 20925602 | rs10946412 |
| 20925812 | rs11759888 |
| 20925967 | rs9366360 |
| 20926372 | rs12665734 |
| 20926457 | rs4712552 |
| 20926516 | rs9465903 |
| 20926518 | rs4712553 |
| 20927237 | rs12190831 |
| 20927408 | rs4334968 |
| 20927432 | rs34237934 |
| 20927804 | rs11961950 |
| 20927955 | rs10806925 |
| 20929468 | rs7753109 |
| 20929641 | rs9465904 |
| 20929820 | rs6931788 |
| 20929846 | rs11304808 |
| 20930110 | rs9465905 |
| 20930489 | rs9465906 |
| 20930866 | rs9465907 |
| 20931188 | rs9465908 |
| 20931485 | rs6938757 |
| 20931735 | rs6923264 |
| 20931827 | rs6900509 |
| 20932202 | rs9460561 |
| 20932400 | rs10946413 |
| 20932413 | rs9465909 |
| 20932808 | rs9460562 |
| 20932809 | rs9465910 |
| 20932976 | rs10806926 |
| 20932997 | rs9460563 |
| 20933029 | rs9465911 |
| 20933338 | rs11404073 |
| 20933339 | rs34199366 |
| 20933375 | rs4712554 |
| 20933483 | rs4712555 |
| 20933542 | rs35893096 |
| 20933749 | rs34260649 |
| 20933990 | rs9465912 |
| 20934012 | rs9368240 |
| 20934024 | rs9368241 |
| 20934147 | rs9368242 |
| 20934260 | rs4395717 |
| 20934729 | rs12209113 |
| 20935051 | rs4446563 |
| 20935807 | rs9348447 |
| 20936313 | rs10806927 |
| 20936483 | rs35622501 |
| 20936512 | rs12211426 |
| 20936992 | rs9368243 |
| 20937713 | rs7768091 |
| 20937751 | rs9356755 |
| 20938085 | rs6917412 |
| 20938283 | rs12215186 |
| 20938644 | rs9348448 |
| 20938681 | rs10570889 |
| 20938807 | rs34799469 |
| 20938927 | rs12190001 |
| 20938932 | rs9465913 |
| 20939133 | rs35997536 |
| 20939251 | rs6901110 |
| 20939636 | rs35810189 |
| 20940144 | rs9368244 |
| 20940807 | rs9350288 |
| 20940851 | rs12210496 |
| 20941839 | rs4568440 |
| 20942876 | rs11964964 |
| 20943072 | rs28616599 |
| 20943192 | rs12201027 |
| 20943239 | rs12194825 |
| 20943369 | rs34826961 |
| 20943724 | rs5874788 |
| 20943727 | rs34317828 |
| 20944192 | rs9465914 |
| 20944328 | rs9465915 |
| 20946192 | rs10592623 |
| 20946236 | rs7749282 |
| 20946253 | rs7769205 |
| 20946255 | rs7749021 |
| 20946263 | rs7769209 |
| 20946275 | rs7749034 |
| 20946424 | rs9295484 |
| 20946948 | rs6456377 |
| 20947467 | rs34883184 |
| 20948065 | rs9465917 |
| 20948650 | rs12210803 |
| 20948719 | rs4345385 |
| 20949055 | rs7764980 |
| 20949271 | rs7765060 |
| 20949459 | rs11967047 |
| 20949540 | rs35943413 |
| 20949572 | rs11967068 |
| 20949779 | rs9460564 |
| 20950591 | rs9465919 |
| 20950992 | rs35578988 |
| 20951013 | rs35344109 |
| 20952130 | rs9356756 |
| 20952309 | rs34656248 |
| 20952720 | rs17234378 |
| 20952897 | rs35541206 |
| 20952909 | rs9366361 |
| 20952918 | rs35392381 |
| 20953053 | rs7751431 |
| 20953657 | rs4336435 |
| 20953903 | rs36065886 |
| 20954005 | rs6920045 |
| 20954509 | rs6941354 |
| 20954569 | rs6941809 |
| 20954582 | rs6941508 |
| 20954975 | rs6903445 |
| 20955367 | rs7742468 |
| 20956366 | rs11417705 |
| 20956496 | rs11281215 |
| 20956497 | rs28533427 |
| 20956497 | rs9295485 |
| 20956498 | rs28588709 |
| 20956498 | rs34068549 |
| 20956738 | rs34367682 |
| 20957391 | rs7753271 |
| 20957513 | rs9460566 |
| 20957564 | rs34204682 |
| 20957693 | rs11962974 |
| 20957793 | rs35879846 |
| 20957849 | rs6921177 |
| 20958215 | rs6456378 |
| 20958359 | rs12663147 |
| 20958432 | rs13192874 |
| 20958637 | rs11294607 |
| 20958873 | rs7763216 |
| 20959240 | rs5874789 |
| 20959954 | rs11966086 |
| 20960487 | rs13208940 |
| 20960793 | rs4397214 |
| 20961601 | rs6908918 |
| 20961601 | rs34610909 |
| 20961612 | rs6908922 |
| 20961613 | rs6908779 |
| 20962352 | rs7771981 |
| 20962545 | rs9465921 |
| 20962984 | rs4236002 |
| 20963120 | rs4236003 |
| 20963450 | rs9350289 |
| 20963535 | rs9350290 |
| 20963662 | rs10558810 |
| 20963671 | rs10608340 |
| 20963756 | rs7761330 |
| 20963966 | rs13209243 |
| 20964017 | rs35463313 |
| 20964570 | rs12211628 |
| 20964682 | rs4712556 |
| 20965069 | rs4710955 |
| 20965780 | rs34481273 |
| 20965904 | rs16884229 |
| 20966215 | rs11965417 |
| 20966239 | rs4712558 |
| 20966335 | rs4712559 |
| 20966543 | rs34865786 |
| 20966637 | rs17826361 |
| 20966708 | rs9366362 |
| 20966831 | rs9366363 |
| 20967583 | rs11966194 |
| 20968463 | rs12524318 |
| 20968532 | rs28412953 |
| 20968698 | rs9358374 |
| 20968821 | rs9350291 |
| 20968927 | rs9350292 |
| 20969034 | rs9350293 |
| 20969131 | rs16901585 |
| 20969205 | rs5007237 |
| 20969579 | rs4383807 |
| 20969839 | rs4330531 |
| 20970113 | rs12662176 |
| 20970590 | rs9356757 |
| 20970850 | rs34873202 |
| 20970884 | rs9368245 |
| 20970889 | rs34101377 |
| 20971175 | rs12201353 |
| 20971267 | rs12214755 |
| 20972435 | rs11269210 |
| 20973576 | rs9465922 |
| 20973608 | rs12191898 |
| 20973841 | rs35647500 |
| 20973851 | rs5874790 |
| 20973852 | rs35372258 |
| 20974027 | rs7749397 |
| 20974223 | rs7454011 |
| 20974715 | rs35244703 |
| 20974836 | rs9368246 |
| 20974986 | rs34062103 |
| 20975000 | rs35487666 |
| 20975596 | rs16884250 |
| 20975795 | rs10554344 |
| 20976043 | rs4370355 |
| 20976276 | rs10588127 |
| 20976276 | rs34382383 |
| 20977459 | rs7766346 |
| 20977678 | rs7766681 |
| 20977795 | rs7770607 |
| 20978072 | rs9350294 |
| 20978617 | rs9350295 |
| 20978719 | rs9366364 |
| 20978768 | rs34447575 |
| 20978882 | rs9366365 |
| 20980033 | rs35505816 |
| 20980715 | rs34113324 |
| 20981367 | rs10946414 |
| 20981827 | rs4645410 |
| 20982392 | rs16884254 |
| 20982541 | rs13197235 |
| 20983561 | rs9350296 |
| 20983706 | rs9368247 |
| 20984381 | rs12665789 |
| 20984489 | rs4077403 |
| 20984592 | rs4077404 |
| 20984662 | rs4077405 |
| 20984894 | rs4529295 |
| 20984917 | rs35499986 |
| 20985132 | rs4618519 |
| 20985379 | rs36002274 |
| 20985482 | rs9368248 |
| 20985514 | rs7742802 |
| 20985952 | rs4236004 |
| 20986261 | rs4712561 |
| 20986658 | rs9358375 |
| 20986864 | rs12194583 |
| 20986999 | rs34923632 |
| 20987073 | rs4537128 |
| 20987237 | rs4626408 |
| 20987282 | rs12194813 |
| 20987312 | rs6915746 |
| 20988259 | rs9350297 |
| 20988570 | rs12210231 |
| 20988821 | rs12198243 |
| 20989365 | rs10946415 |
| 20989383 | rs7763704 |
| 20989596 | rs10589878 |
| 20990154 | rs9465923 |
| 20990216 | rs9465924 |
| 20990238 | rs9460567 |
| 20990523 | rs9465925 |
| 20990820 | rs4612156 |
| 20991595 | rs10946416 |
| 20992059 | rs4546483 |
| 20992198 | rs9460569 |
| 20993260 | rs35314952 |
| 20993493 | rs4131667 |
| 20993700 | rs4334975 |
| 20993732 | rs4272215 |
| 20994113 | rs11756312 |
| 20994209 | rs6926335 |
| 20995305 | rs9366367 |
| 20995933 | rs6938256 |
| 20996432 | rs6938755 |
| 20996580 | rs9465928 |
| 20996674 | rs7773564 |
| 20996730 | rs28843472 |
| 20996731 | rs12210522 |
| 20996732 | rs13191608 |
| 20996755 | rs10806929 |
| 20996858 | rs9268994 |
| 20996859 | rs9268995 |
| 20996890 | rs28895225 |
| 20996905 | rs9268996 |
| 20996911 | rs9268997 |
| 20996937 | rs35328517 |
| 20996973 | rs9268998 |
| 20997216 | rs12055384 |
| 20997384 | rs12055630 |
| 20997540 | rs4947247 |
| 20998096 | rs9460571 |
| 20998693 | rs10946417 |
| 20998950 | rs11752477 |
| 20999169 | rs6913323 |
| 20999294 | rs12194053 |
| 20999407 | rs36009649 |
| 20999752 | rs10594050 |
| 20999763 | rs5874791 |
| 20999764 | rs35553046 |
| 20999770 | rs4363020 |
| 21000241 | rs10456008 |
| 21000447 | rs35294796 |
| 21000702 | rs9368251 |
| 21001068 | rs6916036 |
| 21001199 | rs34587331 |
| 21001226 | rs6456379 |
| 21001338 | rs9368252 |
| 21001683 | rs4626415 |
| 21002414 | rs5874792 |
| 21002415 | rs33925173 |
| 21002601 | rs5874793 |
| 21002602 | rs34720816 |
| 21002625 | rs12528945 |
| 21004281 | rs12206985 |
| 21004330 | rs11757050 |
| 21004330 | rs34449480 |
| 21004372 | rs9368253 |
| 21004532 | rs9295486 |
| 21004540 | rs9465930 |
| 21004836 | rs12208719 |
| 21005635 | rs6918643 |
| 21005875 | rs4712562 |
| 21006091 | rs6456380 |
| 21006119 | rs7759877 |
| 21006228 | rs9358376 |
| 21006412 | rs35620702 |
| 21006800 | rs28665148 |
| 21006801 | rs28599540 |
| 21007273 | rs35400949 |
| 21007751 | rs12153898 |
| 21007819 | rs12154181 |
| 21007863 | rs12154184 |
| 21008099 | rs9368254 |
| 21008207 | rs9368255 |
| 21009058 | rs35958016 |
| 21009211 | rs6456381 |
| 21009246 | rs7758467 |
| 21009290 | rs6456382 |
| 21009421 | rs12153811 |
| 21009654 | rs7739013 |
| 21009711 | rs6922768 |
| 21009716 | rs13191241 |
| 21009909 | rs10640864 |
| 21009910 | rs34135721 |
| 21010295 | rs34154507 |
| 21010566 | rs35386545 |
| 21010568 | rs9368256 |
| 21010964 | rs7740094 |
| 21010972 | rs7758155 |
| 21011220 | rs9366368 |
| 21011950 | rs9358377 |
| 21012344 | rs6908429 |
| 21012629 | rs4712563 |
| 21012910 | rs4710957 |
| 21013210 | rs12214079 |
| 21013433 | rs9460572 |
| 21013812 | rs2876583 |
| 21013840 | rs9465931 |
| 21014102 | rs34068097 |
| 21014657 | rs10080699 |
| 21014657 | rs9460573 |
| 21014665 | rs9460574 |
| 21014666 | rs6920918 |
| 21015860 | rs4413596 |
| 21016247 | rs6909682 |
| 21016278 | rs6910032 |
| 21016580 | rs1498427 |
| 21016869 | rs34713988 |
| 21017395 | rs9368257 |
| 21017439 | rs6939006 |
| 21017512 | rs17236121 |
| 21017740 | rs11753331 |
| 21018757 | rs12198263 |
| 21018818 | rs35916632 |
| 21018915 | rs9350298 |
| 21018982 | rs4710958 |
| 21019053 | rs35886192 |
| 21019304 | rs6456385 |
| 21019380 | rs7763674 |
| 21019654 | rs7746308 |
| 21020651 | rs12190631 |
| 21020765 | rs1498425 |
| 21021671 | rs13197106 |
| 21022074 | rs6913538 |
| 21022178 | rs9460575 |
| 21022308 | rs6914232 |
| 21022483 | rs9460576 |
| 21022485 | rs6914586 |
| 21022873 | rs9465932 |
| 21022913 | rs2030082 |
| 21022932 | rs9350299 |
| 21023379 | rs10536841 |
| 21023692 | rs9465933 |
| 21024540 | rs34537557 |
| 21025636 | rs9368258 |
| 21026230 | rs6938184 |
| 21026422 | rs6938415 |
| 21026611 | rs35678799 |
| 21028069 | rs28440883 |
| 21028102 | rs4712564 |
| 21028251 | rs4712565 |
| 21028711 | rs9366369 |
| 21028958 | rs9465934 |
| 21029390 | rs6913658 |
| 21030247 | rs7765765 |
| 21030707 | rs12662218 |
| 21030842 | rs9460577 |
| 21030852 | rs4712566 |
| 21030926 | rs4712567 |
| 21030962 | rs6936002 |
| 21031254 | rs9460578 |
| 21031561 | rs7771782 |
| 21032098 | rs2328550 |
| 21032169 | rs9465935 |
| 21033024 | rs9460579 |
| 21033247 | rs2328551 |
| 21033384 | rs34787947 |
| 21033907 | rs35164658 |
| 21034157 | rs34463604 |
| 21034296 | rs16884302 |
| 21034654 | rs13215945 |
| 21034664 | rs13215949 |
| 21034748 | rs13202822 |
| 21034750 | rs13203127 |
| 21034760 | rs13203130 |
| 21035097 | rs35259543 |
| 21035108 | rs10687937 |
| 21035135 | rs9358378 |
| 21035148 | rs9465936 |
| 21035989 | rs6903176 |
| 21036896 | rs34272344 |
| 21037020 | rs9366370 |
| 21037483 | rs7747830 |
| 21037954 | rs35552655 |
| 21038014 | rs11970010 |
| 21038092 | rs11970030 |
| 21038468 | rs35341341 |
| 21038480 | rs5874794 |
| 21038957 | rs34094116 |
| 21039069 | rs9366371 |
| 21039135 | rs7775995 |
| 21040473 | rs6916666 |
| 21041656 | rs16884308 |
| 21042140 | rs11395158 |
| 21042539 | rs34100038 |
| 21042991 | rs7768647 |
| 21043110 | rs35030815 |
| 21043259 | rs9350300 |
| 21044027 | rs10946419 |
| 21044393 | rs10946420 |
| 21044437 | rs9465937 |
| 21044451 | rs9368259 |
| 21044452 | rs33911838 |
| 21044591 | rs9358379 |
| 21044671 | rs9460580 |
| 21044802 | rs35523552 |
| 21044952 | rs16884310 |
| 21046071 | rs34741537 |
| 21046084 | rs12526287 |
| 21046565 | rs9465938 |
| 21046566 | rs28734969 |
| 21046585 | rs7747773 |
| 21046841 | rs9465939 |
| 21046900 | rs4144650 |
| 21047359 | rs4712569 |
| 21047690 | rs4712570 |
| 21047713 | rs6939250 |
| 21047725 | rs4710959 |
| 21047769 | rs4710960 |
| 21047987 | rs13191844 |
| 21048358 | rs12196595 |
| 21048740 | rs9350301 |
| 21049043 | rs9350302 |
| 21049105 | rs11964084 |
| 21049192 | rs6927419 |
| 21049306 | rs12198512 |
| 21049369 | rs11450413 |
| 21049370 | rs11409601 |
| 21049371 | rs33942714 |
| 21050175 | rs4307160 |
| 21050624 | rs4712571 |
| 21051211 | rs9465941 |
| 21051466 | rs10946423 |
| 21051483 | rs9465942 |
| 21051538 | rs34759060 |
| 21051555 | rs9295487 |
| 21051560 | rs5874795 |
| 21051561 | rs9358380 |
| 21051563 | rs12174149 |
| 21051568 | rs9350303 |
| 21051569 | rs9358381 |
| 21052585 | rs11279071 |
| 21052648 | rs9465943 |
| 21053046 | rs9465944 |
| 21053295 | rs10690950 |
| 21053629 | rs34879403 |
| 21053757 | rs7772447 |
| 21053935 | rs7773189 |
| 21054476 | rs34602677 |
| 21054784 | rs6901153 |
| 21054804 | rs11361870 |
| 21054805 | rs35132815 |
| 21055236 | rs12203876 |
| 21055710 | rs35896536 |
| 21055853 | rs34881630 |
| 21055870 | rs9368260 |
| 21056065 | rs9460581 |
| 21056280 | rs6930879 |
| 21056288 | rs6930887 |
| 21056329 | rs6931047 |
| 21056472 | rs9350305 |
| 21058200 | rs9350306 |
| 21058970 | rs12197923 |
| 21059112 | rs9460582 |
| 21059179 | rs5874796 |
| 21059866 | rs6922187 |
| 21060142 | rs9465945 |
| 21060180 | rs10558500 |
| 21060186 | rs11289942 |
| 21060212 | rs9348451 |
| 21060247 | rs6922914 |
| 21060361 | rs9358382 |
| 21060639 | rs35863080 |
| 21061204 | rs4631270 |
| 21061590 | rs2015426 |
| 21061898 | rs7749498 |
| 21062676 | rs9350307 |
| 21062805 | rs1004172 |
| 21063311 | rs7775523 |
| 21064970 | rs12199174 |
| 21066160 | rs9368261 |
| 21067118 | rs7738201 |
| 21067314 | rs17236645 |
| 21067986 | rs16884319 |
| 21068463 | rs9918311 |
| 21068617 | rs34769285 |
| 21068702 | rs12205321 |
| 21068911 | rs6456388 |
| 21069040 | rs4712572 |
| 21069650 | rs12524678 |
| 21069850 | rs13211056 |
| 21070260 | rs17236694 |
| 21070934 | rs35382761 |
| 21071276 | rs28612346 |
| 21071587 | rs6915209 |
| 21071832 | rs35420176 |
| 21072294 | rs6940952 |
| 21072419 | rs35708433 |
| 21072662 | rs6941808 |
| 21072828 | rs11966306 |
| 21073181 | rs16884325 |
| 21073468 | rs7743030 |
| 21073553 | rs28429494 |
| 21074417 | rs4712573 |
| 21074865 | rs11752712 |
| 21075695 | rs9460583 |
| 21075964 | rs9460584 |
| 21076612 | rs34425275 |
| 21076615 | rs1112255 |
| 21076815 | rs1112256 |
| 21077529 | rs9348452 |
| 21077563 | rs34660637 |
| 21077716 | rs7746754 |
| 21077794 | rs9350308 |
| 21078333 | rs34215872 |
| 21079005 | rs2030081 |
| 21079247 | rs6940649 |
| 21079554 | rs6941043 |
| 21079964 | rs6913817 |
| 21080948 | rs201361 |
| 21081289 | rs34479545 |
| 21081631 | rs1980458 |
| 21082363 | rs13210151 |
| 21082946 | rs34266759 |
| 21083025 | rs34192233 |
| 21083039 | rs11427521 |
| 21083189 | rs35882882 |
| 21083211 | rs11330322 |
| 21083279 | rs1997685 |
| 21083363 | rs10498699 |
| 21083433 | rs201360 |
| 21085426 | rs201359 |
| 21085653 | rs201358 |
| 21085834 | rs34764914 |
| 21087560 | rs201357 |
| 21088272 | rs4712575 |
| 21088448 | rs4712576 |
| 21088450 | rs4712577 |
| 21088472 | rs365001 |
| 21088891 | rs35829025 |
| 21089414 | rs383684 |
| 21089616 | rs444005 |
| 21090258 | rs35693086 |
| 21090584 | rs11961135 |
| 21091067 | rs201356 |
| 21091773 | rs201355 |
| 21091799 | rs12193382 |
| 21091799 | rs34391804 |
| 21091914 | rs35723307 |
| 21091926 | rs35448639 |
| 21093023 | rs35957348 |
| 21093317 | rs201354 |
| 21093369 | rs201353 |
| 21093439 | rs9350309 |
| 21093650 | rs201352 |
| 21094109 | rs201351 |
| 21094143 | rs35853352 |
| 21094861 | rs5874797 |
| 21094872 | rs5874798 |
| 21094931 | rs201350 |
| 21095014 | rs1997684 |
| 21095425 | rs4560627 |
| 21095482 | rs10699637 |
| 21095654 | rs7453577 |
| 21096171 | rs11300877 |
| 21096223 | rs201349 |
| 21096419 | rs10694429 |
| 21096428 | rs3223730 |
| 21096922 | rs201348 |
| 21097026 | rs35010661 |
| 21097035 | rs201347 |
| 21097047 | rs10701919 |
| 21097265 | rs201346 |
| 21098132 | rs6456389 |
| 21099063 | rs12528036 |
| 21099645 | rs201345 |
| 21099698 | rs13199754 |
| 21099916 | rs35504365 |
| 21100226 | rs35586782 |
| 21100247 | rs28418444 |
| 21100248 | rs13196975 |
| 21100263 | rs35586503 |
| 21100320 | rs9356759 |
| 21100418 | rs9366372 |
| 21100871 | rs10946424 |
| 21100949 | rs201344 |
| 21101200 | rs11964880 |
| 21101281 | rs11967584 |
| 21101513 | rs201343 |
| 21102229 | rs201342 |
| 21102283 | rs9350310 |
| 21102377 | rs9368262 |
| 21102486 | rs9368263 |
| 21103236 | rs35996844 |
| 21103858 | rs201341 |
| 21103935 | rs12660707 |
| 21104054 | rs201340 |
| 21104291 | rs201339 |
| 21105034 | rs396308 |
| 21105044 | rs4712578 |
| 21105348 | rs449233 |
| 21107366 | rs9358383 |
| 21107874 | rs36078234 |
| 21108856 | rs9368264 |
| 21109330 | rs9368265 |
| 21109358 | rs9368266 |
| 21109523 | rs9368267 |
| 21109553 | rs6940871 |
| 21109878 | rs35240009 |
| 21110331 | rs6914677 |
| 21110432 | rs9350311 |
| 21110750 | rs12195827 |
| 21110788 | rs9366373 |
| 21110864 | rs34620040 |
| 21111239 | rs9350312 |
| 21111513 | rs2038066 |
| 21111536 | rs13191651 |
| 21111581 | rs13191676 |
| 21111601 | rs2038065 |
| 21112269 | rs7744303 |
| 21112288 | rs7744317 |
| 21112352 | rs7762669 |
| 21113187 | rs6932336 |
| 21113252 | rs34002111 |
| 21113325 | rs6932393 |
| 21113687 | rs2839854 |
| 21114641 | rs9348453 |
| 21115599 | rs9358384 |
| 21115719 | rs9348454 |
| 21115720 | rs2473089 |
| 21115866 | rs2494728 |
| 21116011 | rs9358385 |
| 21116132 | rs1498426 |
| 21116718 | rs12192642 |
| 21116822 | rs13200036 |
| 21116851 | rs9368268 |
| 21116920 | rs7750463 |
| 21116972 | rs7750903 |
| 21117087 | rs374654 |
| 21117572 | rs6914615 |
| 21117867 | rs9465946 |
| 21118124 | rs364334 |
| 21118132 | rs416688 |
| 21118300 | rs11382802 |
| 21118547 | rs1548145 |
| 21118567 | rs201291 |
| 21119018 | rs9465947 |
| 21119021 | rs12197742 |
| 21119236 | rs201292 |
| 21119698 | rs35998561 |
| 21119973 | rs201293 |
| 21120225 | rs9358386 |
| 21120735 | rs35254358 |
| 21120883 | rs6905258 |
| 21121364 | rs201294 |
| 21121603 | rs201295 |
| 21121962 | rs7748932 |
| 21121994 | rs2494729 |
| 21122535 | rs3966676 |
| 21122822 | rs34834774 |
| 21122975 | rs16884376 |
| 21123408 | rs201308 |
| 21123602 | rs8180606 |
| 21123934 | rs201309 |
| 21123964 | rs201310 |
| 21124001 | rs6456390 |
| 21124511 | rs201311 |
| 21124822 | rs5874799 |
| 21124823 | rs34733906 |
| 21124830 | rs400514 |
| 21124882 | rs3061573 |
| 21125298 | rs16884385 |
| 21125848 | rs34894804 |
| 21125898 | rs9460586 |
| 21126295 | rs16884389 |
| 21126495 | rs35678732 |
| 21126634 | rs10946425 |
| 21127516 | rs6899729 |
| 21127744 | rs16884394 |
| 21128114 | rs36115181 |
| 21128124 | rs5874800 |
| 21128307 | rs34639729 |
| 21128419 | rs35260082 |
| 21128533 | rs7744010 |
| 21128746 | rs201290 |
| 21129343 | rs201289 |
| 21129647 | rs34802324 |
| 21129981 | rs201288 |
| 21130208 | rs201287 |
| 21131340 | rs34891710 |
| 21131348 | rs11438527 |
| 21131431 | rs34878496 |
| 21131727 | rs16884396 |
| 21132081 | rs9465948 |
| 21132220 | rs16884399 |
| 21133062 | rs11297091 |
| 21133400 | rs169293 |
| 21133737 | rs201307 |
| 21133815 | rs12206158 |
| 21133964 | rs34317920 |
| 21134374 | rs34305502 |
| 21134401 | rs35570989 |
| 21135019 | rs13218003 |
| 21135740 | rs12209877 |
| 21136038 | rs6921781 |
| 21136172 | rs16884406 |
| 21136940 | rs201306 |
| 21136972 | rs201305 |
| 21137253 | rs201304 |
| 21137477 | rs34243092 |
| 21137698 | rs201303 |
| 21138072 | rs201302 |
| 21138132 | rs201301 |
| 21138504 | rs35592283 |
| 21138790 | rs10946426 |
| 21138980 | rs6919322 |
| 21139058 | rs4710961 |
| 21139068 | rs9465949 |
| 21139350 | rs398913 |
| 21139414 | rs4710962 |
| 21139480 | rs12526927 |
| 21139855 | rs9460587 |
| 21140661 | rs415446 |
| 21141104 | rs35761643 |
| 21141617 | rs11961096 |
| 21141944 | rs201300 |
| 21142185 | rs35161624 |
| 21142199 | rs35206549 |
| 21142635 | rs13210277 |
| 21142726 | rs12198160 |
| 21142744 | rs7766970 |
| 21143715 | rs201299 |
| 21143786 | rs35061829 |
| 21144118 | rs201298 |
| 21144658 | rs201297 |
| 21145049 | rs201296 |
| 21145549 | rs34771308 |
| 21145593 | rs9295488 |
| 21145919 | rs989969 |
| 21146291 | rs34874522 |
| 21146814 | rs201312 |
| 21146901 | rs201313 |
| 21147026 | rs201314 |
| 21147263 | rs9350314 |
| 21147738 | rs6918555 |
| 21147791 | rs6918751 |
| 21147930 | rs9295489 |
| 21148244 | rs13190826 |
| 21148269 | rs13190835 |
| 21148356 | rs17662372 |
| 21148684 | rs201315 |
| 21148701 | rs16884415 |
| 21150019 | rs16884420 |
| 21150116 | rs17730046 |
| 21150139 | rs201316 |
| 21151059 | rs16884425 |
| 21151118 | rs1555052 |
| 21151344 | rs11753092 |
| 21151557 | rs34254730 |
| 21151557 | rs9465950 |
| 21151573 | rs7756338 |
| 21151831 | rs9348455 |
| 21151949 | rs201317 |
| 21152589 | rs6925617 |
| 21152677 | rs201318 |
| 21152873 | rs9460588 |
| 21152874 | rs34536386 |
| 21152874 | rs9460589 |
| 21152898 | rs201319 |
| 21153050 | rs201320 |
| 21153105 | rs201321 |
| 21153294 | rs201322 |
| 21153342 | rs201323 |
| 21153387 | rs201324 |
| 21153587 | rs201325 |
| 21153907 | rs9460590 |
| 21153987 | rs201326 |
| 21154014 | rs201327 |
| 21154069 | rs201328 |
| 21154147 | rs201329 |
| 21154297 | rs201330 |
| 21154380 | rs13217562 |
| 21154919 | rs34472513 |
| 21154958 | rs201331 |
| 21155233 | rs201332 |
| 21155256 | rs34312444 |
| 21155363 | rs201333 |
| 21155639 | rs201334 |
| 21155793 | rs201335 |
| 21156103 | rs201336 |
| 21156840 | rs201337 |
| 21156885 | rs201338 |
| 21157007 | rs34847494 |
| 21157597 | rs9942509 |
| 21157604 | rs34987941 |
| 21158291 | rs10806930 |
| 21158841 | rs4712579 |
| 21160395 | rs7775602 |
| 21160650 | rs11760181 |
| 21160654 | rs2056951 |
| 21160683 | rs7758243 |
| 21160934 | rs9358387 |
| 21160973 | rs6456391 |
| 21160974 | rs9465951 |
| 21161031 | rs6903997 |
| 21162291 | rs6933211 |
| 21162635 | rs7748652 |
| 21162661 | rs6456392 |
| 21162711 | rs13215037 |
| 21162805 | rs7752701 |
| 21163066 | rs6456393 |
| 21163111 | rs2328567 |
| 21163129 | rs6456394 |
| 21163541 | rs12661235 |
| 21163651 | rs7754018 |
| 21163696 | rs9350315 |
| 21163923 | rs9918435 |
| 21165424 | rs9465953 |
| 21165665 | rs35560899 |
| 21165892 | rs34142046 |
| 21166104 | rs9465954 |
| 21166730 | rs12202256 |
| 21166802 | rs2206014 |
| 21167015 | rs13219721 |
| 21167016 | rs13205176 |
| 21167182 | rs13219752 |
| 21167200 | rs13205420 |
| 21167401 | rs9350316 |
| 21167460 | rs9368270 |
| 21167796 | rs9465955 |
| 21169768 | rs28532482 |
| 21170022 | rs17237178 |
| 21170224 | rs13209905 |
| 21170225 | rs13209787 |
| 21170236 | rs13210005 |
| 21170269 | rs13210023 |
| 21170287 | rs13210027 |
| 21170376 | rs7744921 |
| 21170433 | rs9465956 |
| 21170545 | rs9465957 |
| 21170553 | rs9465958 |
| 21170579 | rs16884466 |
| 21170710 | rs11969047 |
| 21170765 | rs16884468 |
| 21170846 | rs11969587 |
| 21171228 | rs9465959 |
| 21171401 | rs34908981 |
| 21172309 | rs9460591 |
| 21173104 | rs35603774 |
| 21173126 | rs9465960 |
| 21173198 | rs34559907 |
| 21173393 | rs4710963 |
| 21173679 | rs9465961 |
| 21173766 | rs1001310 |
| 21173805 | rs4712580 |
| 21174689 | rs9460592 |
| 21174762 | rs9460593 |
| 21176267 | rs12209678 |
| 21177342 | rs35738724 |
| 21177501 | rs7762612 |
| 21177928 | rs36174378 |
| 21177981 | rs13199309 |
| 21178014 | rs9295490 |
| 21178237 | rs12660913 |
| 21178243 | rs10946427 |
| 21178328 | rs12199982 |
| 21178333 | rs34600664 |
| 21178468 | rs6932873 |
| 21178475 | rs9356760 |
| 21178487 | rs9465962 |
| 21178588 | rs9356761 |
| 21178609 | rs10650825 |
| 21179141 | rs4382241 |
| 21179575 | rs2013346 |
| 21179832 | rs10498700 |
| 21180396 | rs9350317 |
| 21180629 | rs11757139 |
| 21180733 | rs11757596 |
| 21181008 | rs11757677 |
| 21181027 | rs9465963 |
| 21181171 | rs13203959 |
| 21181269 | rs9366374 |
| 21181357 | rs11968037 |
| 21181410 | rs6918997 |
| 21181499 | rs9368271 |
| 21181567 | rs9460594 |
| 21181588 | rs9460595 |
| 21182172 | rs9295491 |
| 21182342 | rs4283868 |
| 21182548 | rs4413602 |
| 21183182 | rs10541645 |
| 21183338 | rs10541646 |
| 21183364 | rs11407560 |
| 21183365 | rs35953450 |
| 21183371 | rs7766554 |
| 21183409 | rs7766574 |
| 21183437 | rs10541647 |
| 21183457 | rs7766451 |
| 21183464 | rs7766713 |
| 21183493 | rs7766728 |
| 21183552 | rs4280955 |
| 21183645 | rs7767038 |
| 21183675 | rs7766788 |
| 21183741 | rs7770701 |
| 21184027 | rs16884481 |
| 21184246 | rs9295492 |
| 21184599 | rs4367364 |
| 21184749 | rs2068072 |
| 21184770 | rs2068071 |
| 21185523 | rs11963640 |
| 21185602 | rs11961090 |
| 21185611 | rs11963654 |
| 21185810 | rs35119417 |
| 21186212 | rs9358388 |
| 21186666 | rs4599624 |
| 21187063 | rs10080639 |
| 21187282 | rs7749838 |
| 21187336 | rs11967298 |
| 21187386 | rs34700985 |
| 21187552 | rs9366375 |
| 21187610 | rs4310041 |
| 21187627 | rs4496780 |
| 21187688 | rs7750461 |
| 21187916 | rs6917272 |
| 21188170 | rs6917904 |
| 21188430 | rs34700472 |
| 21188451 | rs9350318 |
| 21189185 | rs4323302 |
| 21189629 | rs9366376 |
| 21189885 | rs10456237 |
| 21189931 | rs13219886 |
| 21190084 | rs9368272 |
| 21190476 | rs35189729 |
| 21191594 | rs6904648 |
| 21191595 | rs11751563 |
| 21191595 | rs35898656 |
| 21192182 | rs4593348 |
| 21192409 | rs4421186 |
| 21192801 | rs6915237 |
| 21193447 | rs7773973 |
| 21193514 | rs17834555 |
| 21193811 | rs6901354 |
| 21194026 | rs4130033 |
| 21194285 | rs35287977 |
| 21194419 | rs10806931 |
| 21194732 | rs11964144 |
| 21194764 | rs10946428 |
| 21196061 | rs12200487 |
| 21196232 | rs4712581 |
| 21196650 | rs6456395 |
| 21196835 | rs6456396 |
| 21197051 | rs9368273 |
| 21197394 | rs4712582 |
| 21198212 | rs34465715 |
| 21198601 | rs6904348 |
| 21199015 | rs11433815 |
| 21199016 | rs36110847 |
| 21199138 | rs9368274 |
| 21199756 | rs12197841 |
| 21199815 | rs12197853 |
| 21199817 | rs12197854 |
| 21200342 | rs7762660 |
| 21200361 | rs7762670 |
| 21200554 | rs7762964 |
| 21200605 | rs13210302 |
| 21200944 | rs34756370 |
| 21201333 | rs7768086 |
| 21201341 | rs13214341 |
| 21202104 | rs33945169 |
| 21202717 | rs6939622 |
| 21202933 | rs4454125 |
| 21203121 | rs4401656 |
| 21204432 | rs11969472 |
| 21204602 | rs9358389 |
| 21204788 | rs4712583 |
| 21205444 | rs11757261 |
| 21205844 | rs34430280 |
| 21206360 | rs11757901 |
| 21206756 | rs34764667 |
| 21206833 | rs35866346 |
| 21207116 | rs9465965 |
| 21207331 | rs7744319 |
| 21207645 | rs9368275 |
| 21207687 | rs9356762 |
| 21208023 | rs9350319 |
| 21208328 | rs9350320 |
| 21208606 | rs9366377 |
| 21208909 | rs5874801 |
| 21209654 | rs9368276 |
| 21210536 | rs4712584 |
| 21210553 | rs34092761 |
| 21210726 | rs9366378 |
| 21211436 | rs11968036 |
| 21211664 | rs9465966 |
| 21211803 | rs4569951 |
| 21212138 | rs4130302 |
| 21212414 | rs35081613 |
| 21212547 | rs9358390 |
| 21212715 | rs6925464 |
| 21212715 | rs9460596 |
| 21212847 | rs17834987 |
| 21213142 | rs12529531 |
| 21213851 | rs36086759 |
| 21214087 | rs6936205 |
| 21215067 | rs4574622 |
| 21215086 | rs9465967 |
| 21215088 | rs9358391 |
| 21215150 | rs35347692 |
| 21215159 | rs5874802 |
| 21215207 | rs4624863 |
| 21215258 | rs9465968 |
| 21215359 | rs4526186 |
| 21215371 | rs34865903 |
| 21215437 | rs7739050 |
| 21215448 | rs4326226 |
| 21216093 | rs4712585 |
| 21216145 | rs9356763 |
| 21216170 | rs4710964 |
| 21216871 | rs9460597 |
| 21218688 | rs11963150 |
| 21218907 | rs6923546 |
| 21219519 | rs6924221 |
| 21220306 | rs10456238 |
| 21222458 | rs34418857 |
| 21222459 | rs34251532 |
| 21222460 | rs34219362 |
| 21222644 | rs35962170 |
| 21223003 | rs16884514 |
| 21223188 | rs7773318 |
| 21223279 | rs4340995 |
| 21223295 | rs9356764 |
| 21224572 | rs35468559 |
| 21224879 | rs3935207 |
| 21224965 | rs35629967 |
| 21225019 | rs6927498 |
| 21225119 | rs10613278 |
| 21225499 | rs35372171 |
| 21225513 | rs10554335 |
| 21225585 | rs7764365 |
| 21225685 | rs35234206 |
| 21225694 | rs11418036 |
| 21225734 | rs7768536 |
| 21226218 | rs4389757 |
| 21227109 | rs6456397 |
| 21227703 | rs35183051 |
| 21228007 | rs7741436 |
| 21228269 | rs9356765 |
| 21228407 | rs9366379 |
| 21228562 | rs6926818 |
| 21228614 | rs16884524 |
| 21228619 | rs34425854 |
| 21228733 | rs4074910 |
| 21228748 | rs4076112 |
| 21229002 | rs35885025 |
| 21229134 | rs6909332 |
| 21229654 | rs4438948 |
| 21229845 | rs6905660 |
| 21230834 | rs9358392 |
| 21231054 | rs9368279 |
| 21231899 | rs4315997 |
| 21231967 | rs4479917 |
| 21232500 | rs10946430 |
| 21232701 | rs34821627 |
| 21232763 | rs13191691 |
| 21232779 | rs13207763 |
| 21232796 | rs13207766 |
| 21232816 | rs13191830 |
| 21233338 | rs34857211 |
| 21234866 | rs7750839 |
| 21235291 | rs7751485 |
| 21235577 | rs11757294 |
| 21235798 | rs34183889 |
| 21236043 | rs12055489 |
| 21236223 | rs28421119 |
| 21237436 | rs9295493 |
| 21237650 | rs12055790 |
| 21237696 | rs35692444 |
| 21237763 | rs7766575 |
| 21237890 | rs9356766 |
| 21237892 | rs34802727 |
| 21237915 | rs35927368 |
| 21238432 | rs10708944 |
| 21238635 | rs6932722 |
| 21238849 | rs6937439 |
| 21238961 | rs9986401 |
| 21239004 | rs9465969 |
| 21239122 | rs9986662 |
| 21239291 | rs9295494 |
| 21239512 | rs35030599 |
| 21240132 | rs9460598 |
| 21240526 | rs28403910 |
| 21241995 | rs9465970 |
| 21242670 | rs7760880 |
| 21242672 | rs7761283 |
| 21242682 | rs11463641 |
| 21242783 | rs7765177 |
| 21242820 | rs7765199 |
| 21242857 | rs7764887 |
| 21243009 | rs7765106 |
| 21243039 | rs28610069 |
| 21243105 | rs7765274 |
| 21243107 | rs2446490 |
| 21243137 | rs2493869 |
| 21243228 | rs7765725 |
| 21243240 | rs7765730 |
| 21243646 | rs16884554 |
| 21243857 | rs13216162 |
| 21244099 | rs2446489 |
| 21244512 | rs2446488 |
| 21244516 | rs7771907 |
| 21245195 | rs9350322 |
| 21245369 | rs2446487 |
| 21246042 | rs35201465 |
| 21246079 | rs9358393 |
| 21246445 | rs959712 |
| 21246447 | rs35541643 |
| 21246451 | rs34848377 |
| 21246456 | rs5874803 |
| 21246457 | rs33951051 |
| 21246458 | rs5874804 |
| 21246459 | rs35717786 |
| 21246466 | rs5874805 |
| 21246467 | rs35384149 |
| 21246474 | rs959711 |
| 21246487 | rs34589183 |
| 21246785 | rs9688559 |
| 21246915 | rs9689353 |
| 21246958 | rs34154291 |
| 21246988 | rs9688564 |
| 21246992 | rs9688565 |
| 21247009 | rs2446486 |
| 21247020 | rs9688569 |
| 21247061 | rs9688573 |
| 21247079 | rs9295495 |
| 21247094 | rs9465971 |
| 21247737 | rs9460599 |
| 21247746 | rs6916667 |
| 21247788 | rs9465972 |
| 21248278 | rs2446485 |
| 21248465 | rs2446484 |
| 21249707 | rs28665959 |
| 21249879 | rs35583136 |
| 21249895 | rs34896971 |
| 21249984 | rs7746383 |
| 21250235 | rs7746846 |
| 21250457 | rs28360550 |
| 21250639 | rs6932702 |
| 21251345 | rs9348456 |
| 21251752 | rs35724409 |
| 21251989 | rs2328573 |
| 21252098 | rs17835633 |
| 21252216 | rs36008085 |
| 21252578 | rs10498701 |
| 21252578 | rs35938718 |
| 21252735 | rs34957382 |
| 21253603 | rs10708192 |
| 21254245 | rs1466340 |
| 21255061 | rs10710231 |
| 21255726 | rs1466339 |
| 21255824 | rs7740358 |
| 21256011 | rs11405792 |
| 21256411 | rs1471205 |
| 21256721 | rs9368280 |
| 21257043 | rs9295496 |
| 21257242 | rs12206028 |
| 21257356 | rs9368281 |
| 21258107 | rs2168985 |
| 21258129 | rs12207912 |
| 21258136 | rs35882470 |
| 21258203 | rs9368282 |
| 21258436 | rs7752602 |
| 21258626 | rs7752788 |
| 21258747 | rs2446483 |
| 21258795 | rs9356768 |
| 21258995 | rs34495814 |
| 21259344 | rs9350323 |
| 21259352 | rs4530843 |
| 21259489 | rs9460600 |
| 21259600 | rs9460601 |
| 21260113 | rs10946431 |
| 21260135 | rs35088240 |
| 21260216 | rs10946432 |
| 21260253 | rs10946433 |
| 21260917 | rs11961031 |
| 21261341 | rs4144175 |
| 21261468 | rs10604354 |
| 21261575 | rs12203853 |
| 21261620 | rs6901380 |
| 21261891 | rs6906201 |
| 21262118 | rs9460602 |
| 21262389 | rs35396145 |
| 21262741 | rs9350324 |
| 21262775 | rs9295497 |
| 21262969 | rs9366381 |
| 21263128 | rs6456398 |
| 21263152 | rs6456399 |
| 21263168 | rs6456400 |
| 21263322 | rs6456401 |
| 21263462 | rs11456476 |
| 21263471 | rs34116986 |
| 21263595 | rs6913868 |
| 21263798 | rs9366382 |
| 21263882 | rs9348457 |
| 21264053 | rs35931974 |
| 21264270 | rs9366383 |
| 21264385 | rs9366384 |
| 21264393 | rs9356769 |
| 21264732 | rs34306955 |
| 21264842 | rs9350325 |
| 21264968 | rs6924598 |
| 21265017 | rs4712586 |
| 21265065 | rs7761116 |
| 21265072 | rs9358395 |
| 21265152 | rs9368283 |
| 21265828 | rs34689265 |
| 21266252 | rs6931316 |
| 21267009 | rs6916577 |
| 21267167 | rs6937555 |
| 21267311 | rs10645059 |
| 21267321 | rs10652396 |
| 21267832 | rs16884591 |
| 21268027 | rs11442196 |
| 21268361 | rs7739578 |
| 21268400 | rs7739596 |
| 21268426 | rs4527692 |
| 21268664 | rs9295498 |
| 21268668 | rs9295499 |
| 21268866 | rs10080292 |
| 21268870 | rs35915482 |
| 21268881 | rs9465976 |
| 21268928 | rs28581582 |
| 21268942 | rs34844023 |
| 21269038 | rs12179712 |
| 21269039 | rs9465977 |
| 21269504 | rs9465978 |
| 21271039 | rs6929437 |
| 21271299 | rs4995985 |
| 21271738 | rs34031561 |
| 21271898 | rs6914598 |
| 21272056 | rs6935079 |
| 21272110 | rs6935117 |
| 21272124 | rs6935124 |
| 21272174 | rs9368284 |
| 21272190 | rs6915161 |
| 21272228 | rs9356770 |
| 21272416 | rs35558562 |
| 21272655 | rs6916053 |
| 21272716 | rs34191499 |
| 21273107 | rs6941714 |
| 21273161 | rs34326160 |
| 21273168 | rs9460603 |
| 21273192 | rs9348458 |
| 21273257 | rs7776158 |
| 21273274 | rs11965768 |
| 21274046 | rs35674401 |
| 21274684 | rs2125570 |
| 21275213 | rs7768526 |
| 21275957 | rs9368285 |
| 21276570 | rs28360551 |
| 21277729 | rs7763700 |
| 21277824 | rs4425589 |
| 21277964 | rs9348459 |
| 21278549 | rs13197595 |
| 21278592 | rs9460604 |
| 21278780 | rs12180174 |
| 21278845 | rs9465979 |
| 21279293 | rs36058161 |
| 21279338 | rs11969929 |
| 21279609 | rs11965049 |
| 21279673 | rs9295500 |
| 21279689 | rs34012677 |
| 21279828 | rs12178179 |
| 21279839 | rs9465980 |
| 21280353 | rs9358396 |
| 21281052 | rs12194541 |
| 21281118 | rs2061441 |
| 21281632 | rs9460605 |
| 21281781 | rs12525339 |
| 21282235 | rs35462438 |
| 21282590 | rs9465981 |
| 21282848 | rs4637624 |
| 21282946 | rs35969558 |
| 21283471 | rs12525940 |
| 21283655 | rs34603118 |
| 21283948 | rs12528104 |
| 21284103 | rs12526391 |
| 21284906 | rs6939148 |
| 21284912 | rs9460606 |
| 21285561 | rs13219281 |
| 21285569 | rs13219285 |
| 21285598 | rs13219506 |
| 21285611 | rs13219193 |
| 21285620 | rs13219198 |
| 21285664 | rs13219637 |
| 21285689 | rs13205078 |
| 21285691 | rs13205079 |
| 21285875 | rs11308599 |
| 21286261 | rs9465982 |
| 21288187 | rs12214946 |
| 21288554 | rs34495587 |
| 21289215 | rs12523755 |
| 21289629 | rs35642303 |
| 21290957 | rs9295501 |
| 21291348 | rs35815279 |
| 21291533 | rs12527222 |
| 21291647 | rs9465983 |
| 21291857 | rs2493868 |
| 21291918 | rs35979352 |
| 21292407 | rs34248538 |
| 21292789 | rs10946434 |
| 21292811 | rs9465984 |
| 21293033 | rs34599800 |
| 21293434 | rs35442433 |
| 21293569 | rs9465985 |
| 21294166 | rs35712201 |
| 21294748 | rs35539626 |
| 21294750 | rs9465986 |
| 21294751 | rs9465987 |
| 21294801 | rs11961469 |
| 21295134 | rs2446482 |
| 21295312 | rs9465988 |
| 21295313 | rs12191416 |
| 21295313 | rs35985333 |
| 21295996 | rs9465989 |
| 21296793 | rs9350327 |
| 21297183 | rs34750271 |
| 21297265 | rs35013686 |
| 21297416 | rs16884616 |
| 21297902 | rs35898446 |
| 21297924 | rs11751020 |
| 21297967 | rs10452581 |
| 21298562 | rs13192000 |
| 21298563 | rs13191669 |
| 21298583 | rs13192011 |
| 21298617 | rs13192029 |
| 21298629 | rs13192143 |
| 21298630 | rs13207866 |
| 21298671 | rs13191819 |
| 21298690 | rs13192164 |
| 21298721 | rs13192173 |
| 21298723 | rs13191845 |
| 21299659 | rs9465990 |
| 21299810 | rs9460607 |
| 21299907 | rs9465991 |
| 21299909 | rs9366386 |
| 21299971 | rs35944981 |
| 21300001 | rs9366387 |
| 21300046 | rs9366388 |
| 21300106 | rs9368287 |
| 21300203 | rs13193222 |
| 21300325 | rs10080974 |
| 21300381 | rs9295502 |
| 21300388 | rs12528974 |
| 21300395 | rs7759646 |
| 21300433 | rs9465992 |
| 21300768 | rs11964193 |
| 21301021 | rs34456723 |
| 21301080 | rs34094109 |
| 21301834 | rs11759448 |
| 21302380 | rs11962770 |
| 21303198 | rs9366389 |
| 21303687 | rs11753415 |
| 21304730 | rs4712587 |
| 21304976 | rs7748091 |
| 21305299 | rs28469715 |
| 21305355 | rs7748766 |
| 21305591 | rs35164470 |
| 21305660 | rs2125571 |
| 21305669 | rs9465993 |
| 21306062 | rs3793090 |
| 21307994 | rs1531303 |
| 21308261 | rs2305955 |
| 21308369 | rs1459047 |
| 21309244 | rs35662535 |
| 21309281 | rs9767650 |
| 21309286 | rs9767186 |
| 21309387 | rs9460608 |
| 21309472 | rs9465994 |
| 21310133 | rs9465995 |
| 21310563 | rs36067162 |
| 21310749 | rs11965158 |
| 21311344 | rs9350328 |
| 21311426 | rs5874806 |
| 21311451 | rs10616274 |
| 21311452 | rs5874807 |
| 21311454 | rs11288843 |
| 21311471 | rs9350329 |
| 21311502 | rs1824330 |
| 21311620 | rs9717950 |
| 21311710 | rs3898487 |
| 21311900 | rs9350330 |
| 21311902 | rs9350331 |
| 21312023 | rs35603064 |
| 21312085 | rs35615714 |
| 21312109 | rs36017220 |
| 21312120 | rs12196363 |
| 21312143 | rs35881379 |
| 21312153 | rs35710688 |
| 21312177 | rs35017881 |
| 21312188 | rs12196418 |
| 21312191 | rs12196419 |
| 21312206 | rs35883368 |
| 21312223 | rs12196423 |
| 21312231 | rs34046809 |
| 21312253 | rs34108390 |
| 21312453 | rs6921264 |
| 21312671 | rs6921652 |
| 21312775 | rs6926388 |
| 21312801 | rs12527588 |
| 21313200 | rs10456240 |
| 21313329 | rs10456241 |
| 21313367 | rs10456242 |
| 21313458 | rs10456243 |
| 21313856 | rs34046046 |
| 21313879 | rs13213969 |
| 21313886 | rs6932316 |
| 21313910 | rs6932752 |
| 21313958 | rs13214311 |
| 21313963 | rs6932914 |
| 21313998 | rs6932635 |
| 21314018 | rs6912407 |
| 21314041 | rs34849597 |
| 21314107 | rs9366390 |
| 21314243 | rs10223539 |
| 21314298 | rs10223540 |
| 21314473 | rs6913302 |
| 21315081 | rs9366391 |
| 21315139 | rs9356771 |
| 21315390 | rs12530254 |
| 21315432 | rs34085972 |
| 21315529 | rs4291091 |
| 21315727 | rs6940465 |
| 21315763 | rs6901748 |
| 21316195 | rs6902505 |
| 21316396 | rs898167 |
| 21316398 | rs898166 |
| 21316408 | rs898165 |
| 21316820 | rs34797264 |
| 21317102 | rs9368288 |
| 21317206 | rs9358397 |
| 21317611 | rs2168984 |
| 21317978 | rs1563728 |
| 21318135 | rs4712588 |
| 21318266 | rs11267610 |
| 21318399 | rs4712589 |
| 21318666 | rs6915037 |
| 21318882 | rs12664336 |
| 21319431 | rs9465998 |
| 21319494 | rs10214790 |
| 21319776 | rs12201217 |
| 21320060 | rs9350332 |
| 21320905 | rs9358398 |
| 21321149 | rs9358399 |
| 21321286 | rs9358400 |
| 21321533 | rs10214694 |
| 21321733 | rs10214716 |
| 21322176 | rs9460609 |
| 21322179 | rs6929219 |
| 21322517 | rs12527686 |
| 21322561 | rs12527673 |
| 21322733 | rs9350333 |
| 21323322 | rs10946436 |
| 21323380 | rs6913136 |
| 21323400 | rs13200114 |
| 21323464 | rs13200422 |
| 21323815 | rs2328572 |
| 21323949 | rs9350334 |
| 21324672 | rs34913347 |
| 21324713 | rs10946437 |
| 21324725 | rs10946438 |
| 21325164 | rs9358401 |
| 21325261 | rs34055473 |
| 21325350 | rs34921405 |
| 21325357 | rs6904880 |
| 21325395 | rs6456403 |
| 21325653 | rs2085654 |
| 21325832 | rs9466000 |
| 21325853 | rs9466001 |
| 21326033 | rs2100707 |
| 21326158 | rs12111402 |
| 21326366 | rs4712590 |
| 21326649 | rs4710965 |
| 21327416 | rs6937610 |
| 21327459 | rs12110862 |
| 21327488 | rs35624914 |
| 21327606 | rs11349673 |
| 21327854 | rs16884681 |
| 21327895 | rs7738425 |
| 21328030 | rs16884685 |
| 21328355 | rs16884688 |
| 21328398 | rs35663664 |
| 21328510 | rs12203389 |
| 21328818 | rs12191541 |
| 21328946 | rs34618548 |
| 21330074 | rs1563726 |
| 21330730 | rs16884693 |
| 21331119 | rs2328574 |
| 21331209 | rs16884699 |
| 21331264 | rs16884705 |
| 21331267 | rs6929141 |
| 21331293 | rs16884709 |
| 21331392 | rs16884713 |
| 21332034 | rs9466002 |
| 21332081 | rs9466003 |
| 21332103 | rs9466004 |
| 21332139 | rs9466005 |
| 21332272 | rs9460610 |
| 21332409 | rs7770316 |
| 21332488 | rs11964983 |
| 21332496 | rs7770752 |
| 21332625 | rs7770637 |
| 21333229 | rs1870421 |
| 21333556 | rs6942273 |
| 21333618 | rs9466006 |
| 21333709 | rs9466007 |
| 21334500 | rs7763249 |
| 21335731 | rs9368289 |
| 21335750 | rs9368290 |
| 21335782 | rs13202305 |
| 21335903 | rs34362358 |
| 21335906 | rs11415596 |
| 21336317 | rs28484932 |
| 21336582 | rs7754027 |
| 21336699 | rs34022115 |
| 21336867 | rs4710966 |
| 21337512 | rs16884722 |
| 21338182 | rs35571136 |
| 21338184 | rs35739791 |
| 21338815 | rs9460611 |
| 21338986 | rs9460612 |
| 21339013 | rs12200511 |
| 21339097 | rs35791563 |
| 21339201 | rs34084405 |
| 21339207 | rs34158326 |
| 21339453 | rs1563727 |
| 21339524 | rs3840416 |
| 21339530 | rs11362523 |
| 21339688 | rs7770664 |
| 21339861 | rs35121088 |
| 21339935 | rs4712591 |
| 21340199 | rs35206923 |
| 21340202 | rs28600127 |
| 21340213 | rs4710967 |
| 21340214 | rs4710968 |
| 21340218 | rs13213171 |
| 21340219 | rs13197226 |
| 21340225 | rs12199601 |
| 21340594 | rs1137970 |

TABLE 10
SNPs within LD block C06 (SEQ ID NO: 1) between positions
20,634,996 and 20,836,710 bp on Chromosome 6 in NCBI Build 35
and NCBI Build 36

| | Position in | |
|---|---|---|
| Position in | SEQ ID | |
| Build 35/36 | NO: 1 | Marker ID |
| 20634996 | 1 | rs4429936 |
| 20635028 | 33 | rs9465780 |
| 20635060 | 65 | rs7743222 |
| 20635066 | 71 | rs7743223 |
| 20635241 | 246 | rs4516938 |
| 20635285 | 290 | rs4628090 |
| 20635339 | 344 | rs9465781 |
| 20635349 | 354 | rs28450063 |
| 20635350 | 355 | rs9465782 |
| 20635834 | 839 | rs4712503 |
| 20635845 | 850 | rs9465783 |
| 20635860 | 865 | rs4712504 |
| 20636037 | 1042 | rs10946388 |
| 20636813 | 1818 | rs9460517 |
| 20636939 | 1944 | rs34086777 |
| 20637089 | 2094 | rs9465785 |
| 20637215 | 2220 | rs7754223 |
| 20637279 | 2284 | rs34173688 |
| 20637287 | 2292 | rs11459684 |
| 20637288 | 2293 | rs35781726 |
| 20637303 | 2308 | rs9460518 |
| 20637450 | 2455 | rs11362835 |
| 20637521 | 2526 | rs7772956 |
| 20637824 | 2829 | rs1883641 |
| 20637875 | 2880 | rs1883640 |
| 20637944 | 2949 | rs11402844 |
| 20638219 | 3224 | rs35198704 |
| 20638372 | 3377 | rs6923683 |
| 20638762 | 3767 | rs12181295 |
| 20638829 | 3834 | rs9465788 |
| 20638961 | 3966 | rs34578766 |
| 20639509 | 4514 | rs2206578 |
| 20639662 | 4667 | rs35530523 |
| 20639708 | 4713 | rs2206577 |
| 20639710 | 4715 | rs34553771 |
| 20639718 | 4723 | rs34581322 |
| 20639719 | 4724 | rs5874771 |
| 20639909 | 4914 | rs6902897 |
| 20640005 | 5010 | rs34607984 |
| 20640118 | 5123 | rs6923201 |
| 20640162 | 5167 | rs6903415 |
| 20640249 | 5254 | rs9465790 |
| 20640425 | 5430 | rs6923750 |
| 20640859 | 5864 | rs10717803 |
| 20641038 | 6043 | rs9465791 |
| 20641141 | 6146 | rs6909467 |
| 20641248 | 6253 | rs34525680 |
| 20641293 | 6298 | rs35457534 |
| 20641299 | 6304 | rs35731703 |
| 20641303 | 6308 | rs10554680 |
| 20641320 | 6325 | rs35239102 |
| 20641362 | 6367 | rs9368197 |
| 20641413 | 6418 | rs9465792 |
| 20641494 | 6499 | rs12212722 |
| 20641581 | 6586 | rs7765611 |
| 20641590 | 6595 | rs10566792 |
| 20641598 | 6603 | rs10566793 |
| 20641718 | 6723 | rs34275610 |
| 20641718 | 6723 | rs10566794 |
| 20641724 | 6729 | rs11347538 |
| 20641733 | 6738 | rs5874772 |
| 20642073 | 7078 | rs16883887 |
| 20642385 | 7390 | rs34088191 |
| 20642428 | 7433 | rs10806920 |
| 20642440 | 7445 | rs11370426 |
| 20642441 | 7446 | rs33915274 |
| 20642442 | 7447 | rs11459775 |
| 20642443 | 7448 | rs34576540 |
| 20642494 | 7499 | rs10708068 |
| 20642586 | 7591 | rs4712505 |
| 20642787 | 7792 | rs41271303 |
| 20642953 | 7958 | rs11963450 |
| 20643397 | 8402 | rs981043 |
| 20643513 | 8518 | rs981042 |
| 20643675 | 8680 | rs16883895 |
| 20643753 | 8758 | rs17512225 |
| 20643840 | 8845 | rs35035071 |
| 20643949 | 8954 | rs6904566 |
| 20644073 | 9078 | rs6927356 |
| 20644093 | 9098 | rs35281412 |
| 20644313 | 9318 | rs35915788 |
| 20644314 | 9319 | rs34025398 |
| 20644320 | 9325 | rs34361235 |
| 20644335 | 9340 | rs6905138 |
| 20644499 | 9504 | rs13194858 |
| 20644717 | 9722 | rs2179551 |
| 20644727 | 9732 | rs2179550 |
| 20644787 | 9792 | rs9465794 |
| 20644787 | 9792 | rs9465795 |
| 20644848 | 9853 | rs7747962 |
| 20644858 | 9863 | rs6910725 |
| 20644918 | 9923 | rs965054 |
| 20644971 | 9976 | rs2143407 |
| 20645032 | 10037 | rs10619380 |
| 20645431 | 10436 | rs2328525 |
| 20645661 | 10666 | rs13199286 |
| 20645841 | 10846 | rs10611252 |
| 20645940 | 10945 | rs7753499 |
| 20646023 | 11028 | rs7753956 |
| 20646024 | 11029 | rs34811195 |
| 20646024 | 11029 | rs7753670 |
| 20646107 | 11112 | rs3060613 |
| 20646107 | 11112 | rs6149468 |
| 20646109 | 11114 | rs11277970 |
| 20646110 | 11115 | rs11280099 |
| 20646139 | 11144 | rs16883900 |
| 20646175 | 11180 | rs7774291 |
| 20646443 | 11448 | rs10612082 |
| 20646476 | 11481 | rs9368198 |
| 20646502 | 11507 | rs13203336 |
| 20646504 | 11509 | rs13203631 |
| 20646619 | 11624 | rs6456355 |
| 20646644 | 11649 | rs10484635 |
| 20647190 | 12195 | rs12204173 |
| 20647320 | 12325 | rs13207544 |
| 20647851 | 12856 | rs12198728 |
| 20647984 | 12989 | rs28396084 |
| 20648327 | 13332 | rs12199073 |
| 20648500 | 13505 | rs9465796 |
| 20648561 | 13566 | rs12212600 |
| 20648596 | 13601 | rs13212040 |
| 20648663 | 13668 | rs35291340 |
| 20648722 | 13727 | rs12199324 |
| 20649085 | 14090 | rs12200871 |
| 20649159 | 14164 | rs9348432 |
| 20649183 | 14188 | rs12200834 |
| 20649236 | 14241 | rs34860173 |
| 20649324 | 14329 | rs11754872 |
| 20649498 | 14503 | rs6456356 |
| 20649517 | 14522 | rs9368199 |
| 20649682 | 14687 | rs2143406 |
| 20650176 | 15181 | rs10484634 |
| 20650200 | 15205 | rs7758851 |
| 20650398 | 15403 | rs34677076 |
| 20651447 | 16452 | rs6928571 |
| 20651461 | 16466 | rs12192584 |
| 20651608 | 16613 | rs34856684 |
| 20652015 | 17020 | rs9350255 |
| 20652091 | 17096 | rs9368200 |
| 20652136 | 17141 | rs12214002 |
| 20652245 | 17250 | rs9465797 |
| 20652300 | 17305 | rs9465798 |
| 20652574 | 17579 | rs28699301 |
| 20652650 | 17655 | rs13215844 |
| 20652678 | 17683 | rs12214315 |
| 20652722 | 17727 | rs11759517 |
| 20652786 | 17791 | rs13218957 |
| 20652806 | 17811 | rs13218962 |
| 20653188 | 18193 | rs10543744 |
| 20653201 | 18206 | rs12216047 |
| 20653447 | 18452 | rs9366354 |
| 20653890 | 18895 | rs9358342 |
| 20654091 | 19096 | rs9368201 |
| 20654382 | 19387 | rs34206163 |
| 20654506 | 19511 | rs9465799 |
| 20654794 | 19799 | rs34187071 |
| 20654867 | 19872 | rs9465800 |
| 20654890 | 19895 | rs6908974 |
| 20654992 | 19997 | rs13197372 |
| 20655361 | 20366 | rs13214145 |
| 20655793 | 20798 | rs16883910 |
| 20655968 | 20973 | rs12194705 |
| 20656271 | 21276 | rs35080661 |
| 20656465 | 21470 | rs7753467 |
| 20656466 | 21471 | rs7773488 |
| 20656986 | 21991 | rs34182285 |
| 20657084 | 22089 | rs34242699 |
| 20657780 | 22785 | rs9348433 |
| 20657942 | 22947 | rs9460519 |
| 20658083 | 23088 | rs12198377 |
| 20658096 | 23101 | rs9465801 |
| 20658195 | 23200 | rs9465802 |
| 20658822 | 23827 | rs28458932 |
| 20658823 | 23828 | rs9465803 |
| 20658981 | 23986 | rs2103682 |
| 20659321 | 24326 | rs9465804 |
| 20659580 | 24585 | rs34611621 |
| 20660058 | 25063 | rs12055423 |
| 20660653 | 25658 | rs9465805 |
| 20660829 | 25834 | rs11365187 |
| 20660836 | 25841 | rs11320714 |
| 20660918 | 25923 | rs9350256 |
| 20661764 | 26769 | rs7756211 |
| 20662069 | 27074 | rs9460520 |
| 20662498 | 27503 | rs34245467 |
| 20662930 | 27935 | rs9350257 |
| 20663855 | 28860 | rs11964554 |
| 20663990 | 28995 | rs9465806 |
| 20664109 | 29114 | rs11964635 |
| 20664190 | 29195 | rs13199421 |
| 20664314 | 29319 | rs6932320 |
| 20664570 | 29575 | rs12200078 |
| 20664659 | 29664 | rs13437555 |
| 20664884 | 29889 | rs9350258 |
| 20665256 | 30261 | rs12176441 |
| 20665260 | 30265 | rs12183826 |
| 20665264 | 30269 | rs9356738 |
| 20665272 | 30277 | rs9348434 |
| 20665343 | 30348 | rs9465807 |
| 20665804 | 30809 | rs4458667 |
| 20665995 | 31000 | rs7739402 |
| 20667590 | 32595 | rs16883914 |
| 20667591 | 32596 | rs16883916 |
| 20667900 | 32905 | rs9654584 |
| 20667999 | 33004 | rs9465808 |
| 20668414 | 33419 | rs17584626 |
| 20668565 | 33570 | rs7751682 |
| 20669667 | 34672 | rs11361279 |
| 20669681 | 34686 | rs34634263 |
| 20670059 | 35064 | rs12214549 |
| 20670364 | 35369 | rs7753519 |
| 20670575 | 35580 | rs28567007 |
| 20670597 | 35602 | rs7772137 |
| 20670719 | 35724 | rs12208597 |
| 20670998 | 36003 | rs9368202 |
| 20671877 | 36882 | rs2328526 |
| 20672452 | 37457 | rs34823358 |
| 20673287 | 38292 | rs28639914 |
| 20673363 | 38368 | rs34233572 |
| 20673415 | 38420 | rs4712506 |
| 20673935 | 38940 | rs13203450 |
| 20674280 | 39285 | rs9350259 |
| 20674435 | 39440 | rs6918457 |
| 20674595 | 39600 | rs35210537 |
| 20674749 | 39754 | rs11329887 |
| 20675016 | 40021 | rs9348435 |
| 20675068 | 40073 | rs35366106 |
| 20675342 | 40347 | rs16901563 |
| 20675352 | 40357 | rs12333229 |
| 20675520 | 40525 | rs9460521 |
| 20676094 | 41099 | rs10589899 |
| 20676351 | 41356 | rs2876573 |
| 20676957 | 41962 | rs6935461 |
| 20676963 | 41968 | rs6935465 |
| 20676968 | 41973 | rs10603174 |
| 20677060 | 42065 | rs12333291 |
| 20677967 | 42972 | rs2064321 |
| 20677985 | 42990 | rs35546893 |
| 20678018 | 43023 | rs4291090 |
| 20678121 | 43126 | rs2064320 |
| 20678268 | 43273 | rs9465810 |
| 20678275 | 43280 | rs9465811 |
| 20678423 | 43428 | rs9358344 |
| 20678756 | 43761 | rs10946390 |
| 20679114 | 44119 | rs6905281 |
| 20679339 | 44344 | rs16883932 |
| 20679612 | 44617 | rs34904067 |
| 20679660 | 44665 | rs7744002 |
| 20679763 | 44768 | rs35142564 |
| 20680095 | 45100 | rs9465812 |
| 20680678 | 45683 | rs7759094 |
| 20680784 | 45789 | rs9460522 |
| 20681538 | 46543 | rs7764551 |
| 20681585 | 46590 | rs10541455 |
| 20682409 | 47414 | rs16883935 |
| 20682542 | 47547 | rs13215603 |
| 20682568 | 47573 | rs962576 |
| 20683235 | 48240 | rs1474720 |
| 20683797 | 48802 | rs16883944 |
| 20684155 | 49160 | rs34538343 |
| 20684269 | 49274 | rs9350260 |
| 20684353 | 49358 | rs16883951 |
| 20684645 | 49650 | rs9358345 |
| 20684862 | 49867 | rs1012627 |
| 20684890 | 49895 | rs9368203 |
| 20684939 | 49944 | rs35894322 |
| 20684965 | 49970 | rs4710932 |
| 20684984 | 49989 | rs6909117 |
| 20685540 | 50545 | rs1012626 |
| 20685748 | 50753 | rs1012625 |
| 20685760 | 50765 | rs7752194 |
| 20685958 | 50963 | rs9465813 |
| 20686014 | 51019 | rs12207923 |
| 20686355 | 51360 | rs16883963 |
| 20686831 | 51836 | rs13205786 |
| 20686887 | 51892 | rs35205364 |
| 20687102 | 52107 | rs10456232 |
| 20687189 | 52194 | rs9465814 |
| 20687201 | 52206 | rs35571892 |
| 20687740 | 52745 | rs9465815 |
| 20687753 | 52758 | rs36119371 |
| 20687921 | 52926 | rs28621813 |
| 20687926 | 52931 | rs9350261 |
| 20687928 | 52933 | rs7341226 |
| 20688175 | 53180 | rs6927481 |
| 20688323 | 53328 | rs35313444 |
| 20688373 | 53378 | rs6928198 |
| 20688404 | 53409 | rs6907897 |
| 20688545 | 53550 | rs6928586 |
| 20688872 | 53877 | rs9368204 |
| 20689021 | 54026 | rs9358346 |
| 20689589 | 54594 | rs11967546 |
| 20689593 | 54598 | rs34134803 |
| 20689772 | 54777 | rs10456233 |
| 20689807 | 54812 | rs7744833 |
| 20690123 | 55128 | rs9460523 |
| 20690123 | 55128 | rs9465816 |
| 20690432 | 55437 | rs6908077 |
| 20690630 | 55635 | rs9465817 |
| 20691069 | 56074 | rs11967445 |
| 20691263 | 56268 | rs34022950 |
| 20691793 | 56798 | rs9460524 |
| 20691994 | 56999 | rs34020592 |
| 20692003 | 57008 | rs11448102 |
| 20692339 | 57344 | rs9465818 |
| 20692402 | 57407 | rs9350262 |
| 20692513 | 57518 | rs13205241 |
| 20693000 | 58005 | rs12153939 |
| 20693100 | 58105 | rs6925593 |
| 20693119 | 58124 | rs4712507 |
| 20693226 | 58231 | rs10558806 |
| 20693267 | 58272 | rs35982532 |
| 20693276 | 58281 | rs11385529 |
| 20693360 | 58365 | rs9348436 |
| 20693416 | 58421 | rs9368206 |
| 20693438 | 58443 | rs13209542 |
| 20693452 | 58457 | rs9368207 |
| 20693630 | 58635 | rs13209907 |
| 20693635 | 58640 | rs6926658 |
| 20694018 | 59023 | rs12213132 |
| 20694182 | 59187 | rs4357125 |
| 20694554 | 59559 | rs6932944 |
| 20694607 | 59612 | rs6932962 |
| 20695026 | 60031 | rs9348437 |
| 20695332 | 60337 | rs12201857 |
| 20695356 | 60361 | rs9465819 |
| 20695447 | 60452 | rs6938955 |
| 20695539 | 60544 | rs9460525 |
| 20695827 | 60832 | rs9465820 |
| 20695964 | 60969 | rs10946391 |
| 20695968 | 60973 | rs9368208 |
| 20696003 | 61008 | rs9465821 |
| 20696183 | 61188 | rs6923790 |
| 20696401 | 61406 | rs10558139 |
| 20697309 | 62314 | rs6907459 |
| 20697320 | 62325 | rs6907767 |
| 20697321 | 62326 | rs9465822 |
| 20697349 | 62354 | rs6930283 |
| 20697706 | 62711 | rs6908042 |
| 20697741 | 62746 | rs6935317 |
| 20697761 | 62766 | rs35370102 |
| 20698266 | 63271 | rs9368209 |
| 20698366 | 63371 | rs13216746 |
| 20698367 | 63372 | rs13216747 |
| 20699007 | 64012 | rs35485532 |
| 20699747 | 64752 | rs13216324 |
| 20699817 | 64822 | rs4336434 |
| 20700046 | 65051 | rs4509107 |
| 20700428 | 65433 | rs9465823 |
| 20700465 | 65470 | rs6936705 |
| 20700679 | 65684 | rs34023799 |
| 20700929 | 65934 | rs6942313 |
| 20701057 | 66062 | rs28869917 |
| 20701318 | 66323 | rs34982231 |
| 20701631 | 66636 | rs9358349 |
| 20701770 | 66775 | rs9460526 |
| 20701829 | 66834 | rs9366356 |
| 20702163 | 67168 | rs36120092 |
| 20702181 | 67186 | rs9465824 |
| 20702519 | 67524 | rs4712512 |
| 20702561 | 67566 | rs4712513 |
| 20702646 | 67651 | rs4710934 |
| 20702658 | 67663 | rs9348438 |
| 20702902 | 67907 | rs9460529 |
| 20703363 | 68368 | rs13199587 |
| 20703470 | 68475 | rs13199384 |
| 20703526 | 68531 | rs10223680 |
| 20703606 | 68611 | rs9350263 |
| 20703768 | 68773 | rs9465825 |
| 20703832 | 68837 | rs10223876 |
| 20704100 | 69105 | rs35702271 |
| 20704171 | 69176 | rs9358350 |
| 20704432 | 69437 | rs12208985 |
| 20704771 | 69776 | rs12210459 |
| 20704892 | 69897 | rs35431707 |
| 20705144 | 70149 | rs36039523 |
| 20705297 | 70302 | rs11758281 |
| 20705350 | 70355 | rs28893199 |
| 20705757 | 70762 | rs34256347 |
| 20706019 | 71024 | rs12192740 |
| 20706282 | 71287 | rs13212326 |
| 20706486 | 71491 | rs12199184 |
| 20706753 | 71758 | rs10456234 |
| 20707009 | 72014 | rs4712514 |
| 20707422 | 72427 | rs9465826 |
| 20707607 | 72612 | rs9366357 |
| 20707867 | 72872 | rs2294809 |
| 20708549 | 73554 | rs2294808 |
| 20708813 | 73818 | rs7762750 |
| 20708976 | 73981 | rs4712515 |
| 20708998 | 74003 | rs10522824 |
| 20708999 | 74004 | rs35660518 |
| 20709002 | 74007 | rs10679950 |
| 20709003 | 74008 | rs34870864 |
| 20709022 | 74027 | rs4712516 |
| 20709145 | 74150 | rs4710935 |
| 20709386 | 74391 | rs9465827 |
| 20709388 | 74393 | rs12204865 |
| 20709672 | 74677 | rs10946393 |
| 20709764 | 74769 | rs12209806 |
| 20709894 | 74899 | rs10946394 |
| 20709921 | 74926 | rs1997778 |
| 20709971 | 74976 | rs35878587 |
| 20710359 | 75364 | rs1997777 |
| 20710378 | 75383 | rs2223622 |
| 20710776 | 75781 | rs11964057 |
| 20711246 | 76251 | rs9460530 |
| 20711344 | 76349 | rs9460531 |
| 20711376 | 76381 | rs34329159 |
| 20711640 | 76645 | rs7764558 |
| 20711804 | 76809 | rs4710936 |
| 20712056 | 77061 | rs12213940 |
| 20712228 | 77233 | rs13215038 |
| 20712739 | 77744 | rs10946395 |
| 20712832 | 77837 | rs6939917 |
| 20712975 | 77980 | rs9358351 |
| 20713800 | 78805 | rs6925097 |
| 20713924 | 78929 | rs9465828 |
| 20713955 | 78960 | rs9465829 |
| 20713961 | 78966 | rs6902661 |
| 20714057 | 79062 | rs34373680 |
| 20714281 | 79286 | rs35051096 |
| 20714508 | 79513 | rs932405 |
| 20714591 | 79596 | rs6926585 |
| 20714635 | 79640 | rs3938395 |
| 20715464 | 80469 | rs11964664 |
| 20715551 | 80556 | rs35964987 |
| 20715663 | 80668 | rs12206413 |
| 20715758 | 80763 | rs35990187 |
| 20715763 | 80768 | rs4991654 |
| 20715910 | 80915 | rs9460532 |
| 20715991 | 80996 | rs13328250 |
| 20716030 | 81035 | rs13328252 |
| 20716194 | 81199 | rs4712517 |
| 20716257 | 81262 | rs4712518 |
| 20717220 | 82225 | rs7758129 |
| 20717475 | 82480 | rs13206462 |
| 20717483 | 82488 | rs13192442 |
| 20717486 | 82491 | rs13206468 |
| 20717492 | 82497 | rs13192445 |
| 20717498 | 82503 | rs13192450 |
| 20717504 | 82509 | rs13206477 |
| 20717510 | 82515 | rs13206483 |
| 20717577 | 82582 | rs12179168 |
| 20717586 | 82591 | rs12180975 |
| 20717611 | 82616 | rs12179172 |
| 20717860 | 82865 | rs12179563 |
| 20718357 | 83362 | rs11355836 |
| 20718696 | 83701 | rs2328527 |
| 20718709 | 83714 | rs11452882 |
| 20718920 | 83925 | rs2876574 |
| 20719905 | 84910 | rs9465831 |
| 20720031 | 85036 | rs34877824 |
| 20720290 | 85295 | rs13212501 |
| 20720647 | 85652 | rs9358352 |
| 20720703 | 85708 | rs9358353 |
| 20720761 | 85766 | rs28756205 |
| 20720889 | 85894 | rs9350265 |
| 20720989 | 85994 | rs7750508 |
| 20721130 | 86135 | rs7771052 |
| 20721141 | 86146 | rs9460533 |
| 20721195 | 86200 | rs13200415 |
| 20721216 | 86221 | rs10550932 |
| 20721312 | 86317 | rs9465832 |
| 20721463 | 86468 | rs9368211 |
| 20721471 | 86476 | rs9350266 |
| 20721507 | 86512 | rs11752592 |
| 20721515 | 86520 | rs9350267 |
| 20721754 | 86759 | rs978988 |
| 20721898 | 86903 | rs978987 |
| 20721906 | 86911 | rs978986 |
| 20722036 | 87041 | rs5874773 |
| 20722196 | 87201 | rs5874774 |
| 20722500 | 87505 | rs7756788 |
| 20722659 | 87664 | rs9348439 |
| 20722661 | 87666 | rs9356739 |
| 20722693 | 87698 | rs9356740 |
| 20722738 | 87743 | rs7760894 |
| 20723021 | 88026 | rs2796913 |
| 20723046 | 88051 | rs2608613 |
| 20723121 | 88126 | rs10456710 |
| 20723139 | 88144 | rs9476286 |
| 20723140 | 88145 | rs9476287 |
| 20723140 | 88145 | rs28368538 |
| 20723141 | 88146 | rs28612622 |
| 20723142 | 88147 | rs9463660 |
| 20723146 | 88151 | rs9463661 |
| 20723162 | 88167 | rs9461022 |
| 20723193 | 88198 | rs9461021 |
| 20723235 | 88240 | rs34980442 |
| 20723239 | 88244 | rs34049080 |
| 20723258 | 88263 | rs35218684 |
| 20723260 | 88265 | rs12175876 |
| 20723270 | 88275 | rs10948323 |
| 20723287 | 88292 | rs34400313 |
| 20723292 | 88297 | rs36081550 |
| 20723304 | 88309 | rs12175878 |
| 20723305 | 88310 | rs34520184 |
| 20723305 | 88310 | rs34756989 |
| 20723322 | 88327 | rs4629736 |
| 20723322 | 88327 | rs9296917 |
| 20723324 | 88329 | rs28562027 |
| 20723333 | 88338 | rs9381823 |
| 20723346 | 88351 | rs34769771 |
| 20723346 | 88351 | rs34774640 |
| 20723346 | 88351 | rs36038896 |
| 20723365 | 88370 | rs13209195 |
| 20723369 | 88374 | rs12213541 |
| 20723369 | 88374 | rs34112320 |
| 20723371 | 88376 | rs34615869 |
| 20723379 | 88384 | rs35949519 |
| 20723381 | 88386 | rs9257498 |
| 20723383 | 88388 | rs34200576 |
| 20723393 | 88398 | rs4960519 |
| 20723396 | 88401 | rs9261905 |
| 20723402 | 88407 | rs9267103 |
| 20723404 | 88409 | rs17367677 |
| 20723416 | 88421 | rs9261906 |
| 20723421 | 88426 | rs9267104 |
| 20723424 | 88429 | rs9267105 |
| 20723434 | 88439 | rs28810763 |
| 20723438 | 88443 | rs12179121 |
| 20723438 | 88443 | rs4714959 |
| 20723439 | 88444 | rs10946636 |
| 20723439 | 88444 | rs28763327 |
| 20723439 | 88444 | rs28847950 |
| 20723439 | 88444 | rs3933247 |
| 20723449 | 88454 | rs28808723 |
| 20723451 | 88456 | rs9767082 |
| 20723452 | 88457 | rs35132675 |
| 20723452 | 88457 | rs35517166 |
| 20723452 | 88457 | rs4714297 |
| 20723456 | 88461 | rs12182737 |
| 20723456 | 88461 | rs36163804 |
| 20723460 | 88465 | rs35790973 |
| 20723464 | 88469 | rs35236694 |
| 20723469 | 88474 | rs35567559 |
| 20723469 | 88474 | rs9267106 |
| 20723471 | 88476 | rs9800557 |
| 20723484 | 88489 | rs12182463 |
| 20723489 | 88494 | rs10948322 |
| 20723489 | 88494 | rs34052284 |
| 20723489 | 88494 | rs9268999 |
| 20723490 | 88495 | rs9258377 |
| 20723495 | 88500 | rs9257499 |
| 20723498 | 88503 | rs9265816 |
| 20723500 | 88505 | rs28771402 |
| 20723501 | 88506 | rs28771401 |
| 20723502 | 88507 | rs12194731 |
| 20723502 | 88507 | rs4451188 |
| 20723502 | 88507 | rs9717323 |
| 20723502 | 88507 | rs9765920 |
| 20723503 | 88508 | rs9265815 |
| 20723504 | 88509 | rs28771400 |
| 20723506 | 88511 | rs12190813 |
| 20723506 | 88511 | rs12215416 |
| 20723507 | 88512 | rs12178527 |
| 20723510 | 88515 | rs35234761 |
| 20723510 | 88515 | rs35887156 |
| 20723512 | 88517 | rs28797321 |
| 20723515 | 88520 | rs28771399 |
| 20723522 | 88527 | rs13207682 |
| 20723522 | 88527 | rs36099432 |
| 20723531 | 88536 | rs28749543 |
| 20723535 | 88540 | rs13196506 |
| 20723535 | 88540 | rs35716308 |
| 20723536 | 88541 | rs28831180 |
| 20723536 | 88541 | rs34938144 |
| 20723537 | 88542 | rs36142967 |
| 20723541 | 88546 | rs35313792 |
| 20723542 | 88547 | rs9260904 |
| 20723543 | 88548 | rs34597832 |
| 20723545 | 88550 | rs9767740 |
| 20723546 | 88551 | rs28771398 |
| 20723550 | 88555 | rs34384951 |
| 20723554 | 88559 | rs12524128 |
| 20723554 | 88559 | rs12665124 |
| 20723554 | 88559 | rs34922643 |
| 20723555 | 88560 | rs9260903 |
| 20723557 | 88562 | rs28771397 |
| 20723557 | 88562 | rs6915279 |
| 20723559 | 88564 | rs9261907 |
| 20723559 | 88564 | rs9766798 |
| 20723559 | 88564 | rs9767242 |
| 20723560 | 88565 | rs6914835 |
| 20723560 | 88565 | rs9260902 |
| 20723560 | 88565 | rs9767101 |
| 20723562 | 88567 | rs9261908 |
| 20723563 | 88568 | rs12207064 |
| 20723563 | 88568 | rs12213193 |
| 20723563 | 88568 | rs35750154 |
| 20723566 | 88571 | rs9260901 |
| 20723570 | 88575 | rs9269000 |
| 20723579 | 88584 | rs12178368 |
| 20723579 | 88584 | rs13197714 |
| 20723579 | 88584 | rs28771396 |
| 20723579 | 88584 | rs34443697 |
| 20723579 | 88584 | rs9267107 |
| 20723582 | 88587 | rs35113301 |
| 20723582 | 88587 | rs3933248 |
| 20723583 | 88588 | rs28819830 |
| 20723586 | 88591 | rs10947899 |
| 20723586 | 88591 | rs9260900 |
| 20723588 | 88593 | rs13204671 |
| 20723588 | 88593 | rs34094007 |
| 20723588 | 88593 | rs34963756 |
| 20723588 | 88593 | rs4304158 |
| 20723589 | 88594 | rs4298351 |
| 20723590 | 88595 | rs12182307 |
| 20723590 | 88595 | rs12201487 |
| 20723590 | 88595 | rs34097573 |
| 20723595 | 88600 | rs13219021 |
| 20723596 | 88601 | rs34456153 |
| 20723596 | 88601 | rs9767597 |
| 20723597 | 88602 | rs9767102 |
| 20723598 | 88603 | rs9269001 |
| 20723601 | 88606 | rs12180097 |
| 20723603 | 88608 | rs12178465 |
| 20723603 | 88608 | rs35128115 |
| 20723607 | 88612 | rs4273681 |
| 20723609 | 88614 | rs12193754 |
| 20723617 | 88622 | rs813814 |
| 20723617 | 88622 | rs9689672 |
| 20723619 | 88624 | rs36173068 |
| 20723623 | 88628 | rs12202172 |
| 20723623 | 88628 | rs28862376 |
| 20723625 | 88630 | rs12178884 |
| 20723625 | 88630 | rs12203117 |
| 20723625 | 88630 | rs34250588 |
| 20723626 | 88631 | rs12182581 |
| 20723632 | 88637 | rs34147797 |
| 20723635 | 88640 | rs9688484 |
| 20723636 | 88641 | rs9269002 |
| 20723636 | 88641 | rs9767733 |
| 20723637 | 88642 | rs10948989 |
| 20723637 | 88642 | rs34732676 |
| 20723637 | 88642 | rs9260899 |
| 20723638 | 88643 | rs35686233 |
| 20723639 | 88644 | rs11753098 |
| 20723639 | 88644 | rs35156647 |
| 20723646 | 88651 | rs35575623 |
| 20723646 | 88651 | rs34719653 |
| 20723648 | 88653 | rs12192046 |
| 20723649 | 88654 | rs11759854 |
| 20723649 | 88654 | rs28771395 |
| 20723649 | 88654 | rs35563402 |
| 20723649 | 88654 | rs36016334 |
| 20723649 | 88654 | rs4555911 |
| 20723653 | 88658 | rs11965757 |
| 20723654 | 88659 | rs10948990 |
| 20723654 | 88659 | rs28808296 |
| 20723654 | 88659 | rs34995142 |
| 20723654 | 88659 | rs809919 |
| 20723657 | 88662 | rs11571978 |
| 20723659 | 88664 | rs12208488 |
| 20723662 | 88667 | rs11751374 |
| 20723663 | 88668 | rs13217613 |
| 20723667 | 88672 | rs12528735 |
| 20723668 | 88673 | rs35160656 |
| 20723668 | 88673 | rs35322569 |
| 20723668 | 88673 | rs9766221 |
| 20723670 | 88675 | rs35120225 |
| 20723670 | 88675 | rs511868 |
| 20723674 | 88679 | rs9269003 |
| 20723675 | 88680 | rs34290316 |
| 20723675 | 88680 | rs35735496 |
| 20723675 | 88680 | rs9689102 |
| 20723676 | 88681 | rs10948991 |
| 20723676 | 88681 | rs9269004 |
| 20723677 | 88682 | rs13220607 |
| 20723677 | 88682 | rs34700647 |
| 20723677 | 88682 | rs9260898 |
| 20723678 | 88683 | rs9260897 |
| 20723684 | 88689 | rs28771394 |
| 20723686 | 88691 | rs12213200 |
| 20723687 | 88692 | rs9717987 |
| 20723688 | 88693 | rs28771393 |
| 20723688 | 88693 | rs9717716 |
| 20723689 | 88694 | rs28771392 |
| 20723690 | 88695 | rs11758009 |
| 20723690 | 88695 | rs34159662 |
| 20723690 | 88695 | rs34683172 |
| 20723690 | 88695 | rs9717331 |
| 20723691 | 88696 | rs9717853 |
| 20723692 | 88697 | rs9765875 |
| 20723696 | 88701 | rs12178974 |
| 20723696 | 88701 | rs36003577 |
| 20723698 | 88703 | rs9767747 |
| 20723700 | 88705 | rs28771391 |
| 20723704 | 88709 | rs13216352 |
| 20723707 | 88712 | rs12200762 |
| 20723707 | 88712 | rs12206373 |
| 20723707 | 88712 | rs34570202 |
| 20723707 | 88712 | rs36197940 |
| 20723707 | 88712 | rs9265814 |
| 20723714 | 88719 | rs34131282 |
| 20723717 | 88722 | rs12175478 |
| 20723720 | 88725 | rs28772692 |
| 20723722 | 88727 | rs35516674 |
| 20723722 | 88727 | rs35704013 |
| 20723727 | 88732 | rs34691406 |
| 20723728 | 88733 | rs28771390 |
| 20723728 | 88733 | rs4374863 |
| 20723730 | 88735 | rs34826149 |
| 20723734 | 88739 | rs12207894 |
| 20723734 | 88739 | rs12662476 |
| 20723736 | 88741 | rs9260896 |
| 20723739 | 88744 | rs28771389 |
| 20723740 | 88745 | rs28771388 |
| 20723741 | 88746 | rs9382592 |
| 20723745 | 88750 | rs12173375 |
| 20723745 | 88750 | rs9268072 |
| 20723745 | 88750 | rs9382110 |
| 20723746 | 88751 | rs4715211 |
| 20723747 | 88752 | rs34128950 |
| 20723747 | 88752 | rs35961188 |
| 20723747 | 88752 | rs4620119 |
| 20723749 | 88754 | rs9767236 |
| 20723752 | 88757 | rs34717143 |
| 20723755 | 88760 | rs13201202 |
| 20723756 | 88761 | rs13201503 |
| 20723762 | 88767 | rs13197088 |
| 20723762 | 88767 | rs35347849 |
| 20723766 | 88771 | rs34663083 |
| 20723766 | 88771 | rs35650828 |
| 20723767 | 88772 | rs13199241 |
| 20723767 | 88772 | rs34205031 |
| 20723768 | 88773 | rs28749541 |
| 20723768 | 88773 | rs34469031 |
| 20723768 | 88773 | rs9279137 |
| 20723769 | 88774 | rs9260895 |
| 20723770 | 88775 | rs12212483 |
| 20723770 | 88775 | rs35446958 |
| 20723775 | 88780 | rs28380829 |
| 20723777 | 88782 | rs12525384 |
| 20723777 | 88782 | rs34576984 |
| 20723782 | 88787 | rs283541 |
| 20723785 | 88790 | rs28380828 |
| 20723788 | 88793 | rs10948698 |
| 20723790 | 88795 | rs9265813 |
| 20723791 | 88796 | rs9689173 |
| 20723792 | 88797 | rs28380827 |
| 20723795 | 88800 | rs35399169 |
| 20723799 | 88804 | rs9260894 |
| 20723800 | 88805 | rs12173388 |
| 20723800 | 88805 | rs9269005 |
| 20723802 | 88807 | rs28359816 |
| 20723803 | 88808 | rs28380826 |
| 20723807 | 88812 | rs280297 |
| 20723807 | 88812 | rs34507582 |
| 20723807 | 88812 | rs9261623 |
| 20723810 | 88815 | rs12174621 |
| 20723813 | 88818 | rs28380825 |
| 20723815 | 88820 | rs35197377 |
| 20723816 | 88821 | rs17362870 |
| 20723816 | 88821 | rs34562190 |
| 20723816 | 88821 | rs35071522 |
| 20723816 | 88821 | rs35179751 |
| 20723816 | 88821 | rs4458721 |
| 20723817 | 88822 | rs12180385 |
| 20723817 | 88822 | rs12206581 |
| 20723817 | 88822 | rs12333308 |
| 20723817 | 88822 | rs9269006 |
| 20723821 | 88826 | rs9688475 |
| 20723829 | 88834 | rs9269007 |
| 20723832 | 88837 | rs9269008 |
| 20723832 | 88837 | rs9269009 |
| 20723834 | 88839 | rs35899754 |
| 20723836 | 88841 | rs13211190 |
| 20723837 | 88842 | rs12216274 |
| 20723838 | 88843 | rs12191544 |
| 20723838 | 88843 | rs4337934 |
| 20723841 | 88846 | rs9279295 |
| 20723842 | 88847 | rs12178577 |
| 20723849 | 88854 | rs12215604 |
| 20723853 | 88858 | rs9395360 |
| 20723854 | 88859 | rs28749540 |
| 20723855 | 88860 | rs28895226 |
| 20723855 | 88860 | rs9260893 |
| 20723861 | 88866 | rs4365925 |
| 20723864 | 88869 | rs9261912 |
| 20723865 | 88870 | rs28895227 |
| 20723870 | 88875 | rs9261913 |
| 20723873 | 88878 | rs12183502 |
| 20723873 | 88878 | rs34332895 |
| 20723873 | 88878 | rs9472692 |
| 20723876 | 88881 | rs35640475 |
| 20723884 | 88889 | rs9269010 |
| 20723888 | 88893 | rs12192337 |
| 20723888 | 88893 | rs35349786 |
| 20723891 | 88896 | rs34978372 |
| 20723893 | 88898 | rs280296 |
| 20723894 | 88899 | rs11755576 |
| 20723903 | 88908 | rs28803616 |
| 20723903 | 88908 | rs35553517 |
| 20723904 | 88909 | rs4358654 |
| 20723910 | 88915 | rs9260892 |
| 20723914 | 88919 | rs13196941 |
| 20723914 | 88919 | rs9717238 |
| 20723915 | 88920 | rs12175553 |
| 20723915 | 88920 | rs12191408 |
| 20723915 | 88920 | rs4460210 |
| 20723915 | 88920 | rs9260891 |
| 20723918 | 88923 | rs9395361 |
| 20723920 | 88925 | rs9766917 |
| 20723926 | 88931 | rs35073779 |
| 20723931 | 88936 | rs9765884 |
| 20723931 | 88936 | rs9767229 |
| 20723932 | 88937 | rs35247846 |
| 20723932 | 88937 | rs9717328 |
| 20723936 | 88941 | rs28805674 |
| 20723936 | 88941 | rs9260890 |
| 20723938 | 88943 | rs34926348 |
| 20723938 | 88943 | rs9717233 |
| 20723945 | 88950 | rs35869128 |
| 20723946 | 88951 | rs12175941 |
| 20723951 | 88956 | rs9260889 |
| 20723952 | 88957 | rs9766686 |
| 20723956 | 88961 | rs10046203 |
| 20723957 | 88962 | rs9260888 |
| 20723958 | 88963 | rs12180540 |
| 20723961 | 88966 | rs9766115 |
| 20723968 | 88973 | rs6912019 |
| 20723969 | 88974 | rs9767458 |
| 20723976 | 88981 | rs13202161 |
| 20723989 | 88994 | rs13218048 |
| 20724000 | 89005 | rs13218040 |
| 20724001 | 89006 | rs13206557 |
| 20724002 | 89007 | rs13202152 |
| 20724013 | 89018 | rs12210049 |
| 20724013 | 89018 | rs34552032 |
| 20724018 | 89023 | rs28890881 |
| 20724018 | 89023 | rs34478537 |
| 20724025 | 89030 | rs35191657 |
| 20724038 | 89043 | rs34450517 |
| 20724043 | 89048 | rs9689655 |
| 20724064 | 89069 | rs12180172 |
| 20724068 | 89073 | rs28865015 |
| 20724071 | 89076 | rs9717839 |
| 20724072 | 89077 | rs9717836 |
| 20724074 | 89079 | rs9885593 |
| 20724082 | 89087 | rs28887572 |
| 20724083 | 89088 | rs13216113 |
| 20724083 | 89088 | rs35254115 |
| 20724084 | 89089 | rs10949190 |
| 20724085 | 89090 | rs6923503 |
| 20724087 | 89092 | rs12214411 |
| 20724091 | 89096 | rs28747986 |
| 20724096 | 89101 | rs1087363 |
| 20724096 | 89101 | rs1091092 |
| 20724104 | 89109 | rs12180759 |
| 20724107 | 89112 | rs11758052 |
| 20724112 | 89117 | rs9261321 |
| 20724113 | 89118 | rs28861738 |
| 20724113 | 89118 | rs36109104 |
| 20724123 | 89128 | rs13208115 |
| 20724129 | 89134 | rs9472693 |
| 20724130 | 89135 | rs12208570 |
| 20724130 | 89135 | rs28832660 |
| 20724131 | 89136 | rs13200482 |
| 20724131 | 89136 | rs9260886 |
| 20724131 | 89136 | rs9472694 |
| 20724159 | 89164 | rs9260885 |
| 20724172 | 89177 | rs280295 |
| 20724172 | 89177 | rs28749538 |
| 20724174 | 89179 | rs13201041 |
| 20724184 | 89189 | rs1087362 |
| 20724187 | 89192 | rs12183850 |
| 20724190 | 89195 | rs12202552 |
| 20724208 | 89213 | rs9260884 |
| 20724210 | 89215 | rs9474341 |
| 20724211 | 89216 | rs9260883 |
| 20724222 | 89227 | rs34669820 |
| 20724225 | 89230 | rs12189886 |
| 20724234 | 89239 | rs12182114 |
| 20724266 | 89271 | rs9688643 |
| 20724272 | 89277 | rs12195237 |
| 20724281 | 89286 | rs35293595 |
| 20724281 | 89286 | rs9381428 |
| 20724306 | 89311 | rs34665644 |
| 20724309 | 89314 | rs12189599 |
| 20724311 | 89316 | rs12208503 |
| 20724312 | 89317 | rs9800791 |
| 20724330 | 89335 | rs12173681 |
| 20724337 | 89342 | rs12182094 |
| 20724343 | 89348 | rs12182119 |
| 20724352 | 89357 | rs12182102 |
| 20724392 | 89397 | rs12174506 |
| 20724393 | 89398 | rs9767809 |
| 20724394 | 89399 | rs12173703 |
| 20724401 | 89406 | rs12202889 |
| 20724435 | 89440 | rs28840538 |
| 20724466 | 89471 | rs9473983 |
| 20724467 | 89472 | rs34220946 |
| 20724487 | 89492 | rs9261322 |
| 20724491 | 89496 | rs9261323 |
| 20724517 | 89522 | rs526092 |
| 20724564 | 89569 | rs9473982 |
| 20724566 | 89571 | rs34307764 |
| 20724626 | 89631 | rs34490559 |
| 20724665 | 89670 | rs4449621 |
| 20724685 | 89690 | rs4714818 |
| 20724698 | 89703 | rs4714817 |
| 20724722 | 89727 | rs682384 |
| 20724738 | 89743 | rs682051 |
| 20724753 | 89758 | rs12530056 |
| 20724756 | 89761 | rs35086683 |
| 20724888 | 89893 | rs12528468 |
| 20725092 | 90097 | rs10650195 |
| 20725092 | 90097 | rs35956126 |
| 20725109 | 90114 | rs6937578 |
| 20725159 | 90164 | rs12530107 |
| 20725173 | 90178 | rs720448 |
| 20725176 | 90181 | rs9368212 |
| 20725250 | 90255 | rs35481531 |
| 20725253 | 90258 | rs720449 |
| 20725262 | 90267 | rs10650196 |
| 20725495 | 90500 | rs2064 |
| 20725499 | 90504 | rs2065 |
| 20725638 | 90643 | rs9368213 |
| 20725732 | 90737 | rs41455744 |
| 20726077 | 91082 | rs34960654 |
| 20726154 | 91159 | rs6900954 |
| 20726168 | 91173 | rs6456359 |
| 20726176 | 91181 | rs7739405 |
| 20726601 | 91606 | rs7760508 |
| 20726684 | 91689 | rs10456007 |
| 20726987 | 91992 | rs9460534 |
| 20727168 | 92173 | rs11966749 |
| 20727353 | 92358 | rs9358354 |
| 20727809 | 92814 | rs9368214 |
| 20727982 | 92987 | rs9350268 |
| 20728095 | 93100 | rs2069013 |
| 20728160 | 93165 | rs2069014 |
| 20728290 | 93295 | rs2069015 |
| 20728292 | 93297 | rs11967475 |
| 20729096 | 94101 | rs10214549 |
| 20729217 | 94222 | rs2223621 |
| 20729281 | 94286 | rs9465835 |
| 20729984 | 94989 | rs35404829 |
| 20729995 | 95000 | rs10687080 |
| 20730233 | 95238 | rs6915936 |
| 20730331 | 95336 | rs9295472 |
| 20730732 | 95737 | rs1569660 |
| 20730929 | 95934 | rs7455009 |
| 20730945 | 95950 | rs1569659 |
| 20730946 | 95951 | rs5874775 |
| 20730948 | 95953 | rs10601252 |
| 20730983 | 95988 | rs1569658 |
| 20731030 | 96035 | rs34142400 |
| 20731063 | 96068 | rs16883996 |
| 20731181 | 96186 | rs35073110 |
| 20731184 | 96189 | rs5028948 |
| 20731263 | 96268 | rs34201758 |
| 20731371 | 96376 | rs6456360 |
| 20731373 | 96378 | rs6456361 |
| 20731380 | 96385 | rs6456362 |
| 20731584 | 96589 | rs6922571 |
| 20731925 | 96930 | rs35145358 |
| 20731950 | 96955 | rs9465836 |
| 20732013 | 97018 | rs7763304 |
| 20732158 | 97163 | rs9465837 |
| 20732218 | 97223 | rs7743314 |
| 20732337 | 97342 | rs714831 |
| 20732361 | 97366 | rs714830 |
| 20732484 | 97489 | rs7743789 |
| 20732584 | 97589 | rs5874776 |
| 20732589 | 97594 | rs5874777 |
| 20732965 | 97970 | rs16884003 |
| 20733079 | 98084 | rs9356741 |
| 20733080 | 98085 | rs9366358 |
| 20733246 | 98251 | rs9356742 |
| 20733430 | 98435 | rs11756987 |
| 20733470 | 98475 | rs9465838 |
| 20733613 | 98618 | rs2206579 |
| 20733920 | 98925 | rs35111339 |
| 20734361 | 99366 | rs6917583 |
| 20734388 | 99393 | rs6917599 |
| 20734630 | 99635 | rs13437429 |
| 20735418 | 100423 | rs34307011 |
| 20735420 | 100425 | rs4515379 |
| 20735756 | 100761 | rs2223620 |
| 20735870 | 100875 | rs9465839 |
| 20735993 | 100998 | rs12664972 |
| 20736790 | 101795 | rs28402356 |
| 20736798 | 101803 | rs6934727 |
| 20736873 | 101878 | rs35493429 |
| 20736881 | 101886 | rs11369825 |
| 20737261 | 102266 | rs4523079 |
| 20737470 | 102475 | rs7771213 |
| 20737568 | 102573 | rs9465840 |
| 20737687 | 102692 | rs9465841 |
| 20737767 | 102772 | rs9465842 |
| 20737837 | 102842 | rs9460535 |
| 20737992 | 102997 | rs34835908 |
| 20738060 | 103065 | rs9465843 |
| 20738139 | 103144 | rs13220465 |
| 20738159 | 103164 | rs13220352 |
| 20738321 | 103326 | rs4710937 |
| 20738375 | 103380 | rs9460536 |
| 20738376 | 103381 | rs13190734 |
| 20738451 | 103456 | rs9465844 |
| 20738712 | 103717 | rs35976895 |
| 20738713 | 103718 | rs11327958 |
| 20738932 | 103937 | rs13194407 |
| 20738990 | 103995 | rs10484633 |
| 20739524 | 104529 | rs2328528 |
| 20739789 | 104794 | rs35958155 |
| 20739809 | 104814 | rs4421185 |
| 20739932 | 104937 | rs2328529 |
| 20740089 | 105094 | rs9460537 |
| 20740745 | 105750 | rs17224527 |
| 20740886 | 105891 | rs34166991 |
| 20741024 | 106029 | rs17823073 |
| 20741276 | 106281 | rs12183074 |
| 20741316 | 106321 | rs9465845 |
| 20741844 | 106849 | rs35148963 |
| 20741886 | 106891 | rs7768642 |
| 20742320 | 107325 | rs9465846 |
| 20742407 | 107412 | rs9465847 |
| 20742551 | 107556 | rs17823127 |
| 20742594 | 107599 | rs16884021 |
| 20742630 | 107635 | rs4533976 |
| 20742865 | 107870 | rs7755830 |
| 20743005 | 108010 | rs7756031 |
| 20743139 | 108144 | rs35650451 |
| 20743192 | 108197 | rs10561117 |
| 20743241 | 108246 | rs6940200 |
| 20743799 | 108804 | rs9465848 |
| 20743808 | 108813 | rs34351919 |
| 20745083 | 110088 | rs4712519 |
| 20745285 | 110290 | rs9368215 |
| 20745566 | 110571 | rs12206285 |
| 20745853 | 110858 | rs34049994 |
| 20745988 | 110993 | rs7751957 |
| 20746702 | 111707 | rs9465849 |
| 20746857 | 111862 | rs7341291 |
| 20746957 | 111962 | rs6921014 |
| 20747388 | 112393 | rs9465850 |
| 20747583 | 112588 | rs7758612 |
| 20747681 | 112686 | rs35602526 |
| 20748295 | 113300 | rs9465851 |
| 20748399 | 113404 | rs34257578 |
| 20748403 | 113408 | rs11339738 |
| 20748516 | 113521 | rs9460538 |
| 20748850 | 113855 | rs4712520 |
| 20748883 | 113888 | rs4710938 |
| 20749084 | 114089 | rs35889049 |
| 20749315 | 114320 | rs9348440 |
| 20749879 | 114884 | rs9465852 |
| 20750054 | 115059 | rs12196009 |
| 20750306 | 115311 | rs11968248 |
| 20750353 | 115358 | rs34797179 |
| 20750764 | 115769 | rs6925328 |
| 20750913 | 115918 | rs36073053 |
| 20751137 | 116142 | rs35462488 |
| 20751140 | 116145 | rs36224625 |
| 20751150 | 116155 | rs3060659 |
| 20751153 | 116158 | rs5874778 |
| 20751155 | 116160 | rs11267861 |
| 20751201 | 116206 | rs4235999 |
| 20751230 | 116235 | rs16884038 |
| 20751706 | 116711 | rs2328530 |
| 20751731 | 116736 | rs2328531 |
| 20751750 | 116755 | rs28360636 |
| 20752162 | 117167 | rs6932676 |
| 20752183 | 117188 | rs9460539 |
| 20752303 | 117308 | rs6932876 |
| 20752346 | 117351 | rs34929755 |
| 20752359 | 117364 | rs28733367 |
| 20752360 | 117365 | rs11348111 |
| 20752473 | 117478 | rs6933219 |
| 20752702 | 117707 | rs6933165 |
| 20752872 | 117877 | rs35255583 |
| 20752901 | 117906 | rs34332316 |
| 20752923 | 117928 | rs4710939 |
| 20753486 | 118491 | rs11970417 |
| 20753659 | 118664 | rs11963217 |
| 20753670 | 118675 | rs11965473 |
| 20753833 | 118838 | rs2876575 |
| 20754049 | 119054 | rs7739974 |
| 20754976 | 119981 | rs7745175 |
| 20755173 | 120178 | rs7765784 |
| 20755178 | 120183 | rs6907731 |
| 20755250 | 120255 | rs35057896 |
| 20755313 | 120318 | rs7746072 |
| 20755639 | 120644 | rs10484632 |
| 20755770 | 120775 | rs35444529 |
| 20755849 | 120854 | rs17823571 |
| 20755941 | 120946 | rs11965062 |
| 20755962 | 120967 | rs6936199 |
| 20755965 | 120970 | rs6913126 |
| 20756178 | 121183 | rs6913509 |
| 20756613 | 121618 | rs34638218 |
| 20756673 | 121678 | rs35746011 |
| 20756741 | 121746 | rs9460540 |
| 20757090 | 122095 | rs36045545 |
| 20757129 | 122134 | rs35392790 |
| 20757233 | 122238 | rs6456364 |
| 20757260 | 122265 | rs2179553 |
| 20757513 | 122518 | rs9350269 |
| 20757531 | 122536 | rs9465854 |
| 20757787 | 122792 | rs2179552 |
| 20757936 | 122941 | rs6925233 |
| 20758033 | 123038 | rs2328532 |
| 20758248 | 123253 | rs7743970 |
| 20758317 | 123322 | rs13209457 |
| 20758318 | 123323 | rs34641285 |
| 20758344 | 123349 | rs13209538 |
| 20758387 | 123392 | rs2876576 |
| 20758479 | 123484 | rs13209572 |
| 20758653 | 123658 | rs28783153 |
| 20758712 | 123717 | rs9969037 |
| 20758800 | 123805 | rs7766844 |
| 20758969 | 123974 | rs7767133 |
| 20759291 | 124296 | rs7749464 |
| 20760100 | 125105 | rs2050225 |
| 20760696 | 125701 | rs9295474 |
| 20760744 | 125749 | rs9295475 |
| 20761529 | 126534 | rs2328545 |
| 20761548 | 126553 | rs28846771 |
| 20761779 | 126784 | rs2876582 |
| 20761899 | 126904 | rs10223446 |
| 20762094 | 127099 | rs13219723 |
| 20762095 | 127100 | rs13203489 |
| 20762154 | 127159 | rs13203583 |
| 20762172 | 127177 | rs13203887 |
| 20762279 | 127284 | rs6456366 |
| 20762876 | 127881 | rs9358355 |
| 20763089 | 128094 | rs9368216 |
| 20763375 | 128380 | rs9465855 |
| 20763384 | 128389 | rs9465856 |
| 20763463 | 128468 | rs16884070 |
| 20763482 | 128487 | rs16884072 |
| 20763647 | 128652 | rs13208604 |
| 20764169 | 129174 | rs9465857 |
| 20764307 | 129312 | rs9368217 |
| 20764559 | 129564 | rs9460541 |
| 20764746 | 129751 | rs9460542 |
| 20764765 | 129770 | rs11969955 |
| 20764779 | 129784 | rs4712522 |
| 20764924 | 129929 | rs16884074 |
| 20765172 | 130177 | rs34489684 |
| 20765324 | 130329 | rs2328546 |
| 20765543 | 130548 | rs4712523 |
| 20765844 | 130849 | rs4712524 |
| 20765898 | 130903 | rs35397753 |
| 20765991 | 130996 | rs4710940 |
| 20766197 | 131202 | rs13190727 |
| 20766215 | 131220 | rs35136485 |
| 20766311 | 131316 | rs35260725 |
| 20766335 | 131340 | rs35939620 |
| 20766566 | 131571 | rs17823996 |
| 20766713 | 131718 | rs16884082 |
| 20767438 | 132443 | rs6906327 |
| 20767566 | 132571 | rs6456367 |
| 20767785 | 132790 | rs6456368 |
| 20768202 | 133207 | rs7749083 |
| 20768344 | 133349 | rs6456369 |
| 20768669 | 133674 | rs10946396 |
| 20768672 | 133677 | rs10946397 |
| 20768710 | 133715 | rs11759505 |
| 20769000 | 134005 | rs13203361 |
| 20769013 | 134018 | rs10946398 |
| 20769122 | 134127 | rs7774594 |
| 20769229 | 134234 | rs7754840 |
| 20769249 | 134254 | rs9460543 |
| 20769508 | 134513 | rs9460544 |
| 20769529 | 134534 | rs9460545 |
| 20769711 | 134716 | rs2206740 |
| 20769806 | 134811 | rs5874779 |
| 20769807 | 134812 | rs33970890 |
| 20769815 | 134820 | rs5874780 |
| 20769816 | 134821 | rs35014292 |
| 20769816 | 134821 | rs35363501 |
| 20770092 | 135097 | rs6456370 |
| 20770102 | 135107 | rs979614 |
| 20770196 | 135201 | rs35456723 |
| 20770571 | 135576 | rs9368218 |
| 20770945 | 135950 | rs4712525 |
| 20771014 | 136019 | rs4712526 |
| 20771314 | 136319 | rs4712527 |
| 20771442 | 136447 | rs35191644 |
| 20771442 | 136447 | rs34470647 |
| 20771611 | 136616 | rs9460546 |
| 20771938 | 136943 | rs9465859 |
| 20772079 | 137084 | rs9465860 |
| 20772291 | 137296 | rs736425 |
| 20772508 | 137513 | rs3060776 |
| 20772509 | 137514 | rs34941928 |
| 20772512 | 137517 | rs5874781 |
| 20772761 | 137766 | rs35778487 |
| 20773060 | 138065 | rs742642 |
| 20773305 | 138310 | rs35248697 |
| 20773436 | 138441 | rs11967127 |
| 20773528 | 138533 | rs7748382 |
| 20773547 | 138552 | rs9688549 |
| 20773548 | 138553 | rs9689351 |
| 20773570 | 138575 | rs28665000 |
| 20773886 | 138891 | rs7752236 |
| 20773925 | 138930 | rs7772603 |
| 20774001 | 139006 | rs7752780 |
| 20774034 | 139039 | rs7752906 |
| 20774160 | 139165 | rs34184260 |
| 20774223 | 139228 | rs2206739 |
| 20774225 | 139230 | rs2206738 |
| 20774250 | 139255 | rs2206737 |
| 20774436 | 139441 | rs11970425 |
| 20774484 | 139489 | rs36034806 |
| 20774899 | 139904 | rs35042364 |
| 20775218 | 140223 | rs35540121 |
| 20775361 | 140366 | rs9358356 |
| 20775667 | 140672 | rs9356743 |
| 20775778 | 140783 | rs9350270 |
| 20776366 | 141371 | rs34929853 |
| 20778035 | 143040 | rs34971765 |
| 20778443 | 143448 | rs11970596 |
| 20779261 | 144266 | rs12527373 |
| 20779367 | 144372 | rs35916847 |
| 20780262 | 145267 | rs11968224 |
| 20780271 | 145276 | rs11968225 |
| 20780276 | 145281 | rs9465861 |
| 20780296 | 145301 | rs11968264 |
| 20780406 | 145411 | rs12189849 |
| 20780413 | 145418 | rs12209627 |
| 20780432 | 145437 | rs12189895 |
| 20780855 | 145860 | rs11968848 |
| 20781135 | 146140 | rs11963945 |
| 20781601 | 146606 | rs35677128 |
| 20781859 | 146864 | rs7451008 |
| 20782670 | 147675 | rs9368219 |
| 20782790 | 147795 | rs1012636 |
| 20782945 | 147950 | rs13217846 |
| 20783274 | 148279 | rs1012635 |
| 20783700 | 148705 | rs35665197 |
| 20783771 | 148776 | rs35261542 |
| 20783828 | 148833 | rs28823314 |
| 20783899 | 148904 | rs28890810 |
| 20784051 | 149056 | rs28871991 |
| 20784393 | 149398 | rs34499031 |
| 20784650 | 149655 | rs13208763 |
| 20784747 | 149752 | rs28719685 |
| 20784789 | 149794 | rs28856096 |
| 20785042 | 150047 | rs11961863 |
| 20785211 | 150216 | rs17824302 |
| 20785289 | 150294 | rs12660618 |
| 20786302 | 151307 | rs11371206 |
| 20786303 | 151308 | rs34152621 |
| 20786409 | 151414 | rs4712528 |
| 20786463 | 151468 | rs13217082 |
| 20786470 | 151475 | rs13217085 |
| 20786481 | 151486 | rs13217090 |
| 20786483 | 151488 | rs13217091 |
| 20786523 | 151528 | rs13200946 |
| 20786772 | 151777 | rs11968032 |
| 20786954 | 151959 | rs9465863 |
| 20787289 | 152294 | rs1569699 |
| 20787386 | 152391 | rs34168173 |
| 20787688 | 152693 | rs7756992 |
| 20788045 | 153050 | rs35312717 |
| 20788657 | 153662 | rs9348441 |
| 20788843 | 153848 | rs9368220 |
| 20788941 | 153946 | rs6931254 |
| 20789327 | 154332 | rs6911742 |
| 20790601 | 155606 | rs35612982 |
| 20791039 | 156044 | rs35816514 |
| 20791123 | 156128 | rs34612860 |
| 20791143 | 156148 | rs9350271 |
| 20791162 | 156167 | rs35657899 |
| 20791179 | 156184 | rs11364854 |
| 20791249 | 156254 | rs9460547 |
| 20791646 | 156651 | rs16884103 |
| 20791961 | 156966 | rs2206736 |
| 20793465 | 158470 | rs9356744 |
| 20794295 | 159300 | rs34987372 |
| 20794427 | 159432 | rs36005020 |
| 20794552 | 159557 | rs7766070 |
| 20794975 | 159980 | rs9368222 |
| 20795290 | 160295 | rs35566695 |
| 20795781 | 160786 | rs10440832 |
| 20796100 | 161105 | rs10440833 |
| 20796237 | 161242 | rs35747076 |
| 20796578 | 161583 | rs6900217 |
| 20797104 | 162109 | rs34433496 |
| 20797924 | 162929 | rs7748720 |
| 20797928 | 162933 | rs34175709 |
| 20798290 | 163295 | rs6911357 |
| 20800493 | 165498 | rs12200791 |
| 20800955 | 165960 | rs5874782 |
| 20800957 | 165962 | rs36119385 |
| 20801341 | 166346 | rs13219682 |
| 20802207 | 167212 | rs4710941 |
| 20802270 | 167275 | rs4620109 |
| 20802272 | 167277 | rs28459626 |
| 20802273 | 167278 | rs4712529 |
| 20802294 | 167299 | rs10577753 |
| 20802504 | 167509 | rs2223683 |
| 20802573 | 167578 | rs2206735 |
| 20802863 | 167868 | rs2206734 |
| 20802910 | 167915 | rs34530846 |
| 20803458 | 168463 | rs16884131 |
| 20804127 | 169132 | rs10806921 |
| 20805104 | 170109 | rs16884133 |
| 20805571 | 170576 | rs17824500 |
| 20805652 | 170657 | rs10946401 |
| 20806114 | 171119 | rs16884135 |
| 20806582 | 171587 | rs35711395 |
| 20807220 | 172225 | rs11969783 |
| 20807364 | 172369 | rs16884137 |
| 20808600 | 173605 | rs11970626 |
| 20809092 | 174097 | rs12190713 |
| 20809106 | 174111 | rs11398905 |
| 20809415 | 174420 | rs11961445 |
| 20809470 | 174475 | rs35982077 |
| 20809486 | 174491 | rs11305935 |
| 20810952 | 175957 | rs9356745 |
| 20811700 | 176705 | rs35043644 |
| 20811842 | 176847 | rs16884140 |
| 20811931 | 176936 | rs6931514 |
| 20812147 | 177152 | rs35443650 |
| 20813281 | 178286 | rs34671712 |
| 20813569 | 178574 | rs11753081 |
| 20814081 | 179086 | rs7739516 |
| 20814209 | 179214 | rs6901559 |
| 20815176 | 180181 | rs13196379 |
| 20815177 | 180182 | rs13212234 |
| 20816204 | 181209 | rs10536170 |
| 20817155 | 182160 | rs9465869 |
| 20817688 | 182693 | rs36070002 |
| 20818288 | 183293 | rs17226450 |
| 20818905 | 183910 | rs1073247 |
| 20819131 | 184136 | rs9465870 |
| 20819386 | 184391 | rs17226492 |
| 20819433 | 184438 | rs13213613 |
| 20819567 | 184572 | rs16884146 |
| 20819958 | 184963 | rs2206733 |
| 20820440 | 185445 | rs3749925 |
| 20821121 | 186126 | rs9460548 |
| 20821619 | 186624 | rs9460549 |
| 20821685 | 186690 | rs1040558 |
| 20821893 | 186898 | rs4712530 |
| 20822083 | 187088 | rs35629277 |
| 20822362 | 187367 | rs7451928 |
| 20822445 | 187450 | rs6456371 |
| 20822589 | 187594 | rs13220116 |
| 20822823 | 187828 | rs2206732 |
| 20823169 | 188174 | rs2179633 |
| 20823483 | 188488 | rs11963770 |
| 20823805 | 188810 | rs10946402 |
| 20823840 | 188845 | rs4712531 |
| 20824098 | 189103 | rs35738288 |
| 20824232 | 189237 | rs9295478 |
| 20824549 | 189554 | rs2328547 |
| 20824763 | 189768 | rs3060781 |
| 20824764 | 189769 | rs34686252 |
| 20824856 | 189861 | rs13215905 |
| 20824884 | 189889 | rs9368223 |
| 20824937 | 189942 | rs2328548 |
| 20825025 | 190030 | rs11427712 |
| 20825074 | 190079 | rs6935599 |
| 20825100 | 190105 | rs13216165 |
| 20825234 | 190239 | rs9465871 |
| 20825383 | 190388 | rs10946403 |
| 20826219 | 191224 | rs2328549 |
| 20826449 | 191454 | rs17226774 |
| 20827124 | 192129 | rs9358357 |
| 20827211 | 192216 | rs9368224 |
| 20827321 | 192326 | rs11756271 |
| 20827372 | 192377 | rs9358358 |
| 20827540 | 192545 | rs9460550 |
| 20827858 | 192863 | rs12110493 |
| 20827866 | 192871 | rs12193125 |
| 20828258 | 193263 | rs9356746 |
| 20828797 | 193802 | rs9350272 |
| 20829322 | 194327 | rs13219444 |
| 20829342 | 194347 | rs12111216 |
| 20829562 | 194567 | rs9350273 |
| 20829700 | 194705 | rs9368225 |
| 20830399 | 195404 | rs17825025 |
| 20831036 | 196041 | rs9368226 |
| 20832213 | 197218 | rs6903175 |
| 20832229 | 197234 | rs6903744 |
| 20832537 | 197542 | rs12111351 |
| 20832754 | 197759 | rs4712536 |
| 20832986 | 197991 | rs9356747 |
| 20833076 | 198081 | rs9356748 |
| 20833219 | 198224 | rs7767391 |
| 20833402 | 198407 | rs7747752 |
| 20833511 | 198516 | rs9350274 |
| 20833853 | 198858 | rs34170041 |
| 20833919 | 198924 | rs6915155 |
| 20834014 | 199019 | rs6914868 |
| 20834472 | 199477 | rs4538697 |
| 20835549 | 200554 | rs4712537 |
| 20836048 | 201053 | rs34097377 |
| 20836492 | 201497 | rs6928012 |
| 20836710 | 201715 | rs6908425 |

TABLE 11
SNPs within LD block C10 (SEQ ID NO: 2) between positions
94,192,885 and 94,490,091 bp on Chromosome 10 in NCBI
Build 35 and NCBI Build 36

| | Position in | |
|---|---|---|
| Position in | SEQ ID | |
| Build 35/36 | NO: 2 | Marker ID |
| 94192885 | 1 | rs2798253 |
| 94193597 | 713 | rs36087110 |
| 94193803 | 919 | rs35771118 |
| 94193950 | 1066 | rs12359552 |
| 94193961 | 1077 | rs11186999 |
| 94194166 | 1282 | rs7916460 |
| 94194775 | 1891 | rs10882065 |
| 94195841 | 2957 | rs11187000 |
| 94196162 | 3278 | rs4933231 |
| 94196306 | 3422 | rs11187001 |
| 94196353 | 3469 | rs4933725 |
| 94196465 | 3581 | rs11187002 |
| 94196477 | 3593 | rs4933726 |
| 94196509 | 3625 | rs4933232 |
| 94196716 | 3832 | rs11187003 |
| 94196844 | 3960 | rs34115369 |
| 94197028 | 4144 | rs10786047 |
| 94197152 | 4268 | rs11814521 |
| 94197347 | 4463 | rs11814555 |
| 94198457 | 5573 | rs7476275 |
| 94198727 | 5843 | rs3118967 |
| 94199011 | 6127 | rs11187004 |
| 94199856 | 6972 | rs7910977 |
| 94199919 | 7035 | rs6583813 |
| 94199932 | 7048 | rs511985 |
| 94200269 | 7385 | rs7911558 |
| 94200789 | 7905 | rs12415807 |
| 94201174 | 8290 | rs35125831 |
| 94201284 | 8400 | rs2251101 |
| 94201876 | 8992 | rs7896688 |
| 94202516 | 9632 | rs5786996 |
| 94202722 | 9838 | rs913648 |
| 94203071 | 10187 | rs5786997 |
| 94203072 | 10188 | rs35771235 |
| 94203255 | 10371 | rs34872659 |
| 94203768 | 10884 | rs34266748 |
| 94204339 | 11455 | rs4646958 |
| 94204560 | 11676 | rs11187007 |
| 94205437 | 12553 | rs11459510 |
| 94205449 | 12565 | rs35832015 |
| 94206153 | 13269 | rs12356364 |
| 94206407 | 13523 | rs11593933 |
| 94206490 | 13606 | rs3781241 |
| 94206524 | 13640 | rs3781240 |
| 94206594 | 13710 | rs10562725 |
| 94206599 | 13715 | rs10617641 |
| 94206609 | 13725 | rs28641489 |
| 94207018 | 14134 | rs11187009 |
| 94207224 | 14340 | rs36119168 |
| 94207391 | 14507 | rs11594562 |
| 94207777 | 14893 | rs3781239 |
| 94208177 | 15293 | rs3824738 |
| 94208228 | 15344 | rs12782629 |
| 94208261 | 15377 | rs12261501 |
| 94208278 | 15394 | rs12781670 |
| 94208383 | 15499 | rs568657 |
| 94208423 | 15539 | rs509954 |
| 94209484 | 16600 | rs489517 |
| 94209509 | 16625 | rs9420586 |
| 94209578 | 16694 | rs11187010 |
| 94209597 | 16713 | rs2247348 |
| 94209748 | 16864 | rs307638 |
| 94210585 | 17701 | rs35118791 |
| 94210625 | 17741 | rs520711 |
| 94211102 | 18218 | rs7098739 |
| 94211382 | 18498 | rs7081224 |
| 94211591 | 18707 | rs7093437 |
| 94212604 | 19720 | rs551266 |
| 94213696 | 20812 | rs1042444 |
| 94213766 | 20882 | rs7087334 |
| 94214145 | 21261 | rs1887922 |
| 94214615 | 21731 | rs7898862 |
| 94214726 | 21842 | rs10882066 |
| 94214869 | 21985 | rs11187011 |
| 94214932 | 22048 | rs7916011 |
| 94214997 | 22113 | rs7899603 |
| 94215212 | 22328 | rs34934289 |
| 94215235 | 22351 | rs12242504 |
| 94215277 | 22393 | rs2275218 |
| 94215373 | 22489 | rs538469 |
| 94215528 | 22644 | rs35640611 |
| 94215823 | 22939 | rs11187012 |
| 94216140 | 23256 | rs11187013 |
| 94216829 | 23945 | rs7893352 |
| 94217818 | 24934 | rs11187014 |
| 94218798 | 25914 | rs544537 |
| 94218805 | 25921 | rs12243622 |
| 94219607 | 26723 | rs11187015 |
| 94219726 | 26842 | rs7920976 |
| 94219892 | 27008 | rs4646957 |
| 94220409 | 27525 | rs11187016 |
| 94221786 | 28902 | rs2250090 |
| 94222227 | 29343 | rs2149632 |
| 94222398 | 29514 | rs35959170 |
| 94222860 | 29976 | rs35551274 |
| 94222881 | 29997 | rs7087153 |
| 94222964 | 30080 | rs12762802 |
| 94223038 | 30154 | rs12763971 |
| 94223085 | 30201 | rs11187017 |
| 94223100 | 30216 | rs2249960 |
| 94223275 | 30391 | rs12262931 |
| 94223719 | 30835 | rs11187018 |
| 94223794 | 30910 | rs11323400 |
| 94223971 | 31087 | rs7092468 |
| 94224735 | 31851 | rs12245118 |
| 94224789 | 31905 | rs35223317 |
| 94226905 | 34021 | rs35637537 |
| 94227236 | 34352 | rs35291821 |
| 94227390 | 34506 | rs7073248 |
| 94227405 | 34521 | rs7091270 |
| 94227647 | 34763 | rs12251346 |
| 94227782 | 34898 | rs6583815 |
| 94227902 | 35018 | rs12411941 |
| 94227937 | 35053 | rs17875326 |
| 94228149 | 35265 | rs7077626 |
| 94228919 | 36035 | rs35864975 |
| 94229152 | 36268 | rs5030982 |
| 94229349 | 36465 | rs3831274 |
| 94229366 | 36482 | rs35611772 |
| 94229773 | 36889 | rs7910605 |
| 94231074 | 38190 | rs12356508 |
| 94231328 | 38444 | rs34093069 |
| 94231497 | 38613 | rs35831196 |
| 94232484 | 39600 | rs35250835 |
| 94232485 | 39601 | rs5786998 |
| 94232486 | 39602 | rs35368064 |
| 94233186 | 40302 | rs12243214 |
| 94233203 | 40319 | rs7917817 |
| 94233597 | 40713 | rs2421940 |
| 94234183 | 41299 | rs35120790 |
| 94234248 | 41364 | rs10882067 |
| 94234880 | 41996 | rs35436518 |
| 94234881 | 41997 | rs34615998 |
| 94234883 | 41999 | rs11595475 |
| 94235591 | 42707 | rs35243007 |
| 94236972 | 44088 | rs35426658 |
| 94237227 | 44343 | rs6583817 |
| 94237240 | 44356 | rs35863982 |
| 94237312 | 44428 | rs35532620 |
| 94238290 | 45406 | rs11187019 |
| 94238346 | 45462 | rs12219139 |
| 94238396 | 45512 | rs12219148 |
| 94238509 | 45625 | rs34930778 |
| 94238512 | 45628 | rs36015364 |
| 94238730 | 45846 | rs11187020 |
| 94239054 | 46170 | rs35650880 |
| 94239749 | 46865 | rs7093418 |
| 94239850 | 46966 | rs11596251 |
| 94239962 | 47078 | rs3737225 |
| 94241364 | 48480 | rs11444132 |
| 94241365 | 48481 | rs34841034 |
| 94242628 | 49744 | rs11187021 |
| 94243163 | 50279 | rs3837333 |
| 94243164 | 50280 | rs34838821 |
| 94243184 | 50300 | rs3781238 |
| 94243185 | 50301 | rs35973022 |
| 94243185 | 50301 | rs3781237 |
| 94243606 | 50722 | rs10882068 |
| 94244183 | 51299 | rs1855917 |
| 94244263 | 51379 | rs1855916 |
| 94245019 | 52135 | rs10882069 |
| 94245021 | 52137 | rs9420151 |
| 94245023 | 52139 | rs11187022 |
| 94245061 | 52177 | rs10882070 |
| 94245384 | 52500 | rs7075073 |
| 94246000 | 53116 | rs11187024 |
| 94246773 | 53889 | rs11598525 |
| 94246972 | 54088 | rs34822156 |
| 94247198 | 54314 | rs7084090 |
| 94247956 | 55072 | rs11187025 |
| 94247994 | 55110 | rs6583818 |
| 94249045 | 56161 | rs34666358 |
| 94249117 | 56233 | rs11187026 |
| 94249160 | 56276 | rs11187027 |
| 94249226 | 56342 | rs34459034 |
| 94249288 | 56404 | rs11187028 |
| 94249316 | 56432 | rs36049328 |
| 94249679 | 56795 | rs7097800 |
| 94249948 | 57064 | rs10786048 |
| 94250085 | 57201 | rs10882071 |
| 94250350 | 57466 | rs12249976 |
| 94250507 | 57623 | rs7068618 |
| 94250611 | 57727 | rs11187029 |
| 94250692 | 57808 | rs10882072 |
| 94250983 | 58099 | rs11187030 |
| 94251771 | 58887 | rs11187031 |
| 94251786 | 58902 | rs11187032 |
| 94252339 | 59455 | rs11187033 |
| 94252515 | 59631 | rs11187034 |
| 94252799 | 59915 | rs11442945 |
| 94253137 | 60253 | rs11187035 |
| 94253203 | 60319 | rs1970244 |
| 94253341 | 60457 | rs11187037 |
| 94253515 | 60631 | rs1970245 |
| 94253764 | 60880 | rs5786999 |
| 94253765 | 60881 | rs34057954 |
| 94253766 | 60882 | rs10716816 |
| 94254606 | 61722 | rs34708742 |
| 94254765 | 61881 | rs35101389 |
| 94254975 | 62091 | rs11187038 |
| 94255082 | 62198 | rs34174850 |
| 94256325 | 63441 | rs34053974 |
| 94256855 | 63971 | rs11296200 |
| 94257747 | 64863 | rs11460471 |
| 94258212 | 65328 | rs11286004 |
| 94258296 | 65412 | rs5787000 |
| 94258297 | 65413 | rs33917554 |
| 94258314 | 65430 | rs1832196 |
| 94258319 | 65435 | rs34194084 |
| 94258381 | 65497 | rs1832195 |
| 94258980 | 66096 | rs35636429 |
| 94259168 | 66284 | rs4256898 |
| 94259346 | 66462 | rs34663898 |
| 94259587 | 66703 | rs6583819 |
| 94259670 | 66786 | rs11324773 |
| 94259792 | 66908 | rs11187039 |
| 94260389 | 67505 | rs34662862 |
| 94260838 | 67954 | rs35891632 |
| 94260859 | 67975 | rs10882073 |
| 94260983 | 68099 | rs11498516 |
| 94261156 | 68272 | rs17445028 |
| 94261438 | 68554 | rs35831688 |
| 94262303 | 69419 | rs11373926 |
| 94262304 | 69420 | rs35405697 |
| 94262311 | 69427 | rs35377675 |
| 94262679 | 69795 | rs34457657 |
| 94262685 | 69801 | rs34774587 |
| 94262844 | 69960 | rs11187040 |
| 94263091 | 70207 | rs7086558 |
| 94263344 | 70460 | rs7910569 |
| 94263586 | 70702 | rs34673600 |
| 94264572 | 71688 | rs35270297 |
| 94264650 | 71766 | rs4646956 |
| 94264789 | 71905 | rs17875327 |
| 94265538 | 72654 | rs9633693 |
| 94266128 | 73244 | rs12780132 |
| 94266506 | 73622 | rs7895832 |
| 94266635 | 73751 | rs6583820 |
| 94267645 | 74761 | rs7093773 |
| 94267750 | 74866 | rs12257226 |
| 94267766 | 74882 | rs7075851 |
| 94267846 | 74962 | rs10509645 |
| 94268591 | 75707 | rs35693308 |
| 94271124 | 78240 | rs11812558 |
| 94271625 | 78741 | rs11187042 |
| 94271665 | 78781 | rs10882074 |
| 94271861 | 78977 | rs11187043 |
| 94272258 | 79374 | rs11187044 |
| 94272698 | 79814 | rs7915971 |
| 94273091 | 80207 | rs4933233 |
| 94273288 | 80404 | rs35361515 |
| 94273349 | 80465 | rs11187045 |
| 94273885 | 81001 | rs35296767 |
| 94273981 | 81097 | rs11187046 |
| 94274088 | 81204 | rs11813097 |
| 94274094 | 81210 | rs10882075 |
| 94274100 | 81216 | rs10882076 |
| 94274121 | 81237 | rs11187047 |
| 94274127 | 81243 | rs11187048 |
| 94274129 | 81245 | rs11187049 |
| 94274135 | 81251 | rs11187050 |
| 94274143 | 81259 | rs11187051 |
| 94274150 | 81266 | rs11187052 |
| 94274183 | 81299 | rs11818981 |
| 94274184 | 81300 | rs11818982 |
| 94274213 | 81329 | rs11187053 |
| 94274245 | 81361 | rs12355280 |
| 94274246 | 81362 | rs12359894 |
| 94274253 | 81369 | rs11187054 |
| 94274787 | 81903 | rs12358677 |
| 94275109 | 82225 | rs35688800 |
| 94275207 | 82323 | rs12261046 |
| 94275338 | 82454 | rs12261114 |
| 94275379 | 82495 | rs12261174 |
| 94275382 | 82498 | rs7894448 |
| 94275487 | 82603 | rs12262694 |
| 94275508 | 82624 | rs4641376 |
| 94275509 | 82625 | rs35586301 |
| 94276174 | 83290 | rs11187055 |
| 94276314 | 83430 | rs7089987 |
| 94276400 | 83516 | rs7073833 |
| 94276465 | 83581 | rs10882077 |
| 94276595 | 83711 | rs11459412 |
| 94276596 | 83712 | rs34975586 |
| 94277360 | 84476 | rs2421942 |
| 94280464 | 87580 | rs7078413 |
| 94280662 | 87778 | rs7079099 |
| 94280746 | 87862 | rs12258487 |
| 94281644 | 88760 | rs34747737 |
| 94281681 | 88797 | rs7901064 |
| 94282086 | 89202 | rs17107709 |
| 94282197 | 89313 | rs868057 |
| 94283137 | 90253 | rs34880105 |
| 94283469 | 90585 | rs35455474 |
| 94283592 | 90708 | rs11819413 |
| 94283667 | 90783 | rs11187056 |
| 94283823 | 90939 | rs1855915 |
| 94283919 | 91035 | rs12268712 |
| 94284271 | 91387 | rs4646955 |
| 94285010 | 92126 | rs7898114 |
| 94285220 | 92336 | rs11450948 |
| 94285221 | 92337 | rs35571064 |
| 94285296 | 92412 | rs7898493 |
| 94285778 | 92894 | rs7077418 |
| 94286057 | 93173 | rs11187057 |
| 94286438 | 93554 | rs34460166 |
| 94286967 | 94083 | rs2275221 |
| 94288311 | 95427 | rs1832197 |
| 94288480 | 95596 | rs17107721 |
| 94288514 | 95630 | rs5787001 |
| 94288515 | 95631 | rs34593706 |
| 94288516 | 95632 | rs11187059 |
| 94288531 | 95647 | rs12249288 |
| 94290376 | 97492 | rs12416180 |
| 94292030 | 99146 | rs10882078 |
| 94292741 | 99857 | rs11815736 |
| 94293623 | 100739 | rs5004594 |
| 94293624 | 100740 | rs1970243 |
| 94293625 | 100741 | rs33928713 |
| 94293625 | 100741 | rs5787002 |
| 94293956 | 101072 | rs12218329 |
| 94294112 | 101228 | rs11187060 |
| 94294428 | 101544 | rs7915349 |
| 94295169 | 102285 | rs17445328 |
| 94295389 | 102505 | rs11187061 |
| 94295397 | 102513 | rs17107734 |
| 94296406 | 103522 | rs11187062 |
| 94296563 | 103679 | rs10218994 |
| 94296625 | 103741 | rs17445419 |
| 94296937 | 104053 | rs12219325 |
| 94297315 | 104431 | rs11286362 |
| 94297863 | 104979 | rs10786049 |
| 94297879 | 104995 | rs7900822 |
| 94298098 | 105214 | rs11187063 |
| 94298233 | 105349 | rs11187064 |
| 94298446 | 105562 | rs10219017 |
| 94299005 | 106121 | rs34494546 |
| 94299843 | 106959 | rs7909636 |
| 94300255 | 107371 | rs34330550 |
| 94300414 | 107530 | rs10882079 |
| 94300889 | 108005 | rs12220493 |
| 94301795 | 108911 | rs2421943 |
| 94301904 | 109020 | rs11187065 |
| 94302165 | 109281 | rs4406744 |
| 94302446 | 109562 | rs35156639 |
| 94303116 | 110232 | rs1987122 |
| 94303124 | 110240 | rs9420144 |
| 94303675 | 110791 | rs35707435 |
| 94304293 | 111409 | rs11418454 |
| 94304299 | 111415 | rs11424864 |
| 94304548 | 111664 | rs6583821 |
| 94304589 | 111705 | rs10882080 |
| 94304784 | 111900 | rs7908111 |
| 94305004 | 112120 | rs10882081 |
| 94306623 | 113739 | rs7902106 |
| 94306803 | 113919 | rs12415874 |
| 94306808 | 113924 | rs7917163 |
| 94307486 | 114602 | rs4933728 |
| 94307610 | 114726 | rs12412249 |
| 94307630 | 114746 | rs3051565 |
| 94307851 | 114967 | rs11187066 |
| 94307892 | 115008 | rs10882082 |
| 94308049 | 115165 | rs7098744 |
| 94308378 | 115494 | rs12777622 |
| 94308408 | 115524 | rs12779093 |
| 94308409 | 115525 | rs12777974 |
| 94309312 | 116428 | rs11187067 |
| 94309594 | 116710 | rs12765408 |
| 94309595 | 116711 | rs34052181 |
| 94309972 | 117088 | rs1999763 |
| 94310119 | 117235 | rs1999764 |
| 94310400 | 117516 | rs11187068 |
| 94310514 | 117630 | rs7078418 |
| 94310644 | 117760 | rs35429533 |
| 94310846 | 117962 | rs11187069 |
| 94311213 | 118329 | rs34630015 |
| 94311876 | 118992 | rs12264361 |
| 94311953 | 119069 | rs10882083 |
| 94312407 | 119523 | rs12264682 |
| 94312615 | 119731 | rs11187070 |
| 94312726 | 119842 | rs12266443 |
| 94312981 | 120097 | rs12776190 |
| 94313002 | 120118 | rs4933729 |
| 94313190 | 120306 | rs12774925 |
| 94313202 | 120318 | rs12774931 |
| 94314015 | 121131 | rs11187071 |
| 94314384 | 121500 | rs35009022 |
| 94314389 | 121505 | rs11187072 |
| 94314566 | 121682 | rs12241107 |
| 94314708 | 121824 | rs11187073 |
| 94314816 | 121932 | rs12763871 |
| 94314968 | 122084 | rs35418143 |
| 94315124 | 122240 | rs12411517 |
| 94315147 | 122263 | rs11308616 |
| 94315157 | 122273 | rs33935672 |
| 94315157 | 122273 | rs5787003 |
| 94315491 | 122607 | rs7076966 |
| 94315748 | 122864 | rs5787004 |
| 94315751 | 122867 | rs35925526 |
| 94315930 | 123046 | rs5787005 |
| 94315944 | 123060 | rs34378303 |
| 94316148 | 123264 | rs2901598 |
| 94316150 | 123266 | rs2421944 |
| 94316175 | 123291 | rs34777726 |
| 94316176 | 123292 | rs3051566 |
| 94316766 | 123882 | rs12253172 |
| 94316799 | 123915 | rs12253177 |
| 94316926 | 124042 | rs11187074 |
| 94317075 | 124191 | rs7082686 |
| 94317136 | 124252 | rs7100623 |
| 94317536 | 124652 | rs12255048 |
| 94317963 | 125079 | rs12355977 |
| 94318001 | 125117 | rs35616984 |
| 94318580 | 125696 | rs35826224 |
| 94318582 | 125698 | rs10584127 |
| 94318582 | 125698 | rs7915220 |
| 94318992 | 126108 | rs11437287 |
| 94318993 | 126109 | rs35875721 |
| 94319093 | 126209 | rs10882084 |
| 94320200 | 127316 | rs7097014 |
| 94321033 | 128149 | rs12415246 |
| 94321119 | 128235 | rs4304670 |
| 94321321 | 128437 | rs2421945 |
| 94321612 | 128728 | rs12252447 |
| 94321742 | 128858 | rs10736067 |
| 94322696 | 129812 | rs7909487 |
| 94323150 | 130266 | rs34093946 |
| 94323222 | 130338 | rs12783634 |
| 94323807 | 130923 | rs4646954 |
| 94323935 | 131051 | rs4646953 |
| 94323955 | 131071 | rs35380063 |
| 94324493 | 131609 | rs17875328 |
| 94324556 | 131672 | rs34867369 |
| 94324758 | 131874 | rs3758505 |
| 94325575 | 132691 | rs11379220 |
| 94325779 | 132895 | rs7099761 |
| 94326009 | 133125 | rs11187075 |
| 94326342 | 133458 | rs35628053 |
| 94326394 | 133510 | rs3758504 |
| 94326684 | 133800 | rs3758503 |
| 94326943 | 134059 | rs11187076 |
| 94327161 | 134277 | rs35376801 |
| 94327403 | 134519 | rs12762388 |
| 94327431 | 134547 | rs12762406 |
| 94327438 | 134554 | rs12764060 |
| 94327683 | 134799 | rs4933730 |
| 94327871 | 134987 | rs7900584 |
| 94328020 | 135136 | rs12414433 |
| 94328211 | 135327 | rs7900991 |
| 94328632 | 135748 | rs7921325 |
| 94328678 | 135794 | rs7905037 |
| 94330087 | 137203 | rs11187077 |
| 94330685 | 137801 | rs11187078 |
| 94330689 | 137805 | rs11273566 |
| 94331181 | 138297 | rs6583822 |
| 94331572 | 138688 | rs11187079 |
| 94331642 | 138758 | rs7069538 |
| 94331770 | 138886 | rs7087423 |
| 94332643 | 139759 | rs2901597 |
| 94332963 | 140079 | rs7092522 |
| 94332992 | 140108 | rs12766927 |
| 94333545 | 140661 | rs11187080 |
| 94334378 | 141494 | rs34210106 |
| 94334395 | 141511 | rs6583823 |
| 94334850 | 141966 | rs7084215 |
| 94334897 | 142013 | rs6583824 |
| 94335207 | 142323 | rs6583825 |
| 94335889 | 143005 | rs2421941 |
| 94336041 | 143157 | rs12262994 |
| 94336117 | 143233 | rs12263054 |
| 94336859 | 143975 | rs12573146 |
| 94337053 | 144169 | rs11187081 |
| 94337295 | 144411 | rs34143873 |
| 94337314 | 144430 | rs11455878 |
| 94337315 | 144431 | rs35480775 |
| 94337549 | 144665 | rs7921210 |
| 94337810 | 144926 | rs6583826 |
| 94338024 | 145140 | rs12776949 |
| 94338180 | 145296 | rs7922041 |
| 94338205 | 145321 | rs34073794 |
| 94338626 | 145742 | rs10701812 |
| 94338815 | 145931 | rs7911019 |
| 94339556 | 146672 | rs7898478 |
| 94340196 | 147312 | rs4611114 |
| 94340487 | 147603 | rs1889894 |
| 94340661 | 147777 | rs12355158 |
| 94341882 | 148998 | rs17875329 |
| 94341915 | 149031 | rs11595187 |
| 94341951 | 149067 | rs17875331 |
| 94342066 | 149182 | rs17875332 |
| 94342135 | 149251 | rs11595260 |
| 94342136 | 149252 | rs11599701 |
| 94342406 | 149522 | rs7092778 |
| 94342485 | 149601 | rs11187083 |
| 94342495 | 149611 | rs17875333 |
| 94342598 | 149714 | rs11187084 |
| 94342803 | 149919 | rs2297743 |
| 94343099 | 150215 | rs36015451 |
| 94343258 | 150374 | rs12256435 |
| 94343418 | 150534 | rs11818332 |
| 94343843 | 150959 | rs3781236 |
| 94344062 | 151178 | rs3824736 |
| 94344184 | 151300 | rs3824735 |
| 94344608 | 151724 | rs11429679 |
| 94344609 | 151725 | rs34491012 |
| 94344692 | 151808 | rs11187085 |
| 94344951 | 152067 | rs11187086 |
| 94345504 | 152620 | rs11817798 |
| 94345575 | 152691 | rs12240557 |
| 94345783 | 152899 | rs35583124 |
| 94345930 | 153046 | rs12262350 |
| 94346261 | 153377 | rs11187087 |
| 94346544 | 153660 | rs7099635 |
| 94346892 | 154008 | rs4604791 |
| 94346932 | 154048 | rs11556038 |
| 94347159 | 154275 | rs7082683 |
| 94348003 | 155119 | rs6583827 |
| 94349194 | 156310 | rs11187089 |
| 94349700 | 156816 | rs12218324 |
| 94349861 | 156977 | rs11498685 |
| 94350652 | 157768 | rs11187090 |
| 94350841 | 157957 | rs12411873 |
| 94351226 | 158342 | rs7068118 |
| 94351364 | 158480 | rs35624581 |
| 94351461 | 158577 | rs35982149 |
| 94352267 | 159383 | rs35452156 |
| 94352383 | 159499 | rs35807258 |
| 94352546 | 159662 | rs10564653 |
| 94352561 | 159677 | rs7095250 |
| 94352562 | 159678 | rs7095369 |
| 94352579 | 159695 | rs10564753 |
| 94352593 | 159709 | rs3980475 |
| 94352908 | 160024 | rs7096101 |
| 94353042 | 160158 | rs9733604 |
| 94353107 | 160223 | rs12266122 |
| 94353183 | 160299 | rs7096520 |
| 94353519 | 160635 | rs35612167 |
| 94353973 | 161089 | rs4933731 |
| 94354724 | 161840 | rs11187091 |
| 94354862 | 161978 | rs10882085 |
| 94354869 | 161985 | rs12763703 |
| 94355005 | 162121 | rs10882086 |
| 94355421 | 162537 | rs11812310 |
| 94355638 | 162754 | rs11187092 |
| 94355673 | 162789 | rs12242681 |
| 94355721 | 162837 | rs12257279 |
| 94356330 | 163446 | rs11393423 |
| 94356331 | 163447 | rs35558317 |
| 94356578 | 163694 | rs12244660 |
| 94356605 | 163721 | rs12244573 |
| 94356815 | 163931 | rs12244809 |
| 94356878 | 163994 | rs12259474 |
| 94356984 | 164100 | rs17875334 |
| 94357013 | 164129 | rs17107758 |
| 94357210 | 164326 | rs10786050 |
| 94357829 | 164945 | rs12246900 |
| 94357847 | 164963 | rs10882087 |
| 94357897 | 165013 | rs12261518 |
| 94357989 | 165105 | rs11187093 |
| 94358032 | 165148 | rs12247024 |
| 94358158 | 165274 | rs11187094 |
| 94358195 | 165311 | rs12261697 |
| 94358568 | 165684 | rs10882088 |
| 94358636 | 165752 | rs12248860 |
| 94359005 | 166121 | rs3835295 |
| 94359040 | 166156 | rs10882089 |
| 94359040 | 166156 | rs34446454 |
| 94359042 | 166158 | rs11187095 |
| 94359042 | 166158 | rs3835294 |
| 94359044 | 166160 | rs12767141 |
| 94359496 | 166612 | rs12250905 |
| 94359568 | 166684 | rs11187096 |
| 94359915 | 167031 | rs12356724 |
| 94360614 | 167730 | rs11599330 |
| 94361073 | 168189 | rs35352564 |
| 94362686 | 169802 | rs2275220 |
| 94362922 | 170038 | rs2275219 |
| 94363760 | 170876 | rs10882090 |
| 94364357 | 171473 | rs10882091 |
| 94364583 | 171699 | rs11187097 |
| 94364871 | 171987 | rs11187098 |
| 94364977 | 172093 | rs11187099 |
| 94365856 | 172972 | rs12252642 |
| 94366308 | 173424 | rs12252836 |
| 94366409 | 173525 | rs17875338 |
| 94366437 | 173553 | rs17875337 |
| 94366604 | 173720 | rs33921491 |
| 94366606 | 173722 | rs10550249 |
| 94366638 | 173754 | rs34075743 |
| 94366848 | 173964 | rs7069647 |
| 94367517 | 174633 | rs11187100 |
| 94367754 | 174870 | rs11187101 |
| 94367943 | 175059 | rs12415792 |
| 94368029 | 175145 | rs3980476 |
| 94368183 | 175299 | rs12767059 |
| 94368830 | 175946 | rs6583828 |
| 94368857 | 175973 | rs11517020 |
| 94369044 | 176160 | rs34515231 |
| 94369203 | 176319 | rs10882092 |
| 94369352 | 176468 | rs6583829 |
| 94369694 | 176810 | rs12260688 |
| 94369814 | 176930 | rs12781567 |
| 94370112 | 177228 | rs7084441 |
| 94371113 | 178229 | rs17875339 |
| 94371489 | 178605 | rs11187102 |
| 94371558 | 178674 | rs11187103 |
| 94371597 | 178713 | rs34942989 |
| 94371629 | 178745 | rs11817621 |
| 94371641 | 178757 | rs11815573 |
| 94371998 | 179114 | rs11187104 |
| 94372020 | 179136 | rs7894183 |
| 94372150 | 179266 | rs12254289 |
| 94372405 | 179521 | rs36031409 |
| 94372432 | 179548 | rs12262063 |
| 94372442 | 179558 | rs7914248 |
| 94372445 | 179561 | rs12254474 |
| 94372736 | 179852 | rs7898027 |
| 94372930 | 180046 | rs7914814 |
| 94372946 | 180062 | rs7898506 |
| 94373054 | 180170 | rs17367408 |
| 94373182 | 180298 | rs10082524 |
| 94373409 | 180525 | rs7091887 |
| 94373424 | 180540 | rs7073868 |
| 94373661 | 180777 | rs7095585 |
| 94373838 | 180954 | rs12772554 |
| 94373970 | 181086 | rs12772956 |
| 94374055 | 181171 | rs10882093 |
| 94374407 | 181523 | rs11187105 |
| 94374449 | 181565 | rs12258463 |
| 94374494 | 181610 | rs35518831 |
| 94374496 | 181612 | rs34197740 |
| 94374497 | 181613 | rs35008887 |
| 94374498 | 181614 | rs35083945 |
| 94375670 | 182786 | rs11187106 |
| 94376102 | 183218 | rs7071905 |
| 94376134 | 183250 | rs7087860 |
| 94376451 | 183567 | rs35368950 |
| 94376836 | 183952 | rs7915915 |
| 94376918 | 184034 | rs12264372 |
| 94377314 | 184430 | rs1573051 |
| 94377656 | 184772 | rs10882094 |
| 94378087 | 185203 | rs12244924 |
| 94378491 | 185607 | rs17875340 |
| 94379385 | 186501 | rs11417254 |
| 94379401 | 186517 | rs7068540 |
| 94379725 | 186841 | rs28692618 |
| 94380110 | 187226 | rs35297001 |
| 94380403 | 187519 | rs7073739 |
| 94380415 | 187531 | rs11595797 |
| 94380535 | 187651 | rs12243009 |
| 94380591 | 187707 | rs7077137 |
| 94381028 | 188144 | rs9731595 |
| 94381254 | 188370 | rs7078403 |
| 94381345 | 188461 | rs7078179 |
| 94381677 | 188793 | rs11187107 |
| 94382267 | 189383 | rs2275217 |
| 94382439 | 189555 | rs17107764 |
| 94382499 | 189615 | rs10639509 |
| 94382499 | 189615 | rs17875342 |
| 94382500 | 189616 | rs34767899 |
| 94382708 | 189824 | rs11818705 |
| 94382761 | 189877 | rs11818708 |
| 94382810 | 189926 | rs11818750 |
| 94382815 | 189931 | rs5787006 |
| 94383610 | 190726 | rs11187108 |
| 94383671 | 190787 | rs3051599 |
| 94383672 | 190788 | rs35996949 |
| 94383672 | 190788 | rs2421946 |
| 94383672 | 190788 | rs5787007 |
| 94383674 | 190790 | rs34710486 |
| 94383765 | 190881 | rs28615450 |
| 94383941 | 191057 | rs10736068 |
| 94383978 | 191094 | rs11819282 |
| 94384075 | 191191 | rs11819301 |
| 94384310 | 191426 | rs11819351 |
| 94384382 | 191498 | rs10882095 |
| 94384580 | 191696 | rs7070990 |
| 94385075 | 192191 | rs7089765 |
| 94385373 | 192489 | rs10736069 |
| 94385475 | 192591 | rs12257072 |
| 94385728 | 192844 | rs7900689 |
| 94386335 | 193451 | rs11812813 |
| 94386492 | 193608 | rs4933732 |
| 94386500 | 193616 | rs11594678 |
| 94386540 | 193656 | rs11813238 |
| 94386568 | 193684 | rs1972360 |
| 94386594 | 193710 | rs11816962 |
| 94386703 | 193819 | rs4558084 |
| 94386890 | 194006 | rs34948996 |
| 94387372 | 194488 | rs1547818 |
| 94387496 | 194612 | rs34497717 |
| 94387741 | 194857 | rs7069680 |
| 94387964 | 195080 | rs7085790 |
| 94388094 | 195210 | rs36015190 |
| 94388098 | 195214 | rs6583830 |
| 94388569 | 195685 | rs7090402 |
| 94388954 | 196070 | rs11187109 |
| 94389669 | 196785 | rs35137137 |
| 94389736 | 196852 | rs35494689 |
| 94390313 | 197429 | rs6583831 |
| 94390339 | 197455 | rs11187110 |
| 94390489 | 197605 | rs11187111 |
| 94390602 | 197718 | rs4933733 |
| 94390673 | 197789 | rs34848251 |
| 94390842 | 197958 | rs7922902 |
| 94391081 | 198197 | rs11282830 |
| 94391082 | 198198 | rs35084253 |
| 94391352 | 198468 | rs6583832 |
| 94391366 | 198482 | rs10882096 |
| 94391873 | 198989 | rs7079583 |
| 94391950 | 199066 | rs7079602 |
| 94391966 | 199082 | rs11187112 |
| 94392278 | 199394 | rs12244429 |
| 94393212 | 200328 | rs35312216 |
| 94393281 | 200397 | rs28576319 |
| 94393578 | 200694 | rs7088685 |
| 94394524 | 201640 | rs17107776 |
| 94394677 | 201793 | rs7902436 |
| 94394804 | 201920 | rs7917359 |
| 94395324 | 202440 | rs36070037 |
| 94395344 | 202460 | rs34647366 |
| 94395706 | 202822 | rs33915127 |
| 94396217 | 203333 | rs11187114 |
| 94396832 | 203948 | rs7069619 |
| 94397097 | 204213 | rs34629164 |
| 94397263 | 204379 | rs11187115 |
| 94397647 | 204763 | rs7073957 |
| 94397851 | 204967 | rs10686721 |
| 94397859 | 204975 | rs34357393 |
| 94397981 | 205097 | rs36046902 |
| 94398388 | 205504 | rs12264712 |
| 94398585 | 205701 | rs34132402 |
| 94398837 | 205953 | rs12358273 |
| 94398866 | 205982 | rs35625726 |
| 94398869 | 205985 | rs11285704 |
| 94399729 | 206845 | rs7067733 |
| 94399780 | 206896 | rs6583833 |
| 94399806 | 206922 | rs35230377 |
| 94400137 | 207253 | rs10430651 |
| 94400806 | 207922 | rs12262781 |
| 94400870 | 207986 | rs12359115 |
| 94401006 | 208122 | rs12359131 |
| 94401155 | 208271 | rs7898318 |
| 94401180 | 208296 | rs12359158 |
| 94401236 | 208352 | rs12359159 |
| 94401240 | 208356 | rs12764793 |
| 94401245 | 208361 | rs12764800 |
| 94401246 | 208362 | rs12241432 |
| 94401250 | 208366 | rs11383128 |
| 94401253 | 208369 | rs7898430 |
| 94401708 | 208824 | rs35738968 |
| 94401710 | 208826 | rs10537782 |
| 94401720 | 208836 | rs36000362 |
| 94401732 | 208848 | rs7893798 |
| 94402105 | 209221 | rs11187116 |
| 94402138 | 209254 | rs11187117 |
| 94402166 | 209282 | rs11187118 |
| 94402579 | 209695 | rs12411448 |
| 94402726 | 209842 | rs35910196 |
| 94402828 | 209944 | rs34963356 |
| 94403360 | 210476 | rs17875344 |
| 94403488 | 210604 | rs34417963 |
| 94403643 | 210759 | rs41290170 |
| 94403647 | 210763 | rs17875345 |
| 94403675 | 210791 | rs41290172 |
| 94403774 | 210890 | rs41290174 |
| 94403917 | 211033 | rs1044146 |
| 94403990 | 211106 | rs17875346 |
| 94404243 | 211359 | rs7078243 |
| 94404370 | 211486 | rs11187119 |
| 94404390 | 211506 | rs36107638 |
| 94404547 | 211663 | rs4933734 |
| 94404618 | 211734 | rs17875347 |
| 94405058 | 212174 | rs1044153 |
| 94405646 | 212762 | rs11187120 |
| 94405813 | 212929 | rs35698103 |
| 94406209 | 213325 | rs7071912 |
| 94406381 | 213497 | rs11597699 |
| 94406501 | 213617 | rs11598250 |
| 94406519 | 213635 | rs35135018 |
| 94406834 | 213950 | rs12242617 |
| 94407185 | 214301 | rs12264496 |
| 94407201 | 214317 | rs11814562 |
| 94407283 | 214399 | rs7916594 |
| 94407322 | 214438 | rs7093043 |
| 94407327 | 214443 | rs7092669 |
| 94407904 | 215020 | rs12244738 |
| 94408110 | 215226 | rs12573394 |
| 94408195 | 215311 | rs34223527 |
| 94408405 | 215521 | rs7921040 |
| 94408480 | 215596 | rs35583259 |
| 94408734 | 215850 | rs2901599 |
| 94409046 | 216162 | rs11187122 |
| 94409276 | 216392 | rs6583834 |
| 94410546 | 217662 | rs7076842 |
| 94411653 | 218769 | rs9804194 |
| 94411998 | 219114 | rs7914504 |
| 94412220 | 219336 | rs35580348 |
| 94412694 | 219810 | rs7079361 |
| 94412743 | 219859 | rs7095377 |
| 94413406 | 220522 | rs7096187 |
| 94413485 | 220601 | rs6583835 |
| 94413764 | 220880 | rs6583836 |
| 94413791 | 220907 | rs7100344 |
| 94414053 | 221169 | rs2153827 |
| 94414178 | 221294 | rs6583837 |
| 94414402 | 221518 | rs35978445 |
| 94414724 | 221840 | rs2497321 |
| 94414758 | 221874 | rs2497320 |
| 94415097 | 222213 | rs11187124 |
| 94415101 | 222217 | rs7913315 |
| 94415116 | 222232 | rs2497319 |
| 94415147 | 222263 | rs7071919 |
| 94416183 | 223299 | rs2488058 |
| 94416216 | 223332 | rs2488057 |
| 94417125 | 224241 | rs34940159 |
| 94417197 | 224313 | rs12776166 |
| 94417669 | 224785 | rs11187125 |
| 94418089 | 225205 | rs7910187 |
| 94418694 | 225810 | rs7914114 |
| 94418703 | 225819 | rs11554568 |
| 94418859 | 225975 | rs7914143 |
| 94418888 | 226004 | rs1832887 |
| 94418890 | 226006 | rs1418391 |
| 94418990 | 226106 | rs7914507 |
| 94419062 | 226178 | rs17875348 |
| 94419074 | 226190 | rs7899695 |
| 94419424 | 226540 | rs12414381 |
| 94419447 | 226563 | rs7918084 |
| 94419491 | 226607 | rs7903302 |
| 94419506 | 226622 | rs11187126 |
| 94419569 | 226685 | rs11187127 |
| 94419688 | 226804 | rs11187128 |
| 94419689 | 226805 | rs12772782 |
| 94419887 | 227003 | rs11187129 |
| 94420477 | 227593 | rs2185756 |
| 94420505 | 227621 | rs9419741 |
| 94420766 | 227882 | rs34709928 |
| 94420766 | 227882 | rs34848369 |
| 94420877 | 227993 | rs35250649 |
| 94421300 | 228416 | rs9419742 |
| 94421540 | 228656 | rs11594482 |
| 94421980 | 229096 | rs2497318 |
| 94422308 | 229424 | rs2488055 |
| 94422330 | 229446 | rs6583838 |
| 94422446 | 229562 | rs2488054 |
| 94423353 | 230469 | rs7909334 |
| 94423442 | 230558 | rs35288203 |
| 94423443 | 230559 | rs7897560 |
| 94423460 | 230576 | rs35413668 |
| 94423462 | 230578 | rs7897565 |
| 94423473 | 230589 | rs12761997 |
| 94423750 | 230866 | rs34831256 |
| 94424074 | 231190 | rs4421685 |
| 94425223 | 232339 | rs28514404 |
| 94425223 | 232339 | rs28591207 |
| 94425307 | 232423 | rs35885794 |
| 94425468 | 232584 | rs7098199 |
| 94425577 | 232693 | rs34985734 |
| 94425653 | 232769 | rs35906730 |
| 94425663 | 232779 | rs4933735 |
| 94425739 | 232855 | rs7098638 |
| 94426831 | 233947 | rs7911264 |
| 94427225 | 234341 | rs11187131 |
| 94427575 | 234691 | rs11187132 |
| 94428318 | 235434 | rs11448446 |
| 94428319 | 235435 | rs34215006 |
| 94428322 | 235438 | rs11439271 |
| 94428328 | 235444 | rs35808040 |
| 94429339 | 236455 | rs34911167 |
| 94429434 | 236550 | rs11599074 |
| 94429447 | 236563 | rs12774356 |
| 94429484 | 236600 | rs12779070 |
| 94430250 | 237366 | rs28757342 |
| 94430530 | 237646 | rs35299744 |
| 94431405 | 238521 | rs7089358 |
| 94431962 | 239078 | rs35828108 |
| 94432143 | 239259 | rs34615602 |
| 94432674 | 239790 | rs7914280 |
| 94432723 | 239839 | rs17875349 |
| 94432994 | 240110 | rs34669790 |
| 94434773 | 241889 | rs10882098 |
| 94434827 | 241943 | rs12778051 |
| 94434836 | 241952 | rs11187133 |
| 94434881 | 241997 | rs11187134 |
| 94435923 | 243039 | rs2497317 |
| 94436021 | 243137 | rs2488087 |
| 94436103 | 243219 | rs12262390 |
| 94436615 | 243731 | rs10786052 |
| 94437076 | 244192 | rs10522178 |
| 94437120 | 244236 | rs10531158 |
| 94437559 | 244675 | rs12251379 |
| 94437891 | 245007 | rs949579 |
| 94438736 | 245852 | rs11187135 |
| 94438875 | 245991 | rs34593856 |
| 94439140 | 246256 | rs12781513 |
| 94439449 | 246565 | rs7475059 |
| 94439530 | 246646 | rs4075563 |
| 94439906 | 247022 | rs34266926 |
| 94439912 | 247028 | rs2229328 |
| 94439992 | 247108 | rs1418387 |
| 94440106 | 247222 | rs2901618 |
| 94440107 | 247223 | rs2901619 |
| 94440209 | 247325 | rs34372348 |
| 94440213 | 247329 | rs9420589 |
| 94441710 | 248826 | rs41290176 |
| 94442254 | 249370 | rs17851141 |
| 94442322 | 249438 | rs2275730 |
| 94442410 | 249526 | rs2275729 |
| 94443456 | 250572 | rs11319879 |
| 94443457 | 250573 | rs34616944 |
| 94444237 | 251353 | rs34433040 |
| 94445202 | 252318 | rs35423905 |
| 94445857 | 252973 | rs34619410 |
| 94448868 | 255984 | rs3076219 |
| 94449333 | 256449 | rs34231544 |
| 94449598 | 256714 | rs35657492 |
| 94450261 | 257377 | rs11187138 |
| 94450630 | 257746 | rs10882099 |
| 94450667 | 257783 | rs10882100 |
| 94451033 | 258149 | rs7100035 |
| 94451324 | 258440 | rs7100357 |
| 94451790 | 258906 | rs12263166 |
| 94451929 | 259045 | rs12263147 |
| 94451954 | 259070 | rs12218257 |
| 94451963 | 259079 | rs2488085 |
| 94451965 | 259081 | rs2497316 |
| 94452407 | 259523 | rs10882101 |
| 94452569 | 259685 | rs5787008 |
| 94452862 | 259978 | rs1111875 |
| 94453437 | 260553 | rs7921459 |
| 94453671 | 260787 | rs35245232 |
| 94453759 | 260875 | rs1977832 |
| 94453925 | 261041 | rs1977833 |
| 94453943 | 261059 | rs35396842 |
| 94453943 | 261059 | rs11309330 |
| 94453950 | 261066 | rs34470242 |
| 94454287 | 261403 | rs12778642 |
| 94454556 | 261672 | rs7911447 |
| 94454563 | 261679 | rs7894796 |
| 94454564 | 261680 | rs7911455 |
| 94454571 | 261687 | rs7895123 |
| 94455183 | 262299 | rs4933234 |
| 94455211 | 262327 | rs34991559 |
| 94455539 | 262655 | rs5015480 |
| 94456086 | 263202 | rs2497315 |
| 94456407 | 263523 | rs11187139 |
| 94456419 | 263535 | rs12219514 |
| 94456475 | 263591 | rs10882102 |
| 94456646 | 263762 | rs12572190 |
| 94456890 | 264006 | rs11187140 |
| 94456999 | 264115 | rs7904159 |
| 94457006 | 264122 | rs7904279 |
| 94457011 | 264127 | rs7904508 |
| 94457018 | 264134 | rs7904513 |
| 94457029 | 264145 | rs7904292 |
| 94457125 | 264241 | rs11187141 |
| 94457267 | 264383 | rs34744311 |
| 94458157 | 265273 | rs9419743 |
| 94458159 | 265275 | rs12254229 |
| 94458272 | 265388 | rs12246641 |
| 94458305 | 265421 | rs12246541 |
| 94458432 | 265548 | rs11813799 |
| 94458665 | 265781 | rs11187142 |
| 94458914 | 266030 | rs11187143 |
| 94459172 | 266288 | rs28590736 |
| 94459519 | 266635 | rs28428943 |
| 94459960 | 267076 | rs11187144 |
| 94460495 | 267611 | rs10882104 |
| 94460975 | 268091 | rs34859807 |
| 94460979 | 268095 | rs2497314 |
| 94461575 | 268691 | rs4933736 |
| 94462185 | 269301 | rs34966020 |
| 94462949 | 270065 | rs34290965 |
| 94463018 | 270134 | rs11817277 |
| 94463116 | 270232 | rs34600815 |
| 94463139 | 270255 | rs34793711 |
| 94463609 | 270725 | rs7087591 |
| 94464077 | 271193 | rs10786053 |
| 94464091 | 271207 | rs10882105 |
| 94464476 | 271592 | rs1578672 |
| 94465084 | 272200 | rs35705157 |
| 94465091 | 272207 | rs2901609 |
| 94465573 | 272689 | rs34405337 |
| 94465640 | 272756 | rs4545448 |
| 94466041 | 273157 | rs4504977 |
| 94466253 | 273369 | rs4262638 |
| 94466275 | 273391 | rs12777206 |
| 94466575 | 273691 | rs12782663 |
| 94466582 | 273698 | rs12782667 |
| 94466623 | 273739 | rs12781544 |
| 94467199 | 274315 | rs10748582 |
| 94467336 | 274452 | rs11378649 |
| 94467337 | 274453 | rs33926570 |
| 94467472 | 274588 | rs2488082 |
| 94467519 | 274635 | rs1832886 |
| 94467850 | 274966 | rs7898054 |
| 94468084 | 275200 | rs11187145 |
| 94468335 | 275451 | rs11187146 |
| 94468644 | 275760 | rs2488081 |
| 94468673 | 275789 | rs2488080 |
| 94469087 | 276203 | rs1112718 |
| 94469812 | 276928 | rs12260097 |
| 94469857 | 276973 | rs34383024 |
| 94470058 | 277174 | rs35816895 |
| 94470314 | 277430 | rs10882106 |
| 94470371 | 277487 | rs2497313 |
| 94470482 | 277598 | rs7086841 |
| 94470666 | 277782 | rs2497312 |
| 94471062 | 278178 | rs9420591 |
| 94471614 | 278730 | rs4933235 |
| 94471897 | 279013 | rs7923837 |
| 94471924 | 279040 | rs10673051 |
| 94471925 | 279041 | rs35275238 |
| 94471968 | 279084 | rs35606816 |
| 94471975 | 279091 | rs2497311 |
| 94472056 | 279172 | rs7923866 |
| 94472115 | 279231 | rs12780253 |
| 94472584 | 279700 | rs35282244 |
| 94473140 | 280256 | rs36026029 |
| 94473357 | 280473 | rs11187147 |
| 94473463 | 280579 | rs2488079 |
| 94473656 | 280772 | rs10529320 |
| 94473689 | 280805 | rs2497310 |
| 94473690 | 280806 | rs2488078 |
| 94473695 | 280811 | rs3221117 |
| 94473956 | 281072 | rs2497309 |
| 94474172 | 281288 | rs870786 |
| 94474324 | 281440 | rs2497308 |
| 94474328 | 281444 | rs2488077 |
| 94474418 | 281534 | rs5787009 |
| 94474440 | 281556 | rs5787010 |
| 94474442 | 281558 | rs5787011 |
| 94474472 | 281588 | rs11187148 |
| 94474486 | 281602 | rs1418390 |
| 94474604 | 281720 | rs2497307 |
| 94474750 | 281866 | rs7081035 |
| 94474783 | 281899 | rs7081294 |
| 94474989 | 282105 | rs7081351 |
| 94475058 | 282174 | rs7081745 |
| 94475060 | 282176 | rs10568596 |
| 94475063 | 282179 | rs7099393 |
| 94475082 | 282198 | rs10665748 |
| 94475086 | 282202 | rs34350311 |
| 94475191 | 282307 | rs2497306 |
| 94475601 | 282717 | rs2497305 |
| 94475656 | 282772 | rs34372918 |
| 94475743 | 282859 | rs11187149 |
| 94476016 | 283132 | rs11597458 |
| 94476061 | 283177 | rs2488076 |
| 94476146 | 283262 | rs11593164 |
| 94476357 | 283473 | rs34848929 |
| 94476358 | 283474 | rs11597547 |
| 94476822 | 283938 | rs11187150 |
| 94477781 | 284897 | rs1544210 |
| 94478596 | 285712 | rs35619602 |
| 94479180 | 286296 | rs10630735 |
| 94479181 | 286297 | rs35097519 |
| 94479192 | 286308 | rs34237492 |
| 94479194 | 286310 | rs11309242 |
| 94480154 | 287270 | rs2488075 |
| 94480323 | 287439 | rs12762754 |
| 94480347 | 287463 | rs11593631 |
| 94480992 | 288108 | rs9730884 |
| 94481595 | 288711 | rs11379031 |
| 94481781 | 288897 | rs10615317 |
| 94481897 | 289013 | rs35598412 |
| 94482696 | 289812 | rs2497304 |
| 94482730 | 289846 | rs9419745 |
| 94482914 | 290030 | rs35849687 |
| 94483045 | 290161 | rs34009238 |
| 94483719 | 290835 | rs2488074 |
| 94484440 | 291556 | rs11187151 |
| 94484498 | 291614 | rs35406218 |
| 94485046 | 292162 | rs34249712 |
| 94485097 | 292213 | rs2497303 |
| 94485259 | 292375 | rs4933738 |
| 94485733 | 292849 | rs947591 |
| 94485978 | 293094 | rs7916355 |
| 94486361 | 293477 | rs2051004 |
| 94488416 | 295532 | rs4933236 |
| 94488811 | 295927 | rs17107841 |
| 94488843 | 295959 | rs33985961 |
| 94488845 | 295961 | rs10578040 |
| 94488955 | 296071 | rs2488073 |
| 94489325 | 296441 | rs2488072 |
| 94489493 | 296609 | rs34209030 |
| 94489557 | 296673 | rs2488071 |
| 94489846 | 296962 | rs7917254 |
| 94490010 | 297126 | rs11318190 |
| 94490015 | 297131 | rs34994435 |
| 94490015 | 297131 | rs10588167 |
| 94490091 | 297207 | rs11187152 |

TABLE 12
SNPs within LD block C17 between positions 66,037,656 and
66,163,076 bp on Chromosome 17 in NCBI build 35 and NCBI Build 36.)

| | Position in | |
|---|---|---|
| Position in | SEQ ID | |
| Build 35/36 | NO: 3 | Marker ID |
| 66037656 | 1 | rs11077501 |
| 66038245 | 590 | rs10445229 |
| 66038446 | 791 | rs8067115 |
| 66038456 | 801 | rs10445230 |
| 66038691 | 1036 | rs10445231 |
| 66039757 | 2102 | rs28569992 |
| 66039800 | 2145 | rs4606755 |
| 66039816 | 2161 | rs4435300 |
| 66039936 | 2281 | rs35154837 |
| 66039942 | 2287 | rs9630701 |
| 66040960 | 3305 | rs12165045 |
| 66040982 | 3327 | rs7359539 |
| 66041088 | 3433 | rs7359543 |
| 66042479 | 4824 | rs365813 |
| 66043002 | 5347 | rs4261590 |
| 66043301 | 5646 | rs7223187 |
| 66043481 | 5826 | rs6146132 |
| 66043562 | 5907 | rs721249 |
| 66043745 | 6090 | rs5821786 |
| 66043746 | 6091 | rs33957619 |
| 66043759 | 6104 | rs5821787 |
| 66043760 | 6105 | rs33961999 |
| 66043764 | 6109 | rs12950870 |
| 66044207 | 6552 | rs350605 |
| 66044302 | 6647 | rs10559381 |
| 66044390 | 6735 | rs11650835 |
| 66044411 | 6756 | rs7209364 |
| 66044489 | 6834 | rs350604 |
| 66044496 | 6841 | rs11657696 |
| 66044614 | 6959 | rs11651554 |
| 66044626 | 6971 | rs11657734 |
| 66045245 | 7590 | rs350603 |
| 66045317 | 7662 | rs2307760 |
| 66045722 | 8067 | rs11653245 |
| 66045948 | 8293 | rs11653355 |
| 66047524 | 9869 | rs34832542 |
| 66047547 | 9892 | rs2567294 |
| 66047580 | 9925 | rs11655558 |
| 66047597 | 9942 | rs184783 |
| 66047621 | 9966 | rs353452 |
| 66047646 | 9991 | rs9897791 |
| 66047700 | 10045 | rs11655611 |
| 66047739 | 10084 | rs1825672 |
| 66047807 | 10152 | rs35941755 |
| 66047887 | 10232 | rs9896649 |
| 66048278 | 10623 | rs34984463 |
| 66048288 | 10633 | rs11374691 |
| 66048300 | 10645 | rs11868103 |
| 66048450 | 10795 | rs7220610 |
| 66048799 | 11144 | rs7216368 |
| 66048942 | 11287 | rs16913 |
| 66049292 | 11637 | rs34941209 |
| 66049692 | 12037 | rs8069108 |
| 66049716 | 12061 | rs420762 |
| 66050080 | 12425 | rs2630640 |
| 66050452 | 12797 | rs34793380 |
| 66050707 | 13052 | rs1817630 |
| 66050903 | 13248 | rs11077502 |
| 66050915 | 13260 | rs7218450 |
| 66051172 | 13517 | rs411602 |
| 66051859 | 14204 | rs16975882 |
| 66051914 | 14259 | rs41450951 |
| 66052282 | 14627 | rs17780198 |
| 66052347 | 14692 | rs10512540 |
| 66052398 | 14743 | rs2630639 |
| 66052474 | 14819 | rs4793432 |
| 66052546 | 14891 | rs34696190 |
| 66052699 | 15044 | rs12952273 |
| 66053325 | 15670 | rs350612 |
| 66053541 | 15886 | rs1284043 |
| 66053695 | 16040 | rs1298182 |
| 66053988 | 16333 | rs1092528 |
| 66054007 | 16352 | rs1091892 |
| 66054019 | 16364 | rs1092390 |
| 66054025 | 16370 | rs1092391 |
| 66054076 | 16421 | rs276805 |
| 66054488 | 16833 | rs164784 |
| 66055098 | 17443 | rs164785 |
| 66056158 | 18503 | rs350611 |
| 66057036 | 19381 | rs9736449 |
| 66057065 | 19410 | rs1161565 |
| 66057065 | 19410 | rs350610 |
| 66057184 | 19529 | rs36160618 |
| 66057341 | 19686 | rs28835946 |
| 66057721 | 20066 | rs350609 |
| 66057907 | 20252 | rs36143257 |
| 66058061 | 20406 | rs164786 |
| 66058223 | 20568 | rs4506943 |
| 66058544 | 20889 | rs164787 |
| 66058598 | 20943 | rs35063328 |
| 66058616 | 20961 | rs35629111 |
| 66058724 | 21069 | rs35654390 |
| 66058733 | 21078 | rs350608 |
| 66058804 | 21149 | rs589894 |
| 66059113 | 21458 | rs512280 |
| 66059121 | 21466 | rs512274 |
| 66059131 | 21476 | rs512241 |
| 66059267 | 21612 | rs35813361 |
| 66059431 | 21776 | rs34292805 |
| 66060049 | 22394 | rs671190 |
| 66060102 | 22447 | rs671117 |
| 66060111 | 22456 | rs35419562 |
| 66060172 | 22517 | rs8080393 |
| 66060192 | 22537 | rs509784 |
| 66060244 | 22589 | rs509924 |
| 66060364 | 22709 | rs510865 |
| 66060367 | 22712 | rs669895 |
| 66060401 | 22746 | rs8075249 |
| 66060403 | 22748 | rs8080759 |
| 66060407 | 22752 | rs511552 |
| 66060416 | 22761 | rs511578 |
| 66060618 | 22963 | rs11326414 |
| 66061168 | 23513 | rs350607 |
| 66061287 | 23632 | rs8081864 |
| 66061435 | 23780 | rs8066762 |
| 66063324 | 25669 | rs10048191 |
| 66063794 | 26139 | rs34162560 |
| 66063983 | 26328 | rs350606 |
| 66064178 | 26523 | rs8078924 |
| 66065291 | 27636 | rs4793451 |
| 66065798 | 28143 | rs350613 |
| 66066258 | 28603 | rs10432003 |
| 66066436 | 28781 | rs350614 |
| 66066465 | 28810 | rs34703743 |
| 66066481 | 28826 | rs35908278 |
| 66066608 | 28953 | rs11654062 |
| 66067303 | 29648 | rs350615 |
| 66067453 | 29798 | rs16975891 |
| 66067482 | 29827 | rs17823280 |
| 66067699 | 30044 | rs350616 |
| 66068320 | 30665 | rs350617 |
| 66068798 | 31143 | rs1991680 |
| 66069274 | 31619 | rs9896037 |
| 66069554 | 31899 | rs16975893 |
| 66069880 | 32225 | rs8081551 |
| 66070068 | 32413 | rs11654475 |
| 66071319 | 33664 | rs11651609 |
| 66071575 | 33920 | rs34132957 |
| 66071603 | 33948 | rs12603169 |
| 66071721 | 34066 | rs8073324 |
| 66072276 | 34621 | rs1431455 |
| 66072384 | 34729 | rs17763769 |
| 66073012 | 35357 | rs350618 |
| 66073300 | 35645 | rs1991679 |
| 66073592 | 35937 | rs34134043 |
| 66073862 | 36207 | rs7208933 |
| 66074000 | 36345 | rs11655478 |
| 66074367 | 36712 | rs35062489 |
| 66074796 | 37141 | rs9900305 |
| 66075575 | 37920 | rs16975908 |
| 66076138 | 38483 | rs7224554 |
| 66076400 | 38745 | rs35795750 |
| 66076402 | 38747 | rs5821788 |
| 66076579 | 38924 | rs350619 |
| 66076797 | 39142 | rs34028570 |
| 66076805 | 39150 | rs5821789 |
| 66076806 | 39151 | rs35251724 |
| 66077103 | 39448 | rs7210525 |
| 66077477 | 39822 | rs2567296 |
| 66077488 | 39833 | rs1843621 |
| 66077930 | 40275 | rs34077265 |
| 66077984 | 40329 | rs528669 |
| 66078111 | 40456 | rs8067160 |
| 66078127 | 40472 | rs350620 |
| 66078527 | 40872 | rs191621 |
| 66079419 | 41764 | rs350621 |
| 66079437 | 41782 | rs350622 |
| 66079660 | 42005 | rs12452538 |
| 66079935 | 42280 | rs17176093 |
| 66079969 | 42314 | rs350623 |
| 66079990 | 42335 | rs11077503 |
| 66080024 | 42369 | rs11077504 |
| 66080067 | 42412 | rs350624 |
| 66080672 | 43017 | rs35072892 |
| 66080920 | 43265 | rs11657749 |
| 66080984 | 43329 | rs16975914 |
| 66081110 | 43455 | rs34693986 |
| 66081370 | 43715 | rs350625 |
| 66081556 | 43901 | rs818765 |
| 66081568 | 43913 | rs415298 |
| 66081576 | 43921 | rs376750 |
| 66081612 | 43957 | rs10652573 |
| 66081700 | 44045 | rs350626 |
| 66082086 | 44431 | rs28590672 |
| 66083526 | 45871 | rs16975922 |
| 66083670 | 46015 | rs481417 |
| 66083783 | 46128 | rs191622 |
| 66083825 | 46170 | rs482515 |
| 66083858 | 46203 | rs367218 |
| 66083931 | 46276 | rs402214 |
| 66084484 | 46829 | rs483543 |
| 66084515 | 46860 | rs484253 |
| 66084734 | 47079 | rs486202 |
| 66084769 | 47114 | rs610662 |
| 66084772 | 47117 | rs11310950 |
| 66084781 | 47126 | rs12936985 |
| 66084782 | 47127 | rs12945927 |
| 66084808 | 47153 | rs610730 |
| 66084932 | 47277 | rs1825669 |
| 66084935 | 47280 | rs1825670 |
| 66084954 | 47299 | rs1825671 |
| 66085342 | 47687 | rs8077690 |
| 66085473 | 47818 | rs12602288 |
| 66086152 | 48497 | rs16975937 |
| 66086744 | 49089 | rs28694321 |
| 66087301 | 49646 | rs35353185 |
| 66087527 | 49872 | rs41486747 |
| 66087994 | 50339 | rs718950 |
| 66088026 | 50371 | rs718951 |
| 66089255 | 51600 | rs11654235 |
| 66089418 | 51763 | rs11077506 |
| 66090535 | 52880 | rs1431454 |
| 66090620 | 52965 | rs5821790 |
| 66090782 | 53127 | rs1367748 |
| 66090958 | 53303 | rs12603995 |
| 66091042 | 53387 | rs16975939 |
| 66091117 | 53462 | rs11434683 |
| 66091324 | 53669 | rs8081186 |
| 66091594 | 53939 | rs149309 |
| 66091687 | 54032 | rs184806 |
| 66091693 | 54038 | rs149380 |
| 66091704 | 54049 | rs151727 |
| 66091719 | 54064 | rs189541 |
| 66091741 | 54086 | rs11651021 |
| 66091844 | 54189 | rs416121 |
| 66091912 | 54257 | rs9302918 |
| 66092080 | 54425 | rs9302919 |
| 66092700 | 55045 | rs35618929 |
| 66092813 | 55158 | rs35870620 |
| 66092904 | 55249 | rs11652089 |
| 66093601 | 55946 | rs12938026 |
| 66093669 | 56014 | rs12948379 |
| 66094196 | 56541 | rs9911671 |
| 66094376 | 56721 | rs16975941 |
| 66094422 | 56767 | rs7220885 |
| 66094832 | 57177 | rs601297 |
| 66094858 | 57203 | rs601615 |
| 66094862 | 57207 | rs601617 |
| 66094892 | 57237 | rs601656 |
| 66095313 | 57658 | rs418402 |
| 66096181 | 58526 | rs35417478 |
| 66097168 | 59513 | rs16975944 |
| 66097631 | 59976 | rs34913709 |
| 66097633 | 59978 | rs9894781 |
| 66097634 | 59979 | rs9914075 |
| 66097640 | 59985 | rs11658937 |
| 66097733 | 60078 | rs8078784 |
| 66097760 | 60105 | rs9915992 |
| 66098070 | 60415 | rs10634138 |
| 66098071 | 60416 | rs34728014 |
| 66098073 | 60418 | rs34864826 |
| 66098076 | 60421 | rs10551730 |
| 66098084 | 60429 | rs5821791 |
| 66098085 | 60430 | rs34310496 |
| 66098092 | 60437 | rs34563419 |
| 66098173 | 60518 | rs7503632 |
| 66098597 | 60942 | rs9902449 |
| 66098928 | 61273 | rs9894881 |
| 66098930 | 61275 | rs9894882 |
| 66098976 | 61321 | rs11656877 |
| 66099163 | 61508 | rs5821792 |
| 66099494 | 61839 | rs16975946 |
| 66099600 | 61945 | rs17779190 |
| 66099816 | 62161 | rs11650015 |
| 66100055 | 62400 | rs10607347 |
| 66100062 | 62407 | rs11372958 |
| 66100081 | 62426 | rs34073356 |
| 66100089 | 62434 | rs36104345 |
| 66100401 | 62746 | rs2109051 |
| 66100605 | 62950 | rs2159312 |
| 66101242 | 63587 | rs990043 |
| 66101267 | 63612 | rs576754 |
| 66101396 | 63741 | rs2035582 |
| 66101665 | 64010 | rs9905624 |
| 66101895 | 64240 | rs693914 |
| 66102064 | 64409 | rs558507 |
| 66102168 | 64513 | rs35142117 |
| 66102168 | 64513 | rs10596869 |
| 66102213 | 64558 | rs560206 |
| 66102221 | 64566 | rs12949221 |
| 66102269 | 64614 | rs560368 |
| 66102315 | 64660 | rs1911969 |
| 66102441 | 64786 | rs35550717 |
| 66102450 | 64795 | rs34779818 |
| 66102555 | 64900 | rs9892329 |
| 66102591 | 64936 | rs11658215 |
| 66102896 | 65241 | rs35815207 |
| 66103027 | 65372 | rs9914225 |
| 66103236 | 65581 | rs9894021 |
| 66103495 | 65840 | rs9891523 |
| 66103561 | 65906 | rs720877 |
| 66103923 | 66268 | rs720876 |
| 66103928 | 66273 | rs35174251 |
| 66104116 | 66461 | rs9892968 |
| 66104437 | 66782 | rs17779357 |
| 66104493 | 66838 | rs34287249 |
| 66105315 | 67660 | rs3042758 |
| 66105827 | 68172 | rs1872599 |
| 66106415 | 68760 | rs7218838 |
| 66106622 | 68967 | rs7209535 |
| 66106911 | 69256 | rs9896809 |
| 66107082 | 69427 | rs28507887 |
| 66107150 | 69495 | rs8067542 |
| 66107151 | 69496 | rs10641487 |
| 66107152 | 69497 | rs33989506 |
| 66107167 | 69512 | rs8081487 |
| 66108291 | 70636 | rs4793495 |
| 66108545 | 70890 | rs8073162 |
| 66108565 | 70910 | rs8072591 |
| 66108901 | 71246 | rs9905537 |
| 66108905 | 71250 | rs8073114 |
| 66108924 | 71269 | rs8072003 |
| 66108980 | 71325 | rs35155940 |
| 66108991 | 71336 | rs11459300 |
| 66108997 | 71342 | rs36029337 |
| 66109457 | 71802 | rs11656223 |
| 66110309 | 72654 | rs6501400 |
| 66110507 | 72852 | rs8074266 |
| 66110586 | 72931 | rs388304 |
| 66110881 | 73226 | rs4544280 |
| 66111138 | 73483 | rs12601471 |
| 66111335 | 73680 | rs12603574 |
| 66111468 | 73813 | rs11077507 |
| 66111545 | 73890 | rs11077508 |
| 66111926 | 74271 | rs28546453 |
| 66112148 | 74493 | rs412877 |
| 66112202 | 74547 | rs391223 |
| 66112205 | 74550 | rs7224183 |
| 66112227 | 74572 | rs173318 |
| 66112234 | 74579 | rs192147 |
| 66112749 | 75094 | rs34361437 |
| 66113023 | 75368 | rs12449913 |
| 66114764 | 77109 | rs28496807 |
| 66114858 | 77203 | rs7220084 |
| 66114926 | 77271 | rs7224857 |
| 66115366 | 77711 | rs7221542 |
| 66115371 | 77716 | rs7221545 |
| 66115377 | 77722 | rs34466876 |
| 66115416 | 77761 | rs10610236 |
| 66115835 | 78180 | rs1979538 |
| 66116621 | 78966 | rs8067103 |
| 66116703 | 79048 | rs7216053 |
| 66116880 | 79225 | rs12949351 |
| 66117903 | 80248 | rs9913650 |
| 66117911 | 80256 | rs1860316 |
| 66118086 | 80431 | rs9914115 |
| 66118200 | 80545 | rs9908443 |
| 66118485 | 80830 | rs8079029 |
| 66118495 | 80840 | rs12601922 |
| 66118737 | 81082 | rs10545098 |
| 66118737 | 81082 | rs12603987 |
| 66119589 | 81934 | rs9897225 |
| 66119616 | 81961 | rs35975623 |
| 66119642 | 81987 | rs9895773 |
| 66119822 | 82167 | rs9898518 |
| 66119823 | 82168 | rs28422091 |
| 66119823 | 82168 | rs36094553 |
| 66119863 | 82208 | rs3220372 |
| 66119992 | 82337 | rs41408048 |
| 66120631 | 82976 | rs28373290 |
| 66120827 | 83172 | rs41459950 |
| 66121181 | 83526 | rs36013413 |
| 66121413 | 83758 | rs10564191 |
| 66121468 | 83813 | rs7221715 |
| 66121873 | 84218 | rs12940023 |
| 66122077 | 84422 | rs4019476 |
| 66122410 | 84755 | rs7222670 |
| 66122508 | 84853 | rs7211934 |
| 66122635 | 84980 | rs7212243 |
| 66122801 | 85146 | rs11655139 |
| 66123046 | 85391 | rs4793317 |
| 66123283 | 85628 | rs171384 |
| 66123342 | 85687 | rs4793496 |
| 66123566 | 85911 | rs507683 |
| 66123595 | 85940 | rs507607 |
| 66123682 | 86027 | rs41528454 |
| 66123714 | 86059 | rs41381246 |
| 66123879 | 86224 | rs192146 |
| 66123955 | 86300 | rs34149626 |
| 66123959 | 86304 | rs35260054 |
| 66125089 | 87434 | rs9908077 |
| 66125154 | 87499 | rs11077509 |
| 66125233 | 87578 | rs35234488 |
| 66125360 | 87705 | rs11871352 |
| 66125993 | 88338 | rs7209850 |
| 66126471 | 88816 | rs421333 |
| 66126699 | 89044 | rs413073 |
| 66126766 | 89111 | rs2035581 |
| 66126795 | 89140 | rs392974 |
| 66126799 | 89144 | rs28532132 |
| 66126840 | 89185 | rs3931227 |
| 66126841 | 89186 | rs16975961 |
| 66126875 | 89220 | rs532348 |
| 66127256 | 89601 | rs11867791 |
| 66127283 | 89628 | rs11871014 |
| 66127312 | 89657 | rs34866225 |
| 66127313 | 89658 | rs35958830 |
| 66128191 | 90536 | rs9904090 |
| 66128304 | 90649 | rs16975968 |
| 66129127 | 91472 | rs9911708 |
| 66129806 | 92151 | rs34448828 |
| 66129814 | 92159 | rs11398461 |
| 66130210 | 92555 | rs9907685 |
| 66130284 | 92629 | rs9914666 |
| 66130455 | 92800 | rs17717654 |
| 66131911 | 94256 | rs16975970 |
| 66131995 | 94340 | rs1981646 |
| 66132156 | 94501 | rs11656723 |
| 66132225 | 94570 | rs16975976 |
| 66132788 | 95133 | rs1981647 |
| 66133032 | 95377 | rs16975979 |
| 66133370 | 95715 | rs16975981 |
| 66134231 | 96576 | rs9909661 |
| 66134831 | 97176 | rs9890554 |
| 66135283 | 97628 | rs11077510 |
| 66135627 | 97972 | rs9302920 |
| 66135758 | 98103 | rs34975186 |
| 66136201 | 98546 | rs11870545 |
| 66136484 | 98829 | rs9906234 |
| 66136910 | 99255 | rs10621796 |
| 66136920 | 99265 | rs11328278 |
| 66136922 | 99267 | rs11328279 |
| 66137329 | 99674 | rs35402203 |
| 66137331 | 99676 | rs6501401 |
| 66137347 | 99692 | rs10596163 |
| 66137437 | 99782 | rs35029611 |
| 66137784 | 100129 | rs35985303 |
| 66137798 | 100143 | rs10595957 |
| 66137965 | 100310 | rs10221271 |
| 66138452 | 100797 | rs10221225 |
| 66138713 | 101058 | rs34005576 |
| 66139441 | 101786 | rs9913463 |
| 66139476 | 101821 | rs9915148 |
| 66139800 | 102145 | rs11650683 |
| 66140108 | 102453 | rs34335723 |
| 66140414 | 102759 | rs11654495 |
| 66141319 | 103664 | rs35607820 |
| 66141543 | 103888 | rs12601304 |
| 66141933 | 104278 | rs1486290 |
| 66142011 | 104356 | rs12452862 |
| 66142106 | 104451 | rs35317540 |
| 66142226 | 104571 | rs11077511 |
| 66142325 | 104670 | rs35278774 |
| 66142329 | 104674 | rs7216457 |
| 66142581 | 104926 | rs11077512 |
| 66142607 | 104952 | rs35634443 |
| 66142648 | 104993 | rs11454851 |
| 66142729 | 105074 | rs11654670 |
| 66142794 | 105139 | rs34926966 |
| 66143200 | 105545 | rs8078302 |
| 66143897 | 106242 | rs562472 |
| 66144028 | 106373 | rs28420303 |
| 66144129 | 106474 | rs28542473 |
| 66144170 | 106515 | rs28526433 |
| 66144232 | 106577 | rs12185220 |
| 66144972 | 107317 | rs7350903 |
| 66145018 | 107363 | rs7350904 |
| 66145022 | 107367 | rs7350905 |
| 66145067 | 107412 | rs35353467 |
| 66145660 | 108005 | rs16975985 |
| 66145765 | 108110 | rs16975987 |
| 66145912 | 108257 | rs412981 |
| 66145914 | 108259 | rs412980 |
| 66145925 | 108270 | rs9907746 |
| 66146236 | 108581 | rs432688 |
| 66146640 | 108985 | rs540331 |
| 66146722 | 109067 | rs473792 |
| 66146816 | 109161 | rs2630644 |
| 66146912 | 109257 | rs12949591 |
| 66146954 | 109299 | rs2109053 |
| 66147095 | 109440 | rs35296857 |
| 66147167 | 109512 | rs17791270 |
| 66147246 | 109591 | rs16975989 |
| 66147343 | 109688 | rs17791282 |
| 66147436 | 109781 | rs17718124 |
| 66147660 | 110005 | rs16975993 |
| 66147678 | 110023 | rs35106633 |
| 66147754 | 110099 | rs34429407 |
| 66147913 | 110258 | rs2240749 |
| 66147960 | 110305 | rs16975998 |
| 66148045 | 110390 | rs3217050 |
| 66148046 | 110391 | rs2240750 |
| 66148178 | 110523 | rs16976000 |
| 66148512 | 110857 | rs16976002 |
| 66148962 | 111307 | rs189580 |
| 66149102 | 111447 | rs1843622 |
| 66149149 | 111494 | rs543765 |
| 66149213 | 111558 | rs434729 |
| 66149222 | 111567 | rs375709 |
| 66149257 | 111602 | rs11656782 |
| 66149348 | 111693 | rs11653519 |
| 66149386 | 111731 | rs190256 |
| 66149859 | 112204 | rs4584866 |
| 66150283 | 112628 | rs16976008 |
| 66150360 | 112705 | rs16976009 |
| 66150511 | 112856 | rs16976011 |
| 66150609 | 112954 | rs17718380 |
| 66150898 | 113243 | rs11652208 |
| 66150909 | 113254 | rs11652209 |
| 66151294 | 113639 | rs10491179 |
| 66151858 | 114203 | rs16976019 |
| 66152747 | 115092 | rs35499697 |
| 66152804 | 115149 | rs17791650 |
| 66152863 | 115208 | rs16976023 |
| 66152963 | 115308 | rs16976024 |
| 66152998 | 115343 | rs9891997 |
| 66153185 | 115530 | rs12942978 |
| 66153557 | 115902 | rs16976027 |
| 66153631 | 115976 | rs17718538 |
| 66154056 | 116401 | rs16976031 |
| 66155045 | 117390 | rs34736208 |
| 66155048 | 117393 | rs12952540 |
| 66155070 | 117415 | rs34389302 |
| 66155101 | 117446 | rs189581 |
| 66155303 | 117648 | rs9910837 |
| 66155784 | 118129 | rs17718586 |
| 66156561 | 118906 | rs544680 |
| 66156593 | 118938 | rs8066818 |
| 66157022 | 119367 | rs408448 |
| 66157073 | 119418 | rs11650843 |
| 66157111 | 119456 | rs367742 |
| 66157179 | 119524 | rs550945 |
| 66157197 | 119542 | rs183590 |
| 66157224 | 119569 | rs551058 |
| 66157327 | 119672 | rs34098284 |
| 66157893 | 120238 | rs183591 |
| 66157917 | 120262 | rs183059 |
| 66157976 | 120321 | rs404774 |
| 66158027 | 120372 | rs11657329 |
| 66158091 | 120436 | rs405068 |
| 66158109 | 120454 | rs35166389 |
| 66158247 | 120592 | rs16976038 |
| 66158414 | 120759 | rs183592 |
| 66159001 | 121346 | rs34458687 |
| 66159262 | 121607 | rs35760966 |
| 66159416 | 121761 | rs2191113 |
| 66159464 | 121809 | rs5821793 |
| 66159546 | 121891 | rs5821794 |
| 66159547 | 121892 | rs35222039 |
| 66159556 | 121901 | rs10648023 |
| 66159562 | 121907 | rs3048626 |
| 66159637 | 121982 | rs8072436 |
| 66159764 | 122109 | rs2215270 |
| 66159891 | 122236 | rs16976043 |
| 66160076 | 122421 | rs8074760 |
| 66160292 | 122637 | rs11657599 |
| 66160331 | 122676 | rs34281212 |
| 66160370 | 122715 | rs36074213 |
| 66160438 | 122783 | rs171385 |
| 66160451 | 122796 | rs412353 |
| 66160480 | 122825 | rs422923 |
| 66160492 | 122837 | rs11654012 |
| 66160668 | 123013 | rs35429609 |
| 66160721 | 123066 | rs2367004 |
| 66161977 | 124322 | rs35222003 |
| 66161994 | 124339 | rs11867678 |
| 66162691 | 125036 | rs12953137 |
| 66162852 | 125197 | rs10642929 |
| 66162854 | 125199 | rs34937331 |
| 66162869 | 125214 | rs10585639 |
| 66163076 | 125421 | rs4793497 |
| 66037656 | 1 | rs11077501 |
| 66038245 | 590 | rs10445229 |
| 66038446 | 791 | rs8067115 |
| 66038456 | 801 | rs10445230 |
| 66038691 | 1036 | rs10445231 |
| 66039757 | 2102 | rs28569992 |
| 66039800 | 2145 | rs4606755 |
| 66039816 | 2161 | rs4435300 |
| 66039936 | 2281 | rs35154837 |

TABLE 13
Key to Sequence listing provided herein.

| SEQ ID NO | Name |
|---|---|
| 1 | LD block C06 |
| 2 | LD block C10 |
| 3 | LD block C17 |
| 4 | rs10882091 |
| 5 | rs1111875 |
| 6 | rs1569699 |
| 7 | rs17763769 |
| 8 | rs17763811 |
| 9 | rs1843622 |
| 10 | rs1860316 |
| 11 | rs1981647 |
| 12 | rs1999763 |
| 13 | rs2191113 |
| 14 | rs2275729 |
| 15 | rs2421943 |
| 16 | rs2497304 |
| 17 | rs3829170 |
| 18 | rs4712527 |
| 19 | rs6583826 |
| 20 | rs6583830 |
| 21 | rs7756992 |
| 22 | rs7758851 |
| 23 | rs7908111 |
| 24 | rs7914814 |
| 25 | rs7915186 |
| 26 | rs7917359 |
| 27 | rs7922112 |
| 28 | rs7923837 |
| 29 | rs9295478 |
| 30 | rs947591 |
| 31 | rs9890889 |
| 32 | rs7752906 |
| 33 | rs9350271 |
| 34 | rs9356744 |
| 35 | rs9368222 |
| 36 | rs10440833 |
| 37 | rs6931514 |
| 38 | rs2009802 |
| 39 | rs17718938 |
| 40 | rs17223216 |
| 41 | rs2109050 |
| 42 | rs1962801 |
| 43 | rs7086285 |
| 44 | rs17234378 |

### Example 2

### Variants in the CDKAL1 Gene Influence Insulin Response and the Risk of Type 2 Diabetes

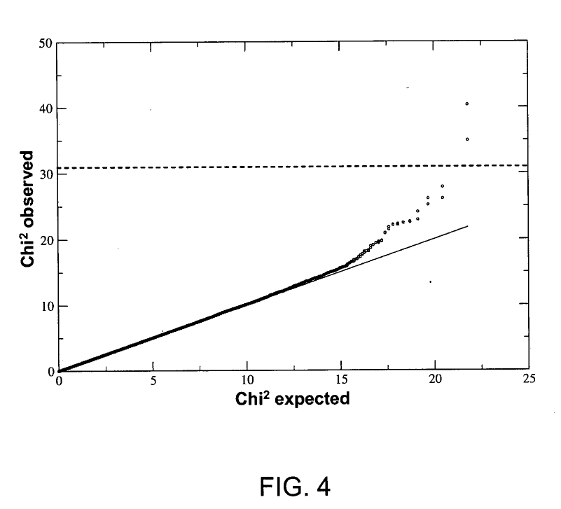

We have recently described a variant in TCF7L2 associated to T2D (Grant, S. F. et al. Nat Genet 38, 320-3 (2006); Helgason, A. et al. Nat Genet (2007)). In the following, we describe a genome-wide association study on Icelandic T2D patients, using the Illumina Hap300 chip. We individually tested 313,179 SNPs for association to T2D in a sample of 1399 T2D patients and 5275 controls. We further tested 339,846 two-marker haplotypes identified as efficient surrogates (r²>0.8) for a set of SNPs which were not included on the Hap300 chip but were typed in the HapMap project (Pe'er, I. et al. Nat Genet 38, 663-7 (2006)). In addition to analyzing the entire group of T2D patients we separately tested 700 non-obese T2D patients and 531 obese T2D patients for association. Overall, a total of 1,959,075 (653,025 variants×3 phenotypes) tests were performed. The results were adjusted for relatedness between individuals and potential population stratification by genomic control (Devlin, B. & Roeder, K. Biometrics 55, 997-1004 (1999)) (see Methods). Specifically, the (unadjusted) chi-square statistics were divided by 1.287, 1.204 and 1.184 respectively for the analyses of all, non-obese and obese T2D cases. A previously identified SNP rs7903146 in the TCF7L2 gene gave the most significant results with OR=1.38 and P=1.82×10⁻¹⁰ in all T2D patients. Although no other SNP or haplotype was significant after adjustment for the number of tests performed, more borderline significant signals were observed than expected by chance alone (FIG. 4). Hence we decided to further pursue the top signals.

### Methods

### Icelandic Study Population

The Icelandic T2D group has been described previously (Reynisdottir, I. et al. Am J Hum Genet 73, 323-35 (2003)). A total of 1500 T2D patients were recruited for this genome-wide association study, using the Infinium II assay method and the Sentrix HumanHap300 BeadChip (Illumina, San Diego, Calif., USA). Thereof, 1399 were successfully genotyped according to our quality control criteria (see Supplementary Methods) and used in the present case control-analysis; 531 of the genotyped cases were obese (BMI≧30). The controls used in this study consisted of 599 controls randomly selected from the Icelandic genealogical database and 4676 individuals from other ongoing genome-wide association studies at deCODE. The study was approved by the Data Protection Commission of Iceland and the National Bioethics Committee of Iceland. Written informed consent was obtained from all cases and controls.

### Other Study Populations

The Danish female study group of 282 cases and 629 controls, herein termed Denmark A, was selected from the Prospective Epidemiological Risk Factor (PERF) study in Denmark (Tanko, L. B., et al. Bone 32, 8-14 (2003)). This is a group of postmenopausal women who took part in various screening placebo-controlled clinical trials and epidemiological studies performed at the Center for Clinical and Basic Research. At a follow-up examination of 5847 women in 2000-2001 medical history including diabetes type I and type II, family history, and current or previous long-term use of drugs were gathered during personal interviews using a preformed questionnaire. If subject was diagnosed as diabetes of either type I or type II, the time of diagnosis or treatment was also collected. The study was approved by the Ethical Committee of Copenhagen County and was in accordance with the principles of the Helsinki Declaration.

The second Danish study population of 1359 T2D cases and 4858 control individuals with normal glucose tolerance was from the Steno Diabetes Center in Copenhagen and from the Inter99 population-based sample of 30- to 60-year-old individuals living in the greater Copenhagen area and sampled at Research Centre for Prevention and Health (Jorgensen, T. et al. Eur J Cardiovasc Prev Rehabil 10, 377-86 (2003)). This dataset is referred to in the text as Denmark B. Diabetes and pre-diabetes categories were diagnosed according to the 1999 World Health Organization (WHO) criteria. An oral glucose tolerance test was performed on participants in the Inter99 study as described (Jorgensen, T. et al. Eur J Cardiovasc Prey Rehabil 10, 377-86 (2003)). Informed written consent was obtained from all subjects before participation. The study was approved by the Ethical Committee of Copenhagen County and was in accordance with the principles of the Helsinki Declaration.

The Philadelphia study population consisted of 468 T2D cases and 1024 control individuals. The study population was selected from the PENN CATH study, a cross-sectional study of the association of biochemical and genetic factors to coronary atherosclerosis in a study population of consecutive individuals undergoing cardiac catheterization at the University of Pennsylvania Medical Center. T2D was defined as a history of fasting blood glucose≧126 mg dl⁻¹, 2 h postprandial glucose≧200 mg dl⁻¹, use of oral hypoglycemic agents, or use of insulin and oral hypoglycemic in a subject older than age 40. The University of Pennsylvania Institutional Review Board approved the study protocol, and all subjects gave written informed consent. All cases and controls were of European ancestry. Ethnicity was determined through self-report.

The Dutch Breda study population consisted of 370 T2D cases and 916 control individuals. The cases were recruited in 1998-1999 in collaboration with the Diabetes Service Breda and 80 general practitioners from the region around Breda. All patients are diagnosed according to WHO criteria (plasma glucose levels>11.1 mmol/l or a fasting plasma glucose level≧7.0 mmol/l), and undergo clinical and laboratory evaluations for their diabetes at regular 3-month intervals. The Medical Ethics Committee of the University Medical Centre in Utrecht approved the study protocol. All probands filled out an informed consent and a questionnaire on clinical data, including their diabetes related medication, height and weight at present and at the age of 20 year. The controls are Dutch blood bank donors with an average age of 48.

The Scottish study population consisted of type 2 diabetic cases and non-diabetic controls from the Wellcome Trust UK T2D case-control collection (Go-DARTS2) which is a sub-study of Diabetes Audit and Research Tayside (DARTS) (Morris, A. D. et al. BMJ 315, 524-8 (1997)). All T2D patients were physician-diagnosed T2D cases recruited at primary or secondary care diabetes clinics, or invited to participate from primary care registers and have not been characterized for GAD anti-bodies or MODY gene mutations. The controls were invited to participate through the primary care physicians or through their workplace occupational health departments. Controls did not have a previous diagnosis of diabetes, but the glucose tolerance status of the controls is unknown. All individuals in this ongoing study were recruited in Tayside between October 2004 and July 2006. This study was approved by the Tayside Medical Ethics Committee and informed consent was obtained from all subjects.

All subjects in the Hong Kong study population were of southern Han Chinese ancestry residing in Hong Kong. The cases consisted of 1500 individuals with T2D selected from the Prince of Wales Hospital Diabetes Registry. Of these, 682 patients had young-onset diabetes (age-at-diagnosis≦40 years) with positive family history. An additional 818 cases were randomly selected from the same registry. The controls consisted of 1000 subjects with normal glucose tolerance (fasting plasma glucose<6.1 mmol/l). Of these, 617 were recruited from the general population participating in a community-based cardiovascular risk screening program as well as hospital staff. In addition, 383 subjects were recruited from a cardiovascular risk screening program for adolescents. Informed consent was obtained for each participating subject. This study was approved by the Clinical Research Ethics Committee of the Chinese University of Hong Kong.

The African study population comes from the Africa America Diabetes Mellitus study, which was originally designed as an affected sibling pair study with enrollment of available spouses as controls. It has since been expanded to include other family members of the affected pairs and population controls. Recruitment strategies and eligibility criteria for the families enrolled in this report have been described previously (Rotimi, C. N. et al. Ann Epidemiol 11, 51-8 (2001)). This West African case-control series consisted of individuals from the Yoruba (233 affected individuals, 432 controls) and Igbo (237 affected individuals, 276 controls) groups from Nigeria and the Akan (257 affected individuals, 248 controls), Ewe (22 affected individuals, 30 controls) and Gaa-Adangbe (123 affected individuals, 141 controls) groups from Ghana.

With the exception of the Scottish Go-DARTS study population the DNA used for genotyping in all replication study populations was the product of whole-genome amplification (GenomiPhi Amplification kit, Amersham) of DNA isolated from the peripheral blood.

### Statistical Analysis

Illumina Genome-Wide Genotyping. All Icelandic case- and control-samples were assayed with the Infinium HumanHap300 SNP chips (Illumina, San Diego, Calif., USA), containing 317,503 haplotype tagging SNPs derived from phase I of the International HapMap project. Of the SNPs assayed on the chip, 4,324 SNPs were excluded as the had (a) yield lower than 95% in cases or controls; (b) minor allele frequency less than 1% in the population; or (c) showed significant distortion from Hardy-Weinberg equilibrium in the controls (P-value<0.001). Any samples with a call rate below 98% were excluded from the analysis. Thus, the final analyses presented in the text utilizes 313,179 SNPs.
Single SNP genotyping. Single SNP genotyping for all population studied, except for the Scottish Go-DARTS population, was carried out at deCODE Genetics in Reykjavik, Iceland by the Centaurus (Nanogen) platform (Kutyavin, I. V. et al. Nucleic Acids Res 34, e128 (2006)). The quality of each Centaurus SNP assay was evaluated by genotyping each assay in the CEU and/or YRI HapMap samples and comparing the results with the HapMap data. Assays with >1.5% mismatch rate were not used and a linkage disequilibrium (LD) test was used for markers known to be in LD. Single SNP genotyping for the Scottish population was carried out at the Biomedical Research Centre, Ninewells Hospital and Medical School, Dundee, Scotland, by the TaqMan® method.
Association analysis. For association analysis we utilized a standard likelihood ratio statistics, implemented in the NEMO software (Gretarsdottir, S. et al. Nat Genet 35, 131-8 (2003)) to calculate two-sided p-values and allele specific OR for each individual allele, assuming a multiplicative model for risk, i.e., that the risks of the two alleles a person carries multiply. Allelic frequencies, rather than carrier frequencies are presented for the markers, and p-values are given after adjustment for the relatedness of the subjects. When estimating genotype specific OR (Table 19) genotype frequencies in the population were estimated assuming HWE.

In general, allele/haplotype frequencies are estimated by maximum likelihood and tests of differences between cases and controls are performed using a generalized likelihood ratio test (Rice, J. A. Mathematical Statistics and Data Analysis, (Wadsworth Inc., Belmont, Calif., 1995)). This method is particularly useful in situations where there are some missing genotypes for the marker of interest and genotypes of another marker, which is in strong LD with the marker of interest, are used to provide some partial information. This was used in the association tests presented in Table 17 to ensure that the comparison of the highly correlated markers was done using the same number of individuals. To handle uncertainties with phase and missing genotypes, maximum likelihood estimates, likelihood ratios and p-values are computed directly for the observed data, and hence the loss of information due to uncertainty in phase and missing genotypes is automatically captured by the likelihood ratios.

Results from multiple case-control groups were combined using a Mantel-Haenszel model (Mantel, N. & Haenszel, W. J Natl Cancer Inst 22, 719-48 (1959)) in which the groups were allowed to have different population frequencies for alleles, and genotypes but were assumed to have common relative risks.

Correction for relatedness of the subjects and Genomic Control. Some of the individuals in both the Icelandic patient and control groups are related to each other, causing the chi-square test statistic to have a mean>1 and median>0.675². We estimated the inflation factor by calculating the average of the 653,025 chi-square statistics, which was a method of genomic control⁴ to adjust for both relatedness and potential population stratification. The inflation factor was estimated as 1.287, 1.204 and 1.184, for the analysis of all, non-obese and obese T2D cases, respectively. The results presented are based on adjusting the chi-square statistics by dividing each of them by the corresponding inflation factor.
Quantitative analysis. Data from oral glucose tolerance test on individuals from the Danish Inter99 study were used to calculate insulin secretion as corrected insulin response (CIR) using the following equation: (100×insulin at 30 minutes)÷[glucose at 30 minutes×(glucose at 30 minutes−3.89 mmol)]. Insulin sensitivity was estimated as the reciprocal of the insulin resistance according to the homeostasis model assessment (HOMA): 22.5/[fasting insulin×fasting glucose] (Matthews, D. R. et al. Diabetologia 28, 412-9 (1985)). The association between CIR (HOMA) and genotype status was tested using a multiple regression where the log-transformed CIR (HOMA) where taken as the response variable and the explanatory variable was either the number of copies of risk allele an individual carries (an additive model) or an indicator variable for homozygous carriers of the risk allele (a recessive model). Adjustment for sex, age and affection status was done by including the appropriate terms as explanatory variables. For comparison insulin secretion was also calculated as (insulin at 30 minutes−insulin at 0 minutes)÷(glucose at 30 minutes−glucose at 0 minutes), yielding comparable results.
Cell lines. The INS1 cells were provided by Hoffmann-LaRoche. They were grown in RPMI1640 (Invitrogen) supplemented with 10% fetal bovine serum (Invitrogen), 50 μg/ml penicillin-streptomycin (Invitrogen), 50 μM 2-mercaptoethanol (SIGMA), 1 mM MEM sodium pyruvate (Invitrogen) and 10 mM Hepes buffer solution (Invitrogen). They were split 1:2 twice per week by washing once in 1× Hanks Balanced Salt Solution (Invitrogen) and then trypsinized (trypsin-EDTA; Invitrogen).
Preparation of RNA and cDNA amplification. INS1 cells were incubated for 48 h in normal growth medium containing 10 mM glucose. At the time of harvest there were 2×10⁷ cells, which were used for the preparation of total RNA. RNA was extracted using RNeasy Midi Kit (Quiagen). cDNA was prepared using High-Capacity cDNA Archive Kit (Applied Biosystems). CDKAL1 cDNA was amplified using two different primer pairs between exons 2 and 8 (forward: 5′-GGGGCTGCTCACATAATAATTCA-3′; reverse: 5′-TGTGCCAATGTCTCTGCCATA-3′) and between exons 7 and 13 (forward: 5′-ACCTGGCCAGCTATCCCATT-3′; reverse: 5′-CCATTTTTCCCATGAATGCAG-3′). Primers from beta-actin served as positive controls (forward 5′-ATCTGGCACCACACCTCCTACAATGAGCTGC-3′; reverse: 5′-CGTCATACTCCTGCTTGCTGATCCACATCTGC-3′).

### Results and Discussion

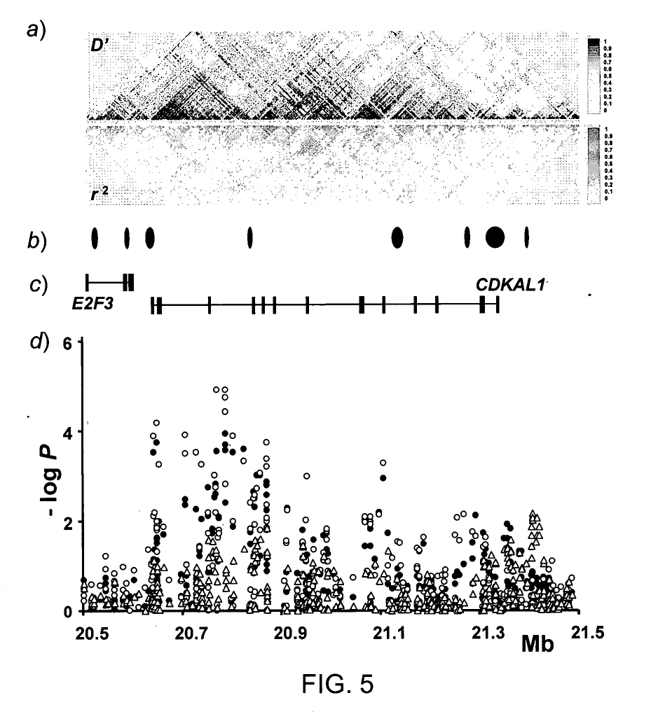

For each phenotype tested we selected all single SNPs and two marker haplotypes with P<0.00005 for replication in a case-control sample from Denmark (Denmark B). After eliminating redundant markers a total of 46 SNPs were taken further for the attempt at replication (Table 14). In addition, we included the five most significant non-synonymous SNPs present on the Illumina Hap300 chip. Out of those 51 SNPs, 47 were successfully genotyped in 1110 Danish T2D cases and 2272 controls. In the Danish group SNPs rs7756992 and rs13266634 stood out and were significantly replicated with P=0.00013 and OR=1.24 and P=0.0012 and OR=1.20, respectively, in the Danish group of all T2D patients (Table 15). This is compared to P=0.00021 and OR=1.23 and P=0.000061 and OR=1.19, respectively in the initial Icelandic study. All of the other SNPs genotyped had P>0.01 in the Danish group and were not pursued further. The first SNP, rs7756992, is located in intron 5 of the CDK5 regulatory subunit associated protein 1-like 1 (CDKAL1) gene on 6p22.3. It resides in a large LD block of 201.7 kb that includes exons 1-5 of the CDKAL1 gene as well as the minimal promoter region but no other known genes (FIG. 5). The second SNP, rs13266634, is a non-synonymous SNP causing an arginine 325 to tryptophan change in the last exon of the solute carrier family 30 (zinc transporter), member 8 (SLC30A8) gene on 8q24. The gene product of SLC30A8 is specific to the pancreas and it is expressed in beta cells where it facilitates the accumulation of zinc from the cytoplasm into intracellular vesicles (Chimienti, F., et al. Diabetes 53, 2330-7 (2004)). The risk allele of rs13266634 on 8q24 has recently been found to confer risk of T2D in a genome wide association study of French T2D cases and controls (Sladek, R. et al. Nature 445, 881-5 (2007)). Of other significantly associated SNPs in that study, we also replicated, in the initial Icelandic samples, association to two SNPs close to the HHEX gene (Table 16). However, we did not replicate the reported association to markers in the LOC387761 and EXT2 genes also described in that study.

We typed the SNPs rs7756992 and rs13266634 in four other T2D case-control groups of European ancestry from Denmark (Denmark A), Scotland, the Netherlands and Philadelphia, US as well as case-control groups from Hong Kong and West Africa. Furthermore, the size of the Denmark B study group was expanded mostly by increasing the number of genotyped controls. The association of the G allele of rs7756992 was replicated with significance in the Scottish (OR=1.11; P=0.0042) and the Hong Kong (OR=1.25; P=0.00018) case-control groups (Table 17). Association in other study groups was not individually significant, but all were in the same direction. The observed association from combining all eight case-control groups gave an OR of 1.15 with a corresponding P of 9.0×10⁻¹² (Table 17). Given that approximately 2 million tests were performed in the initial genome-scan, this association remained highly significant with Bonferonni adjustment (P_{adj}=1.8×10⁻⁵) (Skol, A. D., et al. Nat Genet 38, 209-13 (2006)). Attempts at refining the association observed with rs7756992 by genotyping additional markers that correlate with the original signal in the HapMap CEPH (CEU) dataset, did not yield more significant results (Table 18). As could be expected the linkage disequilibrium observed for the West African population was considerably less than that seen for the Icelandic and Hong Kong groups (Table 19). Further work is needed to determine if an associated variant with a higher OR than observed for rs7756992 can be identified in the West African group. Likewise, for allele C of the non-synonymous SNP rs13266634 the association to T2D was replicated with significance in three of the six additional groups (from Scotland, Philadelphia and Hong Kong) (Table 17). Even though the OR for Denmark B decreased with the larger sample size and the estimated effect was in the opposite direction (only slightly and non-significant) for Denmark A, the combined results from all study group yielded a genome-wide significant P of 2.5×10⁻¹¹ and an OR of 1.16 (Table 17).

In the Icelandic study the association to rs7756992 was more significant in non-obese T2D patients (OR=1.37; P=9.0×10⁻⁶) than in the group of all patients (OR=1.23; P=0.00021) (Table 14 and Table 17). A higher OR in non-obese than in obese T2D patients was also observed for this variant in the other populations studied. For the combined populations of European origin the OR was 1.19; P=7.29×10⁻⁹ for the non-obese T2D patients compared to OR=1.12; P=0.00017 for the obese group. An even stronger effect was seen in the Hong Kong non-obese T2D group (OR=1.36; P=7.48×10⁻⁶), compared to the obese group (OR=1.13; P=0.094), where obesity was defined as BMI≧25. When the results for all groups were combined, relative to controls, OR=1.19; P=1.93×10⁻¹¹ and OR=1.13; P=2.68×10⁻⁵ was obtained for the non-obese and obese T2D patient groups, respectively. These results indicate that this variant does not confer increased risk of T2D through increased BMI.

Genotype odds ratio was estimated for each of the two loci (Table 20). Based on the results for the combined Caucasian study populations rs7756992 deviates significantly from the multiplicative model with OR for the heterozygote=1.09 compared to OR=1.45 for the homozygote, supporting a nearly recessive mode of inheritance. The same trend, although non-significant, was seen for the Hong Kong samples with heterozygote OR=1.13 and OR=1.55 for the homozygote. Conversely, a multiplicative model for the genotype relative risk provided an adequate fit for rs13266634.

The function of the gene product of CDKAL1 is not known. However, as implied in the gene name the protein product is similar to another protein, CDK5 regulatory subunit associated protein 1 (CDK5RAP1). CDK5RAP1 is expressed in neuronal tissues where it inhibits cyclin dependent kinase 5 (CDK5) activity by binding to the CDK5 regulatory subunit p35 (Ching, Y. P., et al. J Bio/Chem 277, 15237-40 (2002)). In pancreatic beta cells, CDK5 has been shown to play a role in the loss of beta cell function under glucotoxic conditions (Wei, F. Y. et al. Nat Med 11, 1104-8 (2005)). Furthermore, inhibition of the CDK5/p35 complex prevents decrease of insulin gene expression that results from glucotoxicity (Ubeda, M., et al. J Biol Chem 281, 28858-64 (2006)). It is tempting to speculate that CDKAL1 might play a role in the inhibition of CDK5/p35 in pancreatic beta cells similar to that of CDK5RAP1 in neuronal tissue. Reduced expression of CDKAL1 or reduced inhibitory function thus could lead to an impaired response to glucotoxicity. In this study we showed that CDKAL1 is expressed in the rat pancreatic beta cell line INS-1 (FIG. 6). Further studies are needed to determine if the effect of CDKAL1 on increasing the risk of T2D is exerted through this pathway.

Based on the predicted function of CDKAL1 and known function of SLC30A8 we would expect both rs7756992 and rs13266634 to affect insulin secretion. To evaluate the effects of the two SNPs on insulin secretion we analyzed the effect of genotype status on corrected insulin response (CIR) in a set of individuals from the Inter99 study (part of Denmark B) that had undergone an oral glucose tolerance test (OGTT). For rs7756992, we demonstrated that the homozygote carriers of the risk allele had an estimated 24% less CIR than the heterozygote carriers or non-carriers (P<0.00001, FIG. 7). This observation is consistent with the variant's nearly recessive mode of inheritance with respect to disease risk. Furthermore, the effect observed on CIR is present in both males and females (FIG. 8) and in T2D patients as well as controls, and adjusting for BMI status did not affect the results (Table 21). The effect of rs13266634 on insulin response was smaller but significant and for this risk variant the reduction in CIR was consistent with an additive effect. No effect on insulin sensitivity was observed for either variant (Table 21).

The identification of CDKAL1 as a susceptibility gene for T2D adds a new piece to the puzzle of how genetic factors may predispose to T2D. Although the function of this gene remains to be elucidated we have shown that it is expressed in pancreatic beta cells and that a variant within the gene is correlated with insulin secretion. The similarity to CDK5RAP1 further indicates that CDKAL1 may facilitate insulin production under glucotoxic conditions through interaction with CDK5. In conclusion, we have identified a variant in the CDKAL1 gene that in a nearly recessive manner blunts the insulin response and predisposes to T2D.

TABLE 14
Association to T2D in the Icelandic discovery group.

| | | | | | | All T2D cases (1399) | | |
|---|---|---|---|---|---|---|---|---|
| Chr | Position | Markers | Allele | | Con. frq | Case. frq | OR | P^{b} |
| | | | | Surrogate^{a} (r²) | | | | |
| C01 | 29602516 | rs4949283 rs502545 | TC | rs10798895 G (1) | 0.149 | 0.117 | 0.76 | 0.00016 |
| C01 | 104461151 | rs7553985 | C | — | 0.394 | 0.430 | 1.16 | 0.0023 |
| C01 | 104467009 | rs2166890 | T | — | 0.393 | 0.430 | 1.16 | 0.0018 |
| C01 | 104468502 | rs7552405 | T | — | 0.317 | 0.355 | 1.19 | 0.00078 |
| C01 | 151915609 | rs3738028 | G | — | 0.360 | 0.407 | 1.22 | 0.000046 |
| C02 | 40632580 | rs13414307 rs1990609 | AG | — | 0.517 | 0.571 | 1.24 | 0.0000089 |
| C02 | 40623619 | rs13414307 | A | — | 0.543 | 0.593 | 1.22 | 0.000033 |
| C02 | 55036788 | rs930493 rs10173697 | GT | — | 0.281 | 0.335 | 1.29 | 0.0000017 |
| C02 | 55040844 | rs10173697 | T | — | 0.503 | 0.553 | 1.22 | 0.000040 |
| C03 | 89162181 | rs12486049 | T | — | 0.872 | 0.904 | 1.38 | 0.000035 |
| C03 | 146863467 | rs7630694 | G | — | 0.060 | 0.070 | 1.20 | 0.065 |
| C03 | 196904151 | rs9858622 | A | — | 0.668 | 0.701 | 1.17 | 0.0028 |
| C04 | 140508134 | rs13116075 rs6824182 | AA | rs10033117 C (1) | 0.741 | 0.763 | 1.13 | 0.036 |
| C04 | 140604420 | rs2292837 rs11725721 | TC | — | 0.254 | 0.232 | 0.89 | 0.038 |
| C04 | 140621178 | rs3762864 rs11725721 | GC | — | 0.254 | 0.233 | 0.89 | 0.042 |
| C05 | 76637396 | rs832785 rs2859576 | AA | — | 0.510 | 0.470 | 0.85 | 0.00082 |
| C05 | 76635083 | rs4704400 | T | — | 0.490 | 0.530 | 1.18 | 0.0008 |
| C05 | 87882885 | rs10505855 rs12514611 | GC | rs10452479 G | 0.188 | 0.224 | 1.25 | 0.00023 |
| | | | | (0.94) | | | | |
| C06 | 6967990 | rs490213 rs814174 | AG | rs12201780 A (1) | 0.044 | 0.072 | 1.71 | 0.000016 |
| C06 | 9509965 | rs214447 | T | — | 0.424 | 0.449 | 1.11 | 0.034 |
| C06 | 20779501 | rs4712527 rs7756992 | AG | — | 0.232 | 0.270 | 1.23 | 0.00021 |
| C06 | 20805960 | rs7756992 rs9295478 | AG | — | 0.743 | 0.701 | 0.81 | 0.000089 |
| C06 | 20787688 | rs7756992 | G | — | 0.232 | 0.270 | 1.23 | 0.00021 |
| C06 | 31552682 | rs2516424 | C | — | 0.325 | 0.372 | 1.23 | 0.000039 |
| C06 | 31592562 | rs2516424 rs4947324 | CC | — | 0.320 | 0.368 | 1.24 | 0.000027 |
| C06 | 41130207 | rs10456499 | A | — | 0.563 | 0.597 | 1.15 | 0.0040 |
| C06 | 132387934 | rs9483377 rs997607 | GC | — | 0.234 | 0.278 | 1.26 | 0.000040 |
| C06 | 132379686 | rs9483377 rs7745875 | GG | — | 0.233 | 0.276 | 1.25 | 0.000048 |
| C06 | 132361238 | rs9483377 | G | — | 0.307 | 0.356 | 1.25 | 0.000013 |
| C06 | 150399255 | rs11155700 | A | — | 0.749 | 0.794 | 1.29 | 0.0000095 |
| C06 | 150399954 | rs12213837 | C | — | 0.749 | 0.794 | 1.29 | 0.0000097 |
| C06 | 164421443 | rs206732 rs933251 | TC | rs10085202 A (1) | 0.531 | 0.479 | 0.81 | 0.000037 |
| C08 | 124084183 | rs952656 | G | — | 0.673 | 0.721 | 1.25 | 0.000019 |
| C08 | 124092339 | rs13252935 rs7824293 | TG | — | 0.143 | 0.108 | 0.72 | 0.000010 |
| C08 | 128249239 | rs283710 rs412835 | CC | — | 0.254 | 0.222 | 0.84 | 0.0024 |
| C08 | 128250055 | rs185852 | G | — | 0.755 | 0.791 | 1.22 | 0.00050 |
| C08 | 128265112 | rs283718 rs283720 | CA | — | 0.255 | 0.223 | 0.84 | 0.0026 |
| C09 | 88426790 | rs10993008 | A | — | 0.154 | 0.192 | 1.30 | 0.000027 |
| C09 | 93768899 | rs10818991 rs10990303 | CC | rs10985640 A | 0.537 | 0.490 | 0.83 | 0.00019 |
| | | | | (0.85) | | | | |
| C09 | 93802193 | rs10990568 rs4743148 | GG | — | 0.263 | 0.309 | 1.25 | 0.000032 |
| C09 | 93810412 | rs4743148 | G | — | 0.315 | 0.365 | 1.25 | 0.000010 |
| C09 | 124790974 | rs3814120 | T | — | 0.093 | 0.113 | 1.25 | 0.0046 |
| C10 | 52735263 | rs7915186 rs3829170 | TT | — | 0.328 | 0.377 | 1.24 | 0.000021 |
| C10 | 52746400 | rs3829170 rs7922112 | TG | rs12247188 T (0.9) | 0.336 | 0.386 | 1.24 | 0.000021 |
| C10 | 93976392 | rs2421943 | G | — | 0.555 | 0.614 | 1.28 | 9.1 × 10⁻⁷ |
| C10 | 94022896 | rs2421943 rs7917359 | GC | — | 0.521 | 0.585 | 1.30 | 1.3 × 10⁻⁸ |
| C10 | 94068337 | rs7908111 rs2497304 | GG | — | 0.499 | 0.443 | 0.80 | 0.0000034 |
| C10 | 94011761 | rs1999763 rs10882091 | GT | — | 0.517 | 0.455 | 0.78 | 2.9 × 10⁻⁷ |
| C10 | 94023632 | rs1999763 rs6583830 | GG | — | 0.517 | 0.455 | 0.78 | 2.9 × 10⁻⁷ |
| C10 | 94012407 | rs6583826 | G | — | 0.467 | 0.518 | 1.23 | 0.000020 |
| C10 | 94025680 | rs6583826 rs10882091 | GC | — | 0.393 | 0.449 | 1.26 | 0.0000021 |
| C10 | 94092724 | rs10882091 rs7923837 | CG | — | 0.410 | 0.466 | 1.26 | 0.0000022 |
| C10 | 94038954 | rs10882091 | C | — | 0.415 | 0.472 | 1.26 | 0.0000024 |
| C10 | 94047527 | rs7914814 | T | — | 0.416 | 0.472 | 1.26 | 0.0000025 |
| C10 | 94062695 | rs6583830 | A | — | 0.415 | 0.472 | 1.26 | 0.0000024 |
| C10 | 94122233 | rs2275729 rs1111875 | AG | — | 0.470 | 0.527 | 1.26 | 0.0000023 |
| C10 | 94157293 | rs2497304 | A | — | 0.530 | 0.473 | 0.80 | 0.0000 |
| C10 | 94160330 | rs947591 | A | — | 0.475 | 0.526 | 1.23 | 0.000023 |
| C10 | 114441018 | rs7895307 rs12255372 | GT | — | 0.257 | 0.308 | 1.29 | 0.0000049 |
| C10 | 114422936 | rs7903146 | T | — | 0.300 | 0.372 | 1.38 | 1.9 × 10⁻¹⁰ |
| C10 | 114434905 | rs7903146 rs11196192 | TT | — | 0.220 | 0.282 | 1.39 | 3.4 × 10⁻⁹ |
| C10 | 114438514 | rs7904519 | G | — | 0.480 | 0.522 | 1.18 | 0.00045 |
| C10 | 114455586 | rs7904519 rs10885409 | GC | — | 0.474 | 0.516 | 1.18 | 0.00055 |
| C10 | 114455586 | rs7904519 rs10885409 | AT | — | 0.510 | 0.471 | 0.86 | 0.0013 |
| C10 | 114472659 | rs10885409 | C | — | 0.484 | 0.523 | 1.17 | 0.0014 |
| C10 | 114473489 | rs12255372 | T | — | 0.294 | 0.351 | 1.29 | 4.9 × 10⁻⁷ |
| C10 | 118261345 | rs1681748 rs2170862 | TT | — | 0.238 | 0.265 | 1.15 | 0.013 |
| C10 | 118285583 | rs2170862 | T | — | 0.256 | 0.281 | 1.13 | 0.020 |
| C10 | 118555280 | rs10787760 | G | — | 0.278 | 0.300 | 1.12 | 0.037 |
| C11 | 23946882 | rs1879230 | T | — | 0.088 | 0.111 | 1.30 | 0.00097 |
| C11 | 106474406 | rs1455593 | T | — | 0.097 | 0.114 | 1.20 | 0.021 |
| C12 | 30390375 | rs1429622 rs1506382 | AG | rs794598 C (0.9) | 0.368 | 0.321 | 0.82 | 0.000083 |
| C12 | 33373479 | rs1905421 | T | — | 0.082 | 0.110 | 1.39 | 0.000044 |
| C13 | 25558690 | rs565707 rs6491198 | AA | — | 0.281 | 0.249 | 0.85 | 0.0039 |
| C13 | 25478564 | rs565707 | C | — | 0.700 | 0.734 | 1.19 | 0.0016 |
| C13 | 25535031 | rs7984685 | C | — | 0.540 | 0.582 | 1.19 | 0.00043 |
| C13 | 25537643 | rs7998347 | C | — | 0.540 | 0.582 | 1.19 | 0.00046 |
| C13 | 25715179 | rs1333350 rs7987436 | GT | — | 0.254 | 0.216 | 0.81 | 0.00030 |
| C14 | 80759910 | rs799099 rs4899801 | AG | — | 0.365 | 0.390 | 1.11 | 0.037 |
| C14 | 80763881 | rs2066041 | G | — | 0.367 | 0.394 | 1.12 | 0.021 |
| C14 | 80820260 | rs10483957 | A | — | 0.459 | 0.493 | 1.15 | 0.0042 |
| C15 | 98094991 | rs9920347 rs11635811 | AG | rs2045107 C (0.9) | 0.521 | 0.469 | 0.81 | 0.000044 |
| C16 | 12811478 | rs6498353 rs9941146 | CG | — | 0.105 | 0.080 | 0.74 | 0.00054 |
| C16 | 22764405 | rs724466 | T | — | 0.738 | 0.781 | 1.26 | 0.000038 |
| C16 | 24353768 | rs11074618 rs985729 | AC | rs11644596 G (1) | 0.299 | 0.342 | 1.21 | 0.00044 |
| C16 | 73296557 | rs1862773 rs825842 | CT | — | 0.059 | 0.038 | 0.63 | 0.000048 |
| C16 | 73311680 | rs2432543 rs4887826 | TG | — | 0.069 | 0.043 | 0.61 | 0.000010 |
| C17 | 69180675 | rs17763769 rs1860316 | GA | — | 0.511 | 0.564 | 1.24 | 0.000013 |
| C17 | 69203439 | rs1860316 | A | — | 0.653 | 0.707 | 1.28 | 0.0000020 |
| C17 | 69242752 | rs1860316 rs17763811 | GC | — | 0.335 | 0.282 | 0.78 | 0.0000028 |
| C17 | 69218316 | rs1981647 | C | — | 0.513 | 0.563 | 1.23 | 0.000026 |
| C17 | 69234630 | rs1843622 | T | — | 0.615 | 0.665 | 1.24 | 0.000021 |
| C17 | 69244944 | rs2191113 | A | — | 0.696 | 0.744 | 1.27 | 0.000013 |
| C17 | 69259003 | rs9890889 | A | — | 0.839 | 0.869 | 1.27 | 0.00053 |
| C18 | 41051796 | rs10502860 | G | — | 0.167 | 0.194 | 1.20 | 0.0035 |
| C18 | 63451377 | rs764133 rs7237209 | TT | — | 0.167 | 0.132 | 0.76 | 0.00010 |
| C18 | 63463071 | rs7237209 | C | — | 0.819 | 0.852 | 1.27 | 0.00028 |
| C19 | 3316583 | rs3810420 | A | — | 0.176 | 0.189 | 1.09 | 0.16 |
| C20 | 37651862 | rs4592915 rs2232580 | GC | rs6127771 C (1) | 0.495 | 0.550 | 1.25 | 0.0000048 |
| C21 | 13769165 | rs468601 | A | — | 0.888 | 0.908 | 1.25 | 0.0054 |
| C21 | 33296778 | rs2834061 | G | — | 0.249 | 0.291 | 1.24 | 0.000076 |
| C21 | 39373432 | rs369906 | T | — | 0.566 | 0.613 | 1.21 | 0.00010 |
| | | | | Gene | | | | |
| C03 | 69453958 | rs10510980 | A | ENST00000343145 | 0.808 | 0.840 | 1.25 | 0.00065 |
| | | | | (K211R) | | | | |
| C08 | 118141371 | rs13266634 | C | SLC30A8 (R325W) | 0.646 | 0.685 | 1.19 | 0.00060 |
| C10 | 124472418 | rs2495774 | G | LOC390009 | 0.547 | 0.594 | 1.21 | 0.00011 |
| | | | | (Q27H) | | | | |
| C11 | 3624302 | rs2271586 | T | ART5 (T284K) | 0.176 | 0.208 | 1.23 | 0.00059 |
| C19 | 8669900 | rs10410943 | G | MGC33407 (A51V) | 0.674 | 0.714 | 1.20 | 0.00043 |

| | NonObese T2D cases (700) | | | Obese T2D cases (531) | | |
|---|---|---|---|---|---|---|
| Chr | Case. frq | OR | P^{b} | Case. frq | OR | P^{b} |
| C01 | 0.104 | 0.66 | 0.000033 | 0.133 | 0.88 | 0.21 |
| C01 | 0.419 | 1.11 | 0.11 | 0.466 | 1.34 | 0.000027 |
| C01 | 0.419 | 1.11 | 0.091 | 0.466 | 1.35 | 0.000024 |
| C01 | 0.346 | 1.14 | 0.047 | 0.386 | 1.35 | 0.000030 |
| C01 | 0.417 | 1.27 | 0.00016 | 0.409 | 1.23 | 0.0038 |
| C02 | 0.568 | 1.23 | 0.0011 | 0.582 | 1.30 | 0.00026 |
| C02 | 0.589 | 1.21 | 0.0028 | 0.603 | 1.28 | 0.00056 |
| C02 | 0.325 | 1.23 | 0.0024 | 0.333 | 1.27 | 0.0016 |
| C02 | 0.545 | 1.18 | 0.0086 | 0.560 | 1.25 | 0.0014 |
| C03 | 0.907 | 1.43 | 0.00043 | 0.901 | 1.34 | 0.0095 |
| C03 | 0.056 | 0.93 | 0.60 | 0.097 | 1.70 | 0.000033 |
| C03 | 0.682 | 1.07 | 0.34 | 0.737 | 1.40 | 0.000016 |
| C04 | 0.734 | 0.96 | 0.60 | 0.804 | 1.43 | 0.000024 |
| C04 | 0.259 | 1.03 | 0.69 | 0.194 | 0.71 | 0.000047 |
| C04 | 0.262 | 1.04 | 0.60 | 0.194 | 0.70 | 0.000038 |
| C05 | 0.489 | 0.92 | 0.18 | 0.438 | 0.75 | 0.000043 |
| C05 | 0.511 | 1.09 | 0.18 | 0.562 | 1.33 | 0.000043 |
| C05 | 0.244 | 1.39 | 0.000015 | 0.200 | 1.08 | 0.38 |
| C06 | 0.080 | 1.89 | 0.000037 | 0.063 | 1.48 | 0.033 |
| C06 | 0.416 | 0.97 | 0.61 | 0.495 | 1.34 | 0.000035 |
| C06 | 0.292 | 1.37 | 0.0000090 | 0.250 | 1.11 | 0.21 |
| C06 | 0.682 | 0.74 | 0.000013 | 0.718 | 0.88 | 0.11 |
| C06 | 0.292 | 1.37 | 0.0000090 | 0.250 | 1.11 | 0.20 |
| C06 | 0.375 | 1.25 | 0.00080 | 0.376 | 1.25 | 0.0020 |
| C06 | 0.370 | 1.25 | 0.00074 | 0.373 | 1.26 | 0.0016 |
| C06 | 0.575 | 1.05 | 0.43 | 0.637 | 1.36 | 0.000018 |
| C06 | 0.272 | 1.22 | 0.0067 | 0.276 | 1.25 | 0.0065 |
| C06 | 0.271 | 1.22 | 0.0052 | 0.273 | 1.23 | 0.0087 |
| C06 | 0.348 | 1.20 | 0.0052 | 0.354 | 1.24 | 0.0040 |
| C06 | 0.786 | 1.23 | 0.0049 | 0.801 | 1.35 | 0.00039 |
| C06 | 0.786 | 1.23 | 0.0049 | 0.801 | 1.35 | 0.00040 |
| C06 | 0.469 | 0.78 | 0.00015 | 0.497 | 0.87 | 0.058 |
| C08 | 0.706 | 1.17 | 0.021 | 0.725 | 1.28 | 0.0012 |
| C08 | 0.116 | 0.78 | 0.0099 | 0.104 | 0.69 | 0.00067 |
| C08 | 0.245 | 0.95 | 0.51 | 0.190 | 0.69 | 0.000025 |
| C08 | 0.764 | 1.05 | 0.49 | 0.822 | 1.49 | 0.0000046 |
| C08 | 0.256 | 1.01 | 0.94 | 0.189 | 0.68 | 0.0000092 |
| C09 | 0.181 | 1.21 | 0.019 | 0.194 | 1.32 | 0.0020 |
| C09 | 0.469 | 0.76 | 0.000037 | 0.513 | 0.91 | 0.18 |
| C09 | 0.314 | 1.28 | 0.00038 | 0.306 | 1.23 | 0.0076 |
| C09 | 0.371 | 1.28 | 0.00013 | 0.358 | 1.21 | 0.0092 |
| C09 | 0.094 | 1.01 | 0.91 | 0.140 | 1.59 | 0.000014 |
| C10 | 0.374 | 1.22 | 0.0021 | 0.375 | 1.23 | 0.0049 |
| C10 | 0.381 | 1.22 | 0.0027 | 0.387 | 1.24 | 0.0027 |
| C10 | 0.600 | 1.20 | 0.0043 | 0.621 | 1.31 | 0.00017 |
| C10 | 0.565 | 1.19 | 0.0052 | 0.602 | 1.39 | 0.0000041 |
| C10 | 0.456 | 0.84 | 0.0072 | 0.427 | 0.75 | 0.000039 |
| C10 | 0.472 | 0.83 | 0.0038 | 0.442 | 0.74 | 0.000019 |
| C10 | 0.472 | 0.83 | 0.0038 | 0.442 | 0.74 | 0.000019 |
| C10 | 0.508 | 1.18 | 0.0080 | 0.527 | 1.28 | 0.00048 |
| C10 | 0.435 | 1.19 | 0.0062 | 0.469 | 1.36 | 0.000012 |
| C10 | 0.452 | 1.19 | 0.0063 | 0.486 | 1.36 | 0.000011 |
| C10 | 0.456 | 1.18 | 0.0079 | 0.491 | 1.36 | 0.000014 |
| C10 | 0.456 | 1.18 | 0.0081 | 0.491 | 1.35 | 0.000014 |
| C10 | 0.456 | 1.18 | 0.0079 | 0.491 | 1.36 | 0.000014 |
| C10 | 0.519 | 1.22 | 0.0018 | 0.534 | 1.29 | 0.00025 |
| C10 | 0.481 | 0.82 | 0.00 | 0.466 | 0.77 | 0.000251 |
| C10 | 0.521 | 1.21 | 0.0028 | 0.545 | 1.33 | 0.000053 |
| C10 | 0.330 | 1.42 | 4.5 × 10⁻⁷ | 0.269 | 1.06 | 0.45 |
| C10 | 0.396 | 1.53 | 2.4 × 10⁻¹¹ | 0.342 | 1.21 | 0.010 |
| C10 | 0.298 | 1.51 | 9.4 × 10⁻⁹ | 0.263 | 1.27 | 0.0042 |
| C10 | 0.553 | 1.34 | 0.0000026 | 0.483 | 1.01 | 0.84 |
| C10 | 0.549 | 1.35 | 0.0000018 | 0.476 | 1.01 | 0.90 |
| C10 | 0.441 | 0.76 | 0.000011 | 0.510 | 1.00 | 0.99 |
| C10 | 0.555 | 1.33 | 0.0000060 | 0.483 | 0.99 | 0.94 |
| C10 | 0.371 | 1.41 | 1.6 × 10⁻⁷ | 0.317 | 1.11 | 0.15 |
| C10 | 0.245 | 1.04 | 0.59 | 0.302 | 1.38 | 0.000041 |
| C10 | 0.259 | 1.02 | 0.82 | 0.320 | 1.37 | 0.000043 |
| C10 | 0.269 | 0.96 | 0.53 | 0.347 | 1.38 | 0.000017 |
| C11 | 0.128 | 1.53 | 0.000021 | 0.093 | 1.07 | 0.57 |
| C11 | 0.087 | 0.89 | 0.29 | 0.142 | 1.54 | 0.000040 |
| C12 | 0.341 | 0.89 | 0.092 | 0.296 | 0.72 | 0.000023 |
| C12 | 0.116 | 1.47 | 0.00020 | 0.107 | 1.35 | 0.011 |
| C13 | 0.220 | 0.72 | 0.000016 | 0.274 | 0.97 | 0.69 |
| C13 | 0.763 | 1.38 | 0.0000073 | 0.710 | 1.05 | 0.53 |
| C13 | 0.606 | 1.31 | 0.000022 | 0.568 | 1.12 | 0.11 |
| C13 | 0.606 | 1.31 | 0.000024 | 0.568 | 1.12 | 0.11 |
| C13 | 0.195 | 0.71 | 0.000010 | 0.251 | 0.98 | 0.82 |
| C14 | 0.359 | 0.97 | 0.64 | 0.439 | 1.36 | 0.000022 |
| C14 | 0.368 | 1.01 | 0.92 | 0.437 | 1.34 | 0.000038 |
| C14 | 0.476 | 1.07 | 0.28 | 0.530 | 1.33 | 0.000042 |
| C15 | 0.475 | 0.84 | 0.0056 | 0.468 | 0.81 | 0.0041 |
| C16 | 0.068 | 0.62 | 0.000047 | 0.082 | 0.75 | 0.026 |
| C16 | 0.781 | 1.27 | 0.0012 | 0.783 | 1.29 | 0.0025 |
| C16 | 0.332 | 1.16 | 0.040 | 0.372 | 1.39 | 0.000032 |
| C16 | 0.041 | 0.67 | 0.0075 | 0.039 | 0.64 | 0.0072 |
| C16 | 0.042 | 0.60 | 0.00046 | 0.049 | 0.69 | 0.019 |
| C17 | 0.585 | 1.35 | 0.0000023 | 0.543 | 1.14 | 0.069 |
| C17 | 0.734 | 1.46 | 3.2 × 10⁻⁸ | 0.687 | 1.17 | 0.039 |
| C17 | 0.254 | 0.68 | 2.6 × 10⁻⁸ | 0.301 | 0.86 | 0.039 |
| C17 | 0.583 | 1.33 | 0.0000065 | 0.544 | 1.14 | 0.071 |
| C17 | 0.684 | 1.35 | 0.0000043 | 0.640 | 1.11 | 0.14 |
| C17 | 0.771 | 1.47 | 9.5 × 10⁻⁸ | 0.713 | 1.08 | 0.30 |
| C17 | 0.885 | 1.47 | 0.000032 | 0.857 | 1.14 | 0.17 |
| C18 | 0.218 | 1.39 | 0.000028 | 0.174 | 1.05 | 0.61 |
| C18 | 0.121 | 0.69 | 0.000048 | 0.135 | 0.78 | 0.014 |
| C18 | 0.867 | 1.44 | 0.000029 | 0.847 | 1.22 | 0.037 |
| C19 | 0.227 | 1.37 | 0.000045 | 0.146 | 0.80 | 0.021 |
| C20 | 0.558 | 1.29 | 0.000051 | 0.543 | 1.21 | 0.0060 |
| C21 | 0.927 | 1.60 | 0.000026 | 0.895 | 1.08 | 0.48 |
| C21 | 0.311 | 1.36 | 0.0000094 | 0.271 | 1.12 | 0.15 |
| C21 | 0.631 | 1.31 | 0.000028 | 0.587 | 1.09 | 0.24 |
| C03 | 0.836 | 1.22 | 0.019 | 0.845 | 1.30 | 0.0061 |
| C08 | 0.678 | 1.16 | 0.030 | 0.697 | 1.26 | 0.0020 |
| C10 | 0.592 | 1.20 | 0.0039 | 0.597 | 1.22 | 0.0043 |
| C11 | 0.212 | 1.26 | 0.0033 | 0.203 | 1.20 | 0.042 |
| C19 | 0.713 | 1.20 | 0.0076 | 0.708 | 1.17 | 0.035 |

The upper table includes association results for all SNPs or two-marker haplotypes that have an adjusted P value less than 10⁻⁵ for either all T2D cases, non-obese T2D cases or obese T2D cases. Included in the table is the chromosome, the position of the markers (or the midpoint for two-marker haplotypes) in NCBI Build 34, the markers and alleles tested, the corresponding surrogate SNP for two-markers haplotypes selected for replication, the frequency in controls and the frequency in cases, the odds ratio (OR) and adjusted P-value for the three case groups tested. The number of T2D cases in each of the three groups is included in parenthesis and the same set of 5275 controls is used in all tests. Note that information on BMI is missing for 168 of the cases. The lower table includes the corresponding values for the five most significant non-synonymous SNPs selected for replication. Included in column five are the corresponding genes and the codon changes. In both tables markers selected for further testing in the first replication group (Denmark B) are indicated with bold typesetting. Other markers/haplotypes were excluded from the replication study as they were a) highly correlated with another marker selected for replication, or b) belong to the TCF7L2 locus that has been studied previously.
^{a}A surrogate of the corresponding two marker haplotype with a correlation coefficient r².
^{b}P values adjusted for relatedness and population stratification using genomic control (see Methods).

TABLE 15
Association to T2D in the primary replication group (Denmark B).

| | | | | | | | | NonObese | | | | | |
|---|---|---|---|---|---|---|---|---|---|---|---|---|---|
| | | | | Con. | All T2D cases (1110) | | | T2D cases (640) | | | Obese T2D cases (470) | | |
| Chr | Position | Marker | Allele | frq | Case. frq | OR | P | Case. frq | OR | P | Case. frq | OR | P |
| C01 | 29589307 | rs10798895 | A | 0.832 | 0.828 | 0.97 | 0.68 | 0.831 | 0.99 | 0.94 | 0.824 | 0.94 | 0.55 |
| C01 | 104461151 | rs7553985 | C | 0.367 | 0.379 | 1.05 | 0.34 | 0.375 | 1.03 | 0.62 | 0.385 | 1.08 | 0.30 |
| C01 | 151915609 | rs3738028 | G | 0.385 | 0.410 | 1.11 | 0.050 | 0.419 | 1.15 | 0.029 | 0.397 | 1.05 | 0.47 |
| C02 | 40623619 | rs13414307 | A | 0.537 | 0.540 | 1.01 | 0.84 | 0.544 | 1.03 | 0.67 | 0.534 | 0.99 | 0.86 |
| C03 | 69453958 | rs10510980 | A | 0.826 | 0.833 | 1.05 | 0.50 | 0.835 | 1.06 | 0.50 | 0.831 | 1.03 | 0.74 |
| C03 | 89162181 | rs12486049 | T | 0.878 | 0.872 | 0.94 | 0.47 | 0.871 | 0.93 | 0.49 | 0.873 | 0.96 | 0.70 |
| C03 | 146863467 | rs7630694 | G | 0.053 | 0.054 | 1.02 | 0.85 | 0.051 | 0.95 | 0.72 | 0.059 | 1.12 | 0.46 |
| C03 | 196904151 | rs9858622 | A | 0.656 | 0.667 | 1.05 | 0.39 | 0.662 | 1.02 | 0.73 | 0.674 | 1.08 | 0.29 |
| C04 | 140660180 | rs10033117 | C | 0.740 | 0.746 | 1.03 | 0.65 | 0.747 | 1.04 | 0.65 | 0.744 | 1.02 | 0.81 |
| C05 | 76635083 | rs4704400 | T | 0.472 | 0.456 | 0.94 | 0.23 | 0.452 | 0.92 | 0.22 | 0.461 | 0.96 | 0.55 |
| C05 | 87825021 | rs10452479 | G | 0.229 | 0.238 | 1.05 | 0.43 | 0.240 | 1.06 | 0.43 | 0.235 | 1.04 | 0.68 |
| C06 | 6971276 | rs12201780 | A | 0.043 | 0.048 | 1.12 | 0.36 | 0.049 | 1.16 | 0.32 | 0.045 | 1.07 | 0.71 |
| C06 | 9509965 | rs214447 | T | 0.418 | 0.427 | 1.03 | 0.52 | 0.432 | 1.06 | 0.39 | 0.419 | 1.00 | 0.95 |
| C06 | 20787688 | rs7756992 | G | 0.276 | 0.322 | 1.24 | 0.00013 | 0.321 | 1.24 | 0.0021 | 0.323 | 1.25 | 0.0044 |
| C06 | 31552682 | rs2516424 | C | 0.363 | 0.380 | 1.07 | 0.19 | 0.374 | 1.05 | 0.48 | 0.387 | 1.11 | 0.18 |
| C06 | 41130207 | rs10456499 | A | 0.581 | 0.579 | 0.99 | 0.92 | 0.576 | 0.98 | 0.78 | 0.583 | 1.01 | 0.87 |
| C06 | 132361238 | rs9483377 | G | 0.306 | 0.331 | 1.12 | 0.039 | 0.334 | 1.14 | 0.061 | 0.327 | 1.10 | 0.20 |
| C06 | 150399255 | rs11155700 | A | 0.758 | 0.734 | 0.88 | 0.043 | 0.737 | 0.90 | 0.14 | 0.731 | 0.87 | 0.089 |
| C06 | 164425224 | rs10085202 | G | 0.430 | 0.426 | 0.99 | 0.78 | 0.424 | 0.98 | 0.73 | 0.428 | 0.99 | 0.94 |
| C08 | 118141371 | rs13266634 | C | 0.664 | 0.704 | 1.20 | 0.0012 | 0.701 | 1.19 | 0.013 | 0.707 | 1.22 | 0.012 |
| C08 | 124084183 | rs952656 | G | 0.672 | 0.672 | 1.00 | 0.98 | 0.680 | 1.04 | 0.56 | 0.660 | 0.95 | 0.51 |
| C08 | 128250055 | rs185852 | G | 0.796 | 0.797 | 1.01 | 0.92 | 0.794 | 0.99 | 0.88 | 0.801 | 1.03 | 0.72 |
| C09 | 88426790 | rs10993008 | A | 0.146 | 0.150 | 1.03 | 0.66 | 0.151 | 1.04 | 0.64 | 0.149 | 1.02 | 0.84 |
| C09 | 93745181 | rs10985640 | G | 0.430 | 0.434 | 1.01 | 0.78 | 0.421 | 0.96 | 0.57 | 0.451 | 1.09 | 0.25 |
| C09 | 93810412 | rs4743148 | G | 0.382 | 0.381 | 1.00 | 0.94 | 0.370 | 0.95 | 0.41 | 0.398 | 1.07 | 0.39 |
| C09 | 124790974 | rs3814120 | T | 0.089 | 0.090 | 1.02 | 0.84 | 0.076 | 0.85 | 0.16 | 0.109 | 1.27 | 0.052 |
| C10 | 52758344 | rs12247188 | T | 0.331 | 0.315 | 0.93 | 0.19 | 0.312 | 0.92 | 0.22 | 0.318 | 0.94 | 0.45 |
| C10 | 94047527 | rs7914814 | T | 0.413 | 0.432 | 1.08 | 0.14 | 0.434 | 1.09 | 0.18 | 0.429 | 1.07 | 0.35 |
| C10 | 118555280 | rs10787760 | G | 0.294 | 0.276 | 0.91 | 0.15 | 0.268 | 0.88 | 0.080 | 0.288 | 0.97 | 0.73 |
| C10 | 124472418 | rs2495774 | G | 0.524 | 0.540 | 1.07 | 0.22 | 0.542 | 1.07 | 0.27 | 0.538 | 1.06 | 0.46 |
| C11 | 23946882 | rs1879230 | T | 0.127 | 0.115 | 0.89 | 0.13 | 0.118 | 0.91 | 0.36 | 0.110 | 0.85 | 0.14 |
| C11 | 3624302 | rs2271586 | T | 0.190 | 0.201 | 1.07 | 0.28 | 0.194 | 1.02 | 0.77 | 0.211 | 1.14 | 0.13 |
| C11 | 106474406 | rs1455593 | T | 0.081 | 0.080 | 0.98 | 0.81 | 0.081 | 0.99 | 0.92 | 0.078 | 0.96 | 0.77 |
| C12 | 30434349 | rs794598 | T | 0.623 | 0.600 | 0.91 | 0.063 | 0.594 | 0.88 | 0.058 | 0.608 | 0.94 | 0.37 |
| C12 | 33373479 | rs1905421 | T | 0.099 | 0.097 | 0.98 | 0.79 | 0.086 | 0.85 | 0.17 | 0.113 | 1.16 | 0.24 |
| C14 | 80763881 | rs2066041 | G | 0.427 | 0.415 | 0.95 | 0.35 | 0.427 | 1.00 | 1.00 | 0.398 | 0.89 | 0.11 |
| C15 | 98060278 | rs2045107 | G | 0.524 | 0.527 | 1.01 | 0.78 | 0.522 | 0.99 | 0.92 | 0.534 | 1.04 | 0.55 |
| C16 | 12756032 | rs6498353 | C | 0.136 | 0.134 | 0.98 | 0.80 | 0.140 | 1.04 | 0.68 | 0.124 | 0.90 | 0.35 |
| C16 | 22764405 | rs724466 | T | 0.695 | 0.715 | 1.10 | 0.085 | 0.719 | 1.12 | 0.10 | 0.710 | 1.08 | 0.34 |
| C16 | 24356412 | rs11644596 | G | 0.324 | 0.323 | 1.00 | 0.94 | 0.336 | 1.06 | 0.43 | 0.305 | 0.92 | 0.27 |
| C16 | 73314817 | rs4887826 | G | 0.064 | 0.052 | 0.82 | 0.068 | 0.054 | 0.84 | 0.21 | 0.050 | 0.78 | 0.11 |
| C17 | 69203439 | rs1860316 | A | 0.679 | 0.682 | 1.01 | 0.82 | 0.684 | 1.02 | 0.74 | 0.679 | 1.00 | 1.00 |
| C18 | 41051796 | rs10502860 | G | 0.222 | 0.197 | 0.86 | 0.044 | 0.198 | 0.87 | 0.12 | 0.196 | 0.86 | 0.13 |
| C18 | 63463071 | rs7237209 | C | 0.861 | 0.852 | 0.92 | 0.29 | 0.848 | 0.89 | 0.22 | 0.857 | 0.97 | 0.74 |
| C19 | 3316583 | rs3810420 | A | 0.181 | 0.191 | 1.07 | 0.30 | 0.188 | 1.05 | 0.54 | 0.195 | 1.10 | 0.30 |
| C20 | 37645161 | rs6127771 | C | 0.447 | 0.451 | 1.02 | 0.77 | 0.442 | 0.98 | 0.77 | 0.462 | 1.06 | 0.39 |
| C21 | 33296778 | rs2834061 | G | 0.250 | 0.255 | 1.03 | 0.66 | 0.267 | 1.09 | 0.23 | 0.239 | 0.94 | 0.48 |

Association results for the 47 SNPs tested in the primary replication cohort (Denmark B), consisting of 1110 T2D cases and 2272 controls. Included in the table is the chromosome, the position of the SNPs in NCBI Build 34, the marker and allele tested, frequency in controls and the frequency in cases, odds ratio (OR) and P value in all T2D cases, non-obese T2D cases and obese T2D cases, respectively. For all three groups of cases, the same group of controls is used and the number of cases is included in the parentheses. The two SNPs selected for replication in additional T2D case-control groups are highlighted with bold typesetting.

TABLE 16
Association results for SNPs with reported association to T2D in Sladek et al.

| | | | | Icelandic study group | | | | Sladek et al | | | | |
|---|---|---|---|---|---|---|---|---|---|---|---|---|
| Chr | Position | Marker | Allele | Controls | Cases | OR | P | Controls | Cases | OR^{a} | P^{b} | Nearest gene |
| C08 | 118141371 | rs13266634 | C | 0.646 | 0.685 | 1.19 | 0.00060 | 0.699 | 0.746 | 1.26 | 5.0 × 10⁻⁷ | SLC30A8 |
| C10 | 94127459 | rs1111875 | G | 0.550 | 0.588 | 1.17 | 0.0014 | 0.598 | 0.642 | 1.21 | 9.1 × 10⁻⁶ | HHEX |
| C10 | 94146494 | rs7923837 | G | 0.583 | 0.624 | 1.19 | 0.00058 | 0.623 | 0.665 | 1.20 | 2.2 × 10⁻⁵ | HHEX |
| C10 | 114422936 | rs7903146 | T | 0.300 | 0.372 | 1.38 | 1.9 × | 0.293 | 0.406 | 1.65 | <1.0 × 10⁻⁷ | TCF7L2 |
| | | | | | | | 10⁻¹⁰ | | | | | |
| C11 | 42211027 | rs7480010 | G | 0.273 | 0.271 | 0.95 | 0.33 | 0.301 | 0.336 | 1.18 | 2.9 × 10⁻⁴ | LOC387761 |
| C11 | 44207712 | rs1113132 | C | — | — | — | — | 0.733 | 0.763 | 1.17 | 8.1 × 10⁻⁴ | EXT2 |
| C11 | 44219923 | rs11037909 | T | — | — | — | — | 0.729 | 0.760 | 1.18 | 4.5 × 10⁻⁴ | EXT2 |
| C11 | 44222111 | rs3740878 | A | — | — | — | — | 0.728 | 0.760 | 1.18 | 2.8 × 10⁻⁴ | EXT2 |
| C11 | 44244399 | rs729287 | C | 0.748 | 0.759 | 1.06 | 0.33 | — | — | — | — | EXT2 |

Shown are association results for T2D in the Icelandic study group for the eight SNPs identified by Sladek et al (Nature 445, 881-5 (2007)) to associate with T2D. For the Icelandic group the table includes the frequency in cases and controls, odds ratio (OR) and adjusted P value for five of the eight SNP's. Corresponding values are shown for the replication cohort used in Sladek et al. Three of the markers, rs1113132, rs11037909 and rs3740878, are not on the Illumina 300K chip; however, a surrogate SNP rs729287 which has a correlation r² = 1 to rs11037909 and rs3740878 (based on HapMap CEU data) has been typed in the Icelandic study group and results for this marker are included in the table.
^{a}Allelic OR calculated from frequency information provided in Table 1 of Sladek et al.
^{b}P value (based on permutation) for Stage 2 in Table 1 in Sladek et al.

TABLE 17
Association results for the SNPs rs7756992 and rs13266634
in six Caucasian T2D case-control groups and in case-control
groups from Hong Kong and from West-Africa.

| Study population (n/m) | Frequency | | | |
|---|---|---|---|---|
| Variant (allele) | Controls | Cases | OR (95% CI) | P value |
| Iceland (1399/5275) | | | | |
| rs7756992 (G) | 0.232 | 0.270 | 1.23 (1.10-1.37) | 0.00021 |
| rs13266634 (C) | 0.646 | 0.685 | 1.19 (1.08-1.31) | 0.0006 |
| Denmark A (263/597) | | | | |
| rs7756992 (G) | 0.297 | 0.331 | 1.17 (0.93-1.47) | 0.18 |
| rs13266634 (C) | 0.686 | 0.672 | 0.94 (0.75-1.17) | 0.58 |
| Denmark B (1359/4825) | | | | |
| rs7756992 (G) | 0.279 | 0.320 | 1.21 (1.10-1.33) | 0.000054 |
| rs13266634 (C) | 0.673 | 0.692 | 1.09 (0.99-1.19) | 0.073 |
| Philadelphia (447/950) | | | | |
| rs7756992 (G) | 0.262 | 0.295 | 1.18 (0.98-1.42) | 0.073 |
| rs13266634 (C) | 0.678 | 0.760 | 1.51 (1.25-1.81) | 1.5 × 10⁻⁵ |
| Scotland (3742/3718) | | | | |
| rs7756992 (G) | 0.267 | 0.288 | 1.11 (1.03-1.19) | 0.0042 |
| rs13266634 (C) | 0.682 | 0.710 | 1.14 (1.06-1.22) | 0.00025 |
| The Netherlands (368/915) | | | | |
| rs7756992 (G) | 0.270 | 0.280 | 1.05 (0.86-1.27) | 0.64 |
| rs13266634 (C) | 0.717 | 0.736 | 1.10 (0.91-1.33) | 0.33 |
| Caucasian combined^{a} (7578/16280) | | | | |
| rs7756992 (G) | 0.264 | 0.293 | 1.16 (1.09-1.22) | 3.9 × 10⁻¹⁰ |
| rs13266634 (C) | 0.675 | 0.700 | 1.15 (1.10-1.20) | 3.3 × 10⁻⁹ |
| Hong Kong(1457/986) | | | | |
| rs7756992 (G) | 0.462 | 0.517 | 1.25 (1.11-1.40) | 0.00018 |
| rs13266634 (C) | 0.523 | 0.566 | 1.19 (1.06-1.33) | 0.0035 |
| West Africa^{a} (865/1106) | | | | |
| rs7756992 (G) | 0.612 | 0.625 | 1.02 (0.92-1.14) | 0.72 |
| rs13266634 (C) | 0.962 | 0.971 | 1.26 (0.88-1.81) | 0.21 |
| All groups combined (9900/18372) | | | | |
| rs7756992 (G) | | | 1.15 (1.11-1.20) | 9..0 × 10⁻¹² |
| rs13266634 (C) | | | 1.16 (1.11-1.21) | 2.5 × 10⁻¹¹ |

Shown are the number of T2D cases and controls (n/m), the allelic frequency in the affected and control individuals, the allelic odds-ratio (OR) with 95 confidence intervals (CI 95%) and two-sided P values based on the multiplicative model.
^{a}When combining results for the Caucasian groups and for the five West-African groups, OR's and P values are combined using a Mantel-Haenzsel model, while the frequency in cases and controls is estimated as a weighted average over the different study groups.

TABLE 18
Association of eight SNP's in CDKAL1 to T2D in Iceland, Hong Kong and West-Africa.

| | | | Combined^{a} | | Iceland | | | |
|---|---|---|---|---|---|---|---|---|
| SNP | Allele | Position^{b} | OR (95% CI) | P | Con. frq | Case. frq | OR | P |
| rs7752906 | A | 20774034 | 1.19 (1.11-1.28) | 6.5 × 10⁻⁷ | 0.296 | 0.338 | 1.22 | 0.00076 |
| rs1569699 | C | 20787289 | 1.19 (1.12-1.27) | 1.4 × 10⁻⁷ | 0.257 | 0.297 | 1.22 | 0.00018 |
| rs7756992 | G | 20787688 | 1.17 (1.09-1.25) | 3.1 × 10⁻⁶ | 0.232 | 0.270 | 1.23 | 0.00023 |
| rs9350271 | A | 20791143 | 1.18 (1.11-1.26) | 9.6 × 10⁻⁷ | 0.257 | 0.298 | 1.23 | 0.00016 |
| rs9356744 | C | 20793465 | 1.18 (1.11-1.26) | 7.9 × 10⁻⁷ | 0.256 | 0.297 | 1.23 | 0.00014 |
| rs9368222 | A | 20794975 | 1.20 (1.12-1.28) | 4.8 × 10⁻⁷ | 0.231 | 0.269 | 1.22 | 0.00029 |
| rs10440833 | A | 20796100 | 1.18 (1.11-1.27) | 1.4 × 10⁻⁶ | 0.233 | 0.269 | 1.22 | 0.00046 |
| rs6931514 | G | 20811931 | 1.19 (1.11-1.27) | 7.8 × 10⁻⁷ | 0.231 | 0.267 | 1.22 | 0.00047 |

| | Hong Kong | | | | West-Africa^{c} | | | |
|---|---|---|---|---|---|---|---|---|
| SNP | Con. frq | Case. frq | OR | P | Con. frq | Case. frq | OR | P |
| rs7752906 | 0.362 | 0.422 | 1.29 | 3.2 × 10⁻⁵ | 0.654 | 0.674 | 1.06 | 0.43 |
| rs1569699 | 0.463 | 0.519 | 1.25 | 0.00019 | 0.627 | 0.656 | 1.10 | 0.17 |
| rs7756992 | 0.462 | 0.517 | 1.25 | 0.00018 | 0.612 | 0.625 | 1.02 | 0.72 |
| rs9350271 | 0.356 | 0.406 | 1.23 | 0.00055 | 0.695 | 0.712 | 1.07 | 0.38 |
| rs9356744 | 0.357 | 0.407 | 1.24 | 0.00045 | 0.696 | 0.713 | 1.06 | 0.39 |
| rs9368222 | 0.355 | 0.405 | 1.24 | 0.00041 | 0.184 | 0.203 | 1.10 | 0.27 |
| rs10440833 | 0.354 | 0.407 | 1.25 | 0.00024 | 0.213 | 0.226 | 1.06 | 0.48 |
| rs6931514 | 0.464 | 0.520 | 1.25 | 0.00015 | 0.231 | 0.249 | 1.07 | 0.41 |

Association to T2D for eight SNP's in the CDKAL1 gene for three of the eight study groups; from Iceland, Hong Kong and West-Africa. The seven additional SNP's are all highly correlated to rs7756992.
^{a}Results for the three groups were combined using a Mantel-Haenszel model.
^{b}Basepair position in NCBI Build 34.
^{c}Results for the five West-African groups were combined using Mantel-Haenszel model and the allele frequencies shown are a weighted average of the frequency for the five groups.

TABLE 19
Pair-wise correlation for SNP's typed in CDKAL1.

| | r² | | | | | | | |
|---|---|---|---|---|---|---|---|---|
| D′ | rs7752906 | rs1569699 | rs7756992 | rs9350271 | rs9356744 | rs9368222 | rs10440833 | rs6931514 |

Iceland

| | | | | | | | | |
|---|---|---|---|---|---|---|---|---|
| rs7752906 | — | 0.55 | 0.66 | 0.56 | 0.56 | 0.67 | 0.66 | 0.65 |
| rs1569699 | 0.83 | — | 0.87 | 0.99 | 0.98 | 0.85 | 0.83 | 0.83 |
| rs7756992 | 0.98 | 1.00 | — | 0.86 | 0.86 | 0.99 | 0.97 | 0.96 |
| rs9350271 | 0.84 | 1.00 | 1.00 | — | 1.00 | 0.86 | 0.85 | 0.84 |
| rs9356744 | 0.84 | 1.00 | 1.00 | 1.00 | — | 0.87 | 0.86 | 0.85 |
| rs9368222 | 0.99 | 1.00 | 1.00 | 1.00 | 1.00 | — | 0.98 | 0.97 |
| rs10440833 | 0.96 | 0.97 | 1.00 | 0.98 | 0.99 | 1.00 | — | 0.99 |
| rs6931514 | 0.96 | 0.97 | 0.99 | 0.98 | 0.99 | 0.99 | 1.00 | — |

Hong Kong

| | | | | | | | | |
|---|---|---|---|---|---|---|---|---|
| rs7752906 | — | 0.45 | 0.46 | 0.77 | 0.76 | 0.77 | 0.77 | 0.46 |
| rs1569699 | 0.84 | — | 0.99 | 0.63 | 0.63 | 0.62 | 0.62 | 0.98 |
| rs7756992 | 0.84 | 1.00 | — | 0.63 | 0.62 | 0.64 | 0.64 | 0.99 |
| rs9350271 | 0.89 | 1.00 | 0.99 | — | 1.00 | 0.99 | 0.99 | 0.62 |
| rs9356744 | 0.88 | 0.99 | 0.99 | 1.00 | — | 0.99 | 0.99 | 0.62 |
| rs9368222 | 0.89 | 0.99 | 1.00 | 1.00 | 1.00 | — | 1.00 | 0.63 |
| rs10440833 | 0.89 | 1.00 | 1.00 | 1.00 | 1.00 | 1.00 | — | 0.63 |
| rs6931514 | 0.84 | 0.99 | 1.00 | 0.99 | 0.99 | 1.00 | 1.00 | — |

West-Africa

| | | | | | | | | |
|---|---|---|---|---|---|---|---|---|
| rs7752906 | — | 0.16 | 0.32 | 0.13 | 0.14 | 0.12 | 0.07 | 0.08 |
| rs1569699 | 0.42 | — | 0.61 | 0.72 | 0.72 | 0.12 | 0.07 | 0.09 |
| rs7756992 | 0.62 | 0.84 | — | 0.67 | 0.67 | 0.14 | 0.08 | 0.10 |
| rs9350271 | 0.40 | 0.96 | 0.99 | — | 0.99 | 0.10 | 0.04 | 0.05 |
| rs9356744 | 0.41 | 0.96 | 1.00 | 1.00 | — | 0.10 | 0.04 | 0.06 |
| rs9368222 | 1.00 | 0.96 | 0.95 | 1.00 | 1.00 | — | 0.86 | 0.76 |
| rs10440833 | 0.68 | 0.68 | 0.68 | 0.59 | 0.60 | 1.00 | — | 0.87 |
| rs6931514 | 0.73 | 0.72 | 0.73 | 0.63 | 0.65 | 0.99 | 1.00 | — |

Pair-wise correlation, D′ (lower left corner) and r² (upper right corner), for the eight SNP's in CDKAL1 that were tested for association to T2D. The correlation is estimated for control individuals from the Icelandic, Hong Kong and West-African study groups, respectively.

TABLE 20
Genotype specific odds ratio for rs7756992 and rs13266634.

| Study population | Allelic | Genotype odds ratio^{a} | | | |
|---|---|---|---|---|---|
| Variant (allele) | OR (95% CI) | 00 | 0X (95% CI) | XX (95% CI) | P^{b} |
| Caucasian | | | | | |
| rs7756992 (G) | 1.16 (1.09-1.22) | 1 | 1.09 (1.03-1.16) | 1.45 (1.31-1.61) | 0.00052 |
| rs13266634 (C) | 1.15 (1.11-1.20) | 1 | 1.12 (1.03-1.23) | 1.30 (1.18-1.43) | 0.63 |
| Hong Kong | | | | | |
| rs7756992 (G) | 1.25 (1.11-1.40) | 1 | 1.13 (0.97-1.31) | 1.55 (1.23-1.95) | 0.071 |
| rs13266634 (C) | 1.19 (1.06-1.33) | 1 | 1.13 (0.96-1.34) | 1.40 (1.11-1.76) | 0.43 |

^{a}Genotype odds ratio for heterozygous (0X) and homozygous carrier (XX) compared with non-carriers (00).
^{b}Test of the multiplicative model (the null hypotheses) versus the full model, one degree of freedom.

TABLE 21
Association to insulin secretion and insulin sensitivity.

| | | Analysis | | | | | | |
|---|---|---|---|---|---|---|---|---|
| | | Combined group | | | Controls | | T2D | |
| Trait | Group (n/m) | Effect (se) | P | P^{a} | Effect (se) | P | Effect (se) | P |
| Insulin | rs7756992 (add) | | | | | | | |
| Response | All (3715/223) | −0.083 (0.018) | 4.0E−06 | 9.1E−06 | −0.080 (0.018) | 1.3E−05 | −0.142 (0.095) | 0.14 |
| (CIR) | Males (1742/139) | −0.056 (0.025) | 0.025 | 0.042 | −0.058 (0.025) | 0.021 | −0.028 (0.119) | 0.82 |
| | Females (1973/84) | −0.100 (0.025) | 6.8E−05 | 0.00012 | −0.088 (0.025) | 0.00049 | −0.342 (0.144) | 0.02 |
| | rs7756992 (rec) | | | | | | | |
| | All (3715/223) | −0.243 (0.041) | 3.3E−09 | 4.9E−09 | −0.230 (0.042) | 3.7E−08 | −0.417 (0.199) | 0.037 |
| | Males (1742/139) | −0.225 (0.055) | 4.9E−05 | 0.00014 | −0.222 (0.056) | 7.5E−05 | −0.250 (0.250) | 0.32 |
| | Females (1973/84) | −0.232 (0.059) | 7.5E−05 | 7.6E−05 | −0.204 (0.060) | 0.00063 | −0.696 (0.301) | 0.023 |
| | rs13266634 (add) | | | | | | | |
| | All (3698/228) | −0.061 (0.017) | 0.0005 | 0.00056 | −0.059 (0.018) | 0.00075 | −0.083 (0.094) | 0.38 |
| | Males (1736/143) | −0.079 (0.024) | 0.0011 | 0.00091 | −0.062 (0.024) | 0.011 | −0.262 (0.109) | 0.017 |
| | Females (1962/85) | −0.048 (0.024) | 0.047 | 0.052 | −0.058 (0.024) | 0.016 | 0.233 (0.166) | 0.16 |
| HOMA | rs7756992 (add) | | | | | | | |
| | All (4430/1164) | −0.013 (0.013) | 0.33 | 0.7 | 0.002 (0.013) | 0.85 | −0.065 (0.038) | 0.082 |
| | Males (2062/691) | −0.002 (0.019) | 0.94 | 0.51 | 0.022 (0.020) | 0.26 | −0.070 (0.049) | 0.15 |
| | Females (2368/473) | −0.026 (0.018) | 0.14 | 0.22 | −0.018 (0.018) | 0.31 | −0.061 (0.059) | 0.3 |
| | rs13266634 (add) | | | | | | | |
| | All (4411/1166) | −0.015 (0.013) | 0.24 | 0.19 | −0.013 (0.013) | 0.31 | −0.024 (0.039) | 0.55 |
| | Males (2058/697) | −0.003 (0.019) | 0.88 | 0.81 | −0.010 (0.019) | 0.61 | 0.019 (0.050) | 0.7 |
| | Females (2353/469) | −0.028 (0.017) | 0.11 | 0.087 | −0.016 (0.017) | 0.34 | −0.092 (0.063) | 0.14 |

Association of the risk variants rs7756992 (G) and rs13266634 (C) to insulin secretion, estimated by corrected insulin response (CIR), and insulin sensitivity estimated the reciprocal of HOMA (homeostasis model assessment). The table includes number of T2D cases (n) and controls (m) used, the estimated effect and standard error and the P value obtained by regressing the log-transformed trait values on age, sex and either the number of risk alleles an individual carries (additive model) or an indicator variable for homozygous carriers of the risk allele (recessive model). When controls and T2D cases are analysed together an indicator variable for the affection status is included in the analysis. Also shown, for the combined group, is the corresponding P value obtained by adjusting for BMI status of the individuals in the analysis.
^{a}P value after adjusting for BMI by including a log(BMI) term among the explanatory variables.

TABLE 22
Surrogate markers for marker rs7756992 on chromosome 6.
Surrogates for rs7756992 on chromosome 6

| | | | | Pos SEQ ID |
|---|---|---|---|---|
| SNP | D′ | R2 | Pos B36 | NO: 1 |
| rs9460517 | 0.82 | 0.30 | 20636813 | 1818 |
| rs7772956 | 0.72 | 0.29 | 20637521 | 2526 |
| rs6904566 | 0.73 | 0.32 | 20643949 | 8954 |
| rs6927356 | 0.73 | 0.32 | 20644073 | 9078 |
| rs6905138 | 0.73 | 0.32 | 20644335 | 9340 |
| rs13194858 | 0.73 | 0.32 | 20644499 | 9504 |
| rs6456356 | 1.00 | 0.22 | 20649498 | 14503 |
| rs9366354 | 0.84 | 0.40 | 20653447 | 18452 |
| rs9368201 | 0.84 | 0.41 | 20654091 | 19096 |
| rs9348433 | 0.84 | 0.40 | 20657780 | 22785 |
| rs13203450 | 0.73 | 0.32 | 20673935 | 38940 |
| rs1012626 | 0.82 | 0.39 | 20685540 | 50545 |
| rs9460523 | 0.55 | 0.23 | 20690122 | 55127 |
| rs9350262 | 0.55 | 0.23 | 20692402 | 57407 |
| rs4712507 | 0.56 | 0.24 | 20693119 | 58124 |
| rs9366357 | 0.56 | 0.23 | 20707607 | 72612 |
| rs1997777 | 1.00 | 0.22 | 20710359 | 75364 |
| rs11964057 | 0.56 | 0.23 | 20710776 | 75781 |
| rs12206413 | 1.00 | 0.22 | 20715663 | 80668 |
| rs4515379 | 0.66 | 0.20 | 20735420 | 100425 |
| rs9465841 | 0.66 | 0.20 | 20737687 | 102692 |
| rs13190734 | 0.62 | 0.31 | 20738376 | 103381 |
| rs2328528 | 0.67 | 0.21 | 20739524 | 104529 |
| rs2328529 | 0.67 | 0.21 | 20739932 | 104937 |
| rs7768642 | 0.67 | 0.21 | 20741886 | 106891 |
| rs9465846 | 0.67 | 0.21 | 20742320 | 107325 |
| rs9465847 | 0.67 | 0.21 | 20742407 | 107412 |
| rs7755830 | 1.00 | 0.32 | 20742865 | 107870 |
| rs6940200 | 0.67 | 0.21 | 20743241 | 108246 |
| rs9465850 | 0.67 | 0.22 | 20747388 | 112393 |
| rs4710938 | 1.00 | 0.34 | 20748883 | 113888 |
| rs9348440 | 0.79 | 0.23 | 20749315 | 114320 |
| rs4235999 | 1.00 | 0.33 | 20751201 | 116206 |
| rs4710939 | 1.00 | 0.35 | 20752923 | 117928 |
| rs11965062 | 1.00 | 0.33 | 20755941 | 120946 |
| rs9460540 | 1.00 | 0.33 | 20756741 | 121746 |
| rs6456364 | 0.79 | 0.23 | 20757233 | 122238 |
| rs9295474 | 0.95 | 0.68 | 20760696 | 125701 |
| rs2328545 | 0.79 | 0.23 | 20761529 | 126534 |
| rs9368216 | 0.79 | 0.23 | 20763089 | 128094 |
| rs16884072 | 0.66 | 0.33 | 20763482 | 128487 |
| rs9460541 | 0.66 | 0.33 | 20764559 | 129564 |
| rs9460542 | 0.66 | 0.33 | 20764746 | 129751 |
| rs4712522 | 0.95 | 0.68 | 20764779 | 129784 |
| rs16884074 | 0.66 | 0.32 | 20764924 | 129929 |
| rs4712523 | 0.95 | 0.68 | 20765543 | 130548 |
| rs4710940 | 0.95 | 0.52 | 20765991 | 130996 |
| rs13190727 | 0.66 | 0.33 | 20766197 | 131202 |
| rs6906327 | 0.95 | 0.52 | 20767438 | 132443 |
| rs6456367 | 0.95 | 0.68 | 20767566 | 132571 |
| rs6456368 | 0.95 | 0.67 | 20767785 | 132790 |
| rs7749083 | 0.66 | 0.33 | 20768202 | 133207 |
| rs6456369 | 0.95 | 0.52 | 20768344 | 133349 |
| rs13203361 | 0.66 | 0.33 | 20769000 | 134005 |
| rs10946398 | 0.95 | 0.68 | 20769013 | 134018 |
| rs7774594 | 0.95 | 0.67 | 20769122 | 134127 |
| rs7754840 | 0.95 | 0.68 | 20769229 | 134234 |
| rs9460544 | 0.95 | 0.68 | 20769508 | 134513 |
| rs9460545 | 0.95 | 0.68 | 20769529 | 134534 |
| rs979614 | 1.00 | 0.34 | 20770102 | 135107 |
| rs4712525 | 0.95 | 0.68 | 20770945 | 135950 |
| rs4712526 | 0.95 | 0.68 | 20771014 | 136019 |
| rs9460546 | 0.95 | 0.68 | 20771611 | 136616 |
| rs736425 | 0.66 | 0.33 | 20772291 | 137296 |
| rs742642 | 0.79 | 0.23 | 20773060 | 138065 |
| rs7748382 | 0.95 | 0.68 | 20773528 | 138533 |
| rs7772603 | 0.95 | 0.68 | 20773925 | 138930 |
| rs7752780 | 0.95 | 0.68 | 20774001 | 139006 |
| rs7752906 | 0.95 | 0.70 | 20774034 | 139039 |
| rs11970425 | 0.66 | 0.33 | 20774436 | 139441 |
| rs9358356 | 0.95 | 0.67 | 20775361 | 140366 |
| rs9356743 | 0.79 | 0.23 | 20775667 | 140672 |
| rs9368219 | 1.00 | 0.53 | 20782670 | 147675 |
| rs1012635 | 1.00 | 0.42 | 20783274 | 148279 |
| rs1569699 | 1.00 | 0.72 | 20787289 | 152294 |
| rs9350271 | 1.00 | 0.78 | 20791143 | 156148 |
| rs9356744 | 1.00 | 0.75 | 20793465 | 158470 |
| rs7766070 | 1.00 | 1.00 | 20794552 | 159557 |
| rs9368222 | 1.00 | 1.00 | 20794975 | 159980 |
| rs10440833 | 1.00 | 1.00 | 20796100 | 161105 |
| rs2206734 | 1.00 | 0.53 | 20802863 | 167868 |
| rs6931514 | 1.00 | 1.00 | 20811931 | 176936 |
| rs11753081 | 1.00 | 0.53 | 20813569 | 178574 |
| rs1040558 | 1.00 | 0.53 | 20821685 | 186690 |
| rs9295478 | 0.62 | 0.30 | 20824232 | 189237 |
| rs2328548 | 1.00 | 0.53 | 20824937 | 189942 |
| rs6935599 | 1.00 | 0.53 | 20825074 | 190079 |
| rs9465871 | 1.00 | 0.53 | 20825234 | 190239 |
| rs10946403 | 1.00 | 0.53 | 20825383 | 190388 |
| rs2328549 | 1.00 | 0.30 | 20826219 | 191224 |
| rs9358357 | 1.00 | 0.53 | 20827124 | 192129 |
| rs9368224 | 1.00 | 0.53 | 20827211 | 192216 |
| rs9358358 | 1.00 | 0.30 | 20827372 | 192377 |
| rs9460550 | 1.00 | 0.53 | 20827540 | 192545 |
| rs9356746 | 1.00 | 0.30 | 20828258 | 193263 |
| rs9368226 | 1.00 | 0.50 | 20831036 | 196041 |
| rs12111351 | 0.61 | 0.29 | 20832537 | 197542 |
| rs9356747 | 0.60 | 0.29 | 20832986 | 197991 |
| rs9356748 | 1.00 | 0.30 | 20833076 | 198081 |
| rs7767391 | 1.00 | 0.50 | 20833219 | 198224 |
| rs7747752 | 0.62 | 0.30 | 20833402 | 198407 |
| rs17234378 | 0.80 | 0.24 | 20952720 | |

The table shows markers with values for r² of greater than 0.2 in the HapMap Caucasian CEPH samples. The search was performed over a 2 Mb region flanking rs77566992 (1 Mb upstream and 1 Mb downstream).

TABLE 23
Surrogate markers for marker rs10882091 on chromosome 10.
Surrogates for rs10882091 on chromosome 10

| | | | | Pos SEQ ID |
|---|---|---|---|---|
| SNP | D′ | R2 | Pos B36 | NO: 2 |
| rs7086285 | 0.71 | 0.23 | 94166068 | |
| rs2798253 | 0.93 | 0.32 | 94192885 | 1 |
| rs6583813 | 1.00 | 0.33 | 94199919 | 7035 |
| rs11187007 | 1.00 | 0.35 | 94204560 | 11676 |
| rs2149632 | 1.00 | 0.35 | 94222227 | 29343 |
| rs11187025 | 0.95 | 0.48 | 94247956 | 55072 |
| rs11187033 | 1.00 | 0.35 | 94252339 | 59455 |
| rs10509645 | 1.00 | 0.35 | 94267846 | 74962 |
| rs7078413 | 0.49 | 0.23 | 94280464 | 87580 |
| rs4646955 | 0.75 | 0.37 | 94284271 | 91387 |
| rs17445328 | 0.68 | 0.32 | 94295169 | 102285 |
| rs11187064 | 0.68 | 0.31 | 94298233 | 105349 |
| rs2421943 | 1.00 | 0.45 | 94301795 | 108911 |
| rs11187065 | 0.95 | 0.48 | 94301904 | 109020 |
| rs11187078 | 1.00 | 0.35 | 94330685 | 137801 |
| rs6583823 | 1.00 | 0.52 | 94334395 | 141511 |
| rs2421941 | 0.96 | 0.93 | 94335889 | 143005 |
| rs6583826 | 0.95 | 0.57 | 94337810 | 144926 |
| rs3824735 | 1.00 | 0.36 | 94344184 | 151300 |
| rs10786050 | 1.00 | 1.00 | 94357210 | 164326 |
| rs11187094 | 1.00 | 0.21 | 94358158 | 165274 |
| rs11187096 | 1.00 | 0.35 | 94359568 | 166684 |
| rs7914814 | 1.00 | 1.00 | 94372930 | 180046 |
| rs12772554 | 1.00 | 0.23 | 94373838 | 180954 |
| rs10882094 | 1.00 | 1.00 | 94377656 | 184772 |
| rs10882095 | 1.00 | 0.37 | 94384382 | 191498 |
| rs10736069 | 1.00 | 1.00 | 94385373 | 192489 |
| rs7900689 | 1.00 | 1.00 | 94385728 | 192844 |
| rs6583830 | 1.00 | 1.00 | 94388098 | 195214 |
| rs10882096 | 1.00 | 0.35 | 94391366 | 198482 |
| rs11187114 | 1.00 | 0.36 | 94396217 | 203333 |
| rs6583833 | 1.00 | 0.76 | 94399780 | 206896 |
| rs7078243 | 1.00 | 0.78 | 94404243 | 211359 |
| rs4933734 | 1.00 | 1.00 | 94404547 | 211663 |
| rs7911264 | 1.00 | 0.73 | 94426831 | 233947 |
| rs2488087 | 1.00 | 0.74 | 94436021 | 243137 |
| rs10882100 | 1.00 | 0.74 | 94450667 | 257783 |
| rs1111875 | 1.00 | 0.51 | 94452862 | 259978 |
| rs12778642 | 1.00 | 0.55 | 94454287 | 261403 |
| rs5015480 | 1.00 | 0.51 | 94455539 | 262655 |
| rs10882102 | 1.00 | 0.52 | 94456475 | 263591 |
| rs11187144 | 1.00 | 0.40 | 94459960 | 267076 |
| rs7087591 | 1.00 | 0.39 | 94463609 | 270725 |
| rs10748582 | 1.00 | 0.39 | 94467199 | 274315 |
| rs7923837 | 1.00 | 0.39 | 94471897 | 279013 |
| rs7923866 | 1.00 | 0.39 | 94472056 | 279172 |
| rs2497306 | 1.00 | 0.58 | 94475191 | 282307 |
| rs2488075 | 1.00 | 0.60 | 94480154 | 287270 |
| rs2497304 | 0.96 | 0.63 | 94482696 | 289812 |
| rs947591 | 0.81 | 0.57 | 94485733 | 292849 |
| rs2488071 | 0.62 | 0.24 | 94489557 | 296673 |

The table shows markers with values for r² of greater than 0.2 in the HapMap Caucasian CEPH samples. The search was performed over a 2 Mb region flanking rs10882091 (1 Mb upstream and 1 Mb downstream).

TABLE 24
Surrogate markers for marker rs2191113 on chromosome 17.
Surrogates for rs2191113 on chromosome 17

| | | | | POS SEQ ID |
|---|---|---|---|---|
| SNP | D′ | R2 | Pos B36 | NO: 3 |
| rs350605 | 0.82 | 0.54 | 66044207 | 6552 |
| rs350603 | 0.80 | 0.22 | 66045245 | 7590 |
| rs420762 | 0.80 | 0.24 | 66049716 | 12061 |
| rs350615 | 0.86 | 0.58 | 66067303 | 29648 |
| rs350616 | 0.81 | 0.25 | 66067699 | 30044 |
| rs350621 | 0.86 | 0.58 | 66079419 | 41764 |
| rs350624 | 0.86 | 0.58 | 66080067 | 42412 |
| rs12602288 | 1.00 | 0.36 | 66085473 | 47818 |
| rs1431454 | 0.82 | 0.26 | 66090535 | 52880 |
| rs9302918 | 1.00 | 0.23 | 66091912 | 54257 |
| rs9302919 | 0.81 | 0.26 | 66092080 | 54425 |
| rs9911671 | 0.86 | 0.61 | 66094196 | 56541 |
| rs1911969 | 0.86 | 0.60 | 66102315 | 64660 |
| rs9894021 | 1.00 | 0.21 | 66103236 | 65581 |
| rs720877 | 1.00 | 0.23 | 66103561 | 65906 |
| rs720876 | 1.00 | 0.23 | 66103923 | 66268 |
| rs7218838 | 0.86 | 0.61 | 66106415 | 68760 |
| rs9896809 | 1.00 | 0.21 | 66106911 | 69256 |
| rs7220084 | 0.82 | 0.26 | 66114858 | 77203 |
| rs1860316 | 0.86 | 0.61 | 66117911 | 80256 |
| rs8079029 | 0.90 | 0.62 | 66118485 | 80830 |
| rs4019476 | 0.87 | 0.63 | 66122077 | 84422 |
| rs1981647 | 0.82 | 0.26 | 66132788 | 95133 |
| rs9890554 | 0.80 | 0.21 | 66134831 | 97176 |
| rs10221225 | 0.80 | 0.22 | 66138452 | 100797 |
| rs11650683 | 0.84 | 0.22 | 66139800 | 102145 |
| rs1486290 | 0.82 | 0.27 | 66141933 | 104278 |
| rs8078302 | 0.85 | 0.23 | 66143200 | 105545 |
| rs12949591 | 1.00 | 0.20 | 66146912 | 109257 |
| rs1843622 | 1.00 | 0.61 | 66149102 | 111447 |
| rs9891997 | 1.00 | 0.28 | 66152998 | 115343 |
| rs9910837 | 1.00 | 0.28 | 66155303 | 117648 |
| rs4793497 | 0.94 | 0.58 | 66163076 | 125421 |
| rs9890889 | 0.89 | 0.24 | 66173475 | |
| rs2009802 | 0.71 | 0.23 | 66178475 | |
| rs17718938 | 1.00 | 0.28 | 66184700 | |
| rs17223216 | 0.89 | 0.24 | 66207685 | |
| rs2109050 | 0.89 | 0.24 | 66228633 | |
| rs1962801 | 1.00 | 0.31 | 66236090 | |

The table shows markers with values for r² of greater than 0.2 in the HapMap Caucasian CEPH samples. The search was performed over a 2 Mb region flanking rs2191113 (1 Mb upstream and 1 Mb downstream).

1. A method of determining a susceptibility to Type 2 diabetes in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of the markers set forth in Tables 10-12, and markers in linkage disequilibrium therewith, and wherein determination of the presence or absence of the at least one allele is indicative of a susceptibility to Type 2 diabetes.

2. The method of claim 1, wherein the at least one polymorphic marker is present within SEQ ID NO:1, SEQ ID NO:2 or SEQ ID NO:3.

3. The method of claim 1, wherein the at least one polymorphic marker comprises at least one marker selected from markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID 35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith.

4. The method of claim 1, wherein the at least one polymorphic marker comprises at least one marker in strong linkage disequilibrium, as defined by numeric values for |D′| of greater than 0.8 and/or r² of greater than 0.2, with one or more markers selected from the group consisting of the markers set forth in Table 22, Table 23 and Table 24.

5. The method of claim 1, wherein the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), and markers in linkage disequilibrium therewith.

6. The method of claim 5, wherein the at least one polymorphic marker is selected from markers rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), and rs6931514 (SEQ ID NO:37).

7. The method of claim 1, wherein the at least one polymorphic marker is selected from marker rs7756992 (SEQ ID NO: 21), and markers in linkage disequilibrium therewith.

8. The method of claim 7, wherein the at least one polymorphic markers is selected from the markers set forth in Table 22.

9. The method of claim 1, wherein the at least one polymorphic marker is selected from marker rs10882091 (SEQ ID NO: 4), and markers in linkage disequilibrium therewith.

10. The method of claim 9, wherein the at least one polymorphic marker is selected from the markers set forth in Table 23.

11. The method of claim 1, wherein the at least one marker is selected from marker rs2191113 (SEQ ID NO: 13), and markers in linkage disequilibrium therewith.

12. The method of claim 11, wherein the at least one marker is selected from the markers set forth in Table 24.

13-16. (canceled)

17. The method of claim 3, wherein the presence of rs2497304 allele A, rs947591 allele A, rs10882091 allele C rs7914814 allele T, rs6583830 allele A, rs2421943 allele G, rs6583826 allele G, rs7752906 allele A, rs1569699 allele C, rs7756992 allele G, rs9350271 allele A, rs9356744 allele C, rs9368222 allele A, rs10440833 allele A, rs6931514 allele G, rs1860316 allele A, rs1981647 allele C, rs1843622 allele T, rs2191113 allele A, and/or rs9890889 allele A is indicative of increased susceptibility of Type 2 diabetes.

18-22. (canceled)

23. A method of assessing a susceptibility to Type 2 diabetes in a human individual, comprising screening a nucleic acid from the individual, or a genotype dataset for the individual, for at least one polymorphic marker or haplotype in SEQ ID NO:1, SEQ ID NO:2 or SEQ ID NO:3, that correlates with increased occurrence of Type 2 diabetes in a human population, wherein the presence of an at-risk marker allele in the at least one polymorphism or an at-risk haplotype in the nucleic acid identifies the individual as having elevated susceptibility to Type 2 diabetes, and wherein the absence of the at least one at-risk marker allele or at-risk haplotype in the nucleic acid identifies the individual as not having the elevated susceptibility.

24. The method of claim 23, wherein the polymorphism or haplotype is selected from markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, as characterized by numeric values for |D′| of greater than 0.8 and/or r² of greater than 0.2.

25. The method of claim 24, further comprising screening the nucleic acid for the presence of at least one at-risk genetic variant for Type 2 diabetes not associated with LD Block C06 (SEQ ID NO:1), LD Block C10 (SEQ ID NO:2) and LD Block C17 (SEQ ID NO:3).

26. The method of claim 25, comprising screening the nucleic acid for the presence or absence of at least one at-risk allele of at least one at-risk variant for Type 2 diabetes in the TCF7L2 gene, wherein determination of the presence of the at least one at-risk allele is indicative of increased susceptibility of Type 2 diabetes.

27. The method of claim 25, wherein the at least one at-risk variant in the TCF7L2 gene is selected from marker DG10S478, rs12255372, rs7895340, rs11196205, rs7901695, rs7903146, rs12243326 and rs4506565, and markers in linkage disequilibrium therewith.

28. (canceled)

29. The method of claim 23, wherein the individual is of a specific human ancestry selected from the group consisting of: black African ancestry, European ancestry, Caucasian ancestry and Chinese ancestry.

30-34. (canceled)

35. The method of claim 27, wherein the ancestry is determined by genetic determination comprising detecting at least one allele of at least one polymorphic marker in a nucleic acid sample from the individual, wherein the presence or absence of the allele is indicative of the ancestry of the individual.

36-37. (canceled)

38. A method of identification of a marker for use in assessing susceptibility to Type 2 diabetes in human individuals, the method comprising
a) identifying at least one polymorphic marker within SEQ ID NO:1, SEQ ID NO:2 or SEQ ID NO:3, or at least one polymorphic marker in linkage disequilibrium therewith;
b) determining the genotype status of a sample of individuals diagnosed with, or having a susceptibility to, Type 2 diabetes; and
c) determining the genotype status of a sample of control individuals;
wherein a significant difference in frequency of at least one allele in at least one polymorphism in individuals diagnosed with, or having a susceptibility to, Type 2 diabetes, as compared with the frequency of the at least one allele in the control sample is indicative of the at least one polymorphism being useful for assessing susceptibility to Type 2 diabetes.

39. (canceled)

40. The method of claim 38, wherein the at least one polymorphic marker is in linkage disequilibrium, as characterized by numerical values of r² of greater than 0.2 and/or |D′| of greater than 0.8 with at least one marker selected from marker rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), or rs9890889 (SEQ ID NO:31).

41-52. (canceled)

53. The method of claim 38, wherein the individual is of a specific human ancestry selected from the group consisting of: black African ancestry, European ancestry, Caucasian ancestry and Chinese ancestry.

54-58. (canceled)

59. The method of claim 53, wherein the ancestry is determined by genetic determination comprising detecting at least one allele of at least one polymorphic marker in a nucleic acid sample from the individual, wherein the presence or absence of the allele is indicative of the ancestry of the individual.

60-61. (canceled)

62. A method of assessing an individual for probability of response to a therapeutic agent for preventing and/or ameliorating symptoms associated with Type 2 diabetes, comprising: determining the presence or absence of at least one allele of at least one polymorphic marker in a nucleic acid sample obtained from the individual, wherein the at least one polymorphic marker is selected from markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele of the at least one marker is indicative of a probability of a positive response to the Type 2 diabetes therapeutic agent.

63. The method of claim 62, wherein the Type 2 diabetes therapeutic agent is selected from the group consisting of: the agents set forth in Agent Table 1 and Agent Table 2.

64. A method of predicting prognosis of an individual diagnosed with, Type 2 diabetes, the method comprising determining the presence or absence of at least one allele of at least one polymorphic marker in a nucleic acid sample obtained from the individual, wherein the at least one polymorphic marker is selected from the markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele is indicative of a worse prognosis of the Type 2 diabetes in the individual.

65. A method of monitoring progress of a treatment of an individual undergoing treatment for Type 2 diabetes, the method comprising determining the presence or absence of at least one allele of at least one polymorphic marker in a nucleic acid sample obtained from the individual, wherein the at least one polymorphic marker is selected from the markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, wherein determination of the presence of the at least one allele is indicative of the treatment outcome of the individual.

66-70. (canceled)

71. A kit for assessing susceptibility to Type 2 diabetes in a human individual, the kit comprising reagents for selectively detecting the presence or absence of at least one allele of at least one polymorphic marker in the genome of the individual, wherein the polymorphic marker is selected from the polymorphic markers within the nucleic acid segments whose sequences are set forth in SEQ ID NO:1, SEQ ID NO:2 and SEQ ID NO:3, and markers in linkage disequilibrium therewith, and wherein the presence of the at least one allele is indicative of a susceptibility to Type 2 diabetes.

72. (canceled)

73. The kit of claim 71, wherein the at least one polymorphic markers is selected from markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith.

74-82. (canceled)

83. A computer-readable medium on which is stored:
a) an identifier for at least one polymorphic marker;
b) an indicator of the frequency of at least one allele of said at least one polymorphic marker in a plurality of individuals diagnosed with Type 2 diabetes; and
c) an indicator of the frequency of the least one allele of said at least one polymorphic markers in a plurality of reference individuals;
wherein the at least one polymorphic marker is selected from the polymorphic markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and polymorphisms in linkage disequilibrium therewith, as defined by numerical values of r² of at least 0.2 and/or values of |D′| at least 0.8.

84. The medium of claim 83, further comprising information about the ancestry of the plurality of individuals.

85. (canceled)

86. An apparatus for determining a genetic indicator for Type 2 diabetes in a human individual, comprising:
a computer readable memory; and
a routine stored on the computer readable memory;
wherein the routine is adapted to be executed on a processor to analyze marker and/or haplotype information for at least one human individual with respect to at least one polymorphic marker selected from the markers rs2497304 (SEQ ID NO:16), rs947591 (SEQ ID NO:30), rs10882091 (SEQ ID NO:4), rs7914814 (SEQ ID NO:24), rs6583830 (SEQ ID NO:20), rs2421943 (SEQ ID NO:15), rs6583826 (SEQ ID NO:19), rs7752906 (SEQ ID NO:32), rs1569699 (SEQ ID NO:6), rs7756992 (SEQ ID NO:21), rs9350271 (SEQ ID NO:33), rs9356744 (SEQ ID NO:34), rs9368222 (SEQ ID NO:35), rs10440833 (SEQ ID NO:36), rs6931514 (SEQ ID NO:37), rs1860316 (SEQ ID NO:10), rs1981647 (SEQ ID NO:11), rs1843622 (SEQ ID NO:9), rs2191113 (SEQ ID NO:13), rs9890889 (SEQ ID NO:31), and markers in linkage disequilibrium therewith, as defined by numerical values of r² of at least 0.2 and/or values of |D′| of at least 0.8, and generate an output based on the marker or haplotype information, wherein the output comprises a risk measure of the at least one marker or haplotype as a genetic indicator of Type 2 diabetes for the human individual.

87. The apparatus of claim 86, wherein the routine further comprises an indicator of the frequency of at least one allele of at least one polymorphic marker or at least one haplotype in a plurality of individuals diagnosed with Type 2 diabetes, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with Type 2 diabetes.

88-108. (canceled)

109. A method of assessing a susceptibility to Type 2 diabetes in a human individual, comprising screening the individual for at least one polymorphic marker in the CDKAL1 gene that correlates with increased occurrence of Type 2 diabetes in a human population, wherein determination of the presence of an at-risk allele in the at least one polymorphic marker identifies the individual as having an increased susceptibility to Type 2 diabetes, and wherein the absence of the at-risk allele identifies the individual as not having the elevated susceptibility.

110. The method of claim 109, wherein screening the individual comprises screening a nucleic acid from the individual.

111. The method of claim 109, wherein screening the individual comprises screening a genotype dataset derived from the individual.

112. The method of claim 109, wherein the at least one polymorphic marker is selected from the markers set forth in Table 9.

113. The method of claim 109, wherein the at least one polymorphic marker is marker rs7756992, or markers in linkage disequilibrium therewith.
